SEQUENCE LISTING (215 sequences)

SEQ ID NO: 1 - protein, 12 aa, Artificial Sequence
  Description of Artificial Sequence Synthetic peptide
RRVPLGNANLIS

SEQ ID NO: 2 - protein, 12 aa, Artificial Sequence
  Description of Artificial Sequence Synthetic peptide
RRVPLAKQGVIS

SEQ ID NO: 3 - protein, 12 aa, Artificial Sequence
  Description of Artificial Sequence Synthetic peptide
RTEPLLKEGFVS

SEQ ID NO: 4 - protein, 12 aa, Artificial Sequence
  Description of Artificial Sequence Synthetic peptide
YQRYARGSQAKV

SEQ ID NO: 5 - protein, 12 aa, Artificial Sequence
  Description of Artificial Sequence Synthetic peptide
YQRYLKGSQAAV

SEQ ID NO: 6 - protein, 12 aa, Artificial Sequence
  Description of Artificial Sequence Synthetic peptide
RYQKLLGTQYIS

SEQ ID NO: 7 - protein, 12 aa, Artificial Sequence
  Description of Artificial Sequence Synthetic peptide
RYVPLVGTKYIS

SEQ ID NO: 8 - protein, 12 aa, Artificial Sequence
  Description of Artificial Sequence Synthetic peptide
RQASLLKTNYVS

SEQ ID NO: 9 - protein, 12 aa, Artificial Sequence
  Description of Artificial Sequence Synthetic peptide
RYQQLAKTNLVS

SEQ ID NO: 10 - protein, 11 aa, Artificial Sequence
  Description of Artificial Sequence Synthetic peptide
RRNRLGVQAMS

SEQ ID NO: 11 - protein, 11 aa, Artificial Sequence
  Description of Artificial Sequence Synthetic peptide
RRRHLSQNFIS

SEQ ID NO: 12 - protein, 12 aa, Artificial Sequence
  Description of Artificial Sequence Synthetic peptide
RQQGLWKSRTIS

SEQ ID NO: 13 - protein, 12 aa, Artificial Sequence
  Description of Artificial Sequence Synthetic peptide
RSRSLAQRGAIS

SEQ ID NO: 14 - protein, 12 aa, Artificial Sequence
  Description of Artificial Sequence Synthetic peptide
RQQRLAQTKAVS

SEQ ID NO: 15 - protein, 332 aa, Artificial Sequence
  Description of Artificial Sequence Synthetic polypeptide
MMKKPVVIGLAVVVLAAVVAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDYAQNFYNRQQGLWKSRTISANDLENARSSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 16 - protein, 355 aa, Artificial Sequence
  Description of Artificial Sequence Synthetic polypeptide
MDKSKRHLAWWVVGLLAVAAIVAWWLLRPAGVPEGFAVSNGRIEATEVDIASKIAGRIDTILVKEGKFVREGEVLAKMDTRVLQEQRLEAIAQIKEAQSAVAAAQALLEQRQSETRAAQSLVNQRQAELDSVAKRHTRSRSLAQRGAISAQQLDDDRAAAESARAALESAKAQVSASKAAIEAARTNIIQAQTRVEAAQATERRIAADIDDSELKAPRDGRVQYRVAEPGEVLAAGGRVLNMVDLSDVYMTFFLPTEQAGTLKLGGEARLILDAAPDLRIPATISFVASVAQFTPKTVETSDERLKLMFRVKARIPPELLQQHLEYVKTGLPGVAWVRVNEELPWPDDLVVRLPQ

SEQ ID NO: 17 - protein, 22 aa, Synechococcus sp.
MRFFWFFLTLLTLSTWQLPAWA

SEQ ID NO: 18 - protein, 26 aa, Synechococcus sp.
MFAFRDFLTFSTGGLVVLSGGGVAIA

SEQ ID NO: 19 - DNA, 999 bp, Escherichia coli
gtgatgaaaaaacctgtcgtgatcggattggcggtagtggtacttgccgccgtggttgccggaggctactggtggtatcaaagccgccaggataacggcctgacgctgtatggcaacgtggatattcgtacggtaaatcttagtttccgtgttggggggcgcgttgaatcgctggcggtggacgaaggtgatgctatcaaagcgggccaggtgctgggcgaactggatcacaagccgtatgagattgccctgatgcaggcgaaagcgggtgtttcggtggcacaggcgcagtatgacctgatgcttgccgggtatcgcaatgaagaaatcgctcaggccgccgcagcggtgaaacaggcgcaagccgcctatgactatgcgcagaacttctataaccgccagcaagggttgtggaaaagccgcactatttcggcaaatgacctggaaaatgcccgctcctcgcgcgaccaggcgcaggcaacgctgaaatcagcacaggataaattgcgtcagtaccgttccggtaaccgtgaacaggacatcgctcaggcgaaagccagcctcgaacaggcgcaggcgcaactggcgcaggcggagttgaatttacaggactcaacgttgatagccccgtctgatggcacgctgttaacgcgcgcggtggagccaggcacggtcctcaatgaaggtggcacggtgtttaccgtttcactaacgcgtccggtgtgggtgcgcgcttatgttgatgaacgtaatcttgaccaggcccagccggggcgcaaagtgctgctttataccgatggtcgcccggacaagccgtatcacgggcagattggtttcgtttcgccgactgctgaatttaccccgaaaaccgtcgaaacgccggatctgcgtaccgacctcgtctatcgcctgcgtattgtggtgaccgacgccgatgatgcgttacgccagggaatgccagtgacggtacaattcggtgacgaggcaggacatgaatga

SEQ ID NO: 20 - DNA, 1737 bp, Escherichia coli
atgaatgatgccgttatcacgctgaacggcctggaaaaacgctttccgggcatggacaagcccgccgtcgcgccgctcgattgtaccattcacgccggttatgtgacggggttggtggggccggacggtgcaggtaaaaccacgctgatgcggatgttggcgggattactgaaacccgacagcggcagtgccacggtgattggctttgatccgatcaaaaacgacggcgcgctgcacgccgtgctcggttatatgccgcagaaatttggtctgtatgaagatctcacggtgatggagaacctcaatctgtacgcggatttgcgcagcgtcaccggcgaggcacgtaagcaaacttttgctcgcctgctggagtttacgtctcttgggccgtttaccggacgcctggcgggcaagctctccggtgggatgaaacaaaaactcggtctggcctgtaccctggtgggcgaaccgaaagtgttgctgctcgatgaacccggcgtcggcgttgaccctatctcacggcgcgaactgtggcagatggtgcatgagctggcgggcgaagggatgttaatcctctggagtacctcgtatctcgacgaagccgagcagtgccgtgacgtgttactgatgaacgaaggcgagttgctgtatcagggagaaccaaaagccctgacacaaaccatggccggacgcagctttctgatgaccagtccacacgagggcaaccgcaaactgttgcaacgcgccttgaaactgccgcaggtcagcgacggcatgattcaggggaaatcggtacgtctgatcctcaaaaaagaggccacaccagacgatattcgccatgccgacgggatgccggaaatcaacatcaacgaaactacgccgcgttttgaagatgcgtttattgatttgctgggcggtgccggaacctcggaatcgccgctgggcgcaatattacatacggtagaaggcacacccggcgagacggtgatcgaagcgaaagaactgaccaagaaatttggggattttgccgccaccgatcacgtcaactttgccgttaaacgtggggagatttttggtttgctggggccaaacggcgcgggtaaatcgaccacctttaagatgatgtgcggtttgctggtgccgacttccggccaggcgctggtgctggggatggatctgaaagagagttccggtaaagcgcgccagcatctcggctatatggcgcaaaaattttcgctctacggtaacctgacggtcgaacagaatttacgctttttctctggtgtgtatggcttacgcggtcgggcgcagaacgaaaaaatctcccgcatgagcgaggcgttcggcctgaaaagtatcgcctcccacgccaccgatgaactgccattaggttttaaacagcggctggcgctggcctgttcgctgatgcatgaaccggacattctgtttctcgacgaaccgacttccggcgttgaccccctcacccgccgtgaattttggctgcacatcaacagcatggtagagaaaggcgtcacggtgatggtcaccacccactttatggatgaagcggaatattgcgaccgcatcggcctggtgtaccgcgggaaattaatcgccagcggcacgccggacgatttgaaagcacagtcggctaacgatgagcaacccgatcccaccatggagcaagcctttattcagttgatccacgactgggataaggagcatagcaatgagtaa

SEQ ID NO: 21 - DNA, 1134 bp, Escherichia coli
atgagtaacccgatcctgtcctggcgtcgcgtacgggcgctgtgcgttaaagagacgcggcagatcgttcgcgatccgagtagctggctgattgcggtagtgatcccgctgctactgctgtttatttttggttacggcattaacctcgactccagcaagctgcgggtcgggattttactggaacagcgtagcgaagcggcgctggatttcacccacaccatgaccggttcgccctacatcgacgccaccatcagcgataaccgtcaggaactgatcgccaaaatgcaggcggggaaaattcgcggtctggtggttattccggtggattttgcggaacagatggagcgcgccaacgccaccgcaccgattcaggtgatcaccgacggcagtgagccgaataccgctaactttgtacaggggtatgtcgaagggatctggcagatctggcaaatgcagcgagcggaggacaacgggcagacttttgaaccgcttattgatgtacaaacccgctactggtttaacccggcggcgattagccagcacttcattatccccggtgcggtgaccattatcatgacggtcatcggcgcgattctcacctcgctggtggtggcgcgagaatgggaacgcggcaccatggaggctctgctctctacggagattacccgcacggaactgctgctgtgtaagctgatcccttattactttctcgggatgctggcgatgttgctgtgtatgctggtgtcagtgtttattctcggcgtgccgtatcgcgggtcgctgctgattctgttttttatctccagcctgtttttactcagtaccctggggatggggctgctgatttccacgattacccgcaaccagttcaatgccgctcaggtcgccctgaacgccgcttttctgccgtcgattatgctttccggctttatttttcagatcgacagtatgcccgcggtgatccgcgcggtgacgtacattattcccgctcgttatttcgtcagcaccctgcaaagcctgttcctcgccgggaatattccagtggtgctggtggtaaacgtgctgtttttgatcgcttcggcggtgatgtttatcggcctgacgtggctgaaaaccaaacgtcggctggattag

SEQ ID NO: 22 - DNA, 1107 bp, Escherichia coli
atgtttcatcgcttatggacgttaatccgcaaagagttgcagtcgttgctgcgcgaaccgcaaacccgcgcgattctgattttacccgtgctaattcaggtgatcctgttcccgttcgccgccacgctggaagtgactaacgccaccatcgccatctacgatgaagataacggcgagcattcggtggagctgacccaacgttttgcccgcgccagcgcctttactcatgtgctgctgctgaaaagcccacaggagatccgcccaaccatcgacacacaaaaggcgttactactggtgcgtttcccggctgacttctcgcgcaaactggataccttccagaccgcgcctttgcagttgatcctcgacgggcgtaactccaacagtgcgcaaattgccgccaactacctgcaacagatcgtcaaaaattatcagcaggagctgctggaaggaaaaccgaaacctaacaacagcgagctggtggtacgcaactggtataacccgaatctcgactacaaatggtttgtggtgccgtcactgatcgccatgatcaccactatcggcgtaatgatcgtcacttcactttccgtcgcccgcgaacgtgaacaaggtacgctcgatcagctactggtttcgccgctcaccacctggcagatcttcatcggcaaagccgtaccggcgttaattgtcgccaccttccaggccaccattgtgctggcgattggtatctgggcgtatcaaatccccttcgccggatcgctggcgctgttctactttacgatggtgatttatggtttatcgctggtgggattcggtctgttgatttcatcactctgttcaacacaacagcaggcgtttatcggcgtgtttgtctttatgatgcccgccattctcctttccggttacgtttctccggtggaaaacatgccggtatggctgcaaaacctgacgtggattaaccctattcgccactttacggacattaccaagcagatttatttgaaggatgcgagtctggatattgtgtggaatagtttgtggccgctactggtgataacggccacgacagggtcagcggcgtacgcgatgtttagacgtaaggtgatgtaa

SEQ ID NO: 23 - DNA, 1482 bp, Escherichia coli
atgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 24 - DNA, 1068 bp, Escherichia coli
atggataagagtaagcgccatctggcgtggtgggttgtcgggttactggcggtggcggctatcgtggcgtggtggctgttgcgcccggcaggtgtgccggaaggctttgctgtcagtaatgggcgcattgaagcgacggaagtggatattgccagcaaaattgccgggcgtatcgacaccattctggtgaaagaaggcaagtttgttcgcgaaggtgaagtgctggcgaagatggatactcgcgtgttgcaggaacagcgactggaagccatcgcgcaaatcaaagaggcacaaagcgccgttgctgccgcgcaggctttgctggagcaacgacaaagcgaaactcgtgccgcacagtcgctggttaatcaacgccaggcagaactggactccgtagcaaaacgtcatacgcgttcccgttcactggcccaacgaggggctatttctgcgcaacagctggatgacgatcgcgccgccgctgagagcgcccgagctgcgctggaatcggcgaaagctcaggtatcggcttctaaagcggctatagaagcggcacgcaccaatatcattcaggcgcaaacccgcgtcgaagcggcacaagccactgaacggcgcattgccgcagatatcgatgacagcgaactgaaagccccgcgtgacggacgcgtgcagtatcgggttgccgagccaggcgaagtgctggcggcaggcggtcgggtgctgaatatggtcgatctcagcgacgtctatatgactttcttcctgccaaccgaacaggcgggcacgctgaaactgggcggtgaagcccggctgatcctcgatgccgcgccagatctgcgtattcctgcaaccatcagttttgtcgccagtgtcgcccagttcacgccaaaaaccgtcgaaaccagcgatgaacggctgaaactgatgttccgcgtcaaagcgcgtatcccaccggaattactccagcagcatctggaatatgtcaaaaccggtttgccgggcgtagcgtgggtgcgggtgaatgaagaacttccgtggcctgacgacctcgtggtgaggttgccgcaatga

SEQ ID NO: 25 - DNA, 2736 bp, Escherichia coli
atgacgcatctggaactggttcccgtcccgcctgtcgcgcaactggcgggcgtgagccagcattatggaaaaaccgttgcgctgaacaatatcactctcgatattccggcccgctgtatggtcgggctgattggcccggacggcgtcgggaagtcgagcttgttgtcgttgatttccggtgcccgcgtcattgaacagggcaatgtgatggtgctgggcggcgatatgcgcgacccgaagcatcgccgcgacgtctgcccgcgcatcgcctggatgccgcaggggctgggcaaaaacctctaccacaccttgtcggtgtatgaaaacgtcgattttttcgctcgcctgttcggtcacgacaaagcggagcgggaagtgcgaatcaatgagctgctgaccagcaccgggttagcaccgtttcgcgatcgtccggcagggaaactctccggcgggatgaagcaaaaacttgggctgtgctgcgcgttaatccacgacccggaactgttgatccttgatgagccaacaacgggggttgacccgctctcccgctcccagttctgggatctgatcgacagtattcgccagcggcagagcaatatgagcgtgctggtcgccaccgcctatatggaagaggccgaacgcttcgactggctggtagcgatgaatgccggagaagtgctggcaactggcagcgccgaagagctacggcagcaaacgcaaagcgctacgctggaagaagcatttataaatctgttaccgcaagcgcaacgccaggcgcatcaggcggtagtgatcccaccgtatcaacctgaaaacgcagagattgccatcgaagcgcgcgatctgaccatgcgttttggttccttcgttgccgttgatcacgttaatttccgcattccacgcggggagatttttggttttcttggttcgaacggctgcggtaaatccaccaccatgaaaatgctcaccggactgctgcccgccagcgaaggtgaggcgtggctgttcgggcaaccggttgatccaaaagatatcgatacccgccgtcgggtgggctatatgtcgcaggcgttttcgctctataacgaactcaccgtgcggcaaaaccttgagttacatgcccgtttgtttcacatcccggaagcggaaattcccgcaagagtggctgaaatgagcgagcgttttaagctcaacgacgttgaagatattctgccggagtcattgccgctcggcattcgccagcggctttcgctggcggtggcggtgattcatcgcccggagatgttaatcctcgatgagcctacttctggtgtcgatccggtggcgagggatatgttctggcagttgatggtcgatctctcgcgccaggacaaagtgactatcttcatctccacccactttatgaacgaagcggaacgttgcgaccgcatctcactgatgcacgccggaaaagtgcttgccagcggtacaccgcaggaactggttgagaaacgcggagccgccagtctggaagaggcatttatcgcctatttgcaggaagcggcagggcagagcaacgaagccgaagcgccgcccgtggtacacgacaccacccacgcgccgcgtcagggatttagcctgcgccgtctgtttagctacagccgccgcgaagcgctggaactgcgacgcgatccagtacgttcgacgctggcgctgatgggaacggtgatcctgatgctgataatgggttacggcatcagtatggatgtggaaaacctgcgctttgcggtgctcgaccgcgaccagaccgtcagtagccaggcgtggacactcaacctctccggttcccgttactttatcgaacagccgccgctcaccagttatgacgagcttgatcgtcggatgcgtgcgggcgatatcacggtggcgattgagatcccgcccaatttcgggcgcgatatcgcgcgtggtacgcctgtggaactcggcgtctggatcgacggagcgatgccgagccgtgctgaaacggtaaaaggttacgtgcaggccatgcaccagagctggttacaggatgtggcgagccgacaatcgacacccgccagccaaagcgggctgatgaatattgagacgcgctatcgctataacccggacgtaaaaagcctgccagcgattgttccggcggtgatcccgcttctgctgatgatgatcccgtcaatgctaagcgcccttagcgtggtgcgggaaaaagagcttgggtcgattatcaacctttacgtgacccccaccacgcgtagtgaatttttgcttggtaaacagttgccatacatcgcgctggggatgctgaactttttcctgctctgcggcctgtcggtgtttgtgtttggcgtaccgcataaaggcagtttcctgacgctcaccctggcggcgctgctgtatatcatcattgccaccggaatggggctgctgatctccacctttatgaaaagccagattgccgccattttcggaacggcgattatcacgttgatcccggcgacacagttttccgggatgatcgatccggtagcttcgctggaagggcctggacgttggatcggcgaggtttacccgaccagtcattttctgactatcgcccgcgggacgttctcgaaagcgctggatctgactgatttgtggcaactttttatcccgttactgatagccatcccgctggtgatgggcttaagtatcctgctgctgaaaaaacaggagggatga

SEQ ID NO: 26 - DNA, 1125 bp, Escherichia coli
atgcgccatttacgcaatatttttaatctgggtatcaaagagttgcgcagtctgctcggtgataaagcgatgctgacgctgattgtcttctcgtttacggtgtcggtgtattcgtcagcgaccgttacgccaggatcgttgaacctcgcgccgatcgccattgccgatatggatcaatcgcagttatcgaaccggatcgttaacagcttctatcgtccgtggtttttgccaccggagatgatcaccgccgatgagatggatgccggactggacgccggacgctataccttcgcgataaatattccgcctaattttcagcgtgatgtcctcgccggacgccagccggatattcaggtgaacgtcgatgccacgcgcatgagccaggcatttaccggcaatgggtatatccagaatattatcaacggtgaagtgaacagctttgtcgcgcgctaccgtgataacagcgaaccgttggtatcgctggaaacccggatgcgctttaacccgaacctcgatcccgcgtggtttggcggggtgatggcgatcatcaacaacattaccatgctggcgattgtattgaccggatcggcgctgatccgcgagcgtgaacacggcacggtggaacacttactggtgatgccgataacgccgtttgagatcatgatggcgaagatctggtcgatggggctggtggtgctggtggtatcgggattatcgctggtgctgatggtgaaaggtgtactgggcgtaccgattgaaggctcgatcccgctgtttatgctgggcgtggcgctcagtctgtttgccaccacgtcaatcggcatttttatggggacgatagcgcgttcaatgccgcaactggggctgctggtgattctggtgctgctgccgctgcaaatgctttccggtggttccacgccgcgcgaaagtatgccgcagatggtgcaggacattatgctgaccatgccgacgacacactttgttagcctcgcgcaggccatcctctaccggggtgccggattcgaaatcgtctggccgcagtttctgacgctgatggcaattggcggcgcatttttcaccattgcgctgctgcgattcaggaagacgattgggacaatggcgtaa

SEQ ID NO: 27 - protein, 332 aa, Escherichia coli; identical to SEQ ID NO: 15

SEQ ID NO: 28 - protein, 578 aa, Escherichia coli
MNDAVITLNGLEKRFPGMDKPAVAPLDCTIHAGYVTGLVGPDGAGKTTLMRMLAGLLKPDSGSATVIGFDPIKNDGALHAVLGYMPQKFGLYEDLTVMENLNLYADLRSVTGEARKQTFARLLEFTSLGPFTGRLAGKLSGGMKQKLGLACTLVGEPKVLLLDEPGVGVDPISRRELWQMVHELAGEGMLILWSTSYLDEAEQCRDVLLMNEGELLYQGEPKALTQTMAGRSFLMTSPHEGNRKLLQRALKLPQVSDGMIQGKSVRLILKKEATPDDIRHADGMPEININETTPRFEDAFIDLLGGAGTSESPLGAILHTVEGTPGETVIEAKELTKKFGDFAATDHVNFAVKRGEIFGLLGPNGAGKSTTFKMMCGLLVPTSGQALVLGMDLKESSGKARQHLGYMAQKFSLYGNLTVEQNLRFFSGVYGLRGRAQNEKISRMSEAFGLKSIASHATDELPLGFKQRLALACSLMHEPDILFLDEPTSGVDPLTRREFWLHINSMVEKGVTVMVTTHFMDEAEYCDRIGLVYRGKLIASGTPDDLKAQSANDEQPDPTMEQAFIQLIHDWDKEHSNE

SEQ ID NO: 29 - protein, 377 aa, Escherichia coli
MSNPILSWRRVRALCVKETRQIVRDPSSWLIAVVIPLLLLFIFGYGINLDSSKLRVGILLEQRSEAALDFTHTMTGSPYIDATISDNRQELIAKMQAGKIRGLVVIPVDFAEQMERANATAPIQVITDGSEPNTANFVQGYVEGIWQIWQMQRAEDNGQTFEPLIDVQTRYWFNPAAISQHFIIPGAVTIIMTVIGAILTSLVVAREWERGTMEALLSTEITRTELLLCKLIPYYFLGMLAMLLCMLVSVFILGVPYRGSLLILFFISSLFLLSTLGMGLLISTITRNQFNAAQVALNAAFLPSIMLSGFIFQIDSMPAVIRAVTYIIPARYFVSTLQSLFLAGNIPVVLVVNVLFLIASAVMFIGLTWLKTKRRLD

SEQ ID NO: 30 - protein, 368 aa, Escherichia coli
MFHRLWTLIRKELQSLLREPQTRAILILPVLIQVILFPFAATLEVTNATIAIYDEDNGEHSVELTQRFARASAFTHVLLLKSPQEIRPTIDTQKALLLVRFPADFSRKLDTFQTAPLQLILDGRNSNSAQIAANYLQQIVKNYQQELLEGKPKPNNSELVVRNWYNPNLDYKWFVVPSLIAMITTIGVMIVTSLSVAREREQGTLDQLLVSPLTTWQIFIGKAVPALIVATFQATIVLAIGIWAYQIPFAGSLALFYFTMVIYGLSLVGFGLLISSLCSTQQQAFIGVFVFMMPAILLSGYVSPVENMPVWLQNLTWINPIRHFTDITKQIYLKDASLDIVWNSLWPLLVITATTGSAAYAMFRRKVM

SEQ ID NO: 31 - protein, 490 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAAGYSVGTRTIVDVLDATTTLYNAKQELANARYNYLINQLNKSALGTLNEQDLLALNNALSKPVSTNPENVAPQTPEQNAIADGYAPDSPAPVVQQTSARTTTSNGHNPFRN

SEQ ID NO: 32 - protein, 355 aa, Escherichia coli; identical to SEQ ID NO: 16

SEQ ID NO: 33 - protein, 911 aa, Escherichia coli
MTHLELVPVPPVAQLAGVSQHYGKTVALNNITLDIPARCMVGLIGPDGVGKSSLLSLISGARVIEQGNVMVLGGDMRDPKHRRDVCPRIAWMPQGLGKNLYHTLSVYENVDFFARLFGHDKAEREVRINELLTSTGLAPFRDRPAGKLSGGMKQKLGLCCALIHDPELLILDEPTTGVDPLSRSQFWDLIDSIRQRQSNMSVLVATAYMEEAERFDWLVAMNAGEVLATGSAEELRQQTQSATLEEAFINLLPQAQRQAHQAVVIPPYQPENAEIAIEARDLTMRFGSFVAVDHVNFRIPRGEIFGFLGSNGCGKSTTMKMLTGLLPASEGEAWLFGQPVDPKDIDTRRRVGYMSQAFSLYNELTVRQNLELHARLFHIPEAEIPARVAEMSERFKLNDVEDILPESLPLGIRQRLSLAVAVIHRPEMLILDEPTSGVDPVARDMFWQLMVDLSRQDKVTIFISTHFMNEAERCDRISLMHAGKVLASGTPQELVEKRGAASLEEAFIAYLQEAAGQSNEAEAPPVVHDTTHAPRQGFSLRRLFSYSRREALELRRDPVRSTLALMGTVILMLIMGYGISMDVENLRFAVLDRDQTVSSQAWTLNLSGSRYFIEQPPLTSYDELDRRMRAGDITVAIEIPPNFGRDIARGTPVELGVWIDGAMPSRAETVKGYVQAMHQSWLQDVASRQSTPASQSGLMNIETRYRYNPDVKSLPAIVPAVIPLLLMMIPSMLSALSVVREKELGSIINLYVTPTTRSEFLLGKQLPYIALGMLNFFLLCGLSVFVFGVPHKGSFLTLTLAALLYIIIATGMGLLISTFMKSQIAAIFGTAIITLIPATQFSGMIDPVASLEGPGRWIGEVYPTSHFLTIARGTFSKALDLTDLWQLFIPLLIAIPLVMGLSILLLKKQEG

SEQ ID NO: 34 - protein, 374 aa, Escherichia coli
MRHLRNIFNLGIKELRSLLGDKAMLTLIVFSFTVSVYSSATVTPGSLNLAPIAIADMDQSQLSNRIVNSFYRPWFLPPEMITADEMDAGLDAGRYTFAINIPPNFQRDVLAGRQPDIQVNVDATRMSQAFTGNGYIQNIINGEVNSFVARYRDNSEPLVSLETRMRFNPNLDPAWFGGVMAIINNITMLAIVLTGSALIREREHGTVEHLLVMPITPFEIMMAKIWSMGLVVLVVSGLSLVLMVKGVLGVPIEGSIPLFMLGVALSLFATTSIGIFMGTIARSMPQLGLLVILVLLPLQMLSGGSTPRESMPQMVQDIMLTMPTTHFVSLAQAILYRGAGFEIVWPQFLTLMAIGGAFFTIALLRFRKTIGTMA

SEQ ID NO: 35 - DNA, 5816 bp, Artificial Sequence
  Description of Artificial Sequence Synthetic polynucleotide
ctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttaattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttaggaggtaaaacatatgccgcagcttgaagccagccttgaactggactttcaaagcgagtcctacaaagacgcttacagccgcatcaacgcgatcgtgattgaaggcgaacaagaggcgttcgacaactacaatcgccttgctgagatgctgcccgaccagcgggatgagcttcacaagctagccaagatggaacagcgccacatgaaaggctttatggcctgtggcaaaaatctctccgtcactcctgacatgggttttgcccagaaatttttcgagcgcttgcacgagaacttcaaagcggcggctgcggaaggcaaggtcgtcacctgcctactgattcaatcgctaatcatcgagtgctttgcgatcgcggcttacaacatctacatcccagtggcggatgcttttgcccgcaaaatcacggagggggtcgtgcgcgacgaatacctgcaccgcaacttcggtgaagagtggctgaaggcgaattttgatgcttccaaagccgaactggaagaagccaatcgtcagaacctgcccttggtttggctaatgctcaacgaagtggccgatgatgctcgcgaactcgggatggagcgtgagtcgctcgtcgaggactttatgattgcctacggtgaagctctggaaaacatcggcttcacaacgcgcgaaatcatgcgtatgtccgcctatggccttgcggccgtttgatccaggaaatctgaatgttcggtcttatcggtcatctcaccagtttggagcaggcccgcgacgtttctcgcaggatgggctacgacgaatacgccgatcaaggattggagttttggagtagcgctcctcctcaaatcgttgatgaaatcacagtcaccagtgccacaggcaaggtgattcacggtcgctacatcgaatcgtgtttcttgccggaaatgctggcggcgcgccgcttcaaaacagccacgcgcaaagttctcaatgccatgtcccatgcccaaaaacacggcatcgacatctcggccttggggggctttacctcgattattttcgagaatttcgatttggccagtttgcggcaagtgcgcgacactaccttggagtttgaacggttcaccaccggcaatactcacacggcctacgtaatctgtagacaggtggaagccgctgctaaaacgctgggcatcgacattacccaagcgacagtagcggttgtcggcgcgactggcgatatcggtagcgctgtctgccgctggctcgacctcaaactgggtgtcggtgatttgatcctgacggcgcgcaatcaggagcgtttggataacctgcaggctgaactcggccggggcaagattctgcccttggaagccgctctgccggaagctgactttatcgtgtgggtcgccagtatgcctcagggcgtagtgatcgacccagcaaccctgaagcaaccctgcgtcctaatcgacgggggctaccccaaaaacttgggcagcaaagtccaaggtgagggcatctatgtcctcaatggcggggtagttgaacattgcttcgacatcgactggcagatcatgtccgctgcagagatggcgcggcccgagcgccagatgtttgcctgctttgccgaggcgatgctcttggaatttgaaggctggcatactaacttctcctggggccgcaaccaaatcacgatcgagaagatggaagcgatcggtgaggcatcggtgcgccacggcttccaacccttggcattggcaatttgagaattcaaaacgtttcaattggctaataggatcctagacgtcgctaatacggccggccacccttttttaggtagcgctagcatagggccctaactcgagccccaagggcgacaccccataattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 36 - DNA, 1323 bp, Artificial Sequence
  Description of Artificial Sequence Synthetic polynucleotide
ctgtcaaacatgagaattaattccggggatccgtcgacctgcagttcgaagttcctattctctagaaagtataggaacttcagagcgcttttgaagctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaataaggggatcttgaagttcctattccgaagttcctattctctagaaagtataggaacttcgaagcagctccagcctacac

SEQ ID NO: 37 - DNA, 33 bp, Artificial Sequence
  Description of Artificial Sequence Synthetic primer
aatacatatgatgaaaaaacctgtcgtgatcgg

SEQ ID NO: 38 - DNA, 41 bp, Artificial Sequence
  Description of Artificial Sequence Synthetic primer
aataaggccggccttacatcaccttacgtctaaacatcgcg

SEQ ID NO: 39 - DNA, 5780 bp, Artificial Sequence
  Description of Artificial Sequence Synthetic polynucleotide
ttaagacccactttcacatttaagttgtttttctaatccgcaaatgatcaattcaaggccgaataagaaggctggctctgcaccttggtgttcaaataattcgatagcttgtcgtaataatgctggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacccagcttctgggcgagtttacgggtttttaaaccttcgattccgacctcattaagcagctctaatgcgctgttaatcactttacttttatctaatctggacatcatttggttttcctccagcaaaatgtacagcaaccattatcaccgccagaggtaaaatagtcaacacgcacggtgttagagctctccctatcagtgatagagattgacatccctatcagtgatagagatactgagcacatcagcaggacgcactgacccaattcattaaagaggagaaaggtcatatgatgaaaaaacctgtcgtgatcggattggcggtagtggtacttgccgccgtggttgccggaggctactggtggtatcaaagccgccaggataacggcctgacgctgtatggcaacgtggatattcgtacggtaaatcttagtttccgtgttggggggcgcgttgaatcgctggcggtggacgaaggtgatgctatcaaagcgggccaggtgctgggcgaactggatcacaagccgtatgagattgccctgatgcaggcgaaagcgggtgtttcggtggcacaggcgcagtatgacctgatgcttgccgggtatcgcaatgaagaaatcgctcaggccgccgcagcggtgaaacaggcgcaagccgcctatgactatgcgcagaacttctataaccgccagcaagggttgtggaaaagccgcactatttcggcaaatgacctggaaaatgcccgctcctcgcgcgaccaggcgcaggcaacgctgaaatcagcacaggataaattgcgtcagtaccgttccggtaaccgtgaacaggacatcgctcaggcgaaagccagcctcgaacaggcgcaggcgcaactggcgcaggcggagttgaatttacaggactcaacgttgatagccccgtctgatggcacgctgttaacgcgcgcggtggagccaggcacggtcctcaatgaaggtggcacggtgtttaccgtttcactaacgcgtccggtgtgggtgcgcgcttatgttgatgaacgtaatcttgaccaggcccagccggggcgcaaagtgctgctttataccgatggtcgcccggacaagccgtatcacgggcagattggtttcgtttcgccgactgctgaatttaccccgaaaaccgtcgaaacgccggatctgcgtaccgacctcgtctatcgcctgcgtattgtggtgaccgacgccgatgatgcgttacgccagggaatgccagtgacggtacaattcggtgacgaggcaggacatgaatgatgccgttatcacgctgaacggcctggaaaaacgctttccgggcatggacaagcccgccgtcgcgccgctcgattgtaccattcacgccggttatgtgacggggttggtggggccggacggtgcaggtaaaaccacgctgatgcggatgttggcgggattactgaaacccgacagcggcagtgccacggtgattggctttgatccgatcaaaaacgacggcgcgctgcacgccgtgctcggttatatgccgcagaaatttggtctgtatgaagatctcacggtgatggagaacctcaatctgtacgcggatttgcgcagcgtcaccggcgaggcacgtaagcaaacttttgctcgcctgctggagtttacgtctcttgggccgtttaccggacgcctggcgggcaagctctccggtgggatgaaacaaaaactcggtctggcctgtaccctggtgggcgaaccgaaagtgttgctgctcgatgaacccggcgtcggcgttgaccctatctcacggcgcgaactgtggcagatggtgcatgagctggcgggcgaagggatgttaatcctctggagtacctcgtatctcgacgaagccgagcagtgccgtgacgtgttactgatgaacgaaggcgagttgctgtatcagggagaaccaaaagccctgacacaaaccatggccggacgcagctttctgatgaccagtccacacgagggcaaccgcaaactgttgcaacgcgccttgaaactgccgcaggtcagcgacggcatgattcaggggaaatcggtacgtctgatcctcaaaaaagaggccacaccagacgatattcgccatgccgacgggatgccggaaatcaacatcaacgaaactacgccgcgttttgaagatgcgtttattgatttgctgggcggtgccggaacctcggaatcgccgctgggcgcaatattacatacggtagaaggcacacccggcgagacggtgatcgaagcgaaagaactgaccaagaaatttggggattttgccgccaccgatcacgtcaactttgccgttaaacgtggggagatttttggtttgctggggccaaacggcgcgggtaaatcgaccacctttaagatgatgtgcggtttgctggtgccgacttccggccaggcgctggtgctggggatggatctgaaagagagttccggtaaagcgcgccagcatctcggctatatggcgcaaaaattttcgctctacggtaacctgacggtcgaacagaatttacgctttttctctggtgtgtatggcttacgcggtcgggcgcagaacgaaaaaatctcccgcatgagcgaggcgttcggcctgaaaagtatcgcctcccacgccaccgatgaactgccattaggttttaaacagcggctggcgctggcctgttcgctgatgcatgaaccggacattctgtttctcgacgaaccgacttccggcgttgaccccctcacccgccgtgaattttggctgcacatcaacagcatggtagagaaaggcgtcacggtgatggtcaccacccactttatggatgaagcggaatattgcgaccgcatcggcctggtgtaccgcgggaaattaatcgccagcggcacgccggacgatttgaaagcacagtcggctaacgatgagcaacccgatcccaccatggagcaagcctttattcagttgatccacgactgggataaggagcatagcaatgagtaacccgatcctgtcctggcgtcgcgtacgggcgctgtgcgttaaagagacgcggcagatcgttcgcgatccgagtagctggctgattgcggtagtgatcccgctgctactgctgtttatttttggttacggcattaacctcgactccagcaagctgcgggtcgggattttactggaacagcgtagcgaagcggcgctggatttcacccacaccatgaccggttcgccctacatcgacgccaccatcagcgataaccgtcaggaactgatcgccaaaatgcaggcggggaaaattcgcggtctggtggttattccggtggattttgcggaacagatggagcgcgccaacgccaccgcaccgattcaggtgatcaccgacggcagtgagccgaataccgctaactttgtacaggggtatgtcgaagggatctggcagatctggcaaatgcagcgagcggaggacaacgggcagacttttgaaccgcttattgatgtacaaacccgctactggtttaacccggcggcgattagccagcacttcattatccccggtgcggtgaccattatcatgacggtcatcggcgcgattctcacctcgctggtggtggcgcgagaatgggaacgcggcaccatggaggctctgctctctacggagattacccgcacggaactgctgctgtgtaagctgatcccttattactttctcgggatgctggcgatgttgctgtgtatgctggtgtcagtgtttattctcggcgtgccgtatcgcgggtcgctgctgattctgttttttatctccagcctgtttttactcagtaccctggggatggggctgctgatttccacgattacccgcaaccagttcaatgccgctcaggtcgccctgaacgccgcttttctgccgtcgattatgctttccggctttatttttcagatcgacagtatgcccgcggtgatccgcgcggtgacgtacattattcccgctcgttatttcgtcagcaccctgcaaagcctgttcctcgccgggaatattccagtggtgctggtggtaaacgtgctgtttttgatcgcttcggcggtgatgtttatcggcctgacgtggctgaaaaccaaacgtcggctggattagggagaagagcatgtttcatcgcttatggacgttaatccgcaaagagttgcagtcgttgctgcgcgaaccgcaaacccgcgcgattctgattttacccgtgctaattcaggtgatcctgttcccgttcgccgccacgctggaagtgactaacgccaccatcgccatctacgatgaagataacggcgagcattcggtggagctgacccaacgttttgcccgcgccagcgcctttactcatgtgctgctgctgaaaagcccacaggagatccgcccaaccatcgacacacaaaaggcgttactactggtgcgtttcccggctgacttctcgcgcaaactggataccttccagaccgcgcctttgcagttgatcctcgacgggcgtaactccaacagtgcgcaaattgccgccaactacctgcaacagatcgtcaaaaattatcagcaggagctgctggaaggaaaaccgaaacctaacaacagcgagctggtggtacgcaactggtataacccgaatctcgactacaaatggtttgtggtgccgtcactgatcgccatgatcaccactatcggcgtaatgatcgtcacttcactttccgtcgcccgcgaacgtgaacaaggtacgctcgatcagctactggtttcgccgctcaccacctggcagatcttcatcggcaaagccgtaccggcgttaattgtcgccaccttccaggccaccattgtgctggcgattggtatctgggcgtatcaaatccccttcgccggatcgctggcgctgttctactttacgatggtgatttatggtttatcgctggtgggattcggtctgttgatttcatcactctgttcaacacaacagcaggcgtttatcggcgtgtttgtctttatgatgcccgccattctcctttccggttacgtttctccggtggaaaacatgccggtatggctgcaaaacctgacgtggattaaccctattcgccactttacggacattaccaagcagatttatttgaaggatgcgagtctggatattgtgtggaatagtttgtggccgctactggtgataacggccacgacagggtcagcggcgtacgcgatgtttagacgtaaggtgatgtaa

SEQ ID NO: 40 - DNA, 70 bp, Artificial Sequence
  Description of Artificial Sequence Synthetic primer
gcgtactggaagaactcaacgcgctattgttacaagaggaagcctgacgggtgtaggctggagctgcttc

SEQ ID NO: 41 - DNA, 70 bp, Artificial Sequence
  Description of Artificial Sequence Synthetic primer
gtgtaaggtttcaatgaatgaagtttaaaggatgttagcatgttttacctctgtcaaacatgagaattaa

SEQ ID NO: 42 - DNA, 1160 bp, Artificial Sequence
  Description of Artificial Sequence Synthetic polynucleotide
tgacgctgcggagggttatcaccagagcttaatcgacattactccccagcgcgtactggaagaactcaacgcgctattgttacaagaggaagcctgacggatgcgggttttgatcgttaaaacatcgtcgatgggcgatgttctccatacgttgcccgcactcactgatgcccagcaggcaatcccagggattaagtttgactgggtggtggaagaagggttcgcacagattccttcctggcacgctgccgttgagcgagttattcctgtggcaatacgtcgctggcgtaaagcctggttctcggcccccataaaagcggaacgcaaagcgtttcgtgaagcgctacaagcagagaactatgacgcagttatcgacgctcaggggctggtaaaaagcgcggcgctggtgacgcgtctggcgcatggcgtaaagcatggcatggactggcaaaccgctcgcgaacctttagccagcctgttttacaatcgtaagcatcatattgcaaaacagcagcacgccgtagaacgcacccgcgaactgtttgccaaaagtttgggctatagcaaaccgcaaacccagggcgattatgctatcgcacagcattttctgacgaacctgcctacagatgctggcgaatatgccgtatttcttcatgcgacgacccgtgatgataaacactggccggaagaacactggcgagaattgattggtttactggctgattcaggaatacggattaaacttccgtggggcgcgccgcatgaggaagaacgggcgaaacgactggcggaaggatttgcttatgttgaagtattgccgaagatgagtctggaaggcgttgcccgcgtgctggccggggctaaatttgtagtgtcggtggatacggggttaagccatttaacggcggcactggatagacccaatatcacggtttatggaccaaccgatccgggattaattggtgggtatgggaagaatcagatggtatgtagggctccaagagaaaatttaattaacctcaacagtcaagcagttttggaaaagttatcatcattataaaggtaaaacatgctaacatcctttaaacttcattcattgaaaccttacactctgaaatcatcaatgattttagagataataacttatatattatgttttt

SEQ ID NO: 43 - DNA, 301 bp, Artificial Sequence
  Description of Artificial Sequence Synthetic polynucleotide
tgacgctgcggagggttatcaccagagcttaatcgacattactccccagcgcgtactggaagaactcaacgcgctattgttacaagaggaagcctgacgggtgtaggctggagctgcttcgaagttcctatactttctagagaataggaacttcgaactgcaggtcgacggatccccggaattaattctcatgtttgacagaggtaaaacatgctaacatcctttaaacttcattcattgaaaccttacactctgaaatcatcaatgattttagagataataacttatatattatgttttt

SEQ ID NO: 44 - DNA, 211 bp, Artificial Sequence
  Description of Artificial Sequence Synthetic polynucleotide
gcccctatattatgcatttatacccccacaatcatgtcaagaattcaagcatcttaaataatgttaattatcggcaaagtctgtgctccccttctataatgctgaattgagcattcgcctcctgaacggtctttattcttccattgtgggtctttagattcacgattcttcacaatcattgatctaaagatctttctagattctcgaggca

SEQ ID NO: 45 - DNA, 225 bp, Artificial Sequence
  Description of Artificial Sequence Synthetic polynucleotide
ggccgcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggaaaacca

SEQ ID NO: 46 - DNA, 596 bp, Artificial Sequence
  Description of Artificial Sequence Synthetic polynucleotide
gctactcattagttaagtgtaatgcagaaaacgcatattctctattaaacttacgcattaatacgagaattttgtagctacttatactattttacctgagatcccgacataaccttagaagtatcgaaatcgttacataaacattcacacaaaccacttgacaaatttagccaatgtaaaagactacagtttctccccggtttagttctagagttaccttcagtgaaacatcggcggcgtgtcagtcattgaagtagcataaatcaattcaaaataccctgcgggaaggctgcgccaacaaaattaaatatttggtttttcactattagagcatcgattcattaatcaaaaaccttaccccccagcccccttcccttgtagggaagtgggagccaaactcccctctccgcgtcggagcgaaaagtctgagcggaggtttcctccgaacagaacttttaaagagagaggggttgggggagaggttctttcaagattactaaattgctatcactagacctcgtagaactagcaaagactacgggtggattgatcttgagcaaaaaaactttatgagaactttagcaggaggaaaacca

SEQ ID NO: 47 - DNA, 960 bp, Artificial Sequence
  Description of Artificial Sequence Synthetic polynucleotide
atgagcctgaatttcctggactttgaacaacctattgctgaactggaggcaaaaatcgattccctgactgccgttagccgccaggacgaaaagctggatatcaacatcgacgaagaagtacatcgcctgcgtgagaaatctgttgaactgacccgtaaaatcttcgccgatctgggcgcctggcagatcgcgcagctggctcgccacccacaacgtccgtataccctggactacgtacgtctggctttcgatgagttcgacgagctggcgggcgatcgtgcctacgcggacgacaaagctatcgtgggcggtatcgctcgtctggacggtcgtccggtaatgatcatcggccatcaaaagggtcgtgaaaccaaagagaaaatccgtcgtaacttcggtatgcctgcaccggaaggctatcgtaaagccctgcgtctgatgcaaatggcggagcgtttcaaaatgccgattatcacctttatcgatactcctggtgcttacccaggtgtcggtgcggaagaacgtggccagtccgaggctatcgcccgtaacctgcgtgaaatgtcccgcctgggtgtcccggttgtttgcaccgttattggcgagggtggctccggtggtgcgctggcaatcggtgttggtgacaaagttaacatgctgcagtactctacctacagcgtcatctctccggagggctgcgcttctatcctgtggaaatccgctgacaaagctccgctggcagctgaagctatgggcatcatcgcaccgcgcctgaaagagctgaaactgatcgactctatcatccctgagccgctgggtggtgctcaccgcaacccagaagcgatggcagcgtccctgaaagcacaactgctggctgacctggcggatctggatgttctgtctactgaggatctgaaaaatcgtcgttaccaacgtctgatgtcctatggttacgcttga

SEQ ID NO: 48 - DNA, 915 bp, Artificial Sequence
  Description of Artificial Sequence Synthetic polynucleotide
atgtcgtggatcgagcgtattaaatctaacatcaccccaactcgtaaggcatccattccggaaggcgtttggacgaaatgtgattcttgcggccaggttctgtatcgcgccgaactggaacgtaacctggaggtttgtccgaagtgtgaccaccacatgcgtatgaccgcgcgcaatcgtctgcatagcctgctggatgagggcagcctggtcgaactgggttccgagctggagccgaaagatgttctgaaattccgtgattctaaaaagtataaagaccgtctggcgtctgctcaaaaggaaaccggcgagaaggatgcactggtagttatgaaaggcactctgtatggcatgccggtggttgcagcggcttttgagttcgcttttatgggcggtagcatgggtagcgtagttggtgctcgttttgtacgtgcggtggaacaggccctggaggacaactgcccgctgatctgcttctccgcttctggcggtgcgcgtatgcaggaagcactgatgtccctgatgcagatggctaaaacctctgctgcactggcgaaaatgcaggagcgtggcctgccatacatctctgttctgacggacccgacgatgggtggtgtttccgcttctttcgcgatgctgggcgacctgaacattgccgaaccgaaggcgctgatcggtttcgcgggtccgcgtgttatcgaacagacggtacgcgaaaaactgccgccaggtttccaacgcagcgagtttctgatcgaaaaaggtgcaatcgacatgatcgttcgtcgccctgagatgcgtctgaagctggcttccatcctggcgaaactgatgaacctgccagccccgaatccggaagcgccgcgtgaaggcgttgttgtcccaccagtaccagaccaggaaccggaggcgtaa

SEQ ID NO: 49 - DNA, 471 bp, Artificial Sequence
  Description of Artificial Sequence Synthetic polynucleotide
atggacatccgtaaaatcaagaaactgatcgaactggttgaggagtctggcatcagcgagctggagatttccgaaggcgaagaatccgtccgtatcagccgtgctgccccggcagccagcttcccggtcatgcaacaggcttatgctgctccgatgatgcagcaaccggcacagagcaacgctgcggctccggcgactgttccgtctatggaggctccggcagctgcagaaatcagcggccacatcgttcgtagccctatggtgggcaccttctaccgtaccccatctccggacgcgaaagcgttcatcgaagtaggccagaaagtcaacgttggtgacaccctgtgtatcgtcgaagcgatgaaaatgatgaaccaaatcgaggcagataaatccggcaccgtaaaggcgatcctggttgaatctggtcagccggttgaatttgatgaaccgctggttgtcatcgaataa

SEQ ID NO: 50 - DNA, 1350 bp, Artificial Sequence
  Description of Artificial Sequence Synthetic polynucleotide
atgctggataaaatcgttattgctaaccgcggcgagattgctctgcgcatcctgcgcgcatgcaaagaactgggtattaaaaccgttgcagttcattcttccgccgatcgcgacctgaagcacgtcctgctggccgatgaaactgtatgcatcggtccagcaccgtccgttaaatcctacctgaacattccggcgatcatctctgccgcggaaatcaccggcgctgtagctatccacccgggttatggttttctgtccgaaaacgccaactttgcggagcaggttgagcgcagcggctttatcttcatcggtccgaaggctgaaaccatccgtctgatgggcgataaagtgtccgctatcgcggcaatgaaaaaggcaggtgttccatgcgttccgggctctgacggcccgctgggcgacgatatggataaaaaccgcgctatcgcaaaacgtatcggttatccggttattatcaaggcatctggcggtggtggtggtcgtggtatgcgcgttgttcgtggtgacgcggaactggctcagagcattagcatgacccgtgcggaagcgaaagcggctttctctaacgatatggtgtatatggaaaagtacctggagaacccgcgtcacgtggaaattcaggtgctggctgatggtcagggtaacgctatctacctggctgagcgcgattgctctatgcagcgtcgtcaccagaaggtggttgaagaagctccggcaccgggcatcactccagagctgcgtcgctacatcggcgaacgttgtgcgaaagcctgcgtggatatcggttaccgtggtgctggcactttcgaatttctgtttgaaaacggtgagttctacttcattgaaatgaacactcgtatccaggttgaacaccctgtcaccgaaatgattaccggcgttgacctgattaaagaacaactgcgtatcgcagcgggtcagccgctgtctattaagcaggaagaagtccatgtccgtggtcacgccgtcgaatgccgtatcaacgcagaagacccgaacaccttcctgccgtccccgggtaaaatcactcgctttcacgcgccaggtggtttcggtgtccgttgggagtcccacatttatgctggttacacggtaccgccgtactacgactccatgatcggtaaactgatctgctatggcgaaaaccgtgacgtagcgatcgcgcgtatgaagaacgctctgcaggagctgattattgatggcatcaaaaccaatgttgacctgcagatccgcattatgaacgacgagaacttccagcacggcggcaccaacatccattatctggagaagaaactgggtctgcaggaaaaataa

SEQ ID NO: 51 - DNA, 5344 bp, Artificial Sequence
  Description of Artificial Sequence Synthetic polynucleotide
gaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaacccctatttgtttatttttctaaatacattcaaatatgtatccgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaaggcgcgcctgatcagttggtgctgcattagctaagaaggtcaggagatattattcgacatctagctgacggccattgcgatcataaacgaggatatcccactggccattttcagcggcttcaaaggcaattttagacccatcagcactaatggttggattacgcacttcttggtttaagttatcggttaaattccgcttttgttcaaactcgcgatcatagagataaatatcagattcgccgcgacgattgaccgcaaagacaatgtagcgaccatcttcagaaacggcaggatgggaggcaatttcatttagggtattgaggcccggtaacagaatcgtttgcctggtgctggtatcaaatagatagatatcctgggaaccattgcggtctgaggcaaaaacgaggtagggttcggcgatcgccgggtcaaattcgagggcccgactatttaaactgcggccaccgggatcaacgggaaaattgacaatgcgcggataaccaacgcagctctggagcagcaaaccgaggctaccgaggaaaaaactgcgtagaaaagaaacatagcgcataggtcaaagggaaatcaaagggcgggcgatcgccaatttttctataatattgtcctaacagcacactaaaacagagccatgctagcaaaaatttggagtgccaccattgtcggggtcgatgccctcagggtcggggtggaagtggatatttccggcggcttaccgaaaatgatggtggtcggactgcggccggccaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggcctgcaggacaactcggcttccgagcttggctccaccatggttatatctggagtaaccagaatttcgacaacttcgacgactatctcggtgcttttacctccaaccaacgcaaaaacattaagcgcgaacgcaaagccgttgacaaagcaggtttatccctcaagatgatgaccggggacgaaattcccgcccattacttcccactcatttatcgtttctatagcagcacctgcgacaaatttttttgggggagtaaatatctccggaaacccttttttgaaaccctagaatctacctatcgccatcgcgttgttctggccgccgcttacacgccagaagatgacaaacatcccgtcggtttatctttttgtatccgtaaagatgattatctttatggtcgttattggggggcctttgatgaatatgactgtctccattttgaagcctgctattacaaaccgatccaatgggcaatcgagcagggaattacgatgtacgatccgggcgctggcggaaaacataagcgacgacgtggtttcccggcaaccccaaactatagcctccaccgtttttatcaaccccgcatgggccaagttttagacgcttatattgatgaaattaatgccatggagcaacaggaaattgaagcgatcaatgcggatattccctttaaacggcaggaagttcaattgaaaatttcctagcttcactagccaaaagcgcgatcgcccaccgaccatcctcccttgggggagatgcggccgc

SEQ ID NO: 52 - DNA, 7520 bp, Artificial Sequence
  Description of Artificial Sequence Synthetic polynucleotide
cgagcatttcaacgatgatgaatgggacggcgaacccactgaacccgtcgccattgacccagaaccgcgcaaagaacgggaaaaaattgatctcgatctggaggatgaaccagaggaaaaccgcaaaccgcaaaaaatcaaagtgaagttagccgatgggaaagagcgggaactcgcccatactcaaaccacaactttttgggatgctgatggtaaacccatttccgcccaagaatttatcgaaaagctatttggcgacctgcccgacctcttcaaggatgaagccgaactacgcaccatctgggggaaacccgatacccgtaaatcgttcctgaccggactcgcggaaaaaggctacggtgacacccaactgaaggcgatcgcacgcattgccgaagcggaaaaaagtgatgtctatgatgtcctgacttgggttgcctacaacaccaaacccattagcagagaagagcgagtaattaagcatcgagatctgattttctcgaagtacaccggaaagcagcaagaatttttagattttgtcctagaccaatacattcgagaaggagtggaggaacttgatcgggggaaactgcctaccctcatcgaaatcaaataccaaaccgttaatgaaggtttagtgatcttgggtcaggatatcggtcaagtattcgcagattttcaggcggatttatataccgaagatgtggcataaaaaaggacggcgatcgccgggggcgttgcctgccttgagcggccgcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggaaaaccatatgcaagaactggccctgagaagcgagctggacttcaatagcgaaacctataaagatgcgtatagccgtattaacgccattgtgatcgaaggcgagcaagaagcataccaaaactacctggacatggcgcaactgctgccggaggacgaggctgagctgattcgtttgagcaagatggagaaccgtcacaaaaagggttttcaagcgtgcggcaagaacctcaatgtgactccggatatggattatgcacagcagttctttgcggagctgcacggcaattttcagaaggctaaagccgagggtaagattgttacctgcctgctcatccaaagcctgatcatcgaggcgtttgcgattgcagcctacaacatttacattccagtggctgatccgtttgcacgtaaaatcaccgagggtgtcgtcaaggatgagtatacccacctgaatttcggcgaagtttggttgaaggaacattttgaagcaagcaaggcggagttggaggacgccaacaaagagaacttaccgctggtctggcagatgttgaaccaggtcgaaaaggatgccgaagtgctgggtatggagaaagaggctctggtggaggactttatgattagctatggtgaggcactgagcaacatcggcttttctacgagagaaatcatgaagatgagcgcgtacggtctgcgtgcagcataactcgagtataagtaggagataaaaacatgttcggcttgattggccacctgactagcctggagcacgcgcacagcgtggcggatgcgtttggctacggcccgtacgcaacccagggtttagacctgtggtgtagcgcaccgccacagtttgttgagcactttcatgtcacgagcattacgggccaaacgattgagggtaaatacattgagagcgcgtttttgccggagatgttgattaaacgtcgtatcaaagcagcgatccgtaagattctgaacgcgatggcatttgcgcagaagaacaatttgaacattaccgcgctgggtggcttcagcagcattatctttgaggagtttaatctgaaggagaatcgtcaggttcgcaatgtgagcttggagtttgaccgcttcaccaccggtaacacccatactgcttacattatctgccgtcaagtcgaacaggcgagcgcgaaactgggtatcgacctgtcccaagcgaccgtggcgatttgcggtgccacgggtgatattggcagcgcagtttgtcgctggctggatcgcaaaaccgacacccaagagctgttcctgattgcgcgcaataaggaacgcttgcaacgtctgcaagatgaactgggtcgcggcaagatcatgggcctggaagaggcactgccggaagcagacattattgtgtgggttgcctccatgccgaagggcgtggagattaatgcggaaaccctgaagaagccgtgtctgatcattgacggtggctacccgaagaatctggacacgaaaatcaagcatccggacgtgcacattttgaagggtggtattgtagagcattcgttggacattgattggaaaatcatggaaaccgtgaacatggacgttccgagccgtcaaatgtttgcgtgcttcgcagaggcgatcttgctggagttcgagcaatggcacacgaacttctcgtggggtcgcaatcaaatcacggtgacgaagatggaacagattggtgaggcgagcgtgaagcatggtctgcaaccgctgctgtcctggtaagaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaggcgcgccacgagaaagagttatgacaaattaaaattctgactcttagattatttccagagaggctgattttcccaatctttgggaaagcctaagtttttagattctatttctggatacatctcaaaagttctttttaaatgctgtgcaaaattatgctctggtttaattctgtctaagagatactgaatacaacataagccagtgaaaattttacggctgtttctttgattaatatcctccaatacttctctagagagccattttccttttaacctatcaggcaatttaggtgattctcctagctgtatattccagagccttgaatgatgagcgcaaatatttctaatatgcgacaaagaccgtaaccaagatataaaaaacttgttaggtaattggaaatgagtatgtattttttgtcgtgtcttagatggtaataaatttgtgtacattctagataactgcccaaaggcgattatctccaaagccatatatgacggcggtagtagaggatttgtgtacttgtttcgataatgcccgataaattcttctacttttttagattggcaatattgagtaatcgaatcgattaattcttgatgcttcccagtgtcataaaataaacttttattcagataccaatgaggatcataatcatgggagtagtgataaatcatttgagttctgactgctacttctatcgactccgtagcattaaaaataagcattctcaaggatttatcaaacttgtatagatttggccggcccgtcaaaagggcgacaccccataattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggtaaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttcgctgaacctgcagg

SEQ ID NO: 53 - protein, 231 aa, Artificial Sequence
  Description of Artificial Sequence Synthetic polypeptide
MQELALRSELDFNSETYKDAYSRINAIVIEGEQEAYQNYLDMAQLLPEDEAELIRLSKMENRHKKGFQACGKNLNVTPDMDYAQQFFAELHGNFQKAKAEGKIVTCLLIQSLIIEAFAIAAYNIYIPVADPFARKITEGVVKDEYTHLNFGEVWLKEHFEASKAELEDANKENLPLVWQMLNQVEKDAEVLGMEKEALVEDFMISYGEALSNIGFSTREIMKMSAYGLRAA

SEQ ID NO: 54 - protein, 340 aa, Artificial Sequence
  Description of Artificial Sequence Synthetic polypeptide
MFGLIGHLTSLEHAHSVADAFGYGPYATQGLDLWCSAPPQFVEHFHVTSITGQTIEGKYIESAFLPEMLIKRRIKAAIRKILNAMAFAQKNNLNITALGGFSSIIFEEFNLKENRQVRNVSLEFDRFTTGNTHTAYIICRQVEQASAKLGIDLSQATVAICGATGDIGSAVCRWLDRKTDTQELFLIARNKERLQRLQDELGRGKIMGLEEALPEADIIVWVASMPKGVEINAETLKKPCLIIDGGYPKNLDTKIKHPDVHILKGGIVEHSLDIDWKIMETVNMDVPSRQMFACFAEAILLEFEQWHTNFSWGRNQITVTKMEQIGEASVKHGLQPLLSW

SEQ ID NO: 55 - DNA, 6543 bp, Artificial Sequence
  Description of Artificial Sequence Synthetic polynucleotide
gtgggtgctgcagtagtcgggcctcgcctcggcaaataccgtgatggtcaagtccacgccattcctggtcacaacatgagtattgcgaccttaggctgtctaattctttggattggctggtttggttttaaccccggttctcaattggcagcagatgctgcggtgccttacatcgcaatcactacaaacctttcggctgcagctgggggaatcaccgcaaccgcaacctcttggatcaaagatgggaagccagacctgtctatgattattaacggtattttggctggtctcgttgggattacagccggttgtgatggcgtcagtttcttttctgctgtgatcatcggggcgatcgccggtgtactcgtcgtcttctctgtggccttcttcgatgctattaaaatcgatgaccccgttggtgcgacctctgtgcacctcgtctgcggtatctggggaactcttgccgttggtctgttcaagatggatgggggtttattcactggcggtggcatccaacagctgattgcccaaatcgtcggaatcctttccattggtggctttaccgtcgcctttagctttattgtttggtatgccctatcggcagtccttggtggcattcgcgtcgaaaaagacgaggaactccggggtctcgacattggtgagcacggcatggaagcttacagcggctttgttaaagagtccgatgttatcttccgagggactgccactggttccgaaaccgaaggataagcggccgcggtactgccctcgatctgtaagagaatataaaaagccagattattaatccggcttttttgttatttctatacatcttatatccgtgggatccgagctctcaggtatccggtacgccgccgcaaaaaaccccgcttcggcggggttttttcgcggcgcgccatcctcccaggaaatccttaaaacaatctaaagaaatttttcctaaccttccttacccaagggaggttttttatgtgagttcacattttgttacgttacccaatcaatacttgagccgctcaaaaagtctgacctagagcagaaagtccctgagtatatcgactcattaatccggtctttccgcttggtttcttgagttgattttctgcgaaattttggaaattcagagatgtaaccttagggggagtccacttaaaaacggctctgctcaaccttgcaaatgccctactcttcttctgtctagcccaagcactccctgagaaaattagcggcgatcgcctataaacatgaagttttatgacagatcattttacaagatgtaatgtttaaatgccggcagacgttgtataacatttacctaagattaagagtcactcgcagtactccttagaaaccccataggttccaaggaactagcatgaactttatctggcaactttaagaatctgagaaattcaatgaatgtaaagtttcttaaatgccaaggtgaaaaacaagcaaaaatagctgacactcttaattggctttggggattaagtttccaactcgaaaacaaaaccttttatcgactctaggattttgttttcagcaagagagcccctcagcacttgcttcactcttgttagtaagcaaaccgcacaaaataaatcccactcatcaaaatataagtaggagataaaaacatgtttgggccggccaaaagagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtcaccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatttttttttcctcctcggcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggcgctgacgaaaaacatattctcaataaaccctttagggaaataggccaggttttcaccgtaacacgccacatcttgcgaatatatgtgtagaaactgccggaaatcgtcgtgtgcactcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatttttttttcctcctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggtgcacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatttttttttcctcctttaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattgaggggtgtcgcccttattcgactcggggcctgcagg

SEQ ID NO: 56 - DNA, 1947 bp, Artificial Sequence
  Description of Artificial Sequence Synthetic polynucleotide
atgtttgccttccgtgacttcctgacgtttagcacgggcggtttggtcgtgttgagcggtggcggtgttgcgattgcacaaaccacccctccgcagatcgccactccggagccgtttatcggtcagacgccgcaggcaccgctgccaccgctggctgcgccgtccgttgaaagcctggacaccgcggctttcctgccgagcctgggcggtctgtcccaaccgaccaccctggccgcactgcctttgccgagcccggagttgaacctgtcgcctacggcgcatctgggtaccatccaggcgccaagcccgctgttggcgcaagtggataccactgcgaccccgagcccgaccaccgcgattgacgtcaccctgccgacggcggaaacgaatcaaaccattccgctggtccagccgctgccgccagaccgcgtcatcaatgaggacctgaaccaactgctggagccgattgataacccggcagttacggtgccgcaggaagcgaccgccgttacgaccgataatgttgtggatgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 57 - protein, 648 aa, Artificial Sequence
  Description of Artificial Sequence Synthetic polypeptide
MFAFRDFLTFSTGGLVVLSGGGVAIAQTTPPQIATPEPFIGQTPQAPLPPLAAPSVESLDTAAFLPSLGGLSQPTTLAALPLPSPELNLSPTAHLGTIQAPSPLLAQVDTTATPSPTTAIDVTLPTAETNQTIPLVQPLPPDRVINEDLNQLLEPIDNPAVTVPQEATAVTTDNVVDENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 58 - DNA, 1494 bp, Artificial Sequence
  Description of Artificial Sequence Synthetic polynucleotide
atgtttgcctttcgtgacttcttgaccttcagcaccggtggcctggttgtcctgtccggcggtggtgttgcgattgcggagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 59 - protein, 497 aa, Artificial Sequence
  Description of Artificial Sequence Synthetic polypeptide
MFAFRDFLTFSTGGLVVLSGGGVAIAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 60 - DNA, 1482 bp, Artificial Sequence; identical to SEQ ID NO: 23
  Description of Artificial Sequence Synthetic polynucleotide

SEQ ID NO: 61 - protein, 493 aa, Artificial Sequence
  Description of Artificial Sequence Synthetic polypeptide
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 62 - DNA, 1311 bp, Artificial Sequence
  Description of Artificial Sequence Synthetic polynucleotide
atgatcacttgtattactgtttatgtaagcagacagttttattgttcatgatgatatatttttatcttgtgcaatgtaacatcagagattttgagacacaacgtggctttcccccccccccccttaattaaacccctatttgtttatttttctaaatacattcaaatatgtatccgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaaggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagcttgggggggggggggaaagccacgttgtgtctcaaaatctctgatgttacattgcacaagataaaaatatatcatcatgaacaataaaactgtctgcttacataaacagtaatacaagtgtacat

SEQ ID NO: 63 - DNA, 1400 bp, Artificial Sequence
  Description of Artificial Sequence Synthetic polynucleotide
atgatcacttgtattactgtttatgtaagcagacagttttattgttcatgatgatatatttttatcttgtgcaatgtaacatcagagattttgagacacaacgtggctttcccccccccccccttaattaaacccctatttgtttatttttctaaatacattcaaatatgtatccgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaaggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagcttgcccctatattatgcatttatacccccacaatcatgtcaagaattcaagcatcttaaataatgttaattatcggcaaagtctgtgctccccttctataatgctgaattgagcattcgcctcctgaacggtctttattcttccattgtgggtctttagattcacgattcttcacaatcattgatctaaggatctttgtagattctctgtacat

SEQ ID NO: 64 - DNA, 1438 bp, Artificial Sequence
  Description of Artificial Sequence Synthetic polynucleotide
atgatcagagaatctacaaagatccttagatcaatgattgtgaagaatcgtgaatctaaagacccacaatggaagaataaagaccgttcaggaggcgaatgctcaattcagcattatagaaggggagcacagactttgccgataattaacattatttaagatgcttgaattcttgacatgattgtgggggtataaatgcataatataggggcttaattaaacccctatttgtttatttttctaaatacattcaaatatgtatccgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaaggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagcttccaaggtggctacttcaacgatagcttaaacttcgctgctccagcgaggggatttcactggtttgaatgcttcaatgcttgccaaaagagtgctactggaacttacaagagtgaccctgcgtcaggggagctagcactcaaaaaagactcctcctgtacat

SEQ ID NO: 65 - DNA, 1504 bp, Artificial Sequence
  Description of Artificial Sequence Synthetic polynucleotide
atgatcaggaggagtcttttttgagtgctagctcccctgacgcagggtcactcttgtaagttccagtagcactcttttggcaagcattgaagcattcaaaccagtgaaatcccctcgctggagcagcgaagtttaagctatcgttgaagtagccaccttggttaattaaacccctatttgtttatttttctaaatacattcaaatatgtatccgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaaggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagctttagtacaaaaagacgattaaccccatgggtaaaagcaggggagccactaaagttcacaggtttacaccgaattttccatttgaaaagtagtaaatcatacagaaaacaatcatgtaaaaattgaatactctaatggtttgatgtccgaaaaagtctagtttcttctattcttcgaccaaatctatggcagggcactatcacagagctggcttaataatttgggagaaatgggtgggggcggactttcgtagaacaatgtagattaaagtactgtacat

SEQ ID NO: 66 - DNA, 1883 bp, Artificial Sequence
  Description of Artificial Sequence Synthetic polynucleotide
atgatcatcctcctcctaaagttctcataaagtttttttgctcaagatcaatccacccgtagtctttgctagttctacgaggtctagtgatagcaatttagtaatcttgaaagaacctctcccccaacccctctctctttaaaagttctgttcggaggaaacctccgctcagacttttcgctccgacgcggagaggggagtttggctcccacttccctacaagggaagggggctggggggtaaggtttttgattaatgaatcgatgctctaatagtgaaaaaccaaatatttaattttgttggcgcagccttcccgcagggtattttgaattgatttatgctacttcaatgactgacacgccgccgatgtttcactgaaggtaactctagaactaaaccggggagaaactgtagtcttttacattggctaaatttgtcaagtggtttgtgtgaatgtttatgtaacgatttcgatacttctaaggttatgtcgggatctcaggtaaaatagtataagtagctacaaaattctcgtattaatgcgtaagtttaatagagaatatgcgttttctgcattacacttaactaatgagtagttaattaaacccctatttgtttatttttctaaatacattcaaatatgtatccgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaaggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagcttgcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggatgtacat

SEQ ID NO: 67 - DNA, 4971 bp, Artificial Sequence
  Description of Artificial Sequence Synthetic polynucleotide
atgatgaaaaaacctgtcgtgatcggattggcggtagtggtacttgccgccgtggttgccggaggctactggtggtatcaaagccgccaggataacggcctgacgctgtatggcaacgtggatattcgtacggtaaatcttagtttccgtgttggggggcgcgttgaatcgctggcggtggacgaaggtgatgctatcaaagcgggccaggtgctgggcgaactggatcacaagccgtatgagattgccctgatgcaggcgaaagcgggtgtttcggtggcacaggcgcagtatgacctgatgcttgccgggtatcgcaatgaagaaatcgctcaggccgccgcagcggtgaaacaggcgcaagccgcctatgactatgcgcagaacttctataaccgccagcaagggttgtggaaaagccgcactatttcggcaaatgacctggaaaatgcccgctcctcgcgcgaccaggcgcaggcaacgctgaaatcagcacaggataaattgcgtcagtaccgttccggtaaccgtgaacaggacatcgctcaggcgaaagccagcctcgaacaggcgcaggcgcaactggcgcaggcggagttgaatttacaggactcaacgttgatagccccgtctgatggcacgctgttaacgcgcgcggtggagccaggcacggtcctcaatgaaggtggcacggtgtttaccgtttcactaacgcgtccggtgtgggtgcgcgcttatgttgatgaacgtaatcttgaccaggcccagccggggcgcaaagtgctgctttataccgatggtcgcccggacaagccgtatcacgggcagattggtttcgtttcgccgactgctgaatttaccccgaaaaccgtcgaaacgccggatctgcgtaccgacctcgtctatcgcctgcgtattgtggtgaccgacgccgatgatgcgttacgccagggaatgccagtgacggtacaattcggtgacgaggcaggacatgaatgatgccgttatcacgctgaacggcctggaaaaacgctttccgggcatggacaagcccgccgtcgcgccgctcgattgtaccattcacgccggttatgtgacggggttggtggggccggacggtgcaggtaaaaccacgctgatgcggatgttggcgggattactgaaacccgacagcggcagtgccacggtgattggctttgatccgatcaaaaacgacggcgcgctgcacgccgtgctcggttatatgccgcagaaatttggtctgtatgaagatctcacggtgatggagaacctcaatctgtacgcggatttgcgcagcgtcaccggcgaggcacgtaagcaaacttttgctcgcctgctggagtttacgtctcttgggccgtttaccggacgcctggcgggcaagctctccggtgggatgaaacaaaaactcggtctggcctgtaccctggtgggcgaaccgaaagtgttgctgctcgatgaacccggcgtcggcgttgaccctatctcacggcgcgaactgtggcagatggtgcatgagctggcgggcgaagggatgttaatcctctggagtacctcgtatctcgacgaagccgagcagtgccgtgacgtgttactgatgaacgaaggcgagttgctgtatcagggagaaccaaaagccctgacacaaaccatggccggacgcagctttctgatgaccagtccacacgagggcaaccgcaaactgttgcaacgcgccttgaaactgccgcaggtcagcgacggcatgattcaggggaaatcggtacgtctgatcctcaaaaaagaggccacaccagacgatattcgccatgccgacgggatgccggaaatcaacatcaacgaaactacgccgcgttttgaagatgcgtttattgatttgctgggcggtgccggaacctcggaatcgccgctgggcgcaatattacatacggtagaaggcacacccggcgagacggtgatcgaagcgaaagaactgaccaagaaatttggggattttgccgccaccgatcacgtcaactttgccgttaaacgtggggagatttttggtttgctggggccaaacggcgcgggtaaatcgaccacctttaagatgatgtgcggtttgctggtgccgacttccggccaggcgctggtgctggggatggatctgaaagagagttccggtaaagcgcgccagcatctcggctatatggcgcaaaaattttcgctctacggtaacctgacggtcgaacagaatttacgctttttctctggtgtgtatggcttacgcggtcgggcgcagaacgaaaaaatctcccgcatgagcgaggcgttcggcctgaaaagtatcgcctcccacgccaccgatgaactgccattaggttttaaacagcggctggcgctggcctgttcgctgatgcatgaaccggacattctgtttctcgacgaaccgacttccggcgttgaccccctcacccgccgtgaattttggctgcacatcaacagcatggtagagaaaggcgtcacggtgatggtcaccacccactttatggatgaagcggaatattgcgaccgcatcggcctggtgtaccgcgggaaattaatcgccagcggcacgccggacgatttgaaagcacagtcggctaacgatgagcaacccgatcccaccatggagcaagcctttattcagttgatccacgactgggataaggagcatagcaatgagtaacccgatcctgtcctggcgtcgcgtacgggcgctgtgcgttaaagagacgcggcagatcgttcgcgatccgagtagctggctgattgcggtagtgatcccgctgctactgctgtttatttttggttacggcattaacctcgactccagcaagctgcgggtcgggattttactggaacagcgtagcgaagcggcgctggatttcacccacaccatgaccggttcgccctacatcgacgccaccatcagcgataaccgtcaggaactgatcgccaaaatgcaggcggggaaaattcgcggtctggtggttattccggtggattttgcggaacagatggagcgcgccaacgccaccgcaccgattcaggtgatcaccgacggcagtgagccgaataccgctaactttgtacaggggtatgtcgaagggatctggcagatctggcaaatgcagcgagcggaggacaacgggcagacttttgaaccgcttattgatgtacaaacccgctactggtttaacccggcggcgattagccagcacttcattatccccggtgcggtgaccattatcatgacggtcatcggcgcgattctcacctcgctggtggtggcgcgagaatgggaacgcggcaccatggaggctctgctctctacggagattacccgcacggaactgctgctgtgtaagctgatcccttattactttctcgggatgctggcgatgttgctgtgtatgctggtgtcagtgtttattctcggcgtgccgtatcgcgggtcgctgctgattctgttttttatctccagcctgtttttactcagtaccctggggatggggctgctgatttccacgattacccgcaaccagttcaatgccgctcaggtcgccctgaacgccgcttttctgccgtcgattatgctttccggctttatttttcagatcgacagtatgcccgcggtgatccgcgcggtgacgtacattattcccgctcgttatttcgtcagcaccctgcaaagcctgttcctcgccgggaatattccagtggtgctggtggtaaacgtgctgtttttgatcgcttcggcggtgatgtttatcggcctgacgtggctgaaaaccaaacgtcggctggattagggagaagagcatgtttcatcgcttatggacgttaatccgcaaagagttgcagtcgttgctgcgcgaaccgcaaacccgcgcgattctgattttacccgtgctaattcaggtgatcctgttcccgttcgccgccacgctggaagtgactaacgccaccatcgccatctacgatgaagataacggcgagcattcggtggagctgacccaacgttttgcccgcgccagcgcctttactcatgtgctgctgctgaaaagcccacaggagatccgcccaaccatcgacacacaaaaggcgttactactggtgcgtttcccggctgacttctcgcgcaaactggataccttccagaccgcgcctttgcagttgatcctcgacgggcgtaactccaacagtgcgcaaattgccgccaactacctgcaacagatcgtcaaaaattatcagcaggagctgctggaaggaaaaccgaaacctaacaacagcgagctggtggtacgcaactggtataacccgaatctcgactacaaatggtttgtggtgccgtcactgatcgccatgatcaccactatcggcgtaatgatcgtcacttcactttccgtcgcccgcgaacgtgaacaaggtacgctcgatcagctactggtttcgccgctcaccacctggcagatcttcatcggcaaagccgtaccggcgttaattgtcgccaccttccaggccaccattgtgctggcgattggtatctgggcgtatcaaatccccttcgccggatcgctggcgctgttctactttacgatggtgatttatggtttatcgctggtgggattcggtctgttgatttcatcactctgttcaacacaacagcaggcgtttatcggcgtgtttgtctttatgatgcccgccattctcctttccggttacgtttctccggtggaaaacatgccggtatggctgcaaaacctgacgtggattaaccctattcgccactttacggacattaccaagcagatttatttgaaggatgcgagtctggatattgtgtggaatagtttgtggccgctactggtgataacggccacgacagggtcagcggcgtacgcgatgtttagacgtaaggtgatgtaa

SEQ ID NO: 68 - DNA, 999 bp, Artificial Sequence
  Description of Artificial Sequence Synthetic polynucleotide
atgatgaaaaaacctgtcgtgatcggattggcggtagtggtacttgccgccgtggttgccggaggctactggtggtatcaaagccgccaggataacggcctgacgctgtatggcaacgtggatattcgtacggtaaatcttagtttccgtgttggggggcgcgttgaatcgctggcggtggacgaaggtgatgctatcaaagcgggccaggtgctgggcgaactggatcacaagccgtatgagattgccctgatgcaggcgaaagcgggtgtttcggtggcacaggcgcagtatgacctgatgcttgccgggtatcgcaatgaagaaatcgctcaggccgccgcagcggtgaaacaggcgcaagccgcctatgactatgcgcagaacttctataaccgccagcaagggttgtggaaaagccgcactatttcggcaaatgacctggaaaatgcccgctcctcgcgcgaccaggcgcaggcaacgctgaaatcagcacaggataaattgcgtcagtaccgttccggtaaccgtgaacaggacatcgctcaggcgaaagccagcctcgaacaggcgcaggcgcaactggcgcaggcggagttgaatttacaggactcaacgttgatagccccgtctgatggcacgctgttaacgcgcgcggtggagccaggcacggtcctcaatgaaggtggcacggtgtttaccgtttcactaacgcgtccggtgtgggtgcgcgcttatgttgatgaacgtaatcttgaccaggcccagccggggcgcaaagtgctgctttataccgatggtcgcccggacaagccgtatcacgggcagattggtttcgtttcgccgactgctgaatttaccccgaaaaccgtcgaaacgccggatctgcgtaccgacctcgtctatcgcctgcgtattgtggtgaccgacgccgatgatgcgttacgccagggaatgccagtgacggtacaattcggtgacgaggcaggacatgaatga

SEQ ID NO: 69 - protein, 332 aa, Artificial Sequence; identical to SEQ ID NO: 15
  Description of Artificial Sequence Synthetic polypeptide

SEQ ID NO: 70 - DNA, 1737 bp, Artificial Sequence; identical to SEQ ID NO: 20
  Description of Artificial Sequence Synthetic polynucleotide

SEQ ID NO: 71 - protein, 578 aa, Artificial Sequence; identical to SEQ ID NO: 28
  Description of Artificial Sequence Synthetic polypeptide

SEQ ID NO: 72 - DNA, 1134 bp, Artificial Sequence; identical to SEQ ID NO: 21
  Description of Artificial Sequence Synthetic polynucleotide

SEQ ID NO: 73 - protein, 377 aa, Artificial Sequence; identical to SEQ ID NO: 29
  Description of Artificial Sequence Synthetic polypeptide

SEQ ID NO: 74 - DNA, 1107 bp, Artificial Sequence; identical to SEQ ID NO: 22
  Description of Artificial Sequence Synthetic polynucleotide

SEQ ID NO: 75 - protein, 368 aa, Artificial Sequence; identical to SEQ ID NO: 30
  Description of Artificial Sequence Synthetic polypeptide

SEQ ID NO: 76 - DNA, 5028 bp, Artificial Sequence
  Description of Artificial Sequence Synthetic polynucleotide
acaactcggcttccgagcttggctccaccatggttatatctggagtaaccagaatttcgacaacttcgacgactatctcggtgcttttacctccaaccaacgcaaaaacattaagcgcgaacgcaaagccgttgacaaagcaggtttatccctcaagatgatgaccggggacgaaattcccgcccattacttcccactcatttatcgtttctatagcagcacctgcgacaaatttttttgggggagtaaatatctccggaaacccttttttgaaaccctagaatctacctatcgccatcgcgttgttctggccgccgcttacacgccagaagatgacaaacatcccgtcggtttatctttttgtatccgtaaagatgattatctttatggtcgttattggggggcctttgatgaatatgactgtctccattttgaagcctgctattacaaaccgatccaatgggcaatcgagcagggaattacgatgtacgatccgggcgctggcggaaaacataagcgacgacgtggtttcccggcaaccccaaactatagcctccaccgtttttatcaaccccgcatgggccaagttttagacgcttatattgatgaaattaatgccatggagcaacaggaaattgaagcgatcaatgcggatattccctttaaacggcaggaagttcaattgaaaatttcctagcttcactagccaaaagcgcgatcgcccaccgaccatcctcccttgggggagatgcggccgcaacgtaaaaaaacccgccccggcgggtttttttataccggtactgccctcgatctgtagaattctgcacgcagatgtgccgaagtaaaaaatgccctcttgggttatcaagagggtcattatatttaattaacgaatccatgtgggagtttattcttgacacagatatttatgatataataactgagtaagcttaacataaggaggaaaaactaatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaggcgcgcctgatcagttggtgctgcattagctaagaaggtcaggagatattattcgacatctagctgacggccattgcgatcataaacgaggatatcccactggccattttcagcggcttcaaaggcaattttagacccatcagcactaatggttggattacgcacttcttggtttaagttatcggttaaattccgcttttgttcaaactcgcgatcatagagataaatatcagattcgccgcgacgattgaccgcaaagacaatgtagcgaccatcttcagaaacggcaggatgggaggcaatttcatttagggtattgaggcccggtaacagaatcgtttgcctggtgctggtatcaaatagatagatatcctgggaaccattgcggtctgaggcaaaaacgaggtagggttcggcgatcgccgggtcaaattcgagggcccgactatttaaactgcggccaccgggatcaacgggaaaattgacaatgcgcggataaccaacgcagctctggagcagcaaaccgaggctaccgaggaaaaaactgcgtagaaaagaaacatagcgcataggtcaaagggaaatcaaagggcgggcgatcgccaatttttctataatattgtcctaacagcacactaaaacagagccatgctagcaaaaatttggagtgccaccattgtcggggtcgatgccctcagggtcggggtggaagtggatatttccggcggcttaccgaaaatgatggtggtcggactgcggccggccaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggcctgcagg

SEQ ID NO: 77 - DNA, 1494 bp, Artificial Sequence
  Description of Artificial Sequence Synthetic polynucleotide
atgtttgcctttcgtgacttcttgaccttcagcaccggtggcctggttgtcctgtccggcggtggtgttgcgattgcggagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaattaa

SEQ ID NO: 78 - protein, 497 aa, Artificial Sequence; identical to SEQ ID NO: 59
  Description of Artificial Sequence Synthetic polypeptide

SEQ ID NO: 79 - DNA, 1512 bp, Artificial Sequence
  Description of Artificial Sequence Synthetic polynucleotide
atgcagaaacaacaaaatctggactactttagcccgcaggccctggccctgtgggctgcgattgcgagcttgggtgttatgtcccctgcgcatgcggagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaattaa

SEQ ID NO: 80 - protein, 503 aa, Artificial Sequence
  Description of Artificial Sequence Synthetic polypeptide
MQKQQNLDYFSPQALALWAAIASLGVMSPAHAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 81 - DNA, 1947 bp, Artificial Sequence
  Description of Artificial Sequence Synthetic polynucleotide
atgtttgccttccgtgacttcctgacgtttagcacgggcggtttggtcgtgttgagcggtggcggtgttgcgattgcacaaaccacccctccgcagatcgccactccggagccgtttatcggtcagacgccgcaggcaccgctgccaccgctggctgcgccgtccgttgaaagcctggacaccgcggctttcctgccgagcctgggcggtctgtcccaaccgaccaccctggccgcactgcctttgccgagcccggagttgaacctgtcgcctacggcgcatctgggtaccatccaggcgccaagcccgctgttggcgcaagtggataccactgcgaccccgagcccgaccaccgcgattgacgtcaccctgccgacggcggaaacgaatcaaaccattccgctggtccagccgctgccgccagaccgcgtcatcaatgaggacctgaaccaactgctggagccgattgataacccggcagttacggtgccgcaggaagcgaccgccgttacgaccgataatgttgtggatgagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaattaa

SEQ ID NO: 82 - protein, 648 aa, Artificial Sequence; identical to SEQ ID NO: 57
  Description of Artificial Sequence Synthetic polypeptide

SEQ ID NO: 83 - DNA, 1779 bp, Artificial Sequence
  Description of Artificial Sequence Synthetic polynucleotide
atgcaaaaacaacagaatctggactactttagcccgcaggcgttggcactgtgggcggctattgcttccctgggtgttatgagcccggcacacgcggagccgcgtagcgagggcagccattctgatccgctggttccgaccgcgacgcaggtcgtggttccggcgctgccggtggaggacgttgcgccgaccgccgcaccggcatcgcagaccccggctcctcagagcgaaaacttggcgcaatccagcacccaagccgtcacgagccctgtggcgcaggcgcaggaagccccgcaagacagcaatctgccgcaactgtatgcccagcagcaaggtaacccaaatgcccaacaggcgaacccggagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaattaa

SEQ ID NO: 84 - protein, 592 aa, Artificial Sequence
  Description of Artificial Sequence Synthetic polypeptide
MQKQQNLDYFSPQALALWAAIASLGVMSPAHAEPRSEGSHSDPLVPTATQVVVPALPVEDVAPTAAPASQTPAPQSENLAQSSTQAVTSPVAQAQEAPQDSNLPQLYAQQQGNPNAQQANPENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 85 - DNA, 1776 bp, Artificial Sequence
  Description of Artificial Sequence Synthetic polynucleotide
atgttcgcttttcgcgactttctgaccttttcgactggcggcctggtcgttctgtccggtggcggtgttgcgattgcgcagaccacccctccgcagatcgcgaccccggaaccgtttatcggtcagacgccgcaagccccgctgcctccgctggccgctccgagcgttgagagcctggataccgcggctttcttgccgtcgctgggcggtctgagccaaccgaccacgctggcagcactgccgctgccgagcccagagctgaatctgtccccgaccgcccacctgggtacgatccaagccccgagcccgttgctggcgcaagtggataccaccgctacgccgagcccgacgaccgccattgatgtgactttgccgaccgcggaaacgaatcaaacgattccgctggttcaaccgctgccgcctgatcgtgtgattaacgaagatctgaaccagctgctggaaccgatcgacaatccggcggtcaccgtcccgcaagaggcaaccgcggtgaccaccgataatgtggttgacctgacgctcgaggaaacgatccgcctggcactggagcgcaacgaaaccttgcaagaggcgcgtctgaactatgaccgcagcgaggagctggtgcgtgaggcgattgcggctgagtacccgaatttgtcgaaccaggtcgacattacccgtactgacagcgcgaacggtgagctgcaagctcgtcgtctgggtggtgacaataatgccaccaccgccatcaatggtcgcctggaagtgagctacgacatctataccggcggtcgccgtagcgcgcagattgaggcggcacagacccagctgcaaattgccgagctggatatcgaacgcctgaccgaggagactcgtctggctgcggcggtgaattactataatctgcaatctgcggacgcgcaggttgttattgaacagagctcagtttttgatgcaacccagcaactggatcaaactactcagcgtttcaacgtgggtctggtggcaattacggacgttcagaacgcgcgtgcagagctggctagcgcccaacagcgtctgacgcgcgctgaagccacccagcgcacggcacgtcgtcaactggcgcagttgctgagcttggagccgaccatcgacccgcgcacggccgacgagatcaacctggcgggtcgttgggagatcagcctggaggaaaccattgttctggccttgcagaatcgtcaagaactgcgtcaacagctgctgcaacgtgaggtggatggctaccaggagcgcatcgcgttggcggcagtccgcccactggtgagcgtctttgcgaattatgacgtcctggaggtatttgacgatagcttgggcccagcggatggtttgactgtcggtgctcgtatgcgttggaacttcttcgacggcggtgctgcggcagcgcgtgccaaccaggaacaagtggatcaggccatcgcggagaatcgctttgcaaaccaacgcaaccagattcgtctggcagtcgaaaccgcatattacgacttcgaagcgagcgaacagaacattaccacggccgcagcggccgtaacgttagcagaagaaagcctggacgcgatggaggctggttactccgttggtacccgcactatcgttgatgtcctggatgcgacgacgggcctgaatacggcccggggtaactacctgcaagcggttaccgattacaaccgtgcgttcgcgcagctgaagcgtgaagttggcctgggcgacgccgtcattgcgcctgcggctccgtaa

SEQ ID NO: 86 - protein, 591 aa, Artificial Sequence
  Description of Artificial Sequence Synthetic polypeptide
MFAFRDFLTFSTGGLVVLSGGGVAIAQTTPPQIATPEPFIGQTPQAPLPPLAAPSVESLDTAAFLPSLGGLSQPTTLAALPLPSPELNLSPTAHLGTIQAPSPLLAQVDTTATPSPTTAIDVTLPTAETNQTIPLVQPLPPDRVINEDLNQLLEPIDNPAVTVPQEATAVTTDNVVDLTLEETIRLALERNETLQEARLNYDRSEELVREAIAAEYPNLSNQVDITRTDSANGELQARRLGGDNNATTAINGRLEVSYDIYTGGRRSAQIEAAQTQLQIAELDIERLTEETRLAAAVNYYNLQSADAQVVIEQSSVFDATQQLDQTTQRFNVGLVAITDVQNARAELASAQQRLTRAEATQRTARRQLAQLLSLEPTIDPRTADEINLAGRWEISLEETIVLALQNRQELRQQLLQREVDGYQERIALAAVRPLVSVFANYDVLEVFDDSLGPADGLTVGARMRWNFFDGGAAAARANQEQVDQAIAENRFANQRNQIRLAVETAYYDFEASEQNITTAAAAVTLAEESLDAMEAGYSVGTRTIVDVLDATTGLNTARGNYLQAVTDYNRAFAQLKREVGLGDAVIAPAAP

SEQ ID NO: 87 - DNA, 1605 bp, Artificial Sequence
  Description of Artificial Sequence Synthetic polynucleotide
atggcggccttcttgtaccgcctgagcttcctgagcgcgctggcaatcgcggctcacggcgttaccccaccgaccgccatcgctgagctcgcggaggcgaccaccgcagaaccaaccccgaccgtcgcccaagctacgaccccaccggctaccacgccgacgaccaccccggctcctggcccggtcaaagaagtcgtgccggacgcgaatctgctgaaggagctgcaagccaacccgaacccgttccagctgccgaaccagccgaatcaggtgaaaaccgaggccctgcaaccgttgaccctcgagcaggctctgaatctggcgcgtttgaataacccgcagattcaggtgcgtcagctgcaagttcagcaacgccaggcggcattgcgtggtacggaagcagccctgtaccctactctgggcctgcaaggtacggcaggctatcagcaaaacggcacgcgcttgaacgtgaccgagggtaccccgacgcagccgaccggcagctccctgttcacgaccctgggtgagagcagcatcggcgcaaccctgaacctgaattacacgatttttgatttcgtccgtggtgcacaactggcggccagccgtgaccaggtgacgcaggcggaattggatctggaggcggcactggaggacctgcaactgactgtttcggaagcgtactatcgtttgcagaatgcggatcaattggtccgcatcgctcgcgagtctgtcgtcgcgtccgagcgtcagttggatcagaccacccaacgctttaatgttggcctggtggcgatcacggatgtgcaaaatgcccgtgcccagctggcacaagaccagcagaatctggtcgactcgatcggtaaccaggacaaggcgcgtcgcgcgctggttcaggcactgaacctgccgcagaatgttaatgtcctgaccgctgatccggttgaactggctgcgccgtggaatctgagcctggatgagtctattgttctggctttccagaaccgtccggagctggagcgcgaggtgttgcaacgtaacattagctataaccaagcgcaagcagctcgcggtcaagttctgccgcagctgggtctgcaagcgagctacggcgtcaacggtgccatcaattctaatctgcgtagcggtagccaagcgctgaccttcccgagcccgactctgacgaacacgagcagctataactactccattggtctggttttgaatgtgccgctgtttgacggcggtctggcgaacgcgaacgcacagcaacaggaattgaacggtcagattgctgaacaaaactttgtgctgacccgcaatcagattcgtacggacgtcgagactgccttttacgacctgcaaaccaatctggcaaatatcggtaccacccgtaaagcggtggaacaagctcgtgaaagcctggacgcgatggaagcgggttatagcgtgggtacccgtaccattgttgacgttctggatgccacgacggatctgacccgtgcagaggcgaatgcgctgaatgccatcaccgcgtataacctggcactggcgcgtattaagcgcgcagtgagcaacgttaacaacctggcgcgtgcgggtggctaa

SEQ ID NO: 88 - protein, 534 aa, Artificial Sequence
  Description of Artificial Sequence Synthetic polypeptide
MAAFLYRLSFLSALAIAAHGVTPPTAIAELAEATTAEPTPTVAQATTPPATTPTTTPAPGPVKEVVPDANLLKELQANPNPFQLPNQPNQVKTEALQPLTLEQALNLARLNNPQIQVRQLQVQQRQAALRGTEAALYPTLGLQGTAGYQQNGTRLNVTEGTPTQPTGSSLFTTLGESSIGATLNLNYTIFDFVRGAQLAASRDQVTQAELDLEAALEDLQLTVSEAYYRLQNADQLVRIARESVVASERQLDQTTQRFNVGLVAITDVQNARAQLAQDQQNLVDSIGNQDKARRALVQALNLPQNVNVLTADPVELAAPWNLSLDESIVLAFQNRPELEREVLQRNISYNQAQAARGQVLPQLGLQASYGVNGAINSNLRSGSQALTFPSPTLTNTSSYNYSIGLVLNVPLFDGGLANANAQQQELNGQIAEQNFVLTRNQIRTDVETAFYDLQTNLANIGTTRKAVEQARESLDAMEAGYSVGTRTIVDVLDATTDLTRAEANALNAITAYNLALARIKRAVSNVNNLARAGG

SEQ ID NO: 89 - DNA, 494 bp, Artificial Sequence
  Description of Artificial Sequence Synthetic polynucleotide
atgaaaatcctcctaagaaattatgtaagcagacagttttattgttcatgatgatatatttttatcttgtgcaatgtaacatcagagattttgagacacaacgtggctttccccccccccccctgtggaagtacatacgtgttgcctggcttttacgagatcgtaagcgttttacgatgtctttgtcgccttatattgcccttcaagagtttgcaacattagaactttggaggaggtgctacaattttgatgacgacactgatgcggcattggatcttatccgcccctatattatgcatttatacccccacaatcatgtcaagaattcaagcatcttaaataatgttaattatcggcaaagtctgtgctccccttctataatgctgaattgagcattcgcctcctgaacggtctttattcttccattgtgggtctttagattcacgattcttcacaatcattgatctaaagatctttcttaggaggattttcat

SEQ ID NO: 90 - DNA, 494 bp, Artificial Sequence
  Description of Artificial Sequence Synthetic polynucleotide
atgaaaatcctcctaagaaattatgtaagcagacagttttattgttcatgatgatatatttttatcttgtgcaatgtaacatcagagattttgagacacaacgtggctttccccccccccccctgccacacgttttgttcgcagcaggagttacggtcgggtttggaacgtagcgcagcgcaggcgaaattttctctgcacatctatgcgtccgcattaggatggatgcgcaagtaccccaaaattatgttaaatcaacactttacgtagtaggtgatacgggagctgccagctatactaatgatccactatcttgactagcaatttcatagagaaaactctccgggtcatgcactcaaaaaccctttatacgctcacctgcgtctcatgttttggtccaatcgaagaacggctcccataacgggaatgttgacaattaatcatcggcatagtatatcggcatagtataatacgtttcttaggaggattttcat

SEQ ID NO: 91 - DNA, 494 bp, Artificial Sequence
  Description of Artificial Sequence Synthetic polynucleotide
atgaaaatcctcctaagaaagatctttagatcaatgattgtgaagaatcgtgaatctaaagacccacaatggaagaataaagaccgttcaggaggcgaatgctcaattcagcattatagaaggggagcacagactttgccgataattaacattatttaagatgcttgaattcttgacatgattgtgggggtataaatgcataatataggggccaccatcatgttatgtccccagagacagtggttttgtgtggattaccagtgacacgagtcgggcgttcaaactagccgccgtaatatagtacgtatcagttcattgcgagagctttggtgaggatcgcatggctccgaagctcgggaacgacaggccacgggttacccgcttcggcctagtataagagtccgtactgagtccttatggcaggcagtgttgacaattaatcatcggcatagtatatcggcatagtataatacgtttcttaggaggattttcat

SEQ ID NO: 92 - DNA, 494 bp, Artificial Sequence
  Description of Artificial Sequence Synthetic polynucleotide
atgaaaatcctcctaagaaagagggtacaaacaagcccggtgttgtaaacaaagggtcagcccaacgccgacaacatctgcttacctcaccgggcaacgaagggaaacgcctattataagaataatgcttgaatctctcctattagcctccgccagcttcggtagtcttactcatgggtgcggcctcgtctaacagttggcgagggcatcgccactaccatgctgtgcggtgagcccactaacacgttaaagcacgaactacgtagacgagagattccaccttcatgctagatagatgtgatcggcgctagttctcagaccatgcgcacccagcagatacaccactccagggactccctattggtcgttcggaataagacgctattgaggtccacctggctagaccagtctgcttcacaatcaagtatgttgacaattaatcatcggcatagtatatcggcatagtataatacgtttcttaggaggattttcat

SEQ ID NO: 93 - DNA, 508 bp, Artificial Sequence
  Description of Artificial Sequence Synthetic polynucleotide
atgatcacttgtattactgtttatgtaagcagacagttttattgttcatgatgatatatttttatcttgtgcaatgtaacatcagagattttgagacacaacgtggctttcccccccccccccttaattaattggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagcttgcccctatattatgcatttatacccccacaatcatgtcaagaattcaagcatcttaaataatgttaattatcggcaaagtctgtgctccccttctataatgctgaattgagcattcgcctcctgaacggtctttattcttccattgtgggtctttagattcacgattcttcacaatcattgatctaaggatctttgtagattctctgtacat

SEQ ID NO: 94 - DNA, 546 bp, Artificial Sequence
  Description of Artificial Sequence Synthetic polynucleotide
atgatcagagaatctacaaagatccttagatcaatgattgtgaagaatcgtgaatctaaagacccacaatggaagaataaagaccgttcaggaggcgaatgctcaattcagcattatagaaggggagcacagactttgccgataattaacattatttaagatgcttgaattcttgacatgattgtgggggtataaatgcataatataggggcttaattaattggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagcttccaaggtggctacttcaacgatagcttaaacttcgctgctccagcgaggggatttcactggtttgaatgcttcaatgcttgccaaaagagtgctactggaacttacaagagtgaccctgcgtcaggggagctagcactcaaaaaagactcctcctgtacat

SEQ ID NO: 95 - DNA, 612 bp, Artificial Sequence
  Description of Artificial Sequence Synthetic polynucleotide
atgatcaggaggagtcttttttgagtgctagctcccctgacgcagggtcactcttgtaagttccagtagcactcttttggcaagcattgaagcattcaaaccagtgaaatcccctcgctggagcagcgaagtttaagctatcgttgaagtagccaccttggttaattaattggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagctttagtacaaaaagacgattaaccccatgggtaaaagcaggggagccactaaagttcacaggtttacaccgaattttccatttgaaaagtagtaaatcatacagaaaacaatcatgtaaaaattgaatactctaatggtttgatgtccgaaaaagtctagtttcttctattcttcgaccaaatctatggcagggcactatcacagagctggcttaataatttgggagaaatgggtgggggcggactttcgtagaacaatgtagattaaagtactgtacat

SEQ ID NO: 96 - DNA, 419 bp, Artificial Sequence
  Description of Artificial Sequence Synthetic polynucleotide
atgatcacttgtattactgtttatgtaagcagacagttttattgttcatgatgatatatttttatcttgtgcaatgtaacatcagagattttgagacacaacgtggctttcccccccccccccttaattaattggcgcgccgagcatctcttcgaagtattccaggcatcaaataaaacgaaaggctcagtcgaaagactgggcctttcgttttatctgttgtttgtcggtgaacgctctctactagagtcacactggctcaccttcgggtgggcctttctgcgtttataaagcttgggggggggggggaaagccacgttgtgtctcaaaatctctgatgttacattgcacaagataaaaatatatcatcatgaacaataaaactgtctgcttacataaacagtaatacaagtgtacat

SEQ ID NO: 97 - DNA, 300 bp, Artificial Sequence
  Description of Artificial Sequence Synthetic polynucleotide
atgtgacttaactcctgattgaacatcaatatatttttttatggttgcttatttttaataacttttttctaaaaataaaattaagttttataaagaatgattaaaagaattacaaaatataaacataatcttcacataaaaatctttacataaagcgtaattctactaacgacagaaacagggtgccttatgttagcctatagttagatttagtccatataaacaatttagattcagaattgattccctgtttcaatatttcctatccttaccatcaattgtattaaatataggtagcat

SEQ ID NO: 98 - DNA, 243 bp, Artificial Sequence
  Description of Artificial Sequence Synthetic polynucleotide
atgagagagttatcctgaatcaaaatttctttgaaaaaaaaagagaaggaaaaaaaagatatttttaacaacaatgtttgaaattaatatcagttcatctattttgattagaagttgacaatagtttgcaattacaaaaaaagatggacgtttggttgatttttagctattcttgaagtagaaagaaatattctaagaataaagtatagcttaagaattttattgggttaggtaaactgacat

SEQ ID NO: 99 - DNA, 262 bp, Artificial Sequence
  Description of Artificial Sequence Synthetic polynucleotide
atgaattttccctaagttatagtgaacttttttcttgtttattaaaacaaaaaatttgcattttgaaaactgtatttatcccttttcacaaaatattaataatacgtaaattctctcaaaggtttccatacaaaaaacccagagtttctactgagttaattaaccatgacgacataaatatttagtgtcaatcttccgattgagtatcagcttgataaactaggagctaagttccctcatcagcaatttctcaggaaaacat

SEQ ID NO: 100 - DNA, 280 bp, Artificial Sequence
  Description of Artificial Sequence Synthetic polynucleotide
atgtggatatgtcctgatatttgcactcaacagctaaaaatatatttacaattcattgagaattgctatacaattttattctgataagaaggggagtagctgctggcaaaagccagtacatctgaatcaacatactggcgatgagcctggttcaggtgacaactagaaaatatttggaagcgagaccttcactaagttcacatttaagatgtggcttggtggggtcttttggcattcatcaagcttcacatcggtaaacatttttcaggagcttgagcat

SEQ ID NO: 101 - DNA, 480 bp, Artificial Sequence
  Description of Artificial Sequence Synthetic polynucleotide
atgctgtaatccttacacaaagagtgaaaaatcctatgagtgttgtctatcgttggctacaactactttaattttgcaacaccaaaatcacgtttatagtgttttctagtctgctggcgtgccaatttatctgcgtccatctggggttaagtgtttcttgttctcatttactgcgtcgtgcgtatctgtcgggagttgtcatgtcagtggtttttgacctggtttaatgctctatccccttgtggtgtatttttagatggctatcactatatgacgttttcatcgccatcccatagaaacttttactcagagaaactttgttttatgttcgactgtaggcgatgatttccggtcggtagcagacggaggctgcgttaatgccaatactcagcatacgaaactctggcaattatggaaaataatatatgtaagtcgagtatcgtaagactcacttgatttcctcatttcctctaggaacat

SEQ ID NO: 102 - DNA, 373 bp, Artificial Sequence
  Description of Artificial Sequence Synthetic polynucleotide
atgagaactagcacctagattggaggagattacagtcatggacaaattctgcgatcggacttgaggactatcgttactgtagcgtcaaggcaacgagaaacaagaggtactgttttgctcaaaagctgattgaacgctcactccttgatcactgtgctaactggctcttgctctgaatgttactgagcatttctaaacccagaagccaatagaaacgggtgatatatctaaagctgttgaaaacagcattgttcattggcagccctagagtcagcgagacagtgcttcgtagctgctcagctagattctgtccggctgagttcattgtctgacccaagctcaatttccctttgccctaaggactggtggccat

SEQ ID NO: 103 - DNA, 356 bp, Artificial Sequence
  Description of Artificial Sequence Synthetic polynucleotide
atgaaccaatccttatggtcatggggctccaaatcttcagctggttttacccagtgagtttgaagcaaggatcttttagtttaccgaaaaatgaggctcagcgatcgcagcaagttcttgccgactgaggaggcgatcgcggcagcagtgtttgcccgaggtggtcaaaggagcagttttggtaaaagtctaaaggaaatataaagactgctgccttgcgggacgagcaatggacttctctaccctagggaaaactgatttagaagtgaactaatcgcatagatgatttaatgcgtaccttcttttccactaactactattggaattaaaggacacttaaatttaggaatcgacat

SEQ ID NO: 104 - DNA, 264 bp, Artificial Sequence
  Description of Artificial Sequence Synthetic polynucleotide
atgaactcctcaaaccacagaaattgttaacgccaatcttactagaactaggctggctttgcccacggccagggatgggcttaccctggggataaatagttttttggtattaaactaaacaggccgtaacggacaatacggaaattgtcgctcccaaaacacaaaatagtcagcacatcgacataattgacggcgatcgcctaaattactagagttgaggccagttttgccgttgcctttttttcttttgtgtgaggagtccat

SEQ ID NO: 105 - DNA, 363 bp, Artificial Sequence
  Description of Artificial Sequence Synthetic polynucleotide
atgtttgaccaacctttatctctggatttcactggaaaatggatctaatcaccccaaaaatccctttaaaaaacttaacaaatacggaactccccaccggcaaaaaccctatgccccccgtcccaacctgtacaatgaagagggcggagacgtaagtttccgttcactcctcacaccacactccgcctggatgatgttcgggcggtttcttcttatctgctccccagggggaaaagtgtgacgccaactgtgacaaaagatgaataaattctaagtttcacgatatttttccatacaggggtcaacaattggttatggtagtattctaatcagcccatcacgaggtttagaaggatttcccat

SEQ ID NO: 106 - DNA, 412 bp, Artificial Sequence
  Description of Artificial Sequence Synthetic polynucleotide
atgcgttgttcctctttaacagtgactgtgccgaatagagcaatctctacgggcaacctttgcaatgggtagtgtgaacgctacgattccccgcaaatggggcaaaattgagcagtgcaaaactcagcgagatgatgcaaccatccgcaagcctgtgatattgtcgtaggtcttatgcttaggatcagcttagttgatacccaatgcaataactgttgctttggagattcttaattattctataggtttgggttatcaatctttagagttgtttataggtttctaattagaggtgtacaactatagtctcccttctattcaacaggcactgatgattgcctgaaatcaatttaatggtcctcatggggggcgatcgctctattgtttttgaaaaaaagggggtggaattcat

SEQ ID NO: 107 - DNA, 317 bp, Artificial Sequence
  Description of Artificial Sequence Synthetic polynucleotide
atgtgtttctatcctcacaccataactcccgcgtagggaatgactaaccctacagccactgagagtctgtgattcaatgtatatcactctatgttcagtcctagggtcaacattcggttcttggtaaaacctgctagagtggcactacagccctttccaagatatacagtccatccaggggaggtctttcttccccagagggcctctggcggttttgagcgggtttcatttccgtaaaaagggcggtagattgactgtggttgccctctttctgaacggggcaaggccatttttgttggtgtgaggtcgagggtcat

SEQ ID NO: 108 - DNA, 294 bp, Artificial Sequence
  Description of Artificial Sequence Synthetic polynucleotide
atgtaataataaccctgaaagtaaccctaagtctgatgatcaagtttcgctatccttaaaaaattctcaatttggtcaaattaaggaaagtggaagtagaattagagtagtagatcctaaagataccacatttgaaaggtatgatggtgatccacctgcacaacgttaattgtaagctaatggttattgattttaaaagttgggttttcttttaccccaacttttagtcaactttaataatacgataaaacattgcaaaatactaatatgatttttaaaatttaggtttccata

SEQ ID NO: 109 - DNA, 296 bp, Artificial Sequence
  Description of Artificial Sequence Synthetic polynucleotide
atgttattgaagaccttttataatataaaaattaccatacttgtgagatacaaaagtgatctcgaagagatccgcttcgcggtgcgctttgaggcagagagaggtgttaggtttaccttatgagtccgagaaaccctatataaatcctattatcataatatcaactaaacttgtgagttatcaatgtctggaaaaagaggcgatcgctgatcatggatcatggtcaaacttatagtaatctaacattaaggctcattactttcattataattccatgttaagtttaagggtaacat

SEQ ID NO: 110 - DNA, 483 bp, Artificial Sequence
  Description of Artificial Sequence Synthetic polynucleotide
atgaatatcttggcctgtgagttcttcccttttaagagtctgccacctgaataggatgtcttgcaagctcaagattagttagttaaccgttgacagttaacggttaactaagtccaatgtcaagatttctgagaaaagttgtgtcagattgtaaaatttctgatattcatagtatttaataggttcgtgtttaatggttgattcacattggatggattaagcaaaagccgaactaatatggtaagttaagaatcattaagttaccacacgctaggtgactagctgatggtgcgtgtaaagacataactctgagaaaagccaatttaactaattggtagcctctcaggaactcagaagttttaagacaactgagaatgtcaaaaaaaacgttatttcctcgcggtagttgccaaaagttgggaaacccagctaaagcactgcttaaagacgttgcaatttttagtaaaagaggattttagtcat

SEQ ID NO: 111 - DNA, 999 bp, Artificial Sequence
  Description of Artificial Sequence Synthetic polynucleotide
atgatgaaaaagccggttgttattggcctggcggttgtcgtgttggcagccgtggtcgcgggtggttactggtggtatcagagccgccaagataacggtctgactctgtacggtaatgttgatatccgcacggtgaacctgagcttccgtgtcggtggtcgtgtagagtctctggctgtcgacgagggcgatgcgatcaaggcgggtcaggtgttgggcgagttggaccataaaccgtatgaaatcgccctgatgcaagcaaaggcgggtgtcagcgtggcccaggcgcaatacgacctgatgctggcaggttaccgtaatgaggagattgcccaggcagcagcggcggtgaagcaggcccaagcggcatacgattatgcgcaaaacttttacaaccgtcagcaaggtctgtggaaaagccgtacgatctccgcgaatgacttggaaaacgcccgtagcagccgcgaccaagcgcaggctacgctgaaaagcgcgcaggacaaactgcgccagtaccgttctggcaatcgcgaacaagacattgcacaggctaaagccagcctggagcaagcgcaagcccaactggcacaggcggaactgaacttgcaggactcgaccctgattgcgccgagcgacggtaccctgctgacccgtgctgtcgaaccaggcaccgttctgaatgaaggtggcaccgtttttaccgtgagcctgacccgtccggtgtgggtccgcgcttatgttgacgaacgcaatctggatcaggcgcagccgggtcgtaaggttctgctgtataccgatggtcgtccggataagccgtaccacggccaaattggctttgtttcccctacggcagagttcaccccgaaaacggtcgagactccggatttgcgtaccgatctggtttatcgcctgcgtatcgtggttaccgatgcggacgatgcgctgcgtcagggtatgccggtgacggtccaattcggcgacgaggcaggccacgagtaa

SEQ ID NO: 112 - DNA, 1056 bp, Artificial Sequence
  Description of Artificial Sequence Synthetic polynucleotide
atgaacaataacgacttgtttcaggcaagccgccgtcgcttcctggcgcagctgggtggcctgacggtggcaggcatgctgggtccgagcttgctgaccccgcgtcgtgccaccgcgggtggttactggtggtatcagagccgccaagataacggtctgactctgtacggtaatgttgatatccgcacggtgaacctgagcttccgtgtcggtggtcgtgtagagtctctggctgtcgacgagggcgatgcgatcaaggcgggtcaggtgttgggcgagttggaccataaaccgtatgaaatcgccctgatgcaagcaaaggcgggtgtcagcgtggcccaggcgcaatacgacctgatgctggcaggttaccgtaatgaggagattgcccaggcagcagcggcggtgaagcaggcccaagcggcatacgattatgcgcaaaacttttacaaccgtcagcaaggtctgtggaaaagccgtacgatctccgcgaatgacttggaaaacgcccgtagcagccgcgaccaagcgcaggctacgctgaaaagcgcgcaggacaaactgcgccagtaccgttctggcaatcgcgaacaagacattgcacaggctaaagccagcctggagcaagcgcaagcccaactggcacaggcggaactgaacttgcaggactcgaccctgattgcgccgagcgacggtaccctgctgacccgtgctgtcgaaccaggcaccgttctgaatgaaggtggcaccgtttttaccgtgagcctgacccgtccggtgtgggtccgcgcttatgttgacgaacgcaatctggatcaggcgcagccgggtcgtaaggttctgctgtataccgatggtcgtccggataagccgtaccacggccaaattggctttgtttcccctacggcagagttcaccccgaaaacggtcgagactccggatttgcgtaccgatctggtttatcgcctgcgtatcgtggttaccgatgcggacgatgcgctgcgtcagggtatgccggtgacggtccaattcggcgacgaggcaggccacgagtaa

SEQ ID NO: 113 - protein, 351 aa, Artificial Sequence
  Description of Artificial Sequence Synthetic polypeptide
MNNNDLFQASRRRFLAQLGGLTVAGMLGPSLLTPRRATAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDYAQNFYNRQQGLWKSRTISANDLENARSSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 114 - DNA, 1005 bp, Artificial Sequence
  Description of Artificial Sequence Synthetic polynucleotide
atgcgtttcttttggttctttctgacgctgctgaccttgagcacctggcaactgccggcgtgggcaggtggttactggtggtatcagagccgccaagataacggtctgactctgtacggtaatgttgatatccgcacggtgaacctgagcttccgtgtcggtggtcgtgtagagtctctggctgtcgacgagggcgatgcgatcaaggcgggtcaggtgttgggcgagttggaccataaaccgtatgaaatcgccctgatgcaagcaaaggcgggtgtcagcgtggcccaggcgcaatacgacctgatgctggcaggttaccgtaatgaggagattgcccaggcagcagcggcggtgaagcaggcccaagcggcatacgattatgcgcaaaacttttacaaccgtcagcaaggtctgtggaaaagccgtacgatctccgcgaatgacttggaaaacgcccgtagcagccgcgaccaagcgcaggctacgctgaaaagcgcgcaggacaaactgcgccagtaccgttctggcaatcgcgaacaagacattgcacaggctaaagccagcctggagcaagcgcaagcccaactggcacaggcggaactgaacttgcaggactcgaccctgattgcgccgagcgacggtaccctgctgacccgtgctgtcgaaccaggcaccgttctgaatgaaggtggcaccgtttttaccgtgagcctgacccgtccggtgtgggtccgcgcttatgttgacgaacgcaatctggatcaggcgcagccgggtcgtaaggttctgctgtataccgatggtcgtccggataagccgtaccacggccaaattggctttgtttcccctacggcagagttcaccccgaaaacggtcgagactccggatttgcgtaccgatctggtttatcgcctgcgtatcgtggttaccgatgcggacgatgcgctgcgtcagggtatgccggtgacggtccaattcggcgacgaggcaggccacgagtaa

SEQ ID NO: 115 - protein, 334 aa, Artificial Sequence
  Description of Artificial Sequence Synthetic polypeptide
MRFFWFFLTLLTLSTWQLPAWAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDYAQNFYNRQQGLWKSRTISANDLENARSSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 116 - DNA, 1035 bp, Artificial Sequence
  Description of Artificial Sequence Synthetic polynucleotide
atgcagaaacaacaaaatctggactactttagcccgcaggccctggccctgtgggctgcgattgcgagcttgggtgttatgtcccctgcgcatgcgggtggttactggtggtatcagagccgccaagataacggtctgactctgtacggtaatgttgatatccgcacggtgaacctgagcttccgtgtcggtggtcgtgtagagtctctggctgtcgacgagggcgatgcgatcaaggcgggtcaggtgttgggcgagttggaccataaaccgtatgaaatcgccctgatgcaagcaaaggcgggtgtcagcgtggcccaggcgcaatacgacctgatgctggcaggttaccgtaatgaggagattgcccaggcagcagcggcggtgaagcaggcccaagcggcatacgattatgcgcaaaacttttacaaccgtcagcaaggtctgtggaaaagccgtacgatctccgcgaatgacttggaaaacgcccgtagcagccgcgaccaagcgcaggctacgctgaaaagcgcgcaggacaaactgcgccagtaccgttctggcaatcgcgaacaagacattgcacaggctaaagccagcctggagcaagcgcaagcccaactggcacaggcggaactgaacttgcaggactcgaccctgattgcgccgagcgacggtaccctgctgacccgtgctgtcgaaccaggcaccgttctgaatgaaggtggcaccgtttttaccgtgagcctgacccgtccggtgtgggtccgcgcttatgttgacgaacgcaatctggatcaggcgcagccgggtcgtaaggttctgctgtataccgatggtcgtccggataagccgtaccacggccaaattggctttgtttcccctacggcagagttcaccccgaaaacggtcgagactccggatttgcgtaccgatctggtttatcgcctgcgtatcgtggttaccgatgcggacgatgcgctgcgtcagggtatgccggtgacggtccaattcggcgacgaggcaggccacgagtaa

SEQ ID NO: 117 - protein, 344 aa, Artificial Sequence
  Description of Artificial Sequence Synthetic polypeptide
MQKQQNLDYFSPQALALWAAIASLGVMSPAHAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDYAQNFYNRQQGLWKSRTISANDLENARSSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 118 - DNA, 4070 bp, Artificial Sequence
  Description of Artificial Sequence Synthetic polynucleotide
caattgtatataaactgcagtataagtaggaggtaaaatcatgaacgacgcagtaatcaccctgaacggcctggaaaaacgcttcccgggcatggacaaaccggctgttgctccattggactgtaccatccacgccggttacgtgacgggtctggttggtccggatggtgcgggcaaaaccaccttgatgcgtatgctggcgggtctgctgaagccggacagcggctccgcgaccgttatcggttttgacccgattaagaatgacggtgcattgcacgcggttttgggctacatgccgcagaaattcggcctgtacgaagatctgaccgtcatggaaaatctgaatctgtatgctgatttgcgctctgttacgggtgaggcgcgtaaacaaacctttgcgcgtttgctggaatttacctctctgggcccgtttacgggtcgtctggcgggtaagctgagcggtggtatgaagcagaaactgggtttggcatgcaccctggtgggcgagccgaaagtcctgctgctggatgagccgggtgtgggcgtcgatccgattagccgtcgtgagctgtggcagatggtccacgaactggctggcgaaggcatgttgatcctgtggagcaccagctatctggatgaagcggagcagtgccgtgatgttctgttgatgaatgagggcgagctgctgtaccaaggcgaaccaaaagcgctgacccaaacgatggcgggtcgcagcttcctgatgaccagcccgcatgagggcaaccgtaaactgctgcaacgcgcattgaaactgccgcaagtcagcgacggcatgattcagggcaaatccgttcgtctgattctgaagaaagaggcaaccccggacgacattcgtcatgcagatggcatgcctgaaatcaatatcaacgaaacgaccccgcgtttcgaggatgccttcatcgatctgctgggtggtgccggtacctctgagagcccgctgggcgcaatcctgcataccgtggaaggtactccgggtgagactgttattgaagcgaaggagctgacgaaaaagttcggtgactttgccgcgaccgatcacgtgaatttcgcggtcaaacgtggtgagatcttcggcctgctgggtcctaacggtgcaggtaaatccaccacttttaagatgatgtgtggtctgttggtgccaacgagcggtcaggcgctggtcctgggtatggacctgaaggaaagcagcggcaaagctcgccaacacctgggttacatggcacaaaagttttctctgtacggcaatttgacggtggagcagaacttgcgctttttcagcggtgtgtatggtctgcgtggtcgcgcccaaaatgaaaagattagccgcatgagcgaagcgttcggtctgaaaagcatcgcgagccacgcaaccgacgagttgccgctgggtttcaaacaacgcctggcgctggcctgtagcctgatgcacgagccggatattctgtttctggacgagccgaccagcggtgtcgatccgctgacgcgtcgtgagttctggctgcacattaacagcatggtcgaaaagggcgttaccgtgatggttactacgcatttcatggacgaagccgagtattgcgatcgtatcggcctggtgtatcgtggcaagttgattgcgtccggtacgccggatgatctgaaggcacagtcggcgaacgacgagcagccggacccgacgatggaacaggcctttatccagctgattcacgactgggacaaggagcatagcaacgagtaaggatcctcaagtaggaggtactagtaatgagcaatccaatcctgagctggcgtcgcgtccgtgcactgtgcgtgaaagaaactcgccaaatcgtccgcgacccgagctcctggctgatcgccgttgtgattccgctgctgctgttgttcatcttcggctatggtatcaacctggatagcagcaaactgcgcgtcggtattctgctggagcagcgtagcgaagctgccctggacttcacccacaccatgacgggctccccgtatatcgacgctaccatttctgataatcgtcaggaactgattgcgaagatgcaagcgggcaagattcgcggtctggttgttattccggttgacttcgcagagcaaatggagcgtgccaatgcgaccgccccaattcaggtgattaccgacggtagcgaaccgaataccgcgaactttgttcaaggttacgtagaaggtatttggcaaatctggcagatgcaacgtgcagaggacaacggtcagaccttcgaaccgctgattgatgtgcagacccgttactggtttaaccctgcggccattagccaacatttcatcatcccgggtgccgtcaccatcattatgacggttatcggcgcgattctgacgagcttggttgtggcgcgtgaatgggagcgtggtacgatggaggcattgctgagcacggagatcacccgtaccgagttgctgttgtgcaagctgattccgtactatttcctgggcatgctggcgatgctgctgtgtatgttggtcagcgtgttcatcctgggcgtgccgtatcgtggtagcctgctgatcttgttctttatctctagcttgtttctgctgtctaccctgggtatgggtctgctgattagcaccatcacgcgcaaccagtttaacgcagcacaggtcgcgctgaacgcggcgtttctgccgagcatcatgctgagcggttttatctttcagattgattccatgccggctgttatccgtgcggtcacttacattattcctgcgcgctacttcgtgtcgacgttgcaaagcctgttcctggcaggcaatattccggtcgtgctggtggttaatgttctgttcctgattgcatccgcggttatgtttatcggcctgacgtggctgaaaaccaaacgccgtctggattaactcgagactcataggaggacatctagatgtttcatagattatggacactaatcagaaaagaactgcaatccctgctgcgtgaacctcagacgcgtgcgatcctgatcttgccggtgctgattcaggtcatcctgttcccgtttgccgctaccttggaagtcacgaatgccactattgcgatctacgacgaggataacggtgaacacagcgtcgagctgacccagcgtttcgcgcgtgcctctgcttttacccacgtgctgttgctgaaaagcccgcaggaaattcgcccgacgattgatacgcaaaaggcgctgctgctggttcgctttccggccgactttagccgtaagctggacacctttcagaccgcacctctgcaactgatcctggatggccgcaactcgaatagcgcgcagattgctgcgaattacctgcaacaaattgtgaaaaactatcagcaagagctgctggagggtaaaccgaagccaaataactccgagctggttgtccgtaactggtataatccgaatttggactataagtggttcgtggttccgagcctgattgcgatgattaccaccattggtgtgatgattgttaccagcttgagcgttgcacgtgaacgtgagcaaggtacgctggatcaactgctggtttctccgctgaccacctggcagattttcatcggtaaagctgttccggcgttgatcgtagcgacctttcaggcgaccatcgtgctggcaatcggtatctgggcgtaccagatcccgttcgccggcagcctggcgctgttctacttcacgatggtgatttatggtctgagcctggtcggcttcggtctgctgattagcagcctgtgcagcacccagcaacaggccttcattggcgtgttcgtgtttatgatgccggcaatcttgctgtcgggctacgtcagcccagtcgagaatatgccggtttggttgcaaaacctgacgtggatcaacccgatccgtcattttacggacatcacgaagcagatttatctgaaagatgcaagcctggacattgtttggaactccctgtggccgctgctggtcatcaccgcaactaccggcagcgcggcatacgctatgttccgccgcaaggttatgtaa

SEQ ID NO: 119 - DNA, 4646 bp, Artificial Sequence
  Description of Artificial Sequence Synthetic polynucleotide
caattgtatataaactgcagtataagtaggaggtaaaatcatgaacgacgcagtaatcaccctgaacggcctggaaaaacgcttcccgggcatggacaaaccggctgttgctccattggactgtaccatccacgccggttacgtgacgggtctggttggtccggatggtgcgggcaaaaccaccttgatgcgtatgctggcgggtctgctgaagccggacagcggctccgcgaccgttatcggttttgacccgattaagaatgacggtgcattgcacgcggttttgggctacatgccgcagaaattcggcctgtacgaagatctgaccgtcatggaaaatctgaatctgtatgctgatttgcgctctgttacgggtgaggcgcgtaaacaaacctttgcgcgtttgctggaatttacctctctgggcccgtttacgggtcgtctggcgggtaagctgagcggtggtatgaagcagaaactgggtttggcatgcaccctggtgggcgagccgaaagtcctgctgctggatgagccgggtgtgggcgtcgatccgattagccgtcgtgagctgtggcagatggtccacgaactggctggcgaaggcatgttgatcctgtggagcaccagctatctggatgaagcggagcagtgccgtgatgttctgttgatgaatgagggcgagctgctgtaccaaggcgaaccaaaagcgctgacccaaacgatggcgggtcgcagcttcctgatgaccagcccgcatgagggcaaccgtaaactgctgcaacgcgcattgaaactgccgcaagtcagcgacggcatgattcagggcaaatccgttcgtctgattctgaagaaagaggcaaccccggacgacattcgtcatgcagatggcatgcctgaaatcaatatcaacgaaacgaccccgcgtttcgaggatgccttcatcgatctgctgggtggtgccggtacctctgagagcccgctgggcgcaatcctgcataccgtggaaggtactccgggtgagactgttattgaagcgaaggagctgacgaaaaagttcggtgactttgccgcgaccgatcacgtgaatttcgcggtcaaacgtggtgagatcttcggcctgctgggtcctaacggtgcaggtaaatccaccacttttaagatgatgtgtggtctgttggtgccaacgagcggtcaggcgctggtcctgggtatggacctgaaggaaagcagcggcaaagctcgccaacacctgggttacatggcacaaaagttttctctgtacggcaatttgacggtggagcagaacttgcgctttttcagcggtgtgtatggtctgcgtggtcgcgcccaaaatgaaaagattagccgcatgagcgaagcgttcggtctgaaaagcatcgcgagccacgcaaccgacgagttgccgctgggtttcaaacaacgcctggcgctggcctgtagcctgatgcacgagccggatattctgtttctggacgagccgaccagcggtgtcgatccgctgacgcgtcgtgagttctggctgcacattaacagcatggtcgaaaagggcgttaccgtgatggttactacgcatttcatggacgaagccgagtattgcgatcgtatcggcctggtgtatcgtggcaagttgattgcgtccggtacgccggatgatctgaaggcacagtcggcgaacgacgagcagccggacccgacgatggaacaggcctttatccagctgattcacgactgggacaaggagcatagcaacgagtaaggatcctcaagtaggaggtactagtaatgcaagcaccaacgcaaagcggcggtctgagcctgagaaacaaagcggtcctgattgcactgctgatcggcctgattccggcaggcgttattggtggtttgaatctgagcagcgttgatcgtctgccggtccctcaaaccgagcagcaggtcaaagatagcaccaccaagcagattcgtgaccagattctgatcggtctgctggtgaccgcagtgggtgcagcgttcgtcgcgtattggatggttggtgagaacaccaaagcgcaaaccgcgctggcgctgaaggctaagtccaatccgattctgagctggcgccgtgtacgcgcgctgtgtgtgaaggaaacccgtcagattgtgcgtgatccgagctcgtggctgattgcggtcgtcatcccgttgttgctgctgttcatttttggctacggtatcaacctggatagcagcaaattgcgcgttggtattttgctggagcagcgtagcgaagcggcgctggattttacccataccatgacgggcagcccgtacattgacgccaccattagcgacaatcgtcaggaactgattgcgaagatgcaagccggtaagatccgtggcctggttgtgatcccggtcgactttgcggagcaaatggagcgcgcgaatgcgaccgcaccgatccaagtcatcacggacggcagcgagccgaacaccgctaacttcgttcagggttatgtcgagggtatctggcaaatttggcagatgcaacgtgcggaggataatggccagaccttcgaaccgctgatcgacgttcagactcgttactggttcaatccagccgctatcagccagcacttcatcattccgggtgcggttacgatcattatgacggtaatcggtgcgattctgacgtccctggttgtcgcccgtgagtgggaacgtggtacgatggaggcactgctgtctaccgaaattacgcgtacggaactgttgctgtgcaaattgatcccgtactacttcctgggtatgttggccatgctgctgtgcatgctggtgagcgtgttcatcctgggtgtgccgtatcgtggttctctgctgatcctgtttttcatctctagcctgtttttgctgtccactctgggcatgggcctgctgattagcactatcacccgcaaccagtttaatgcggcccaggtggccctgaacgcagcatttttgccgagcatcatgctgtccggtttcatctttcaaattgatagcatgccggcagtgatccgcgctgttacctatatcattcctgctcgttacttcgttagcacgctgcaatcgctgttcttggcgggcaacattccggtcgtgctggttgttaacgtgctgtttctgattgccagcgctgtgatgtttattggcctgacctggctgaaaacgaaacgccgcctggactaactcgagactcataggaggacatctagatgcaagcaccaacccaatccggcggcctgagcctgcgcaacaaagcggttctgatcgcgttgctgattggtctgattccggcaggtgtgattggtggcctgaatctgtctagcgtggatcgcctgccggtgccgcagactgaacagcaggtgaaggactccacgaccaagcaaattcgtgaccagattctgattggcctgttggttactgccgtgggtgcggcatttgtcgcgtattggatggttggtgaaaataccaaagcgcaaaccgcgctggctctgaaggcgaaatttcatcgtctgtggaccctgatccgtaaggagctgcaaagcctgttgcgtgagccgcagacccgtgctattctgattctgccggtcttgatccaagtgatcctgttcccgtttgccgctaccctggaagtgacgaatgccacgattgccatttacgatgaggacaatggtgagcactccgttgaactgacccaacgttttgcacgtgcgtccgctttcacccatgtgctgctgttgaaatctccgcaggagattcgtccgaccattgatacgcagaaggcgctgctgctggtgcgctttcctgctgacttcagccgtaagctggacaccttccagaccgcgccattgcagctgatcctggatggccgcaattctaatagcgcacagatcgccgcaaactatctgcaacagattgtgaaaaactaccagcaagaactgctggagggtaaaccgaaaccgaacaatagcgaactggtcgtccgtaactggtataacccgaacctggactacaaatggttcgttgtcccgagcctgatcgcgatgattaccaccatcggcgttatgatcgtcaccagcctgagcgtagcacgtgagcgcgagcaaggcaccctggatcaactgttggtgagccctctgactacgtggcagatcttcatcggtaaggcggttccggcactgatcgtcgccacgttccaggcgaccatcgttttggcaatcggtatttgggcgtatcaaatcccgttcgcgggtagcctggccctgttttacttcacgatggttatctacggcttgagcctggttggcttcggtttgctgattagcagcctgtgcagcacccagcaacaggcgtttatcggtgtttttgtgtttatgatgccggcgattctgctgagcggttacgtcagcccggtcgagaacatgccggtgtggctgcaaaacctgacgtggatcaatccgatccgccacttcacggatattaccaagcagatctacctgaaagacgcgagcctggacattgtctggaacagcttgtggccgttgctggttatcaccgcgacgacgggttcggcagcgtatgccatgttccgccgtaaggtaatgtaa

SEQ ID NO: 120 - protein, 473 aa, Artificial Sequence
  Description of Artificial Sequence Synthetic polypeptide
MQAPTQSGGLSLRNKAVLIALLIGLIPAGVIGGLNLSSVDRLPVPQTEQQVKDSTTKQIRDQILIGLLVTAVGAAFVAYWMVGENTKAQTALALKAKSNPILSWRRVRALCVKETRQIVRDPSSWLIAVVIPLLLLFIFGYGINLDSSKLRVGILLEQRSEAALDFTHTMTGSPYIDATISDNRQELIAKMQAGKIRGLVVIPVDFAEQMERANATAPIQVITDGSEPNTANFVQGYVEGIWQIWQMQRAEDNGQTFEPLIDVQTRYWFNPAAISQHFIIPGAVTIIMTVIGAILTSLVVAREWERGTMEALLSTEITRTELLLCKLIPYYFLGMLAMLLCMLVSVFILGVPYRGSLLILFFISSLFLLSTLGMGLLISTITRNQFNAAQVALNAAFLPSIMLSGFIFQIDSMPAVIRAVTYIIPARYFVSTLQSLFLAGNIPVVLVVNVLFLIASAVMFIGLTWLKTKRRLD

SEQ ID NO: 121 - protein, 464 aa, Artificial Sequence
  Description of Artificial Sequence Synthetic polypeptide
MQAPTQSGGLSLRNKAVLIALLIGLIPAGVIGGLNLSSVDRLPVPQTEQQVKDSTTKQIRDQILIGLLVTAVGAAFVAYWMVGENTKAQTALALKAKFHRLWTLIRKELQSLLREPQTRAILILPVLIQVILFPFAATLEVTNATIAIYDEDNGEHSVELTQRFARASAFTHVLLLKSPQEIRPTIDTQKALLLVRFPADFSRKLDTFQTAPLQLILDGRNSNSAQIAANYLQQIVKNYQQELLEGKPKPNNSELVVRNWYNPNLDYKWFVVPSLIAMITTIGVMIVTSLSVAREREQGTLDQLLVSPLTTWQIFIGKAVPALIVATFQATIVLAIGIWAYQIPFAGSLALFYFTMVIYGLSLVGFGLLISSLCSTQQQAFIGVFVFMMPAILLSGYVSPVENMPVWLQNLTWINPIRHFTDITKQIYLKDASLDIVWNSLWPLLVITATTGSAAYAMFRRKVM

SEQ ID NO: 122 - DNA, 4760 bp, Artificial Sequence
  Description of Artificial Sequence Synthetic polynucleotide
caattgtatataaactgcagtataagtaggaggtaaaatcatgaacgacgcagtaatcaccctgaacggcctggaaaaacgcttcccgggcatggacaaaccggctgttgctccattggactgtaccatccacgccggttacgtgacgggtctggttggtccggatggtgcgggcaaaaccaccttgatgcgtatgctggcgggtctgctgaagccggacagcggctccgcgaccgttatcggttttgacccgattaagaatgacggtgcattgcacgcggttttgggctacatgccgcagaaattcggcctgtacgaagatctgaccgtcatggaaaatctgaatctgtatgctgatttgcgctctgttacgggtgaggcgcgtaaacaaacctttgcgcgtttgctggaatttacctctctgggcccgtttacgggtcgtctggcgggtaagctgagcggtggtatgaagcagaaactgggtttggcatgcaccctggtgggcgagccgaaagtcctgctgctggatgagccgggtgtgggcgtcgatccgattagccgtcgtgagctgtggcagatggtccacgaactggctggcgaaggcatgttgatcctgtggagcaccagctatctggatgaagcggagcagtgccgtgatgttctgttgatgaatgagggcgagctgctgtaccaaggcgaaccaaaagcgctgacccaaacgatggcgggtcgcagcttcctgatgaccagcccgcatgagggcaaccgtaaactgctgcaacgcgcattgaaactgccgcaagtcagcgacggcatgattcagggcaaatccgttcgtctgattctgaagaaagaggcaaccccggacgacattcgtcatgcagatggcatgcctgaaatcaatatcaacgaaacgaccccgcgtttcgaggatgccttcatcgatctgctgggtggtgccggtacctctgagagcccgctgggcgcaatcctgcataccgtggaaggtactccgggtgagactgttattgaagcgaaggagctgacgaaaaagttcggtgactttgccgcgaccgatcacgtgaatttcgcggtcaaacgtggtgagatcttcggcctgctgggtcctaacggtgcaggtaaatccaccacttttaagatgatgtgtggtctgttggtgccaacgagcggtcaggcgctggtcctgggtatggacctgaaggaaagcagcggcaaagctcgccaacacctgggttacatggcacaaaagttttctctgtacggcaatttgacggtggagcagaacttgcgctttttcagcggtgtgtatggtctgcgtggtcgcgcccaaaatgaaaagattagccgcatgagcgaagcgttcggtctgaaaagcatcgcgagccacgcaaccgacgagttgccgctgggtttcaaacaacgcctggcgctggcctgtagcctgatgcacgagccggatattctgtttctggacgagccgaccagcggtgtcgatccgctgacgcgtcgtgagttctggctgcacattaacagcatggtcgaaaagggcgttaccgtgatggttactacgcatttcatggacgaagccgagtattgcgatcgtatcggcctggtgtatcgtggcaagttgattgcgtccggtacgccggatgatctgaaggcacagtcggcgaacgacgagcagccggacccgacgatggaacaggcctttatccagctgattcacgactgggacaaggagcatagcaacgagtaaggatcctcaagtaggaggtactagtaatgttcttaggatggttcaccaacgcatcgctgttccgcaagcaaatctatatggcgattgcgagcggtgtttttagcggctttgctgttctggtgctgggcagcattgtgggtctgggtggtacccctaaggacgttccggcaccgagcggtgaaaccaccaccgaagcaccggcagaaggtgcaccagcggaaggccaagctccgagccagaccccggaagaggaaccgggcaaaccgagcctgctgaacctggcgttcctgacggccattgctacggcgattggtgtctttctgattaaccgcttgctgatgcagcaaatcaaaagcatcattgacgacctgcaaagcaatccgatcctgagctggcgccgtgttcgtgccctgtgcgtgaaggaaacccgtcagattgtgcgtgatccgagctcttggctgatcgcggtcgtcattcctctgctgctgctgttcattttcggttatggtattaacctggatagcagcaaactgcgtgttggtattctgctggaacagcgtagcgaggcggcgttggattttacccataccatgacgggttccccgtacattgacgcgaccatcagcgataaccgccaggagctgatcgcaaagatgcaggccggcaaaattcgtggcctggtggtgattccggttgacttcgcggagcagatggagcgcgcaaacgcaaccgcaccgattcaagtgattaccgatggttccgaaccgaatacggcaaatttcgtgcaaggctatgtggagggtatctggcaaatttggcagatgcaacgcgcggaggataatggccagacctttgaaccgctgatcgacgtccaaactcgttactggtttaatccagcggccatcagccaacactttatcattccgggtgcggtcaccatcattatgacggtcattggcgctatcctgacctctttggtagtcgcccgtgagtgggagcgtggtacgatggaggcgctgctgagcacggagatcactcgtacggaattgctgctgtgcaaactgatcccgtactacttcctgggtatgctggcgatgctgttgtgtatgctggtcagcgttttcattctgggtgtgccataccgcggcagcttgttgattctgttcttcatctcctcgttgtttctgctgtctaccctgggcatgggtctgctgattagcacgatcacccgcaatcagttcaacgcggctcaggtcgcgctgaatgccgccttcctgccgagcatcatgctgagcggctttatctttcagatcgattcgatgccggctgttattcgtgccgttacgtatatcatcccggcacgttacttcgtttccaccttgcagagcctgtttttggccggtaacatcccggtggtgctggttgttaatgtcttgttcctgatcgcgtccgcggttatgtttatcggtctgacttggctgaaaacgaagcgtcgtctggactaactcgagactcataggaggacatctagatgtttttaggctggttcaccaatgcctcgttatttcgcaaacagatctacatggccattgcgagcggtgttttctccggtttcgcggtgctggttctgggttccatcgttggtctgggcggtaccccgaaggacgtccctgcaccgtctggcgaaacgaccacggaggcaccggcggaaggtgctccggcggagggccaagcgccgagccagaccccggaggaagaaccgggcaagccgagcttgttgaatctggccttcttgaccgctatcgccaccgcgatcggtgtctttctgattaaccgtctgctgatgcagcaaatcaagagcatcattgacgatttgcaatttcatcgcctgtggacgctgattcgtaaggagctgcaaagcctgctgcgcgaaccacaaacccgtgccattctgattctgccggtgctgatccaggttattctgttcccgttcgcagcgaccctggaggtgacgaacgccaccattgccatctatgacgaggataacggcgagcacagcgtggagctgacccagcgtttcgctcgtgcaagcgcgtttacgcacgttctgctgctgaaaagcccgcaggagatccgtccgaccattgacactcagaaagcgctgctgctggttcgctttcctgcggattttagccgtaaactggacaccttccagacggcaccgctgcaactgattctggatggtcgtaacagcaacagcgcgcagattgcggccaactacctgcaacagattgttaagaactatcagcaagaattgttggagggcaaaccgaagccgaataacagcgaactggtcgtgcgtaattggtacaatccgaatctggactacaagtggttcgtggttccgagcctgatcgcgatgattaccaccattggcgtaatgatcgttacttccctgagcgtggcacgcgaacgtgaacaaggtacgctggaccagttgctggtcagcccgttgaccacctggcagatcttcatcggtaaagcagttccagcactgatcgttgcgactttccaggcaaccatcgtgctggccatcggtatttgggcgtaccagattccgtttgcgggtagcctggctctgttttacttcactatggtcatttatggcctgtctttggttggttttggtttgctgatctcttccctgtgcagcacccagcaacaagcgttcattggtgtctttgtgtttatgatgccagcaattctgctgagcggctatgtgagcccggtcgagaacatgccggtctggctgcaaaatctgacgtggatcaatccgatccgtcatttcacggatattaccaaacaaatctacctgaaggatgctagcctggatatcgtgtggaacagcttgtggccgctgctggtcattacggcaaccacgggttctgcggcgtatgcgatgttccgtcgcaaagtgatgtaa

SEQ ID NO: 123 - protein, 492 aa, Artificial Sequence
  Description of Artificial Sequence Synthetic polypeptide
MFLGWFTNASLFRKQIYMAIASGVFSGFAVLVLGSIVGLGGTPKDVPAPSGETTTEAPAEGAPAEGQAPSQTPEEEPGKPSLLNLAFLTAIATAIGVFLINRLLMQQIKSIIDDLQSNPILSWRRVRALCVKETRQIVRDPSSWLIAVVIPLLLLFIFGYGINLDSSKLRVGILLEQRSEAALDFTHTMTGSPYIDATISDNRQELIAKMQAGKIRGLVVIPVDFAEQMERANATAPIQVITDGSEPNTANFVQGYVEGIWQIWQMQRAEDNGQTFEPLIDVQTRYWFNPAAISQHFIIPGAVTIIMTVIGAILTSLVVAREWERGTMEALLSTEITRTELLLCKLIPYYFLGMLAMLLCMLVSVFILGVPYRGSLLILFFISSLFLLSTLGMGLLISTITRNQFNAAQVALNAAFLPSIMLSGFIFQIDSMPAVIRAVTYIIPARYFVSTLQSLFLAGNIPVVLVVNVLFLIASAVMFIGLTWLKTKRRLD

SEQ ID NO: 124 - protein, 483 aa, Artificial Sequence
  Description of Artificial Sequence Synthetic polypeptide
MFLGWFTNASLFRKQIYMAIASGVFSGFAVLVLGSIVGLGGTPKDVPAPSGETTTEAPAEGAPAEGQAPSQTPEEEPGKPSLLNLAFLTAIATAIGVFLINRLLMQQIKSIIDDLQFHRLWTLIRKELQSLLREPQTRAILILPVLIQVILFPFAATLEVTNATIAIYDEDNGEHSVELTQRFARASAFTHVLLLKSPQEIRPTIDTQKALLLVRFPADFSRKLDTFQTAPLQLILDGRNSNSAQIAANYLQQIVKNYQQELLEGKPKPNNSELVVRNWYNPNLDYKWFVVPSLIAMITTIGVMIVTSLSVAREREQGTLDQLLVSPLTTWQIFIGKAVPALIVATFQATIVLAIGIWAYQIPFAGSLALFYFTMVIYGLSLVGFGLLISSLCSTQQQAFIGVFVFMMPAILLSGYVSPVENMPVWLQNLTWINPIRHFTDITKQIYLKDASLDIVWNSLWPLLVITATTGSAAYAMFRRKVM

SEQ ID NO: 125 - DNA, 123 bp, Artificial Sequence
  Description of Artificial Sequence Synthetic polynucleotide
gggggggggggggaaagccacgttgtgtctcaaaatctctgatgttacattgcacaagataaaaatatatcatcatgaacaataaaactgtctgcttacataaacagtaatacaagtgtacat

SEQ ID NO: 126 - DNA, 212 bp, Artificial Sequence
  Description of Artificial Sequence Synthetic polynucleotide
gcccctatattatgcatttatacccccacaatcatgtcaagaattcaagcatcttaaataatgttaattatcggcaaagtctgtgctccccttctataatgctgaattgagcattcgcctcctgaacggtctttattcttccattgtgggtctttagattcacgattcttcacaatcattgatctaaggatctttgtagattctctgtacat

SEQ ID NO: 127 - DNA, 161 bp, Artificial Sequence
  Description of Artificial Sequence Synthetic polynucleotide
ccaaggtggctacttcaacgatagcttaaacttcgctgctccagcgaggggatttcactggtttgaatgcttcaatgcttgccaaaagagtgctactggaacttacaagagtgaccctgcgtcaggggagctagcactcaaaaaagactcctcctgtacat

SEQ ID NO: 128 - DNA, 1809 bp, Artificial Sequence
  Description of Artificial Sequence Synthetic polynucleotide
atgcaaaaacaacagaatctggactactttagcccgcaggcgttggcactgtgggcggctattgcttccctgggtgttatgagcccggcacacgcggagccgcgtagcgagggcagccattctgatccgctggttccgaccgcgacgcaggtcgtggttccggcgctgccggtggaggacgttgcgccgaccgccgcaccggcatcgcagaccccggctcctcagagcgaaaacttggcgcaatccagcacccaagccgtcacgagccctgtggcgcaggcgcaggaagccccgcaagacagcaatctgccgcaactgtatgcccagcagcaaggtaacccaaatgcccaacaggcgaacccggagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaatcgtattcactttggtattggtgagcgtttctaa

SEQ ID NO: 129 - protein, 602 aa, Artificial Sequence
  Description of Artificial Sequence Synthetic polypeptide
MQKQQNLDYFSPQALALWAAIASLGVMSPAHAEPRSEGSHSDPLVPTATQVVVPALPVEDVAPTAAPASQTPAPQSENLAQSSTQAVTSPVAQAQEAPQDSNLPQLYAQQQGNPNAQQANPENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRNRIHFGIGERF

SEQ ID NO: 130 - DNA, 1809 bp, Artificial Sequence
  Description of Artificial Sequence Synthetic polynucleotide
atgcaaaaacaacagaatctggactactttagcccgcaggcgttggcactgtgggcggctattgcttccctgggtgttatgagcccggcacacgcggagccgcgtagcgagggcagccattctgatccgctggttccgaccgcgacgcaggtcgtggttccggcgctgccggtggaggacgttgcgccgaccgccgcaccggcatcgcagaccccggctcctcagagcgaaaacttggcgcaatccagcacccaagccgtcacgagccctgtggcgcaggcgcaggaagccccgcaagacagcaatctgccgcaactgtatgcccagcagcaaggtaacccaaatgcccaacaggcgaacccggagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaatggggatgcggtgattgccccggcggctccctaa

SEQ ID NO: 131 - protein, 602 aa, Artificial Sequence
  Description of Artificial Sequence Synthetic polypeptide
MQKQQNLDYFSPQALALWAAIASLGVMSPAHAEPRSEGSHSDPLVPTATQVVVPALPVEDVAPTAAPASQTPAPQSENLAQSSTQAVTSPVAQAQEAPQDSNLPQLYAQQQGNPNAQQANPENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRNGDAVIAPAAP

SEQ ID NO: 132 - DNA, 1524 bp, Artificial Sequence
  Description of Artificial Sequence Synthetic polynucleotide
atgtttgcctttcgtgacttcttgaccttcagcaccggtggcctggttgtcctgtccggcggtggtgttgcgattgcggagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaatcgtattcactttggtattggtgagcgtttctaa

SEQ ID NO: 133 - protein, 507 aa, Artificial Sequence
  Description of Artificial Sequence Synthetic polypeptide
MFAFRDFLTFSTGGLVVLSGGGVAIAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRNRIHFGIGERF

SEQ ID NO: 134 - DNA, 1524 bp, Artificial Sequence
  Description of Artificial Sequence Synthetic polynucleotide
atgtttgcctttcgtgacttcttgaccttcagcaccggtggcctggttgtcctgtccggcggtggtgttgcgattgcggagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaatggggatgcggtgattgccccggcggctccctaa

SEQ ID NO: 135 - protein, 507 aa, Artificial Sequence
  Description of Artificial Sequence Synthetic polypeptide
MFAFRDFLTFSTGGLVVLSGGGVAIAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRNGDAVIAPAAP

SEQ ID NO: 136 - DNA, 1977 bp, Artificial Sequence
  Description of Artificial Sequence Synthetic polynucleotide
atgtttgccttccgtgacttcctgacgtttagcacgggcggtttggtcgtgttgagcggtggcggtgttgcgattgcacaaaccacccctccgcagatcgccactccggagccgtttatcggtcagacgccgcaggcaccgctgccaccgctggctgcgccgtccgttgaaagcctggacaccgcggctttcctgccgagcctgggcggtctgtcccaaccgaccaccctggccgcactgcctttgccgagcccggagttgaacctgtcgcctacggcgcatctgggtaccatccaggcgccaagcccgctgttggcgcaagtggataccactgcgaccccgagcccgaccaccgcgattgacgtcaccctgccgacggcggaaacgaatcaaaccattccgctggtccagccgctgccgccagaccgcgtcatcaatgaggacctgaaccaactgctggagccgattgataacccggcagttacggtgccgcaggaagcgaccgccgttacgaccgataatgttgtggatgagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaatcgtattcactttggtattggtgagcgtttctaa

SEQ ID NO: 137 - protein, 658 aa, Artificial Sequence
  Description of Artificial Sequence Synthetic polypeptide
MFAFRDFLTFSTGGLVVLSGGGVAIAQTTPPQIATPEPFIGQTPQAPLPPLAAPSVESLDTAAFLPSLGGLSQPTTLAALPLPSPELNLSPTAHLGTIQAPSPLLAQVDTTATPSPTTAIDVTLPTAETNQTIPLVQPLPPDRVINEDLNQLLEPIDNPAVTVPQEATAVTTDNVVDENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRNRIHFGIGERF

SEQ ID NO: 138 - DNA, 1977 bp, Artificial Sequence
  Description of Artificial Sequence Synthetic polynucleotide
atgtttgccttccgtgacttcctgacgtttagcacgggcggtttggtcgtgttgagcggtggcggtgttgcgattgcacaaaccacccctccgcagatcgccactccggagccgtttatcggtcagacgccgcaggcaccgctgccaccgctggctgcgccgtccgttgaaagcctggacaccgcggctttcctgccgagcctgggcggtctgtcccaaccgaccaccctggccgcactgcctttgccgagcccggagttgaacctgtcgcctacggcgcatctgggtaccatccaggcgccaagcccgctgttggcgcaagtggataccactgcgaccccgagcccgaccaccgcgattgacgtcaccctgccgacggcggaaacgaatcaaaccattccgctggtccagccgctgccgccagaccgcgtcatcaatgaggacctgaaccaactgctggagccgattgataacccggcagttacggtgccgcaggaagcgaccgccgttacgaccgataatgttgtggatgagaatttgatgcaggtttaccagcaggcgcgtctgtccaatccggagctgcgtaaaagcgctgccgaccgtgatgccgcgtttgagaagattaacgaagcccgcagcccgctgctgccgcagctgggtttgggcgctgactacacctactccaacggctatcgtgacgccaacggtatcaatagcaatgcgaccagcgccagcctgcaactgacccaaagcatttttgatatgagcaaatggcgcgctctgaccctgcaagagaaagcggcaggtatccaggatgtgacctaccaaacggaccagcagaccctgatcttgaacacggcgaccgcgtatttcaatgttttgaacgcaatcgatgtcctgagctatacccaggcccagaaggaagcgatttatcgtcagttggatcagaccacccagcgcttcaatgtgggtctggtggcgattacggatgttcaaaatgcgcgtgcgcaatacgatactgttttggcaaacgaagtgacggcgcgtaacaatctggataatgccgttgaacagctgcgtcaaatcacgggcaactactatccggaactggcagcactgaacgttgagaatttcaagacggataagccgcaacctgtgaacgcgctgctgaaagaggcggaaaagcgcaatctgagcctgctgcaagcccgtctgagccaagacctggcgcgtgagcagattcgtcaggcacaagatggccacctgccaaccctggacttgacggcatccacgggtatctcggacaccagctactccggtagcaagactcgcggtgcagcaggtacgcagtatgacgactctaacatgggtcaaaacaaagtcggcctgtctttcagcctgccgatctaccaaggtggcatggttaattctcaagttaaacaggcgcaatacaactttgtcggcgcgagcgaacagctggagagcgctcaccgtagcgtggtccagaccgtccgttcttcttttaacaacattaacgcgagcatcagcagcattaacgcatacaaacaagcggtggtgagcgcgcaatcgagcctggacgcaatggaggcgggttacagcgtcggtacgcgcaccattgtcgacgtgctggatgcaactaccaccctgtataatgcaaagcaagaactggcaaatgcgcgctacaactatctgattaaccagctgaatatcaaatccgcgctgggcacgctgaacgagcaggatctgctggcattgaacaacgcgctgagcaagccggtaagcacgaatccggagaacgtcgccccacaaaccccggaacagaatgctatcgcggacggctatgccccggacagcccggctccggttgtgcagcagactagcgctcgcaccaccaccagcaatggtcataatccgttccgtaatggggatgcggtgattgccccggcggctccctaa

SEQ ID NO: 139 - protein, 658 aa, Artificial Sequence
  Description of Artificial Sequence Synthetic polypeptide
MFAFRDFLTFSTGGLVVLSGGGVAIAQTTPPQIATPEPFIGQTPQAPLPPLAAPSVESLDTAAFLPSLGGLSQPTTLAALPLPSPELNLSPTAHLGTIQAPSPLLAQVDTTATPSPTTAIDVTLPTAETNQTIPLVQPLPPDRVINEDLNQLLEPIDNPAVTVPQEATAVTTDNVVDENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRNGDAVIAPAAP

SEQ ID NO: 140 - DNA, 1776 bp, Artificial Sequence
  Description of Artificial Sequence Synthetic polynucleotide
atgttcgcttttcgagattttcttactttcagtaccggtggccttgtggttctctctggtggtggggtggcgatcgcccaaacaaccccgccgcaaatcgctactccagaacctttcatcggccagaccccccaggcgccattgccaccattggccgctcctagcgttgaatccctcgatacagcagcctttttaccgagtctcggtggtctcagccaacccacaaccctggccgctttacctctaccttccccagagctcaatttatccccgactgcccacctcggcacaattcaagctccctcgccgctccttgcccaggtagatacaacggcgaccccctccccaacaaccgccattgatgtgaccctgcccaccgcagagacaaaccagacgattccccttgtgcaacccttaccgccggatcgggtgattaatgaagatctaaatcagctcctagagcccatcgataatccggcagtgacagtcccccaggaggccacggcggtgacgactgacaatgttgttgacctcaccctagaagaaacgattcgtctggccctagagcgcaatgaaacgctccaggaagcccgtctgaactacgaccgatcagaggaactggtgcgagaggcgatcgccgccgaatacccaaatctcagcaaccaggttgacattacccgcaccgatagcgccaacggagaactccaggcccgacggctggggggagacaacaatgccaccacagcgatcaatggtcgtctcgaagtcagctatgacatctataccggggggcgtcgctctgcccaaattgaagcagcccagacccaattgcaaattgctgaactagacatcgagcgcctcaccgaagaaactcgtctagccgctgcggtgaactattacaatctccagagtgccgacgcccaggtggttatcgagcaaagttcggtgtttgatgccacccagagtttacgggatgccaccctactagaacaggcaggcttgggcacaaaatttgatgtgttgcgggccgaggtcgaactcgctagtgcccaacagcggctcaccagggctgaagccacccaaagaaccgcccggcgtcaactggctcaactgctgagtttggaaccgaccatcgatccccgcaccgccgatgagattaacctcgctggaagatgggaaatttctttagaagaaaccattgtcctggcattgcaaaaccgccaagaattgcgccagcagctcctccagcgggaagttgatggttaccaggaacggattgcattggctgccgttcgacctttagtcagcgtttttgcgaattatgatgtcttggaagtgtttgatgatagccttggccccgccgatgggttaacggttggggcccggatgcgttggaatttctttgatgggggtgcagcggccgcccgggcaaatcaagagcaagttgatcaggcgatcgccgaaaatcgttttgctaaccaaagaaaccaaattcgcctggcggtggaaacggcctactatgactttgaagccagcgaacaaaacatcacgacggcagccgccgcagtcactttagcagaagaaagtttacgcctggctcgtctgcgctttaatgcaggggtcggcacccaaaccgatgtaatctctgcccaaacgggtctgaatacggcccgggggaactatcttcaggcagtcaccgattacaatcgtgcctttgcccaactgaaacgggaagtcggtttaggggatgcggtgattgccccggcggctccctag

SEQ ID NO: 141 - protein, 591 aa, Artificial Sequence
  Description of Artificial Sequence Synthetic polypeptide
MFAFRDFLTFSTGGLVVLSGGGVAIAQTTPPQIATPEPFIGQTPQAPLPPLAAPSVESLDTAAFLPSLGGLSQPTTLAALPLPSPELNLSPTAHLGTIQAPSPLLAQVDTTATPSPTTAIDVTLPTAETNQTIPLVQPLPPDRVINEDLNQLLEPIDNPAVTVPQEATAVTTDNVVDLTLEETIRLALERNETLQEARLNYDRSEELVREAIAAEYPNLSNQVDITRTDSANGELQARRLGGDNNATTAINGRLEVSYDIYTGGRRSAQIEAAQTQLQIAELDIERLTEETRLAAAVNYYNLQSADAQVVIEQSSVFDATQSLRDATLLEQAGLGTKFDVLRAEVELASAQQRLTRAEATQRTARRQLAQLLSLEPTIDPRTADEINLAGRWEISLEETIVLALQNRQELRQQLLQREVDGYQERIALAAVRPLVSVFANYDVLEVFDDSLGPADGLTVGARMRWNFFDGGAAAARANQEQVDQAIAENRFANQRNQIRLAVETAYYDFEASEQNITTAAAAVTLAEESLRLARLRFNAGVGTQTDVISAQTGLNTARGNYLQAVTDYNRAFAQLKREVGLGDAVIAPAAP

SEQ ID NO: 142 - DNA, 999 bp, Artificial Sequence
  Description of Artificial Sequence Synthetic polynucleotide
atgatgaaaaagccggttgttatcggtttggcggtggtggttctggcagcagtcgttgcgggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatcacaaaccatacgaaatcgccctgatgcaagccaaagcgggtgttagcgtggcacaagcgcagtacgatctgatgttggcgggttaccgcaatgaagagattgcgcaggcggcagcggcggtgaaacaagcgcaagcggcgtatgacctggctaaggccgacggcgaccgtttccaagagctgtatgcaagcggtgtggttagcaagcaacgtctggagcaggcgcagaccagccgtgatcaggcacaggccacgctgaagagcgcgcaggataagctgcgccaatatcgtagcggcaatcgtgaacaagacattgcacaggctaaggcatctctggaacaggcccaagctcaactggcccaggcggaactgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 143 - protein, 332 aa, Artificial Sequence
  Description of Artificial Sequence Synthetic polypeptide
MMKKPVVIGLAVVVLAAVVAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDLAKADGDRFQELYASGVVSKQRLEQAQTSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 144 - DNA, 1326 bp, Artificial Sequence
  Description of Artificial Sequence Synthetic polynucleotide
atgatgaaaaagccggttgttatcggtttggcggtggtggttctggcagcagtcgttgcgggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatagcgccgaactgcaggcatccctggatggtgcacaagcccgtatcaatgcggcgcagcagcaggttaatcaagcacagctgcaaatcaccgtgattgaaaaccagattaccgaggcacagctgacccaacgccaagcacaggatgacactgccggtcgcgttaatgcggcacaagcgaacgtggcggcagccaaggcgcaactggcccaggcgcaagcgcaggtcaagcagctggaagcagagctggccctggcgaaggcagacggtgaccgtttccaagaactgtacgcgagcggtgtggtgagcaaacagcgtctggagcaagctcaaacccaatatctgagcacgaaagagaatctggatgctcgtcgcgcggttgttgcggcagctgcggagcaagtgaaaaccgcggagggtaacctgacgcaaactcaggcgtcccagttcaacccagacattcagtacctgagcaccaaagaaaatctggacgcacgtcgtgctgtcgtcgctgccgctgcagaacaagttaagaccgccgagggtaacttgactcagacccaagcgagccaattcaacccggacattcgtgcagttcaagtgcagcgcctgcaaacgcaactggtccaggcgcaggcccagctgtctgcggcgcaagcacaagttcagaatgctcaggccaactataacgagatcgcggcgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 145 - protein, 441 aa, Artificial Sequence
  Description of Artificial Sequence Synthetic polypeptide
MMKKPVVIGLAVVVLAAVVAGGYWWYQSRQDNGLTLYGNVDIRTVNLSFRVGGRVESLAVDEGDAIKAGQVLGELDSAELQASLDGAQARINAAQQQVNQAQLQITVIENQITEAQLTQRQAQDDTAGRVNAAQANVAAAKAQLAQAQAQVKQLEAELALAKADGDRFQELYASGVVSKQRLEQAQTQYLSTKENLDARRAVVAAAAEQVKTAEGNLTQTQASQFNPDIQYLSTKENLDARRAVVAAAAEQVKTAEGNLTQTQASQFNPDIRAVQVQRLQTQLVQAQAQLSAAQAQVQNAQANYNEIAANLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 146 - DNA, 1326 bp, Artificial Sequence
  Description of Artificial Sequence Synthetic polynucleotide
atgatgaaaaagccggttgttatcggtttggcggtggtggttctggcagcagtcgttgcgggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatagcgccgaactgcaggcatccctggatggtgcacaagcccgtatcaatgcggcgcagcagcaggttaatcaagcacagctgcaaatcaccgtgattgaaaaccagattaccgaggcacagctgacccaacgccaagcacaggatgacactgccggtcgcgttaatgcggcacaagcgaacgtggcggcagccaaggcgcaactggcccaggcgcaagcgcaggtcaagcagctggaagcagagctggcctatgcgcaaaacttttacaatcgccagcaaggtttgtggaagagccgtacgattagcgcaaacgatctggaaaatgcgcgttctcaatatctgagcacgaaagagaatctggatgctcgtcgcgcggttgttgcggcagctgcggagcaagtgaaaaccgcggagggtaacctgacgcaaactcaggcgtcccagttcaacccagacattcagtacctgagcaccaaagaaaatctggacgcacgtcgtgctgtcgtcgctgccgctgcagaacaagttaagaccgccgagggtaacttgactcagacccaagcgagccaattcaacccggacattcgtgcagttcaagtgcagcgcctgcaaacgcaactggtccaggcgcaggcccagctgtctgcggcgcaagcacaagttcagaatgctcaggccaactataacgagatcgcggcgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 147 - protein, 441 aa, Artificial Sequence
  Description of Artificial Sequence Synthetic polypeptide
MMKKPVVIGLAVVVLAAVVAGGYWWYQSRQDNGLTLYGNVDIRTVNLSFRVGGRVESLAVDEGDAIKAGQVLGELDSAELQASLDGAQARINAAQQQVNQAQLQITVIENQITEAQLTQRQAQDDTAGRVNAAQANVAAAKAQLAQAQAQVKQLEAELAYAQNFYNRQQGLWKSRTISANDLENARSQYLSTKENLDARRAVVAAAAEQVKTAEGNLTQTQASQFNPDIQYLSTKENLDARRAVVAAAAEQVKTAEGNLTQTQASQFNPDIRAVQVQRLQTQLVQAQAQLSAAQAQVQNAQANYNEIAANLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 148 - DNA, 999 bp, Artificial Sequence
  Description of Artificial Sequence Synthetic polynucleotide
atgatgaaaaagccggttgttatcggtttggcggtggtggttctggcagcagtcgttgcgggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatcacaaaccatacgaaatcgccctgatgcaagccaaagcgggtgttagcgtggcacaagcgcagtacgatctgatgttggcgggttaccgcaatgaagagattgcgcaggcggcagcggcggtgaaacaagcgcaagcggcgtatgactatgcgcaaaacttttacaatcgtttccaagagctgtatgcaagcggtgtggttagcaagcaagatctggaaaatgcgcgttctagccgtgatcaggcacaggccacgctgaagagcgcgcaggataagctgcgccaatatcgtagcggcaatcgtgaacaagacattgcacaggctaaggcatctctggaacaggcccaagctcaactggcccaggcggaactgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 149 - protein, 332 aa, Artificial Sequence
  Description of Artificial Sequence Synthetic polypeptide
MMKKPVVIGLAVVVLAAVVAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDYAQNFYNRFQELYASGVVSKQDLENARSSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 150 - DNA, 1056 bp, Artificial Sequence
  Description of Artificial Sequence Synthetic polynucleotide
atgaacaacaacgatctgtttcaagcaagccgccgtcgctttctggcgcagctgggcggcttgaccgtcgctggcatgctgggtccgagcctgctgacgccacgccgtgcaaccgctggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatcacaaaccatacgaaatcgccctgatgcaagccaaagcgggtgttagcgtggcacaagcgcagtacgatctgatgttggcgggttaccgcaatgaagagattgcgcaggcggcagcggcggtgaaacaagcgcaagcggcgtatgacctggctaaggccgacggcgaccgtttccaagagctgtatgcaagcggtgtggttagcaagcaacgtctggagcaggcgcagaccagccgtgatcaggcacaggccacgctgaagagcgcgcaggataagctgcgccaatatcgtagcggcaatcgtgaacaagacattgcacaggctaaggcatctctggaacaggcccaagctcaactggcccaggcggaactgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 151 - protein, 351 aa, Artificial Sequence
  Description of Artificial Sequence Synthetic polypeptide
MNNNDLFQASRRRFLAQLGGLTVAGMLGPSLLTPRRATAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDLAKADGDRFQELYASGVVSKQRLEQAQTSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 152 - DNA, 1383 bp, Artificial Sequence
  Description of Artificial Sequence Synthetic polynucleotide
atgaacaacaacgatctgtttcaagcaagccgccgtcgctttctggcgcagctgggcggcttgaccgtcgctggcatgctgggtccgagcctgctgacgccacgccgtgcaaccgctggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatagcgccgaactgcaggcatccctggatggtgcacaagcccgtatcaatgcggcgcagcagcaggttaatcaagcacagctgcaaatcaccgtgattgaaaaccagattaccgaggcacagctgacccaacgccaagcacaggatgacactgccggtcgcgttaatgcggcacaagcgaacgtggcggcagccaaggcgcaactggcccaggcgcaagcgcaggtcaagcagctggaagcagagctggccctggcgaaggcagacggtgaccgtttccaagaactgtacgcgagcggtgtggtgagcaaacagcgtctggagcaagctcaaacccaatatctgagcacgaaagagaatctggatgctcgtcgcgcggttgttgcggcagctgcggagcaagtgaaaaccgcggagggtaacctgacgcaaactcaggcgtcccagttcaacccagacattcagtacctgagcaccaaagaaaatctggacgcacgtcgtgctgtcgtcgctgccgctgcagaacaagttaagaccgccgagggtaacttgactcagacccaagcgagccaattcaacccggacattcgtgcagttcaagtgcagcgcctgcaaacgcaactggtccaggcgcaggcccagctgtctgcggcgcaagcacaagttcagaatgctcaggccaactataacgagatcgcggcgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 153 - protein, 460 aa, Artificial Sequence
  Description of Artificial Sequence Synthetic polypeptide
MNNNDLFQASRRRFLAQLGGLTVAGMLGPSLLTPRRATAGGYWWYQSRQDNGLTLYGNVDIRTVNLSFRVGGRVESLAVDEGDAIKAGQVLGELDSAELQASLDGAQARINAAQQQVNQAQLQITVIENQITEAQLTQRQAQDDTAGRVNAAQANVAAAKAQLAQAQAQVKQLEAELALAKADGDRFQELYASGVVSKQRLEQAQTQYLSTKENLDARRAVVAAAAEQVKTAEGNLTQTQASQFNPDIQYLSTKENLDARRAVVAAAAEQVKTAEGNLTQTQASQFNPDIRAVQVQRLQTQLVQAQAQLSAAQAQVQNAQANYNEIAANLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 154 - DNA, 1383 bp, Artificial Sequence
  Description of Artificial Sequence Synthetic polynucleotide
atgaacaacaacgatctgtttcaagcaagccgccgtcgctttctggcgcagctgggcggcttgaccgtcgctggcatgctgggtccgagcctgctgacgccacgccgtgcaaccgctggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatagcgccgaactgcaggcatccctggatggtgcacaagcccgtatcaatgcggcgcagcagcaggttaatcaagcacagctgcaaatcaccgtgattgaaaaccagattaccgaggcacagctgacccaacgccaagcacaggatgacactgccggtcgcgttaatgcggcacaagcgaacgtggcggcagccaaggcgcaactggcccaggcgcaagcgcaggtcaagcagctggaagcagagctggcctatgcgcaaaacttttacaatcgccagcaaggtttgtggaagagccgtacgattagcgcaaacgatctggaaaatgcgcgttctcaatatctgagcacgaaagagaatctggatgctcgtcgcgcggttgttgcggcagctgcggagcaagtgaaaaccgcggagggtaacctgacgcaaactcaggcgtcccagttcaacccagacattcagtacctgagcaccaaagaaaatctggacgcacgtcgtgctgtcgtcgctgccgctgcagaacaagttaagaccgccgagggtaacttgactcagacccaagcgagccaattcaacccggacattcgtgcagttcaagtgcagcgcctgcaaacgcaactggtccaggcgcaggcccagctgtctgcggcgcaagcacaagttcagaatgctcaggccaactataacgagatcgcggcgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 155 - protein, 460 aa, Artificial Sequence
  Description of Artificial Sequence Synthetic polypeptide
MNNNDLFQASRRRFLAQLGGLTVAGMLGPSLLTPRRATAGGYWWYQSRQDNGLTLYGNVDIRTVNLSFRVGGRVESLAVDEGDAIKAGQVLGELDSAELQASLDGAQARINAAQQQVNQAQLQITVIENQITEAQLTQRQAQDDTAGRVNAAQANVAAAKAQLAQAQAQVKQLEAELAYAQNFYNRQQGLWKSRTISANDLENARSQYLSTKENLDARRAVVAAAAEQVKTAEGNLTQTQASQFNPDIQYLSTKENLDARRAVVAAAAEQVKTAEGNLTQTQASQFNPDIRAVQVQRLQTQLVQAQAQLSAAQAQVQNAQANYNEIAANLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 156 - DNA, 1056 bp, Artificial Sequence
  Description of Artificial Sequence Synthetic polynucleotide
atgaacaacaacgatctgtttcaagcaagccgccgtcgctttctggcgcagctgggcggcttgaccgtcgctggcatgctgggtccgagcctgctgacgccacgccgtgcaaccgctggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatcacaaaccatacgaaatcgccctgatgcaagccaaagcgggtgttagcgtggcacaagcgcagtacgatctgatgttggcgggttaccgcaatgaagagattgcgcaggcggcagcggcggtgaaacaagcgcaagcggcgtatgactatgcgcaaaacttttacaatcgtttccaagagctgtatgcaagcggtgtggttagcaagcaagatctggaaaatgcgcgttctagccgtgatcaggcacaggccacgctgaagagcgcgcaggataagctgcgccaatatcgtagcggcaatcgtgaacaagacattgcacaggctaaggcatctctggaacaggcccaagctcaactggcccaggcggaactgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 157 - protein, 351 aa, Artificial Sequence
  Description of Artificial Sequence Synthetic polypeptide
MNNNDLFQASRRRFLAQLGGLTVAGMLGPSLLTPRRATAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDYAQNFYNRFQELYASGVVSKQDLENARSSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 158 - DNA, 1035 bp, Artificial Sequence
  Description of Artificial Sequence Synthetic polynucleotide
atgcagaagcagcagaacctggactatttcagcccgcaagcgttggcgctgtgggcagctatcgccagcctgggcgttatgtccccagcacacgctggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatcacaaaccatacgaaatcgccctgatgcaagccaaagcgggtgttagcgtggcacaagcgcagtacgatctgatgttggcgggttaccgcaatgaagagattgcgcaggcggcagcggcggtgaaacaagcgcaagcggcgtatgacctggctaaggccgacggcgaccgtttccaagagctgtatgcaagcggtgtggttagcaagcaacgtctggagcaggcgcagaccagccgtgatcaggcacaggccacgctgaagagcgcgcaggataagctgcgccaatatcgtagcggcaatcgtgaacaagacattgcacaggctaaggcatctctggaacaggcccaagctcaactggcccaggcggaactgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 159 - protein, 344 aa, Artificial Sequence
  Description of Artificial Sequence Synthetic polypeptide
MQKQQNLDYFSPQALALWAAIASLGVMSPAHAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDLAKADGDRFQELYASGVVSKQRLEQAQTSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 160 - DNA, 1362 bp, Artificial Sequence
  Description of Artificial Sequence Synthetic polynucleotide
atgcagaagcagcagaacctggactatttcagcccgcaagcgttggcgctgtgggcagctatcgccagcctgggcgttatgtccccagcacacgctggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatagcgccgaactgcaggcatccctggatggtgcacaagcccgtatcaatgcggcgcagcagcaggttaatcaagcacagctgcaaatcaccgtgattgaaaaccagattaccgaggcacagctgacccaacgccaagcacaggatgacactgccggtcgcgttaatgcggcacaagcgaacgtggcggcagccaaggcgcaactggcccaggcgcaagcgcaggtcaagcagctggaagcagagctggccctggcgaaggcagacggtgaccgtttccaagaactgtacgcgagcggtgtggtgagcaaacagcgtctggagcaagctcaaacccaatatctgagcacgaaagagaatctggatgctcgtcgcgcggttgttgcggcagctgcggagcaagtgaaaaccgcggagggtaacctgacgcaaactcaggcgtcccagttcaacccagacattcagtacctgagcaccaaagaaaatctggacgcacgtcgtgctgtcgtcgctgccgctgcagaacaagttaagaccgccgagggtaacttgactcagacccaagcgagccaattcaacccggacattcgtgcagttcaagtgcagcgcctgcaaacgcaactggtccaggcgcaggcccagctgtctgcggcgcaagcacaagttcagaatgctcaggccaactataacgagatcgcggcgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 161 - protein, 453 aa, Artificial Sequence
  Description of Artificial Sequence Synthetic polypeptide
MQKQQNLDYFSPQALALWAAIASLGVMSPAHAGGYWWYQSRQDNGLTLYGNVDIRTVNLSFRVGGRVESLAVDEGDAIKAGQVLGELDSAELQASLDGAQARINAAQQQVNQAQLQITVIENQITEAQLTQRQAQDDTAGRVNAAQANVAAAKAQLAQAQAQVKQLEAELALAKADGDRFQELYASGVVSKQRLEQAQTQYLSTKENLDARRAVVAAAAEQVKTAEGNLTQTQASQFNPDIQYLSTKENLDARRAVVAAAAEQVKTAEGNLTQTQASQFNPDIRAVQVQRLQTQLVQAQAQLSAAQAQVQNAQANYNEIAANLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 162 - DNA, 1362 bp, Artificial Sequence
  Description of Artificial Sequence Synthetic polynucleotide
atgcagaagcagcagaacctggactatttcagcccgcaagcgttggcgctgtgggcagctatcgccagcctgggcgttatgtccccagcacacgctggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatagcgccgaactgcaggcatccctggatggtgcacaagcccgtatcaatgcggcgcagcagcaggttaatcaagcacagctgcaaatcaccgtgattgaaaaccagattaccgaggcacagctgacccaacgccaagcacaggatgacactgccggtcgcgttaatgcggcacaagcgaacgtggcggcagccaaggcgcaactggcccaggcgcaagcgcaggtcaagcagctggaagcagagctggcctatgcgcaaaacttttacaatcgccagcaaggtttgtggaagagccgtacgattagcgcaaacgatctggaaaatgcgcgttctcaatatctgagcacgaaagagaatctggatgctcgtcgcgcggttgttgcggcagctgcggagcaagtgaaaaccgcggagggtaacctgacgcaaactcaggcgtcccagttcaacccagacattcagtacctgagcaccaaagaaaatctggacgcacgtcgtgctgtcgtcgctgccgctgcagaacaagttaagaccgccgagggtaacttgactcagacccaagcgagccaattcaacccggacattcgtgcagttcaagtgcagcgcctgcaaacgcaactggtccaggcgcaggcccagctgtctgcggcgcaagcacaagttcagaatgctcaggccaactataacgagatcgcggcgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 163 - protein, 453 aa, Artificial Sequence
  Description of Artificial Sequence Synthetic polypeptide
MQKQQNLDYFSPQALALWAAIASLGVMSPAHAGGYWWYQSRQDNGLTLYGNVDIRTVNLSFRVGGRVESLAVDEGDAIKAGQVLGELDSAELQASLDGAQARINAAQQQVNQAQLQITVIENQITEAQLTQRQAQDDTAGRVNAAQANVAAAKAQLAQAQAQVKQLEAELAYAQNFYNRQQGLWKSRTISANDLENARSQYLSTKENLDARRAVVAAAAEQVKTAEGNLTQTQASQFNPDIQYLSTKENLDARRAVVAAAAEQVKTAEGNLTQTQASQFNPDIRAVQVQRLQTQLVQAQAQLSAAQAQVQNAQANYNEIAANLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 164 - DNA, 1035 bp, Artificial Sequence
  Description of Artificial Sequence Synthetic polynucleotide
atgcagaagcagcagaacctggactatttcagcccgcaagcgttggcgctgtgggcagctatcgccagcctgggcgttatgtccccagcacacgctggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatcacaaaccatacgaaatcgccctgatgcaagccaaagcgggtgttagcgtggcacaagcgcagtacgatctgatgttggcgggttaccgcaatgaagagattgcgcaggcggcagcggcggtgaaacaagcgcaagcggcgtatgactatgcgcaaaacttttacaatcgtttccaagagctgtatgcaagcggtgtggttagcaagcaagatctggaaaatgcgcgttctagccgtgatcaggcacaggccacgctgaagagcgcgcaggataagctgcgccaatatcgtagcggcaatcgtgaacaagacattgcacaggctaaggcatctctggaacaggcccaagctcaactggcccaggcggaactgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 165 - protein, 344 aa, Artificial Sequence
  Description of Artificial Sequence Synthetic polypeptide
MQKQQNLDYFSPQALALWAAIASLGVMSPAHAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDYAQNFYNRFQELYASGVVSKQDLENARSSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 166 - DNA, 1005 bp, Artificial Sequence
  Description of Artificial Sequence Synthetic polynucleotide
atgcgtttcttttggtttttcctgacgttgctgaccctgagcacctggcagctgccggcgtgggcgggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatcacaaaccatacgaaatcgccctgatgcaagccaaagcgggtgttagcgtggcacaagcgcagtacgatctgatgttggcgggttaccgcaatgaagagattgcgcaggcggcagcggcggtgaaacaagcgcaagcggcgtatgacctggctaaggccgacggcgaccgtttccaagagctgtatgcaagcggtgtggttagcaagcaacgtctggagcaggcgcagaccagccgtgatcaggcacaggccacgctgaagagcgcgcaggataagctgcgccaatatcgtagcggcaatcgtgaacaagacattgcacaggctaaggcatctctggaacaggcccaagctcaactggcccaggcggaactgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 167 - protein, 334 aa, Artificial Sequence
  Description of Artificial Sequence Synthetic polypeptide
MRFFWFFLTLLTLSTWQLPAWAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDLAKADGDRFQELYASGVVSKQRLEQAQTSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 168 - DNA, 1326 bp, Artificial Sequence; identical to SEQ ID NO: 144
  Description of Artificial Sequence Synthetic polynucleotide

SEQ ID NO: 169 - protein, 441 aa, Artificial Sequence; identical to SEQ ID NO: 145
  Description of Artificial Sequence Synthetic polypeptide

SEQ ID NO: 170 - DNA, 1332 bp, Artificial Sequence
  Description of Artificial Sequence Synthetic polynucleotide
atgcgtttcttttggtttttcctgacgttgctgaccctgagcacctggcagctgccggcgtgggcgggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatagcgccgaactgcaggcatccctggatggtgcacaagcccgtatcaatgcggcgcagcagcaggttaatcaagcacagctgcaaatcaccgtgattgaaaaccagattaccgaggcacagctgacccaacgccaagcacaggatgacactgccggtcgcgttaatgcggcacaagcgaacgtggcggcagccaaggcgcaactggcccaggcgcaagcgcaggtcaagcagctggaagcagagctggcctatgcgcaaaacttttacaatcgccagcaaggtttgtggaagagccgtacgattagcgcaaacgatctggaaaatgcgcgttctcaatatctgagcacgaaagagaatctggatgctcgtcgcgcggttgttgcggcagctgcggagcaagtgaaaaccgcggagggtaacctgacgcaaactcaggcgtcccagttcaacccagacattcagtacctgagcaccaaagaaaatctggacgcacgtcgtgctgtcgtcgctgccgctgcagaacaagttaagaccgccgagggtaacttgactcagacccaagcgagccaattcaacccggacattcgtgcagttcaagtgcagcgcctgcaaacgcaactggtccaggcgcaggcccagctgtctgcggcgcaagcacaagttcagaatgctcaggccaactataacgagatcgcggcgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 171 - protein, 443 aa, Artificial Sequence
  Description of Artificial Sequence Synthetic polypeptide
MRFFWFFLTLLTLSTWQLPAWAGGYWWYQSRQDNGLTLYGNVDIRTVNLSFRVGGRVESLAVDEGDAIKAGQVLGELDSAELQASLDGAQARINAAQQQVNQAQLQITVIENQITEAQLTQRQAQDDTAGRVNAAQANVAAAKAQLAQAQAQVKQLEAELAYAQNFYNRQQGLWKSRTISANDLENARSQYLSTKENLDARRAVVAAAAEQVKTAEGNLTQTQASQFNPDIQYLSTKENLDARRAVVAAAAEQVKTAEGNLTQTQASQFNPDIRAVQVQRLQTQLVQAQAQLSAAQAQVQNAQANYNEIAANLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 172 - DNA, 1005 bp, Artificial Sequence
  Description of Artificial Sequence Synthetic polynucleotide
atgcgtttcttttggtttttcctgacgttgctgaccctgagcacctggcagctgccggcgtgggcgggtggctactggtggtatcaaagccgccaggataacggtttgaccctgtatggcaatgttgatattcgcaccgtcaacctgtcgttccgcgtgggtggccgtgtggagagcctggccgtggatgaaggcgatgcgatcaaagcaggtcaggtcctaggtgagctggatcacaaaccatacgaaatcgccctgatgcaagccaaagcgggtgttagcgtggcacaagcgcagtacgatctgatgttggcgggttaccgcaatgaagagattgcgcaggcggcagcggcggtgaaacaagcgcaagcggcgtatgactatgcgcaaaacttttacaatcgtttccaagagctgtatgcaagcggtgtggttagcaagcaagatctggaaaatgcgcgttctagccgtgatcaggcacaggccacgctgaagagcgcgcaggataagctgcgccaatatcgtagcggcaatcgtgaacaagacattgcacaggctaaggcatctctggaacaggcccaagctcaactggcccaggcggaactgaacctgcaggactccactctgatcgcaccttctgacggtactttgctgacgcgtgcggttgaaccgggtaccgtgctgaatgagggcggtacggttttcacggtcagcctgacgcgtccggtctgggttcgtgcctacgtcgatgagcgtaacctggaccaggcgcaaccaggccgtaaggttctgctgtataccgacggtcgcccggataaaccttaccacggtcaaattggctttgtttccccgacggctgagtttaccccgaaaaccgtcgaaacgccggacctgcgtaccgacctggtctaccgtctgcgcatcgtcgtgaccgacgcggatgacgcattgcgtcagggcatgccggtgaccgtgcagttcggcgacgaggctggtcatgagtaa

SEQ ID NO: 173 - protein, 334 aa, Artificial Sequence
  Description of Artificial Sequence Synthetic polypeptide
MRFFWFFLTLLTLSTWQLPAWAGGYWWYQSRQDNGLTLYGNVDIRTVNLSFRVGGRVESLAVDEGDAIKAGQVLGELDHKPYEIALMQAKAGVSVAQAQYDLMLAGYRNEEIAQAAAAVKQAQAAYDYAQNFYNRFQELYASGVVSKQDLENARSSRDQAQATLKSAQDKLRQYRSGNREQDIAQAKASLEQAQAQLAQAELNLQDSTLIAPSDGTLLTRAVEPGTVLNEGGTVFTVSLTRPVWVRAYVDERNLDQAQPGRKVLLYTDGRPDKPYHGQIGFVSPTAEFTPKTVETPDLRTDLVYRLRIVVTDADDALRQGMPVTVQFGDEAGHE

SEQ ID NO: 174 - protein, 591 aa, Artificial Sequence; identical to SEQ ID NO: 141
  Description of Artificial Sequence Synthetic polypeptide

SEQ ID NO: 175 - protein, 332 aa, Artificial Sequence; identical to SEQ ID NO: 143
  Description of Artificial Sequence Synthetic polypeptide

SEQ ID NO: 176 - protein, 441 aa, Artificial Sequence; identical to SEQ ID NO: 145
  Description of Artificial Sequence Synthetic polypeptide

SEQ ID NO: 177 - protein, 332 aa, Artificial Sequence; identical to SEQ ID NO: 149
  Description of Artificial Sequence Synthetic polypeptide

SEQ ID NO: 178 - protein, 351 aa, Artificial Sequence; identical to SEQ ID NO: 151
  Description of Artificial Sequence Synthetic polypeptide

SEQ ID NO: 179 - protein, 460 aa, Artificial Sequence; identical to SEQ ID NO: 153
  Description of Artificial Sequence Synthetic polypeptide

SEQ ID NO: 180 - protein, 351 aa, Artificial Sequence; identical to SEQ ID NO: 157
  Description of Artificial Sequence Synthetic polypeptide

SEQ ID NO: 181 - protein, 344 aa, Artificial Sequence; identical to SEQ ID NO: 159
  Description of Artificial Sequence Synthetic polypeptide

SEQ ID NO: 182 - protein, 453 aa, Artificial Sequence; identical to SEQ ID NO: 161
  Description of Artificial Sequence Synthetic polypeptide

SEQ ID NO: 183 - protein, 344 aa, Artificial Sequence; identical to SEQ ID NO: 165
  Description of Artificial Sequence Synthetic polypeptide

SEQ ID NO: 184 - protein, 334 aa, Artificial Sequence; identical to SEQ ID NO: 167
  Description of Artificial Sequence Synthetic polypeptide

SEQ ID NO: 185 - protein, 441 aa, Artificial Sequence; identical to SEQ ID NO: 145
  Description of Artificial Sequence Synthetic polypeptide

SEQ ID NO: 186 - protein, 334 aa, Artificial Sequence; identical to SEQ ID NO: 173
  Description of Artificial Sequence Synthetic polypeptide

SEQ ID NO: 187 - protein, 591 aa, Artificial Sequence; identical to SEQ ID NO: 86
  Description of Artificial Sequence Synthetic polypeptide

SEQ ID NO: 188 - protein, 534 aa, Artificial Sequence; identical to SEQ ID NO: 88
  Description of Artificial Sequence Synthetic polypeptide

SEQ ID NO: 189 - protein, 493 aa, Artificial Sequence; identical to SEQ ID NO: 61
  Description of Artificial Sequence Synthetic polypeptide

SEQ ID NO: 190 - protein, 497 aa, Artificial Sequence; identical to SEQ ID NO: 59
  Description of Artificial Sequence Synthetic polypeptide

SEQ ID NO: 191 - protein, 507 aa, Artificial Sequence; identical to SEQ ID NO: 133
  Description of Artificial Sequence Synthetic polypeptide

SEQ ID NO: 192 - protein, 507 aa, Artificial Sequence; identical to SEQ ID NO: 135
  Description of Artificial Sequence Synthetic polypeptide

SEQ ID NO: 193 - protein, 648 aa, Artificial Sequence; identical to SEQ ID NO: 57
  Description of Artificial Sequence Synthetic polypeptide

SEQ ID NO: 194 - protein, 658 aa, Artificial Sequence; identical to SEQ ID NO: 137
  Description of Artificial Sequence Synthetic polypeptide

SEQ ID NO: 195 - protein, 658 aa, Artificial Sequence; identical to SEQ ID NO: 139
  Description of Artificial Sequence Synthetic polypeptide

SEQ ID NO: 196 - protein, 503 aa, Artificial Sequence; identical to SEQ ID NO: 80
  Description of Artificial Sequence Synthetic polypeptide

SEQ ID NO: 197 - protein, 592 aa, Artificial Sequence; identical to SEQ ID NO: 84
  Description of Artificial Sequence Synthetic polypeptide

SEQ ID NO: 198 - protein, 602 aa, Artificial Sequence; identical to SEQ ID NO: 129
  Description of Artificial Sequence Synthetic polypeptide

SEQ ID NO: 199 - protein, 602 aa, Artificial Sequence; identical to SEQ ID NO: 131
  Description of Artificial Sequence Synthetic polypeptide

SEQ ID NO: 200 - protein, 332 aa, Artificial Sequence; identical to SEQ ID NO: 15
  Description of Artificial Sequence Synthetic polypeptide

SEQ ID NO: 201 - protein, 351 aa, Artificial Sequence; identical to SEQ ID NO: 113
  Description of Artificial Sequence Synthetic polypeptide

SEQ ID NO: 202 - protein, 334 aa, Artificial Sequence; identical to SEQ ID NO: 115
  Description of Artificial Sequence Synthetic polypeptide

SEQ ID NO: 203 - protein, 344 aa, Artificial Sequence; identical to SEQ ID NO: 117
  Description of Artificial Sequence Synthetic polypeptide

SEQ ID NO: 204 - protein, 441 aa, Artificial Sequence; identical to SEQ ID NO: 147
  Description of Artificial Sequence Synthetic polypeptide

SEQ ID NO: 205 - protein, 460 aa, Artificial Sequence; identical to SEQ ID NO: 155
  Description of Artificial Sequence Synthetic polypeptide

SEQ ID NO: 206 - protein, 453 aa, Artificial Sequence; identical to SEQ ID NO: 163
  Description of Artificial Sequence Synthetic polypeptide

SEQ ID NO: 207 - protein, 443 aa, Artificial Sequence; identical to SEQ ID NO: 171
  Description of Artificial Sequence Synthetic polypeptide

SEQ ID NO: 208 - protein, 578 aa, Artificial Sequence; identical to SEQ ID NO: 28
  Description of Artificial Sequence Synthetic polypeptide

SEQ ID NO: 209 - protein, 377 aa, Artificial Sequence; identical to SEQ ID NO: 29
  Description of Artificial Sequence Synthetic polypeptide

SEQ ID NO: 210 - protein, 368 aa, Artificial Sequence; identical to SEQ ID NO: 30
  Description of Artificial Sequence Synthetic polypeptide

SEQ ID NO: 211 - protein, 473 aa, Artificial Sequence; identical to SEQ ID NO: 120
  Description of Artificial Sequence Synthetic polypeptide

SEQ ID NO: 212 - protein, 464 aa, Artificial Sequence; identical to SEQ ID NO: 121
  Description of Artificial Sequence Synthetic polypeptide

SEQ ID NO: 213 - protein, 492 aa, Artificial Sequence; identical to SEQ ID NO: 123
  Description of Artificial Sequence Synthetic polypeptide

SEQ ID NO: 214 - protein, 483 aa, Artificial Sequence; identical to SEQ ID NO: 124
  Description of Artificial Sequence Synthetic polypeptide

SEQ ID NO: 215 - DNA, 17 bp, Artificial Sequence
  Description of Artificial Sequence Synthetic oligonucleotide
actgccctcgatctgta

(19) United States
(12) Patent Application Publication
(10) Pub. No.: US 2012/0135486 A1
(43) Pub. Date: May 31, 2012

(54) Methods and Compositions for the Extracellular Transport of Biosynthetic Hydrocarbons and Other Molecules

(75) Inventors: Nikos B. Reppas, Cambridge, MA (US); Carolyn Lawrence, Boston, MA (US); Kevin M. Smith, Medford, MA (US); Martha Sholl, (US); Christian P. Ridley, (US)

(73) Assignee: C/O JOULE UNLIMITED TECHNOLOGIES, INC., Cambridge, MA (US)

(21) Appl. No.: 13/232,961

(22) Filed: Sep. 14, 2011

Related U.S. Application Data
(60) Provisional application No. 61/382,917, filed on Sep. 14, 2010.
(60) Provisional application No. 61/414,877, filed on Nov. 17, 2010.
(60) Provisional application No. 61/416,713, filed on Nov. 23, 2010.
(60) Provisional application No. 61/478,045, filed on Apr. 21, 2011.

(51) Int. Cl. C12P 5/02; C12N 1/21; C12N 5/10; C12P 7/02; C12N 1/13

(52) U.S. Cl. 435/157; 435/167; 435/257.2; 435/419; 435/252.33

(57) ABSTRACT

The present disclosure identifies methods and compositions for modifying photoautotrophic organisms as hosts, such that the organisms efficiently convert carbon dioxide and light into hydrocarbons, e.g., n-alkanes and n-alkenes, wherein the n-alkanes are secreted into the culture medium via recombinantly expressed transporter proteins. In particular, the use of such organisms for the commercial production of n-alkanes and related molecules is contemplated.

### CROSS-REFERENCE TO RELATED APPLICATIONS

This application claims priority to earlier filed U.S. Provisional Patent Application No. 61/382,917, filed Sep. 14, 2010, U.S. Provisional Patent Application No. 61/414,877, filed Nov. 17, 2010, U.S. Provisional Patent Application No. 61/416,713, filed Nov. 23, 2010, and U.S. Provisional Patent Application No. 61/478,045, filed Apr. 21, 2011.

This application incorporates by reference the disclosures of the above provisional applications, and in addition incorporates by reference the disclosures of U.S. Provisional Patent Application No. 61/224,463 filed, Jul. 9, 2009, U.S. Provisional Patent Application No. 61/228,937, filed Jul. 27, 2009, U.S. utility application Ser. No. 12/759,657, filed Apr. 13, 2010 (now U.S. Pat. No. 7,794,969), and U.S. utility application Ser. No. 12/833,821, filed Jul. 9, 2010.

### BACKGROUND OF THE INVENTION

Previously, recombinant photosynthetic microorganisms have been engineered to produce hydrocarbons, including alkanes, in amounts that exceed the levels produced naturally by the organism. A need exists for engineered photosynthetic microorganisms which have enhanced secretion capabilities such that greater amounts of the biosynthetic hydrocarbon products are excreted into the culture medium, thereby minimizing downstream processing steps.

### SUMMARY OF THE INVENTION

This invention pertains to compositions and methods for increasing the amount of hydrocarbons (particularly n-alkanes and n-alkenes, but not limited to these compositions) that are secreted by engineered microorganisms which have been modified to biosynthetically produce such hydrocarbons. In certain embodiments, the invention provides engineered microorganisms comprising recombinant enzymes for producing hydrocarbons, wherein said microorganisms are further modified to secrete said hydrocarbons in greater amounts than otherwise identical hydrocarbon-producing microorganisms lacking the modifications.

In certain embodiment, the invention also provides a recombinant multi-subunit prokaryotic efflux pump (YbhGFSR and functional homologs thereof) capable of mediating the export of intracellular n-alkanes and n-alkenes, e.g., n-pentadecane and n-heptadecene, generated by the concerted action of acyl-ACP reductase (AAR) and alkanal deformylative monooxygenase (ADM), and to the heterologous expression of its corresponding structural genes in a microorganism, e.g., a photosynthetic microorganism, such as a JCC138-derived adm-aar⁺ alkanogen, so as to enable said photosynthetic microorganism host to efflux n-alkanes into the growth medium. In certain embodiments, the invention provides a recombinant microorganism comprising recombinant alkane-producing enzymes described herein in addition to a recombinant outer membrane protein described herein (e.g., TolC or a TolC homolog) and an ABC efflux pump described herein (e.g., a YbhGFSR efflux pump or homolog thereof). In related embodiments, the invention provides methods of culturing such microorganisms, wherein said microorganisms secrete biosynthetic alkanes and/or alkanes into the culture medium.

In additional embodiments, the invention provides an engineered microorganism comprising a disrupted S layer or a disrupted glycocalyx, wherein said engineered microorganism comprises (i) one or more recombinant genes encoding enzymes which catalyze the production of n-alkanes or n-alkenes, and (ii) a mutation in a gene involved in the biosynthesis or maintenance of said S layer or said glycocalyx, wherein said mutation leads to the disruption of said S layer or said glycocalyx. In related embodiments, the invention provides methods of culturing such microorganisms, wherein said microorganisms secrete biosynthetic alkanes and/or alkanes into the culture medium.

In other embodiments, the invention provides an engineered photosynthetic microorganism, wherein said engineered photosynthetic microorganism comprises (i) one or more recombinant genes encoding enzymes which catalyze the production of n-alkanes, and (ii) one or more recombinant genes encoding an acetyl-CoA carboxylase. In related embodiments, the invention provides methods for producing hydrocarbons, comprising culturing such an wherein said engineered microorganism produces n-alkanes and/or n-alkenes, and wherein said engineered microorganism secretes increased amounts of n-alkanes and/or n-alkenes into the culture medium relative to an otherwise identical microorganism, cultured under identical conditions, but lacking said one or more genes encoding said acetyl-CoA carboxylase.

Additional embodiments include the following, presented in claim format:

1. An engineered microorganism, wherein said engineered microorganism comprises (i) one or more recombinant genes encoding enzymes which catalyze the production of alkanes, and (ii) one or more recombinant genes encoding one or more protein components of a recombinant hydrocarbon ABC efflux pump system.

2. The engineered microorganism of claim 1, wherein said recombinant genes encoding enzymes which catalyze the production of alkanes are selected from the group consisting of a recombinant acyl-ACP reductase enzyme and a recombinant alkanal deformylative monooxygenase (ADM) enzyme.

3. The engineered microorganism of claim 1, wherein said recombinant hydrocarbon ABC efflux pump system is an E. coli hydrocarbon ABC efflux pump system.

4. The engineered microorganism of claim 3, wherein said recombinant hydrocarbon ABC efflux pump system is selected from the group consisting of the ybhG/ybhF/ybhS/ybhR/tolC and the yhiI/rbbA/yhhJ/tolC pump system.

5. The engineered microorganism of claim 4, wherein said one or more recombinant genes encoding one or more protein components of a recombinant hydrocarbon ABC efflux pump system encode at least one protein listed in Table 5, or a functional homolog of at least one protein listed in Table 5.

6. The engineered microorganism of any of claims 1-5, wherein said microorganism is E. coli.

7. The engineered microorganism of claim 5, wherein expression of an operon comprising ybhG/ybhF/ybhS/ybhR is controlled by a recombinant promoter, and wherein said promoter is constitutive or inducible.

8. The engineered microorganism of claim 7, wherein said operon is integrated into the genome of said microorganism.

9. The engineered microorganism of claim 7, wherein said operon is extrachromosomal.

10. The engineered microorganism of any of claims 1-5, wherein said microorganism is a photosynthetic microorganism.

11. The engineered photosynthetic microorganism of claim 10, wherein said microorganism is a cyanobacterium.

12. The engineered photosynthetic microorganism of claim 11, wherein said microorganism is a Synechococcus species.

13. The engineered photosynthetic microorganism of any of claims 10-12, wherein said one or more protein components are selected from the group consisting of YbhG, YhiI, TolC and homologs of YbhG, YhiI and TolC, wherein the native leader sequences of said YbhG, YhiI and TolC proteins and homologs thereof are replaced with leader sequences native to said photosynthetic microorganism.

14. The engineered photosynthetic microorganism of claim 13, wherein said protein components comprise a YbhG variant selected from Set 1 of Table 20, and wherein said TolC homolog is SYNPCC7002_A0585.

15. The engineered photosynthetic microorganism of claim 13, wherein said protein components comprise a YbhG variant selected from Set 2 of Table 20, and wherein said TolC or TolC homolog is selected from the OMP variants listed in Set 2 of Table 20.

16. The engineered photosynthetic microorganism of any of claims 11-13, wherein said protein components comprise YbhS and YbhR proteins or homologs thereof, and wherein said YbhS and YbhR proteins or homologs thereof comprise pseudo-leader sequences.

17. The engineered photosynthetic microorganism of claim 16, wherein said YbhS and YbhR proteins or homologs thereof are selected from those listed in Table 20.

18. The engineered photosynthetic microorganism of any of claims 11-13, wherein said one or more protein components is a recombinant TolC or homolog of TolC, and wherein said TolC or said homolog of TolC includes a C-terminal modification wherein the C-terminal residues of TolC are replaced with the corresponding C-terminal residues of an outer membrane protein native to said photosynthetic microorganism.

19. The engineered photosynthetic microorganism of claim 19, wherein said TolC or TolC homolog is an OMP variant from Table 20.

20. An engineered photosynthetic microorganism comprising a recombinant outer membrane protein and a recombinant complementary ABC efflux pump, wherein said recombinant outer membrane protein is SYNPCC7002_A0585, and wherein said recombinant complementary ABC efflux pump comprises (i) a YbhG variant selected from Set 1 of Table 20, (ii) YbhF, and (iii) a YbhS/YbhR variant listed in Table 20.

21. An engineered photosynthetic microorganism comprising a recombinant outer membrane protein and a recombinant complementary ABC efflux pump, wherein said recombinant outer membrane protein is selected from the group consisting of the OMP variants listed in Set 2 of Table 20, and wherein said recombinant ABC efflux pump comprises (i) a YbhG variant selected from Set 2 of Table 20, (ii) YbhF, and (iii) a YbhS/YbhR variant listed in Table 20.

22. An engineered photosynthetic microorganism of any of claims 13-21, wherein said engineered photosynthetic microorganism comprises a recombinant outer membrane protein and a recombinant complementary ABC efflux pump, and wherein expression of said recombinant outer membrane protein and said recombinant ABC efflux pump is driven by distinct promoters.

23. An engineered photosynthetic microorganism of claim 22, wherein at least one of said separate promoters is inducible.

24. An engineered photosynthetic microorganism of claim 22, wherein said promoters are divergently oriented.

25. An engineered photosynthetic microorganism of claim 24, wherein said promoters are selected from the promoters listed in Table 19.

26. A method for producing hydrocarbons, comprising:

culturing an engineered microorganism of any of claims 1-25 in a culture medium, wherein said engineered microorganism secretes increased amounts of n-alkanes or n-alkenes into the culture medium relative to an otherwise identical microorganism, cultured under identical conditions, but lacking said recombinant genes.

27. The method of claim 26, wherein said culture medium does not include a surfactant.

28. The method of claim 26, wherein said culture medium does not include EDTA.

29. The method of claim 26, wherein said culture medium does not include Tris buffer.

30. The method of claim 26, wherein said engineered microorganism secretes as least twice the percentage of n-alkanes produced relative to an otherwise identical microorganism, cultured under identical conditions, but lacking said recombinant genes for efflux of n-alkanes or n-alkenes.

31. The method of claim 26, wherein said engineered microorganism secretes as least five times the percentage of n-alkanes produced relative to an otherwise identical microorganism, cultured under identical conditions, but lacking said recombinant genes for the efflux of n-alkanes or n-alkenes.

32. The method of claim 26, wherein said engineered microorganism is an engineered E. coli, and wherein at least 90% of said n-alkanes or n-alkenes are secreted into the culture medium.

33. A method for producing hydrocarbons, comprising:

(i) culturing an engineered photosynthetic microorganism of any of claims 10-25 in a culture medium, and

(ii) exposing said engineered photosynthetic microorganism to light and carbon dioxide, wherein said exposure results in the conversion of said carbon dioxide by said engineered cynanobacterium into n-alkanes, wherein said n-alkanes are secreted into said culture medium in an amount greater than that secreted by an otherwise identical cyanobacterium, cultured under identical conditions, but lacking said recombinant genes.

34. The method of claim 33, wherein said engineered photosynthetic microorganism further produces at least one n-alkene or n-alkanol.

35. The method of claim 33, wherein said engineered photosynthetic microorganism produces at least one n-alkene or n-alkanol selected from the group consisting of n-pentadecene, n-heptadecene, and 1-octadecanol.

36. The method of claim 33, wherein said n-alkanes comprise predominantly n-heptadecane, n-pentadecane or a combination thereof.

37. The method of claim 33, further comprising isolating at least one n-alkane, n-alkene or n-alkanol from said culture medium.

38. The method of claim 33, wherein at least one of said recombinant genes is encoded on a plasmid.

39. The method of claim 33, wherein at least one of said recombinant genes is incorporated into the genome of said engineered photosynthetic microorganism.

40. The method of claim 33, wherein at least one of said recombinant genes is present in multiple copies in said engineered photosynthetic microorganism.

41. The method of claim 33 wherein at least two of said recombinant genes are part of an operon, and wherein the expression of said genes is controlled by a single promoter.

42. The method of claim 33, wherein at least 95% of said n-alkanes are n-pentadecane and n-heptadecane.

43. The method of claim 33, wherein the expression of at least one of said recombinant genes is controlled by one or more inducible promoters.

44. The method of claim 43, wherein at least one promoter is a urea-repressible, nitrate-inducible promoter.

45. The method of claim 44, wherein said promoter is a nirA-type promoter.

46. The method of claim 45, wherein said nirA-type promoter is P(nir07) or P(nir09).

47. A method for producing a hydrocarbon of interest, comprising (i) culturing an engineered Escherichia coli cell in a culture medium, wherein said cell comprises a mutation in a promoter for the ybiH gene or a mutation in the structural gene encoding YbiH activity, wherein said mutation decreases expression of YbiH activity relative to an otherwise identical cell lacking said mutation and, and wherein said mutation increases secretion of said hydrocarbon of interest relative to an otherwise identical cell lacking said hydrocarbon of interest; and (ii) isolating said hydrocarbon of interest from said culture medium.

48. The method of claim 47, wherein said hydrocarbon of interest is a biofuel.

49. An engineered microorganism comprising a disrupted lipopolysaccharide (LPS) layer, wherein said engineered microorganism comprises (i) one or more recombinant genes encoding enzymes which catalyze the production of n-alkanes, and (ii) a mutation in a gene involved in the biosynthesis or maintenance of said LPS layer, wherein said mutation leads to the disruption of said LPS layer.

50. The engineered microorganism of claim 49, wherein said gene involved in the maintenance of said LPS layer encodes ADP-heptose:LPS heptosyl transferase I.

51. The engineered microorganism of claim 49, wherein said microorganism is E. coli.

52. The engineered microorganism of claim 49, wherein said microorganism is a photosynthetic microorganism.

53. The engineered microorganism of claim 52, wherein said microorganism is a cyanobacterium.

54. A method for producing hydrocarbons, comprising: culturing an engineered microorganism of any of claims 49-53 in a culture medium, wherein said engineered microorganism produces n-alkanes or n-alkenes, and wherein said engineered microorganism secretes increased amounts of n-alkanes or n-alkenes into the culture medium relative to an otherwise identical microorganism, cultured under identical conditions, but lacking said mutation in said gene involved in the biosynthesis or maintenance of said LPS layer.

55. The method of claim 54, wherein said engineered microorganism is an engineered E. coli and wherein at least 10% of said n-alkanes or n-alkenes are secreted into the culture medium.

56. The method of claim 54, wherein said engineered microorganism is an engineered E. coli and wherein at least 50% of said n-alkanes or n-alkenes are secreted into the culture medium.

57. The method of claim 54, wherein said engineered microorganism is a photosynthetic microorganism.

58. The method of claim 54, wherein said microorganism is a cyanobacterium.

59. An engineered microorganism comprising a disrupted S layer or a disrupted glycocalyx, wherein said engineered microorganism comprises (i) one or more recombinant genes encoding enzymes which catalyze the production of n-alkanes or n-alkenes, and (ii) a mutation in a gene involved in the biosynthesis or maintenance of said S layer or said glycocalyx, wherein said mutation leads to the disruption of said S layer or said glycocalyx.

60. The engineered photosynthetic microorganism of claim 59, wherein said one or more recombinant genes are selected from the group consisting of an AAR enzyme, an ADM enzyme, or both enzymes.

61. The engineered photosynthetic microorganism of claim 59, wherein said gene involved in the biosynthesis or maintenance of said S layer or said glycocalyx is selected from Table 10B.

62. The engineered microorganism of any of claims 59-61, wherein said microorganism is a cyanobacterium.

63. A method for producing hydrocarbons, comprising: culturing an engineered microorganism of any of claims 59-62 in a culture medium, wherein said engineered microorganism produces n-alkanes or n-alkenes, and wherein said engineered microorganism secretes increased amounts of n-alkanes or n-alkenes into the culture medium relative to an otherwise identical microorganism, cultured under identical conditions, but lacking said mutation in said gene involved in the biosynthesis or maintenance of said S layer or said glycocalyx.

64. An engineered photosynthetic microorganism, wherein said engineered photosynthetic microorganism comprises (i) one or more recombinant genes encoding enzymes which catalyze the production of n-alkanes, and (ii) one or more recombinant genes encoding an acetyl-CoA carboxylase.

65. The engineered photosynthetic microorganism of claim 64, wherein said one or more recombinant genes are selected from the group consisting of an acyl-ACP reductase enzyme, an ADM enzyme, or both enzymes.

66. The engineered photosynthetic microorganism of claim 64 or 65, wherein said recombinant acetyl-CoA carboxylase is E. coli acetyl-CoA carboxylase.

67. The engineered photosynthetic microorganism of any of claims 64-66, wherein said recombinant genes encoding acetyl-CoA carboxylase are controlled by an inducible promoter.

68. The engineered photosynthetic microorganism of claim 67, wherein said inducible promoter is an ammonia-repressible nitrate reductase promoter.

69. The engineered photosynthetic microorganism of claim 68, wherein said ammonia-repressible nitrate reductase promoter is selected from the group consisting of p(nir07) and p(nir09).

70. The engineered photosynthetic microorganism of any of claims 64-69, wherein said photosynthetic microorganism is a cyanobacterium.

71. The engineered photosynthetic microorganism of claim 70, wherein said cyanobacterium is a Synechococcus species.

72. A method for producing hydrocarbons, comprising: culturing an engineered photosynthetic microorganism of any of claims 64-71 in a culture medium, wherein said engineered microorganism produces n-alkanes, and wherein said engineered microorganism secretes increased amounts of n-alkanes into the culture medium relative to an otherwise identical microorganism, cultured under identical conditions, but lacking said one or more genes encoding an acetyl-CoA carboxylase.

73. The method of claim 72, wherein the percent secretion of n-alkanes is between 2-fold and 90-fold greater than that achieved by culturing an otherwise identical strain, under identical conditions, but lacking the recombinant genes encoding acetyl-CoA carboxylase.

74. The method of claim 72, wherein between 1% and 25% of n-alkanes produced by the cell are secreted.

75. The method of claim 72, wherein at least 15% of n-alkanes produced by the cell are secreted.

76. The method of any of claims 72-75, further comprising isolating said n-alkanes from the culture medium.

77. An isolated nucleic acid, wherein said isolated nucleic acid comprises an engineered nucleotide sequence selected from SEQ ID NOs: 1-214.

78. An isolated nucleic acid, wherein said isolated nucleic acid encodes an engineered protein comprising an amino acid sequence selected from SEQ ID NOs: 1-214.

79. An engineered microbe, wherein said engineered microbe comprises a recombinant nucleic acid or recombinant protein comprising a sequence selected from SEQ ID NO: 1-214.

80. The engineered microbe of claim 79, wherein said engineered microbe is a photosynthetic microbe.

81. The engineered microbe of claim 80, wherein said engineered photosynthetic microbe is a cyanobacterium.

In certain embodiments, the invention also provides various nucleic acid constructs and/or vectors and associated methods for engineering the various microorganisms described herein.

Various embodiments of the invention are further described in the Figures, Description, Examples and Claims, herein.

### FIGURES

FIG. 1 Hydrocarbon production by E. coli BL21(DE3) derivatives JCC1169, JCC1170, JCC1214, and JCC1113. #1 and #2 indicate the numbers of each of the two biological replicate cultures used for each strain. T1 represents the time just before addition of 1 mM IPTG; T2 represents a time 3.5 hr after T1. The fraction of total alka(e)ne for each of the JCC1214 and JCC1113 T2 samples that was detected in the medium-associated extractant is indicated.

FIG. 2 The ybhGFSR genomic region in E. coli, encoding the components of the putative YbhGFSR ABC efflux pump for extruding hydrocarbons like n-pentadecane out of the cell. ybhG encodes the membrane fusion protein (MFP), ybhF encodes the ATP-hydrolytic subunit (also referred to herein as the ATP-binding subunit), and ybhS and ybhR encode the inner membrane subunits (also referred to herein as permease subunits). Below the gene map are the fluorescence signals of the Agilent microarray probes corresponding to the gene above each bar graph (the y-axis is the probe fluorescence signal). The first two bars represent JCC1169 T1 and T2, respectively; the next two bars JCC1170 T1 and T2, respectively; the next two bars, JCC1214 T1 and T2, respectively; the next two bars JCC1113 T1 and T2, respectively. Each bar has two sub-bars corresponding to the two replicate cultures of each strain, #1 and #2.

FIG. 3 Sequence logo of the short loop sequence separating the coil-coiled helices in the following known E. coli MFS TolC-interactors: EmrA, EmrK, AcrA, AcrE, MdtE, MdtA, and MacA.

FIG. 4 is a schematic depiction of the fully assembled YbhGFSR-TolC efflux pump.

FIG. 5 depicts schematically the native ybiH/ybhG/ybhF/ybhS/ybhR operon (top) and a recombinant operon wherein ybiH is disrupted and the promoter of the operon is replaced.

FIG. 6 shows the relative alkane production and secretion capabilities of various engineered E. coli strains that recombinantly express ADM and AAR enzyme activities.

FIG. 7 shows alkane production and secretion by overexpression of ybhGFSR in E. coli JCC1880 expressing adm-aar.

FIG. 8 shows production of pentadecane in the medium and cell pellets of JCC2055 derived strains bearing the A0585_ProNTerm_tolC and ybhGFSR transporter. Data are also included from a control strain (JCC2055 1) which did not contain the transporter and produced a similar titre of pentadecane. The % of pentadecane in the medium is indicated above the bar for each strain.

### DETAILED DESCRIPTION OF THE INVENTION

Unless otherwise defined herein or in the above-mentioned utility applications, e.g., U.S. patent application Ser. No. 12/833,821, filed Jul. 9, 2010, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art.

Cyanobacteria contain not only a plasma membrane (PM) like non-photosynthetic prokaryotic hosts (as well as an outer membrane like their Gram-negative non-photosynthetic counterparts), but also, typically, an intracellular thylakoid membrane (TM) system that serves as the site for photosynthetic electron transfer and proton pumping. Given that both the plasma membrane and thylakoid membrane are typically loaded with proteins, both integral and peripheral, and, further, that a significant fraction of experimentally detected membrane proteins, both integral and peripheral, appear to be uniquely localized in each membrane, the question arises as to how differential localization of membrane proteins between the PM and TM is achieved in cyanobacteria (Rajalahti T et al. (2007) J Proteome Res 6:2420-2434). This question is of relevance to cyanobacterial metabolic engineering because certain heterologous enzymatic functions that may be desirable to engineer into said photosynthetic hosts are encoded by heterologous integral plasma membrane proteins (HIPMPs), both prokaryotic and eukaryotic in origin, that must be targeted to the plasma membrane of the cyanobacterial host in order to function as desired. The HIPMPs of interest in this respect comprise proteins that mediate transport, typically efflux, of substrates across the cyanobacterial plasma membrane. HIPMPs of particular interest with respect to the efflux of n-alkanes and n-alkenes are the integral plasma membrane subunits, YbhS and YbhR, of a putative YbhGFSR-TolC efflux pump system from E. coli.

The methods described herein can be extended to integral membrane proteins that are not HIPMPs, i.e., proteins that are derived from membranes other than the plasma membrane. Such alternative membranes include: the thylakoid membrane, the endoplasmic reticulum membrane, the chloroplast inner membrane, and the mitochondrial inner membrane.

In one embodiment, the disclosure provides methods for designing a protein comprising a pseudo-leader sequence (PLS) of defined sequence fused to the N-terminus of an HIPMP of interest, wherein the resulting chimeric protein is expressed in a cyanobacterial host cell, e.g., JCC138 (Synechocystis sp. PCC 7002) or an engineered derivative thereof. The expression of the chimeric protein will increase the amount of hydrocarbon products of interest (e.g., alkanes, alkenes, alkyl alkanoates, etc.) exported from the cynanobacterial host cell. The PLS encodes a contiguous polypeptide sub-fragment of a protein from a different thylakoid-membrane-containing cyanobacterial host, e.g., JCC160 (Synechococcus sp. PCC 6803), that localizes as uniquely as possible to the plasma membrane of that host. The mechanism that this non-JCC138 host natively employs to effect the localization of the protein to the plasma membrane (rather than the thylakoid membrane) should be conserved in order for the localization to occur in the recipient host.

While PLSs are designed to ensure, or at least bias, the targeting of HIPMPs to the plasma membrane of the heterologous cyanobacterial host, they may not always be required. This is because sufficient levels of functional HIPMP may become embedded in the plasma membrane if the cyanobacterial host does, in fact, mechanistically recognize the protein as a native plasma membrane protein—even if some fraction of the protein is targeted to the thylakoid membrane or ends up in neither membrane (e.g., as inclusion bodies).

For HIPMPs with cytoplasmic N-termini (i) the PLS is derived from a plasma-membrane-resident protein that is naturally anchored in the membrane of a different cyanobacterial species (i.e., different than the species into which the PLS will be functionally expressed) via two transmembrane α helices, and (ii) said plasma-membrane-resident protein naturally has its N-terminus within the cytoplasm and its C-terminus within the cytoplasm (Nᵢₙ/Cᵢₙ), spanning the plasma membrane via an in-to-out transmembrane α helix, followed by an (ideally short) periplasmic loop sequence, followed by an out-to-in transmembrane α helix. Correspondingly, for HIPMPs with periplasmic N-termini (Nₒᵤₜ), (i) the PLS is derived from a plasma-membrane-resident protein that is naturally anchored in the membrane of a different cyanobacterial species via one transmembrane α helix, and (ii) said plasma-membrane-resident protein naturally has its N-terminus within the cytoplasm and its C-terminus within the periplasm (Nᵢₙ/Cₒᵤₜ).

In a preferred embodiment, PLSs are derived from host proteins that have most of their mass in either the periplasmic and/or cytoplasmic spaces. In another preferred embodiment, said PLSs should contain only two α helices with Nᵢₙ/Cᵢₙ topology (for creating Nᵢₙ HIPMPs) and only one α helix with Nᵢₙ/Cₒᵤₜ topology (for creating Nₒᵤₜ HIPMPs). In a related embodiment, the potential for intermolecular homomultimerization among the transmembrane helices of the PLSs is minimized.

The terms “fused”, “fusion” or “fusing” used herein in the context of chimeric proteins refers to the joining of one functional protein or protein subunit (e.g., a pseudo-leader sequence) to another functional protein or protein subunit (e.g., an integral plasma membrane protein). Fusing can occur by any method which results in the covalent attachment of the C-terminus of one such protein molecule to the N-terminus of another. For example, one skilled in the art will recognize that fusing occurs when the two proteins to be fused are encoded by a recombinant nucleic acid under control of a promoter and expressed as a single structural gene in vivo or in vitro.

As used herein, the term “non-target” refers to a protein or nucleic acid that is native to a species that is different than the species that will be used to recombinantly express the protein or nucleic acid.

Alkanes, also known as paraffins, are chemical compounds that consist only of the elements carbon (C) and hydrogen (H) (i.e., hydrocarbons), wherein these atoms are linked together exclusively by single bonds (i.e., they are saturated compounds) without any cyclic structure. n-Alkanes are linear, i.e., unbranched, alkanes.

Genes encoding AAR or ADM enzymes are referred to herein as Aar genes (aar) or Adm genes (adm), respectively. Together, AAR and ADM enzymes function to synthesize n-alkanes from acyl-ACP molecules. As used herein, an AAR enzyme refers to an enzyme with the amino acid sequence of the SYNPCC7942_{—}1594 protein or a homolog thereof, wherein a SYNPCC7942_{—}1594 homolog is a protein whose BLAST alignment (i) covers >90% length of SYNPCC7942_{—}1594, (ii) covers >90% of the length of the matching protein, and (iii) has >50% identity with SYNPCC7942_{—}1594 (when optimally aligned using the parameters provided herein), and retains the functional activity of SYNPCC7942_{—}1594, i.e., the conversion of an acyl-ACP (acyl-acyl carrier protein) to an n-alkanal. An ADM enzyme refers to an enzyme with the amino acid sequence of the SYNPCC7942_{—}1593 protein or a homolog thereof, wherein a SYNPCC7942_{—}1593 homolog is defined as a protein whose amino acid sequence alignment (i) covers >90% length of SYNPCC7942_{—}1593, (ii) covers >90% of the length of the matching protein, and (iii) has >50% identity with SYNPCC7942_{—}1593 (when aligned using the preferred parameters provided herein), and retains the functional activity of SYNPCC7942_{—}1593, i.e., the conversion of an n-alkanal to an (n-1)-alkane. Exemplary AAR and ADM enzymes are listed in Table 1 and Table 2, respectively, of U.S. utility application Ser. No. 12/759,657, filed Apr. 13, 2010 (now U.S. Pat. No. 7,794,969), and U.S. utility application Ser. No. 12/833,821, filed Jul. 9, 2010. Other ADM activities are described in U.S. patent application Ser. No. 12/620,328, filed Nov. 17, 2009. Applicants note that in previous related applications, this enzyme was referred to as an alkanal decarboxylative monooxygenase. The protein is referred to herein as an alkanal deformylative monooxygenase or abbreviated as ADM; to be clear, it is the same protein referred to in the related applications

Preferred parameters for BLASTp are: Expectation value: 10 (default); Filter: none; Cost to open a gap: 11 (default); Cost to extend a gap: 1 (default); Maximum alignments: 100 (default); Word size: 11 (default); No. of descriptions: 100 (default); Penalty Matrix: BLOWSUM62.

Functional homologs of other proteins described herein (e.g., TolC homologs, YbhG homologs, YbhF homologs, YbhR homologs, YbhS homologs and SYNPCC7002_A0585 homologs) may share significant amino acid identity (>50%) with the named proteins whose sequences are presented herein. Such homologs may be obtained from other organisms where the proteins are known to share structural and functional characteristics with the named proteins. For example, a functional outer membrane protein that is at least 95% identical to E. coli TolC is considered a TolC homolog. Likewise, a functional outer membrane protein that is at least 95% identical to TolC except for the replacement/addition of leader sequences, C-terminal sequences or other modifications intended to increase its functionality in a particular environment (e.g., a non-native host) are also considered functional homologs of TolC. The same definitions apply to other protein homologs referred to herein.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

One skilled in the art will also recognize, in light of the teachings herein, that the methods and compositions described herein for use in particular organisms, e.g., cyanobacteria, are also applicable other organisms, e.g., gram-negative bacteria such as E. coli. For example, a chimeric integral plasma membrane protein for facilitating alkane efflux in E. coli could be designed by fusing a pseudo leader sequence derived from E. coli or a related bacterium to a heterologous integral plasma membrane protein.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

As used herein, an “isolated” organic molecule (e.g., an alkane, alkene, or alkanal) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this present disclosure may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

The term “deletion” refers to the removal of one or more nucleotides from a nucleic acid molecule or one or more amino acids from a protein, the regions on either side being joined together.

The term “knock out” refers to a gene whose level of expression or activity has been reduced to zero. In some examples, a gene is knocked-out via deletion of some or all of its coding sequence. In other examples, a gene is knocked-out via introduction of one or more nucleotides into its open reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present disclosure have particular utility. The heterologous polypeptide included within the fusion protein of the present disclosure is at least 6 amino acids in length, often at least 8 amino acids in length, and usefully at least 15, 20, and 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′).sub.2, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Intracellular Antibodies: Research and Disease Applications, (Marasco, ed., Springer-Verlag New York, Inc., 1998), the disclosure of which is incorporated herein by reference in its entirety).

As used herein, antibodies can be produced by any known technique, including harvest from cell culture of native B lymphocytes, harvest from culture of hybridomas, recombinant expression systems and phage display.

The term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the present disclosure may be used to produce an equivalent effect and are therefore envisioned to be part of the present disclosure.

A “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

A mutein has at least 85% overall sequence homology to its wild-type counterpart. Even more preferred are muteins having at least 90% overall sequence homology to the wild-type protein.

In an even more preferred embodiment, a mutein exhibits at least 95% sequence identity, even more preferably 98%, even more preferably 99% and even more preferably 99.9% overall sequence identity.

Sequence homology may be measured by any common sequence analysis algorithm, such as Gap or Bestfit.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present disclosure. Examples of unconventional amino acids include: 4-hydroxyproline, γ-carboxyglutamate, ε-N,N,N-trimethyllysine, ε-N-acetyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

“Percent dry cell weight” refers to a measurement of hydrocarbon production obtained as follows: a defined volume of culture is centrifuged to pellet the cells. Cells are washed then dewetted by at least one cycle of microcentrifugation and aspiration. Cell pellets are lyophilized overnight, and the tube containing the dry cell mass is weighed again such that the mass of the cell pellet can be calculated within ±0.1 mg. At the same time cells are processed for dry cell weight determination, a second sample of the culture in question is harvested, washed, and dewetted. The resulting cell pellet, corresponding to 1-3 mg of dry cell weight, is then extracted by vortexing in approximately 1 ml acetone plus butylated hydroxytolune (BHT) as antioxidant and an internal standard, e.g., n-eicosane. Cell debris is then pelleted by centrifugation and the supernatant (extractant) is taken for analysis by GC. For accurate quantitation of n-alkanes, flame ionization detection (FID) is used as opposed to MS total ion count. n-Alkane concentrations in the biological extracts are calculated using calibration relationships between GC-FID peak area and known concentrations of authentic n-alkane standards. Knowing the volume of the extractant, the resulting concentrations of the n-alkane species in the extractant, and the dry cell weight of the cell pellet extracted, the percentage of dry cell weight that comprised n-alkanes can be determined.

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

“Carbon-based Products of Interest” include alcohols such as ethanol, propanol, isopropanol, butanol, fatty alcohols, fatty acid esters, wax esters; hydrocarbons and alkanes such as propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, ε-caprolactone, isoprene, caprolactam, rubber; commodity chemicals such as lactate, docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals.

Biofuel: A biofuel refers to any fuel that derives from a biological source. Biofuel can refer to one or more hydrocarbons, one or more alcohols, one or more fatty esters or a mixture thereof.

Hydrocarbon: The term generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Throughout this specification and claims, the word “comprise” or variations such as “comprises” or “comprising”, will be understood to imply the inclusion of a stated integer or group of integers but not the exclusion of any other integer or group of integers.

In another embodiment, the nucleic acid molecule of the present disclosure encodes a polypeptide having the amino acid sequence of any of the protein sequences provided in SEQ ID NOs: 1-214. Preferably, the nucleic acid molecule of the present disclosure encodes a polypeptide sequence of at least 50%, 60, 70%, 80%, 85%, 90% or 95% identity to one of the protein sequences of SEQ ID NOs: 1-214 and the identity can even more preferably be 96%, 97%, 98%, 99%, 99.9% or even higher.

In yet another embodiment, novel nucleic acid sequences useful for the recombinant expression of ABC efflux pump systems are provided, including the YbhG, YbhF, YbhS and YbhR variants listed in Table 20. The invention also provides the engineered outer membrane proteins listed in Table 20 and the nucleic acid sequences encoding those proteins.

The present disclosure also provides nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

The nucleic acid sequence fragments of the present disclosure display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hydridization to target sequences can be detected and optionally quantified. One of skill in the art will appreciate that the nucleic acid fragments of the present disclosure may be used in a wide variety of blotting techniques not specifically described herein.

It should also be appreciated that the nucleic acid sequence fragments disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosure of each of which is incorporated herein by reference in its entirety.

As is well known in the art, enzyme activities can be measured in various ways. For example, the pyrophosphorolysis of OMP may be followed spectroscopically (Grubmeyer et al., (1993) J. Biol. Chem. 268:20299-20304). Alternatively, the activity of the enzyme can be followed using chromatographic techniques, such as by high performance liquid chromatography (Chung and Sloan, (1986) J. Chromatogr. 371:71-81). As another alternative the activity can be indirectly measured by determining the levels of product made from the enzyme activity. These levels can be measured with techniques including aqueous chloroform/methanol extraction as known and described in the art (Cf. M. Kates (1986) Techniques of Lipidology; Isolation, analysis and identification of Lipids. Elsevier Science Publishers, New York (ISBN: 0444807322)). More modern techniques include using gas chromatography linked to mass spectrometry (Niessen, W. M. A. (2001). Current practice of gas chromatography—mass spectrometry. New York, N.Y.: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G (1997) Am. Chem. Soc. Symp. Series, 666: 172-208), titration for determining free fatty acids (Komers (1997) Fett/Lipid, 99(2): 52-54), enzymatic methods (Bailer (1991) Fresenius J. Anal. Chem. 340(3): 186), physical property-based methods, wet chemical methods, etc. can be used to analyze the levels and the identity of the product produced by the organisms of the present disclosure. Other methods and techniques may also be suitable for the measurement of enzyme activity, as would be known by one of skill in the art.

Also provided by the present disclosure are vectors, including expression vectors, which comprise the above nucleic acid molecules of the present disclosure, as described further herein. In a first embodiment, the vectors include the isolated nucleic acid molecules described above. In an alternative embodiment, the vectors of the present disclosure include the above-described nucleic acid molecules operably linked to one or more expression control sequences. The vectors of the instant disclosure may thus be used to express an Aar and/or Adm polypeptide contributing to n-alkane producing activity by a host cell, and/or a chimeric efflux protein for effluxing n-alkanes and other hydrocarbons out of the cell.

In another aspect of the present disclosure, host cells transformed with the nucleic acid molecules or vectors of the present disclosure, and descendants thereof, are provided. In some embodiments of the present disclosure, these cells carry the nucleic acid sequences of the present disclosure on vectors, which may but need not be freely replicating vectors. In other embodiments of the present disclosure, the nucleic acids have been integrated into the genome of the host cells.

In a preferred embodiment, the host cell comprises one or more AAR or ADM encoding nucleic acids which express AAR or ADM in the host cell.

In an alternative embodiment, the host cells of the present disclosure can be mutated by recombination with a disruption, deletion or mutation of the isolated nucleic acid of the present disclosure so that the activity of the AAR and/or ADM protein(s) in the host cell is reduced or eliminated compared to a host cell lacking the mutation.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A variety of host organisms can be transformed to produce a product of interest. Photoautotrophic organisms include eukaryotic plants and algae, as well as prokaryotic cyanobacteria, green-sulfur bacteria, green non-sulfur bacteria, purple sulfur bacteria, and purple non-sulfur bacteria.

Extremophiles are also contemplated as suitable organisms. Such organisms withstand various environmental parameters such as temperature, radiation, pressure, gravity, vacuum, desiccation, salinity, pH, oxygen tension, and chemicals. They include hyperthermophiles, which grow at or above 80° C. such as Pyrolobus fumarii; thermophiles, which grow between 60-80° C. such as Synechococcus lividis; mesophiles, which grow between 15-60° C. and psychrophiles, which grow at or below 15° C. such as Psychrobacter and some insects. Radiation tolerant organisms include Deinococcus radiodurans. Pressure-tolerant organisms include piezophiles, which tolerate pressure of 130 MPa. Weight-tolerant organisms include barophiles. Hypergravity (e.g., >1 g) and hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt-tolerant organisms include halophiles (e.g., 2-5 M NaCl) Halobacteriacea and Dunaliella salina. pH-tolerant organisms include alkaliphiles such as Natronobacterium, Bacillus firmus OF4, Spirulina spp. (e.g., pH>9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas-tolerant organisms, which tolerate pure CO₂ include Cyanidium caldarium and metal tolerant organisms include metalotolerants such as Ferroplasma acidarmanus (e.g., Cu, As, Cd, Zn), Ralstonia sp. CH34 (e.g., Zn, Co, Cd, Hg, Pb). Gross, Michael. Life on the Edge: Amazing Creatures Thriving in Extreme Environments. New York: Plenum (1998) and Seckbach, J. “Search for Life in the Universe with Terrestrial Microbes Which Thrive Under Extreme Conditions.” In Cristiano Batalli Cosmovici, Stuart Bowyer, and Dan Wertheimer, eds., Astronomical and Biochemical Origins and the Search for Life in the Universe, p. 511. Milan: Editrice Compositori (1997).

Plants include but are not limited to the following genera: Arabidopsis, Beta, Glycine, Jatropha, Miscanthus, Panicum, Phalaris, Populus, Saccharum, Salix, Simmondsia and Zea.

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium. A partial list of cyanobacteria that can be engineered to express the recombinant described herein include members of the genus Chamaesiphon, Chroococcus, Cyanobacterium, Cyanobium, Cyanothece, Dactylococcopsis, Gloeobacter, Gloeocapsa, Gloeothece, Microcystis, Prochlorococcus, Prochloron, Synechococcus, Synechocystis, Cyanocystis, Dermocarpella, Stanieria, Xenococcus, Chroococcidiopsis, Myxosarcina, Arthrospira, Borzia, Crinalium, Geitlerinemia, Leptolyngbya, Limnothrix, Lyngbya, Microcoleus, Oscillatoria, Planktothrix, Prochlorothrix, Pseudanabaena, Spirulina, Starria, Symploca, Trichodesmium, Tychonema, Anabaena, Anabaenopsis, Aphanizomenon, Cyanospira, Cylindrospermopsis, Cylindrospermum, Nodularia, Nostoc, Scylonema, Calothrix, Rivularia, Tolypothrix, Chlorogloeopsis, Fischerella, Geitieria, Iyengariella, Nostochopsis, Stigonema and Thermosynechococcus.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera:

Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis,

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic S-Metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

Preferred organisms for the manufacture of n-alkanes according to the methods discloused herein include: Arabidopsis thaliana, Panicum virgatum, Miscanthus giganteus, and Zea mays (plants); Botryococcus braunii, Chlamydomonas reinhardtii and Dunaliela salina (algae); Synechococcus sp PCC 7002, Synechococcus sp. PCC 7942, Synechocystis sp. PCC 6803, Thermosynechococcus elongatus BP-1 (cyanobacteria); Chlorobium tepidum (green sulfur bacteria), Chloroflexus auranticus (green non-sulfur bacteria); Chromatium tepidum and Chromatium vinosum (purple sulfur bacteria); Rhodospirillum rubrum, Rhodobacter capsulatus, and Rhodopseudomonas palusris (purple non-sulfur bacteria).

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still, other suitable organisms include microorganisms that can be engineered to fix carbon dioxide bacteria such as Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis.

A suitable organism for selecting or engineering is autotrophic fixation of CO₂ to products. This would cover photosynthesis and methanogenesis. Acetogenesis, encompassing the three types of CO₂ fixation; Calvin cycle, acetyl-CoA pathway and reductive TCA pathway is also covered. The capability to use carbon dioxide as the sole source of cell carbon (autotrophy) is found in almost all major groups of prokaryotes. The CO₂ fixation pathways differ between groups, and there is no clear distribution pattern of the four presently-known autotrophic pathways. See, e.g., Fuchs, G. 1989. Alternative pathways of autotrophic CO₂ fixation, p. 365-382. In H. G. Schlegel, and B. Bowien (ed.), Autotrophic bacteria. Springer-Verlag, Berlin, Germany. The reductive pentose phosphate cycle (Calvin-Bassham-Benson cycle) represents the CO₂ fixation pathway in almost all aerobic autotrophic bacteria, for example, the cyanobacteria.

For producing n-alkanes via the recombinant expression of Aar and/or Adm enzymes, an engineered cyanobacterium, e.g., a Synechococcus or Thermosynechococcus species, is preferred. Other preferred organisms include Synechocystis, Klebsiella oxytoca, Escherichia coli or Saccharomyces cerevisiae. Other prokaryotic, archaeal and eukaryotic host cells are also encompassed within the scope of the present disclosure.

In various embodiments of the disclosure, desired hydrocarbons and/or alcohols of certain chain length or a mixture thereof can be produced. In certain aspects, the host cell produces at least one of the following carbon-based products of interest: 1-dodecanol, 1-tetradecanol, 1-pentadecanol, n-tridecane, n-tetradecane, 15:1 n-pentadecene, n-pentadecane, 16:1 n-hexadecene, n-hexadecane, 17:1 n-heptadecene, n-heptadecane, 16:1 n-hexadecen-ol, n-hexadecan-1-ol and n-octadecen-1-ol, as shown in the Examples herein. In other aspects, the carbon chain length ranges from C₁₀ to C₂₀. Accordingly, the disclosure provides production of various chain lengths of alkanes, alkenes and alkanols suitable for use as fuels and chemicals.

In preferred aspects, the methods of the present disclosure include culturing host cells for direct product secretion for easy recovery without the need to extract biomass. These carbon-based products of interest are secreted directly into the medium. Since the disclosure enables production of various defined chain length of hydrocarbons and alcohols, the secreted products are easily recovered or separated. The products of the disclosure, therefore, can be used directly or used with minimal processing.

In various embodiments, compositions produced by the methods of the disclosure are used as fuels. Such fuels comply with ASTM standards, for instance, standard specifications for diesel fuel oils D 975-09b, and Jet A, Jet A-1 and Jet B as specified in ASTM Specification D. 1655-68. Fuel compositions may require blending of several products to produce a uniform product. The blending process is relatively straightforward, but the determination of the amount of each component to include in a blend is much more difficult. Fuel compositions may, therefore, include aromatic and/or branched hydrocarbons, for instance, 75% saturated and 25% aromatic, wherein some of the saturated hydrocarbons are branched and some are cyclic. Preferably, the methods of the disclosure produce an array of hydrocarbons, such as C₁₃-C₁₇ or C₁₀-C₁₅ to alter cloud point. Furthermore, the compositions may comprise fuel additives, which are used to enhance the performance of a fuel or engine. For example, fuel additives can be used to alter the freezing/gelling point, cloud point, lubricity, viscosity, oxidative stability, ignition quality, octane level, and flash point. Fuels compositions may also comprise, among others, antioxidants, static dissipater, corrosion inhibitor, icing inhibitor, biocide, metal deactivator and thermal stability improver.

In addition to many environmental advantages of the disclosure such as CO₂ conversion and renewable source, other advantages of the fuel compositions disclosed herein include low sulfur content, low emissions, being free or substantially free of alcohol and having high cetane number.

The following examples are for illustrative purposes and are not intended to limit the scope of the present disclosure.

### EXAMPLES

### Example 1

### Identification of a Multi-Subunit Prokaryotic Efflux Pump Capable of Mediating the Export of Intracellular N-Alkanes and N-Alkenes

E. coli, upon expression of ADM and AAR, not only produces hydrocarbons, mostly n-pentadecane and n-heptadecene, but also secretes them into the growth medium (Schirmer A et al. (2010) Science 329:559-562). This is because E. coli expresses one or more efflux pump(s), entirely absent in wild-type JCC138 (a cyanobacteria) and derivatives therefrom expressing ADM and AAR, described in, e.g., U.S. Pat. No. 7,794,969. The one or more efflux pump(s) are capable of catalyzing the transport of hydrocarbons from inside the cell through the inner membrane, then through the periplasmic space, and then through the outer membrane into the bulk phase and/or cell surface. This Example describes the identification of one such alk(a/e)ne efflux pump in E. coli.

RNA samples from the following four strains—each in replicate and each replicate before (T1) and 3.5 hr after (T2) addition of 1 mM IPTG—were analyzed using Agilent E. coli arrays: (1) JCC1169, E. coli BL21(DE3) carrying pCDFDuet-1::adm_PCC7942 (non-hydrocarbon producing control), (2) JCC1170, E. coli BL21(DE3) carrying pCDFDuet-1::aar_PCC7942 (n-alkanal-, n-alkanol-producing control strain), (3) JCC1214, E. coli BL21(DE3) carrying pCDFDuet-1::adm_Pmarinus-aar_Pmarinus (n-pentadecane-, n-heptadecene-producing strain), and (4) JCC1113, E. coli BL21(DE3) carrying pCDFDuet-1::adm_PCC7942-aar_PCC7942 (n-pentadecane-, n-heptadecene-producing strain). In one embodiment, the invention provides each of these four engineered strains of E. coli. In another embodiment, the invention provides methods of culturing each of these four engineered strains of E. coli and determining the level of secreted n-alkanes and n-alkenes in the culture medium.

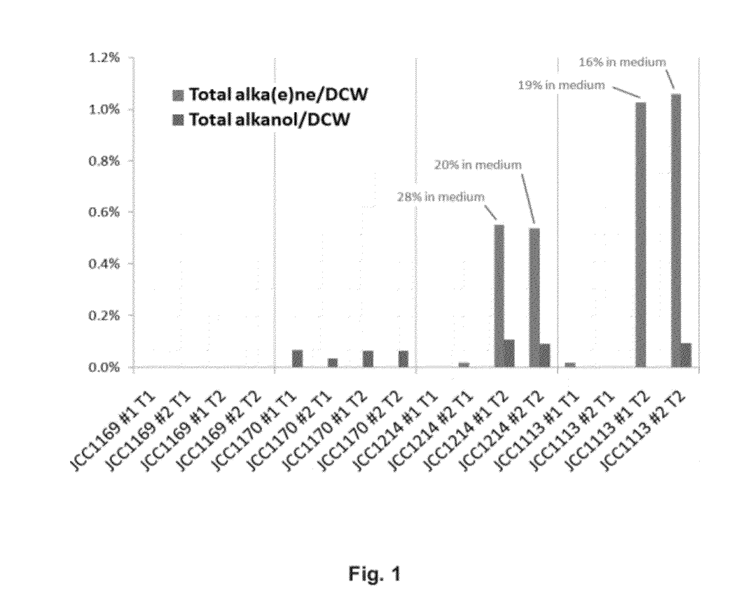

At the same time as cell pellets were sampled from each of the eight cultures for transcriptomic analysis, an additional cell pellet sample was extracted in acetone and the cell-free culture supernatant was extracted in ethyl acetate. Following GC-FID analysis of these acetone and ethyl acetate extractants, the concentrations of cell-associated and medium-associated (i.e., exported) hydrocarbons were quantitated (FIG. 1), confirming the different total hydrocarbon productivities of JCC1113 and JCC1214, as well as the fact that for both strains, at least 20% of the n-alka(e)ne produced was medium-associated.

The microarray data were processed and 17 genes of interest were selected. Twelve genes were immediately excluded from further analysis given the high probability that they were involved in a general stress response brought about by hydrocarbon production (Table 1).

TABLE 1
Table 1 Genes specifically up-regulated in JCC1214 and
JCC1113 that are likely involved in a general stress
response to intracellular hydrocarbon production, and
were therefore excluded from further analysis.

| Gene | Annotation | Putative stress response |
|---|---|---|
| chaA | IM Ca²⁺/Na⁺: H⁺ antiporter | ionic/proton motive force |
| slyB | OM lipoprotein induced upon Mg²⁺ | ionic/proton motive force |
| | starvation | |
| ycgW | Mg²⁺ starvation anti-sigma factor | ionic/proton motive force |
| mgtA | IM Mg²⁺ transporter | ionic/proton motive force |
| yqaE | Stress-induced IM protein | ionic/proton motive force |
| asr | Acid sock protein whose expression | cytosolic, periplasmic stress |
| | is dependent on RstA | |
| rstA | Transcriptional regulator of asr | cytosolic, periplasmic stress |
| spy | Periplasmic protein induced by | cytosolic, periplasmic stress |
| | envelope stress | |
| marA | Transcriptional regulator of | cytosolic, periplasmic stress |
| | stress-response genes | |
| marB | Co-expressed with marA | cytosolic, periplasmic stress |
| yfeT | Repressor of cell wall sugar | peptidoglycan |
| | catabolic genes | |
| ycfS | Transpeptidase that links | peptidoglycan |
| | peptidoglycan to OM | |

IM, inner membrane; OM, outer membrane.

The five remaining genes are presented in Table 2.

TABLE 2
Table 2 Non-stress-associated genes specifically
up-regulated in JCC1214 and JCC1113.

| | | Phylogenetic |
|---|---|---|
| Gene | Annotation | distribution |
| yqjA | Conserved IM protein; operonic with mzrA, | Narrow (excludes |
| | encoding a regulator of EnvZ/OmpR osmoreg- | Pseudomonas) |
| | ulation; regulated transcriptionally by CpxR, | |
| | a regulator involved in mediating the re- | |
| | sponse to envelope stress and multidrug efflux | |
| yebE | Conserved IM protein | Narrow (excludes |
| | | Pseudomonas) |
| yjbF | OM lipoprotein, possibly a porin; part of the | Narrow (excludes |
| | yjbEFGH operon whose overexpression causes | Pseudomonas) |
| | altered EPS production; regulated by RcsAB, a | |
| | regulator that involved in controlling capsule | |
| | biosynthesis | |
| ybiH | TetR-family transcriptional regulator; 1^{st} gene | Broad (includes |
| | of yibH-ybhGFSR gene cluster | Pseudomonas) |
| ybhG | Membrane fusion protein; part of ybhGFSR | Broad (includes |
| | operon encoding an ABC efflux pump | Pseudomonas) |

IM, inner membrane; OM, outer membrane; EPS, exopolysaccharide; ABC, ATP-binding cassette.

The other two genes, ybiH and ybhG, however, are notable in that (i) they are adjacent on the chromosome, (ii) they are of broad phylogenetic distribution (occurring in Pseudomonas), and, most importantly, (iii) are part of a cluster/operon of genes that encode a putative efflux pump of the ATP-binding cassette (ABC) superfamily. ybiH encodes a TetR-family transcriptional regulator, and therefore almost certainly cannot be involved directly in hydrocarbon efflux. In one embodiment of the invention, altering ybiH expression can be used to modulate expression of the ybhGFSR operon.

ybhG encodes a polypeptide of the membrane fusion protein (MFP) family. MFPs are periplasmic/extracellular subunits of multi-component efflux transporters that perform a diverse array of extrusion functions in both Gram-positive and Gram-negative prokaryotes, with substrates from heavy metal ions to whole proteins (Zgurskaya H et al. (2009) BBA 1794:794-807). MFPs are components of three major classes of bacterial efflux pumps: Resistance-Nodulation-cell Division (RND), ATP-Binding Cassette (ABC), and Major Facilitator superfamilies.

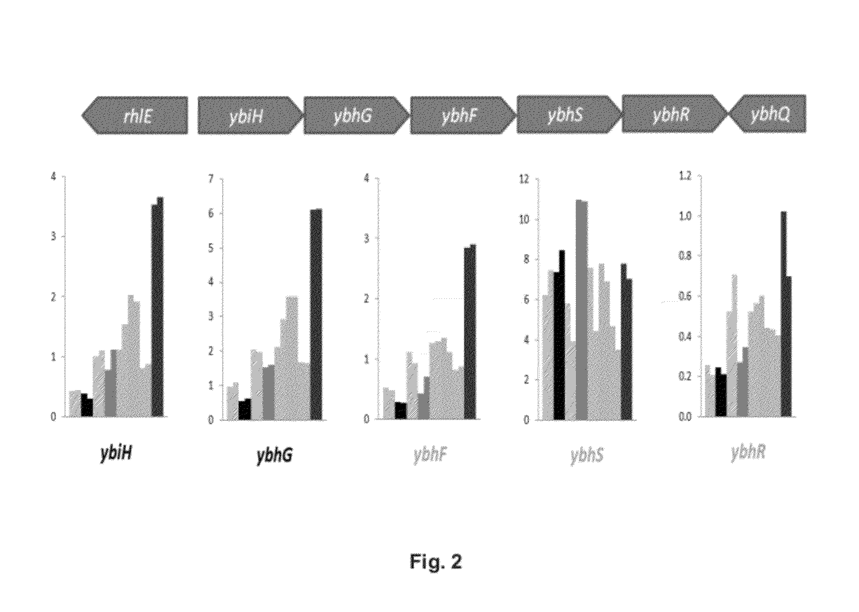

In Gram-negative bacteria such as E. coli, MFPs are known to mediate the interaction between inner membrane pump subunits and an outer membrane channel protein partner, such that substrates can be expelled from the cytosol and/or from the periplasmic space and/or from the inner membrane to the cell exterior in a seamless fashion. ybhG is part of what appears to be operon, ybhGFSR, encoding all the components required of an ABC-family efflux pump i.e., the MFP (ybhG), the cytosolic ATP-hydrolysis subunit (ybhF), and the two inner membrane subunits (ybhS and ybhR) (FIG. 2) (Davidson A et al. (2008) MMBR 72:317-364). Further bolstering this hypothesis, ybhF, ybhS, and ybhR manifest gene expression profiles largely concordant with those of ybiH and ybhG, albeit not as clean (FIG. 2).

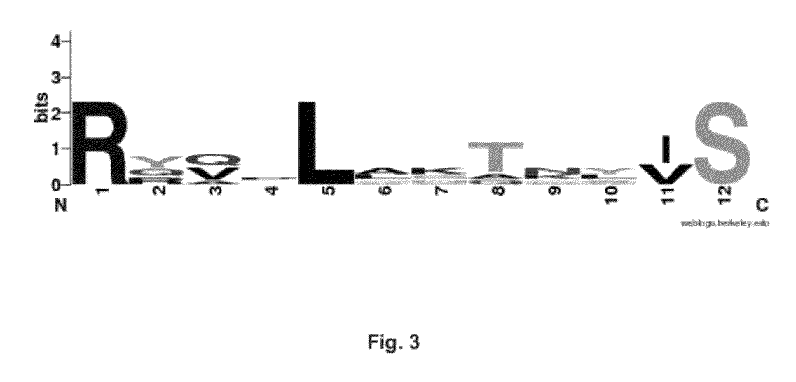

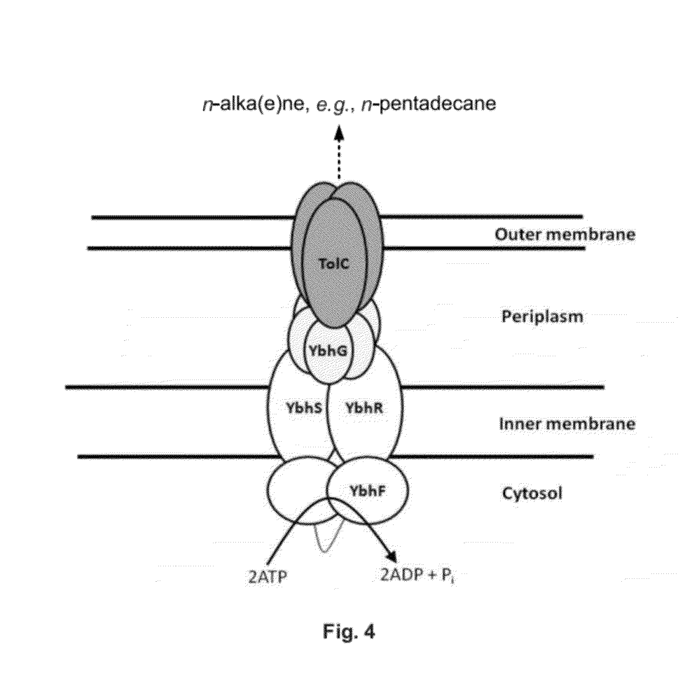

TolC, an outer membrane protein (OMP) is known to function promiscuously with several different inner membrane/periplasm efflux pump components in the extrusion of a wide range of lipophilic species and is thus the most likely candidate for the outer membrane partner of the YbhGFSR complex. To further support an interaction between YbhGFSR and TolC, the amino acid sequences of the 15 known and predicted MFS proteins of E. coli K12 MG1655 were compared, focusing in on the sequence of the loop joining the two α-helices of the coiled-coil domain that is one of the structural signatures of MFS proteins (Table 3). This loop sequence is significant in that in MFPs known to interact with TolC, there are conserved R, L, and S residues known to be critical for interaction with TolC (Hong-Man K et al. (2010) J Bacteriol 192:4498-4503). FIG. 3 shows the consensus sequence of the loop sequence of the seven MPS proteins known to interact with TolC (Table 3): the conserved R, L, and S are apparent, as is a conserved I/V residue preceding the conserved S. Further evidence that YbhG does indeed interact with TolC, the loop sequence of YbhG (Table 3) matches this consensus sequence of MFS proteins known to interact with TolC. A schematic of the fully assembled YbhGFSR-TolC efflux pump is shown in FIG. 4.

Note also, that the YbhG paralog YhiI also matches this consensus, suggesting that this MFP, too, interacts with TolC. Importantly, the MFPs known not to depend on TolC (AaeA and CusB) do not conform to this consensus sequence. YhiI is encoded within an operon paralogous to ybhGFSR, yhiI-rbbA-yhhJ, that encodes another uncharacterized ABC efflux system (rbbA encoding a putative ATP-hydrolyzing/IM subunit fusion and yhhJ the other IM protein). The evidence shows that this operon is also an inner membrane/periplasm component of a hydrocarbon efflux system.

TABLE 3
Table 3 Comparison of the coiled-coil loop sequences of the
15 known and predicted MFS proteins in E. coli K12.

| | | | | Loop between | |
|---|---|---|---|---|---|
| MFS protein | TCDB | Family name | OM component | coiled coil | Loop sequence |
| EmrA | 8.A.1.1.1 | Membrane Fusion Protein | TolC | short | RrvpLgnanlIS |
| | | | | | (SEQ ID NO: 1) |
| EmrK | 8.A.1.1.1 | Membrane Fusion Protein | TolC | short | RrvpLakqgvIS |
| | | | | | (SEQ ID NO: 2) |
| SdsR | 8.A.1.1.3 | Membrane Fusion Protein | SdsP | short | RtepLlkegfVS |
| | | | | | (SEQ ID NO: 3) |
| YiaV | 8.A.1.1.3 | Membrane Fusion Protein | ? | long | yqryargsqakv |
| | | | | | (SEQ ID NO: 4) |
| YibH | 8.A.1.1.3 | Membrane Fusion Protein | ? | long | yqryLkgsqaav |
| | | | | | (SEQ ID NO: 5) |
| AcrA | 8.A.1.6.1 | Membrane Fusion Protein | TolC | short | RyqkLlgtqyIS |
| | | | | | (SEQ ID NO: 6) |
| AcrE | 8.A.1.6.1 | Membrane Fusion Protein | TolC | short | RyvpLvgtkyIS |
| | | | | | (SEQ ID NO: 7) |
| MdtE | 8.A.1.6.1 | Membrane Fusion Protein | TolC | short | RqasLlktnyVS |
| | | | | | (SEQ ID NO: 8) |
| MdtA | 8.A.1.6.2 | Membrane Fusion Protein | TolC | short | RyqqLaktnlVS |
| | | | | | (SEQ ID NO: 9) |
| AaeA | 8.A.1.7.1 | Membrane Fusion Protein | not TolC, none? | short | RrnrL-gvqamS |
| | | | | | (SEQ ID NO: 10) |
| YhdJ | 8.A.1.7.1 | Membrane Fusion Protein | ? | short | RrrhL-sqnfIS |
| | | | | | (SEQ ID NO: 11) |
| CusB | 2.A.6.1.4 | Heavy Metal Efflux | CusC | na | na |
| YhbG | 3.A.1.105.4 | ATP-binding Cassette | ? | short | RqqgLwksrtIS |
| | | | | | (SEQ ID NO: 12) |
| YhiI | 3.A.1.105.4 | ATP-binding Cassette | ? | short | RsrsLaqrgaIS |
| | | | | | (SEQ ID NO: 13) |
| MacA | 3.A.1.122.1 | ATP-binding Cassette | TolC | short | RqqrLaqtkaVS |
| | | | | | (SEQ ID NO: 14) |

The TCDB column indicates the membrane protein family class according to the Transporter Classification Database (www.tcdb.org); the Family name column indicates the corresponding TCDB protein family name. AaeA is known to be TolC-independent (Van Dyk T K et al. (2004) J Bacteriol 186: 7196-7204). A loop between the coiled coil domain is considered “long” if it is >30 amino acids; short loops are of uniform size. CusB lacks a conventional coiled-coil domain. MFS, membrane fusion superfamily; OM, outer membrane; na, not applicable.

### Example 2

### Recombinant Expression of Hydrocarbon ABC Efflux Pump Systems in an N-Alkane Producing Non-Photosynthetic or Photosynthetic Microbe

Engineered photosynthetic microbes expressing ADM and AAR, e.g., the adm-aar⁺ JCC138 alkanogen JCC2055, have been and continue to be engineered to express hydrocarbon ABC efflux pump systems, e.g., ybhG/ybhF/ybhS/ybhR/tolC and homologous variants thereof or (prophetically) yhiI/rbbA/yhhJ/tolC and homologous variants thereof. This Example describes the creation of some exemplary constructs and microbes for alk(a/e)ne production and secretion. Many other examples of constructs and strains are provided elsewhere, herein.

The E. coli leader sequences of YbhG was replaced with a native JCC138 leader sequence associated with periplasmic localization; TolC had its E. coli leader sequence replaced with a native JCC138 leader sequence associated with outer membrane localization. In this Example, the cytosolic ATP-binding subunits (e.g., YbhF) and inner membrane subunits (YbhR/YbhS) will retain their entire native E. coli sequence.

A variety of standard standard promoters are used to drive expression of these efflux pump genes in the JCC138 host (see, e.g., U.S. patent application Ser. No. 12/833,821, filed Jul. 9, 2010, and U.S. patent application Ser. No. 12/876,056, filed Sep. 3, 2010). The DNA and protein sequences of the E. coli efflux pump components are shown in Table 4 and Table 5, respectively. The resulting strains are compared relative to an otherwise unmodified JCC138 alkanogen control strain to demonstrate the improved ability of strains expressing recombinant hydrocarbon ABC efflux pump systems to extrude hydrocarbons, e.g., n-pentadecane and/or n-heptadecane, into the growth medium.

Exemplary perisplasmic leader sequences that will be deleted from YbhG and YhiI are as follows:

| YbhG | | |
|---|---|---|
| (SEQ ID NO: 15) | | |
| 1 | MMKKPVVIGL AVVVLAAVVA GGYWWYQSRQ DNGLTLYGNV DIRTVNLSFR VGGRVESLAV | 60 |
| 61 | DEGDAIKAGQ VLGELDHKPY EIALMQAKAG VSVAQAQYDL MLAGYRNEEI AQAAAAVKQA | 120 |
| 121 | QAAYDYAQNF YNRQQGLWKS RTISANDLEN ARSSRDQAQA TLKSAQDKLR QYRSGNREQD | 180 |
| 181 | IAQAKASLEQ AQAQLAQAEL NLQDSTLIAP SDGTLLTRAV EPGTVLNEGG TVFTVSLTRP | 240 |
| 241 | VWVRAYVDER NLDQAQPGRK VLLYTDGRPD KPYHGQIGFV SPTAEFTPKT VETPDLRTDL | 300 |
| 301 | VYRLRIVVTD ADDALRQGMP VTVQFGDEAG HE | |
| YhiI | | |
| (SEQ ID NO: 16) | | |
| 1 | MDKSKRHLAW WVVGLLAVAA IVAWWLLRPA GVPEGFAVSN GRIEATEVDI ASKIAGRIDT | 60 |
| 61 | ILVKEGKFVR EGEVLAKMDT RVLQEQRLEA IAQIKEAQSA VAAAQALLEQ RQSETRAAQS | 120 |
| 121 | LVNQRQAELD SVAKRHTRSR SLAQRGAISA QQLDDDRAAA ESARAALESA KAQVSASKAA | 180 |
| 181 | IEAARTNIIQ AQTRVEAAQA TERRIAADID DSELKAPRDG RVQYRVAEPG EVLAAGGRVL | 240 |
| 241 | NMVDLSDVYM TFFLPTEQAG TLKLGGEARL ILDAAPDLRI PATISFVASV AQFTPKTVET | 300 |
| 301 | SDERLKLMFR VKARIPPELL QQHLEYVKTG LPGVAWVRVN EELPWPDDLV VRLPQ | |

An exemplary native JCC138 leader sequence associated with periplasmic location that will be swapped into YbhG and YhiI includes the first 22 amino acids of periplasmically SYNPCC7002_A0578 (http://www.ncbi.nlm.nih.gov/protein/169884872#comment_{—}169884872):

| | |
|---|---|
| MRFFWFFLTLLTLSTWQLPAWA | (SEQ ID NO: 17) |

An exemplary native JCC138 leader sequence associated with outer membrane location that will be swapped into TolC includes the first 25 amino acids of JCC138 TolC homolog SYNPCC7002_A0585 (http://www.ncbi.nlm.nih.gov/protein/169884879):

| | |
|---|---|
| MFAFRDFLTFSTGGLVVLSGGGVAIA | (SEQ ID NO: 18) |

The leader sequence of TolC is described elsewhere in the art, e.g., U.S. patent application Ser. No. 12/876,056, filed Sep. 3, 2010.

TABLE 4

| Gene | ORF sequence |
|---|---|
| ybhG | SEQ ID NO: 19 |
| ybhF | SEQ ID NO: 20 |
| ybhS | SEQ ID NO: 21 |
| ybhR | SEQ ID NO: 22 |
| tolC | SEQ ID NO: 23 |
| yhiI | SEQ ID NO: 24 |
| rbbA | SEQ ID NO: 25 |
| yhhJ | SEQ ID NO: 26 |

TABLE 5

| Gene | Protein sequence |
|---|---|
| ybhG | SEQ ID NO: 27 |
| ybhF | SEQ ID NO: 28 |
| ybhS | SEQ ID NO: 29 |
| ybhR | SEQ ID NO: 30 |
| tolC | SEQ ID NO: 31 |
| yhiI | SEQ ID NO: 32 |
| rbbA | SEQ ID NO: 33 |
| yhhJ | SEQ ID NO: 34 |

In one embodiment, the invention provides recombinant E. coli cells comprising a modification to a gene listed in Table 4, wherein said modification is selected from the group consisting of (1) a modification that eliminates or reduces the activity of the gene, wherein said modification includes a whole or partial deletion of the gene or a point mutation; and (2) a modification that increases expression of a gene listed in Table 4, wherein said modification includes an additional copy of the gene and/or expression of the gene from a stronger promoter than the native promoter. In another embodiment, the invention provides an engineered cyanobacterium recombinantly expressing one or more genes listed in Table 4. In a related embodiment, the engineered cyanobacterium further comprises recombinant genes for n-alkane biosynthesis, e.g., aar and/or adm genes, which render it capable of synthesizing increased levels of n-alkanes (and/or n-alkenes) relative to an engineered cyanobacterium lacking said recombinant genes for n-alkane biosynthesis.

### Example 3

### Construction of ADM-AAR Expression Vector and Bacterial Strains for Alkane Synthesis

To express the alkane pathway in E. coli K12 strains, pJexpress404™ was purchased from DNA 2.0 (Menlo Park, Calif.). pJexpress404™ contains a high copy number pUC origin of replication, the bla gene for carbenicillin/ampicillin resistance, a multiple cloning site, a modified T5 promoter for high expression and tight transcriptional control, and lad as a repressor of the modified T5 promoter. adm (gene Synpcc7942_{—}1593) and aar (gene Synpcc7942_{—}1594) of Synechococcus elongatus PCC 7942 were cloned as an operon from pJB853 into pJexpress404 to generate pJB1440. The sequence of pJB1440 is presented in Table 6, below.

TABLE 6

pJB1440: SEQ ID NO: 35

A fadE knockout strain in E. coli BW25113 (an E. coli K12 strain) which contains a kanamycin marker in place of fadE was obtained from the Yale strain collection (http://cgsc.biology.yale.edu; New Haven, Conn.). This marker was removed using pCP20™ which expresses a FLP recombinase vector as previously described (Datsenko et al., PNAS (2000) 97:6640-5) to yield strain JCC1880 (E. coli BW25113ΔfadE). To knockout tolC, ybiH or any gene encoding a subunit of the YbhGFSR efflux pump, P1 transduction was used to transduce the knockout (kanamycin marker in place of targeted gene for knockout) from a donor strain of the Yale strain collection to the E. coli production strain JCC1880 (BW25113ΔfadE). The derivative knockout strains were then transformed with the alkane production vector pJB1440 to express adm-aar.

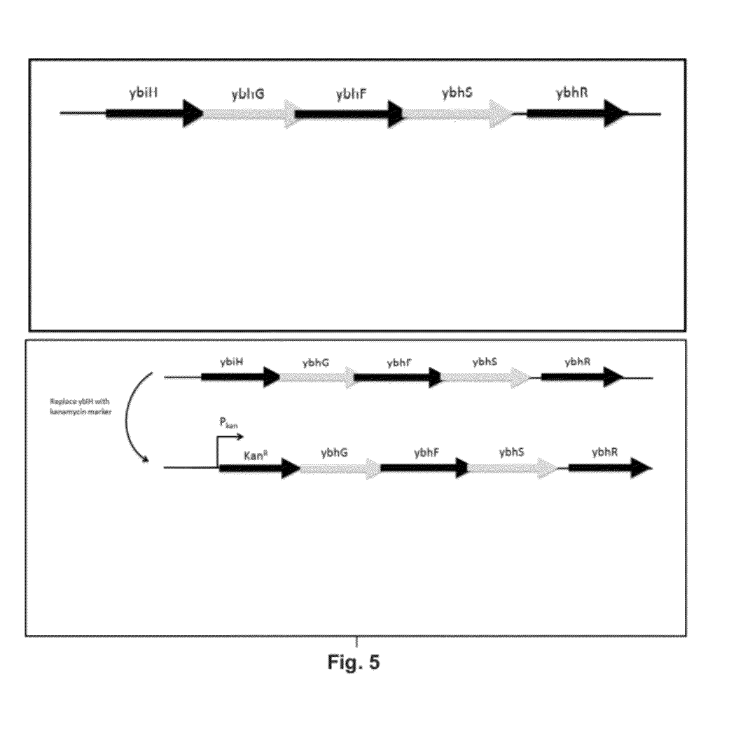

JCC1880 derivative strains with the following genotypes were prepared: ΔfadEΔybiH, ΔfadEΔybhF, ΔfadEΔybhG, ΔfadEΔybhS, ΔfadEΔybhR and ΔfadE_ybiH::kan (replacing the ybiH gene with an insert comprising a constitutive promoter and a kanamycin resistance gene, wherein expression of both the kanamycin gene and the ybhGFSR operon are driven by the promoter; see FIG. 5, bottom, and Table 7 which provides the kanamycin resistance gene coding sequence and constitutive promoter sequence). All strains were transformed with the alkane production vector pJB1440, described above. Each of these strains was cultured in minimal media+3% glucose+30 mg/L FeCl₃.6H₂O at 37° C., 250 rpm for 24 hours. Expression of the adm-aar operon was induced from the T5 promoter with 1 mM IPTG at an OD₆₀₀ of about 0.4 (approximately six hours after inoculation). The cells were harvested and cell-free supernatant samples were obtained after 18 hours of induction. Cell pellets were extracted with acetone and supernatants with ethyl acetate. Measurements were taken by GC-FID.

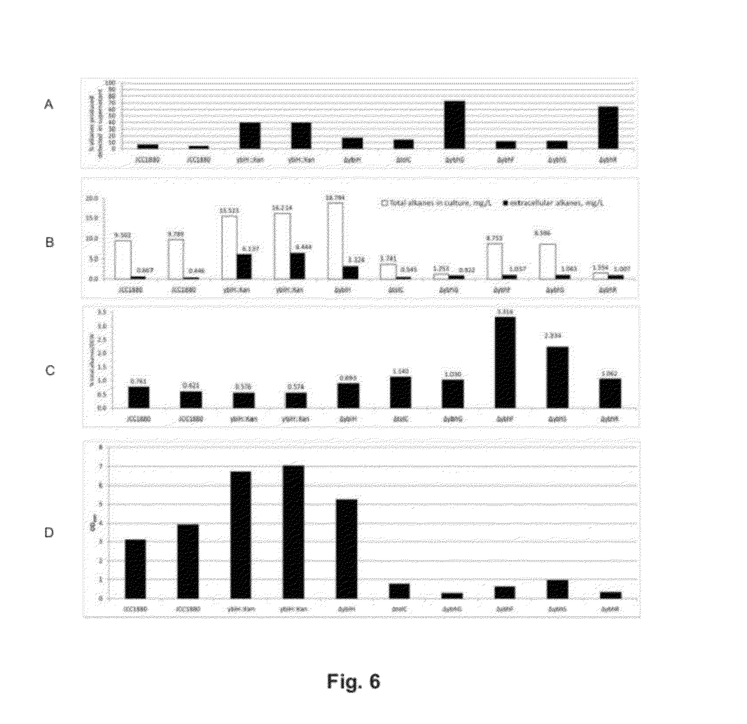

The effects of the genotypes on cell growth and alkane secretion are depicted in FIG. 6. FIG. 6 confirms that inactivation of YbiH expression promotes alkane secretion (see FIG. 6A and FIG. 6B; compare ΔybiH to JCC11880). FIG. 6 also confirms that constitutive expression of the YbhGFSR transporter increases secretion (see FIG. 6A and FIG. 6B; compare ybiH::Kan to JCC1180 and ΔybiH), with 40% of total alkanes being secreted into the supernatant. This level of secretion efficiency occurs in the absence of any agents added to the growth medium which are known to affect membrane permeability (e.g., Tris buffer, EDTA, Triton X-100 detergent and other surfactants). FIG. 6C and FIG. 6D show that cell growth is inhibited when cells produce alkanes in the absence of a transporter capable of efficiently transporting alkanes, e.g., TolC or the YbhGFSR transporter.

TABLE 7

Kanamycin promoter and gene coding sequence: SEQ ID NO: 36

### Example 4

### Overexpression of ybhGFSR in E. coli Improves Alkane Efflux

To construct plasmid pJB1932, containing the ybhGFSR operon under control of an inducible promoter, plasmid pCDFDuet-1 (EMD4Biosciences) was digested with AscI and MluI to remove a T7 promoter and the 5′ end of lad present on pCDFDuet-1. The remaining plasmid backbone containing the CLODF13 origin, truncated lad, and aadA (encoding spectinomycin resistance) was gel purified and self-ligated together using NEB Quick Ligase. The resulting plasmid was then digested with restriction enzymes NotI and NdeI to serve as an open vector for insertion of a tetracycline inducible promoter (P_{LtetO1}). A tetR-P_{LtetO1} insert was isolated by digestion of pJB800 (DNA 2.0) with NdeI and NotI followed by agarose gel purification. This tetR-P_{LtetO1} insert was then ligated into the open vector cut with the same enzymes to create plasmid pJB1918. Following construction of pJB1918, the ybhGFSR operon was amplified by PCR from E. coli MG1655 genomic DNA using Phusion HF DNA polymerase (NEB) and primers KS202 (5′ aataCATATGATGAAAAAACCTGTCGTGATCGG 3′) (SEQ ID NO: 37) and KS416 (5′ aataaGGCCGGCCttaCATCACCTTACGTCTAAACATCGCG 3′) (SEQ ID NO: 38). The resulting PCR product was column purified, digested with NdeI and FseI and ligated into plasmid pJB1918 also digested with NdeI and FseI to create pJB1932.

TABLE 8

| Sequence description | SEQ ID NO: |
|---|---|
| tetR_P_{Ltet01}-ybhGFSR DNA sequence (start | SEQ ID NO: 39 |
| codon of ybhG changed from native ‘GTG’ | |
| sequence to ‘ATG’) | |

Plasmids pJB1932 (P_{LtetO1}-ybhGFSR) and pJB1440 (P(T5)-adm-aar) were co-transformed into JCC1880 (ΔfadE) by electroporation and transformants were isolated on LB agar plates containing carbenicillin (100 μg/ml) and spectinomycin (50 μg/ml). Likewise, plasmids pJB1918 and pJB1440 were co-transformed into JCC2359 (ΔfadEΔybhGFSR) to serve as a negative control strain. 2 unique, single colonies for each strain were picked to inoculate two 3-ml LB seed cultures in test tubes (containing appropriate antibiotics), which were incubated at 37° C. and 260 rpm for ˜16 hours.

Alkane production and efflux of each strain was tested in 250 ml screw-cap shake flasks containing 25 ml M9f media (M9 minimal media+30 g/L glucose+30 mg/L FeCl₃.6H₂O+A5 metals (27 mg/L FeCl₃.6H₂O, 2 mg/L ZnCl₂.4H₂O, 2 mg/L CaCl₂.2H₂O, 2 mg/L Na₂MoO₄.2H₂O, 1.9 mg/L CuSO₄.5H₂O, 0.5 mg/L H₃BO₃)) with carbenicillin (100 μg/ml), spectinomycin (50 μg/ml), and a 5 ml DBE (25 mg/L BHT+25 mg/L eicosane in dodecane) overlay for extraction of alkanes from the aqueous phase that were secreted by the cells. Cells were harvested from LB seed cultures and used to inoculate shake flask cultures containing 25 ml M9f to an OD₆₀₀ of 0.4. Following inoculation, 5 ml DBE was added to each culture and all flasks were incubated at 37° C. and 260 rpm for 1 hour; at which point 1.0 mM IPTG and 100 ng/ml ahydrotetracycline (aTc) were added to each culture to induce gene expression from the T5 and P_{LtetO-1} promoters, respectively. After induction with IPTG and aTc, all cultures were returned to 37° C., 250 rpm and incubated for another 23 hours.

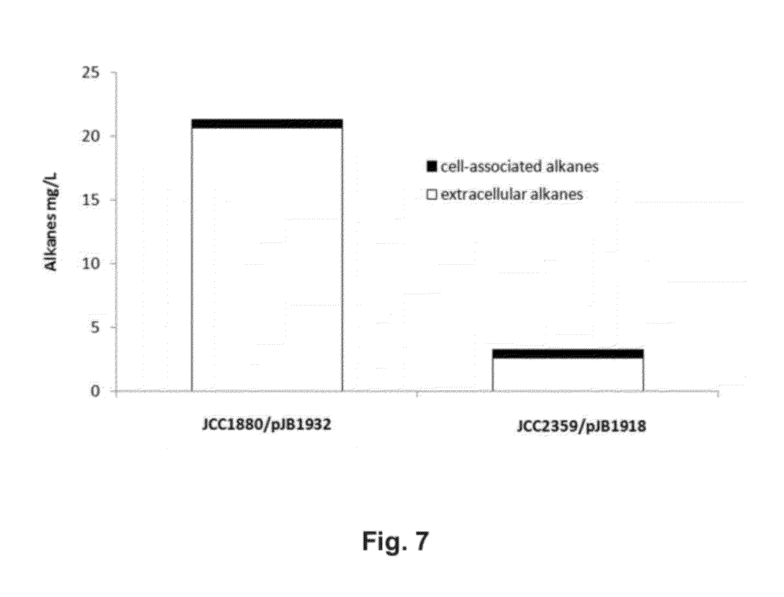

All flasks were sampled at 24 hours for alkane detection by GC-FID and to determine culture density. 20D-ml of cells from each flask culture were extracted with acetone containing 25 μg/ml butylated hydroxytoluene (BHT) and 25 μg/ml eicosane (ABE) by resuspension of the de-wetted cell pellet in 1 ml ABE, vortexing for 30 seconds, and centrifugation at 15,000 rpm for 4 minutes. Following the removal of cells for ABE extraction, the entire contents of the culture was centrifuged at 6000 rpm for 15 minutes in a 50-ml Falcon tube to separate the aqueous and organic layer (DBE plus secreted hydrocarbons). 200 μl of the organic layer was then analyzed for alkanes and alkenes by GC-FID. Results showed that overexpression of ybhGFSR (an ABC efflux pump) in an E. coli alkanogen (JCC1880/pJB1932) increases total alkane and alkene production in comparison with the E. coli alkanogen lacking ybhGFSR (JCC2359/pJB1918). Further, ˜97% of the total alkanes and alkenes produced with JCC1880/pJB1932 were detected extracellularly (FIG. 7).

### Example 5

### Improved Efflux of Alkanes and Alkenes in Strains with a Genetically Disrupted Lipopolysaccharide (LPS) Layer

To obtain an E. coli strain with a disrupted LPS, rfaC (encoding ADP-heptose:LPS heptosyl transferase I) in JCC1880 (ΔfadE) was knocked out. A knockout cassette was constructed by amplification of a kanamycin marker from pKD13 (obtained from the Coli Genetic Stock Center, http://cgsc.biology.yale.edu/GDK.php) using Phusion HF DNA polymerase and primers KS140 (5′GCGTACTGGAAGAACTCAACGCGCTATTGTTACAAGAGGAAGCCTGACGGgtgtaggctggagctgcttc 3′) (SEQ ID NO:40) and KS141 (5′GTGTAAGGTTTCAATGAATGAAGTTTAAAGGATGTTAGCATGTTTTACCTctgtcaaacatgagaattaa 3′) (SEQ ID NO:41). The PCR product generated here contains a constitutively expressed kanamycin resistance marker flanked by 2 regions of homology, H1 and H2, which flank the rfaC ORF in the E. coli genome. Electrocompetent cells of JCC1880 harboring pKD46 and actively expressing Red Recombinase were transformed with 300 ng of purified PCR product and transformants were isolated isolated on LB agar plates containing 50 μg/ml kanamycin at 37° C. Successful insertion of the kanamycin resistance cassette in place of rfaC was confirmed by colony PCR (strain JCC1880_rfaC::kan). To remove the kanamycin resistance marker, JCC1880_rfaC::kan was transformed with pCP20 and cultured as previously described (Datsenko et. al, 2000). Successful removal of the kanamycin marker was confirmed by colony PCR, resulting in strain JCC1999.

TABLE 9

| Sequence description | SEQ ID NO: |
|---|---|
| DNA sequence of rfaC locus in JCC1880 (ΔfadE) | SEQ ID NO: 42 |
| DNA sequence of rfaC locus in JCC1999 | SEQ ID NO: 43 |
| (ΔfadEΔrfaC) | |

Plasmids pJB1932 (P_{LtetO-1}-ybhGFSR) and pJB1440 (P(T5)-adm-aar) were co-transformed into JCC1880 (ΔfadE) and JCC1999 by electroporation. Transformants were isolated on LB agar plates containing carbenicillin (100 μg/ml) and spectinomycin (50 μg/ml). 2 unique, single colonies for each strain were picked to inoculate two 3-ml LB seed cultures in test tubes (containing appropriate antibiotics), which were incubated at 37° C. and 260 rpm for ˜16 hours.

Hydrocarbon production and efflux of each strain was tested in 250 ml screw-cap shake flasks containing 25 ml M9f media (M9 minimal media+30 g/L glucose+30 mg/L FeCl₃.6H₂O+A5 metals (27 mg/L FeCl₃.6H₂O, 2 mg/L ZnCl₂.4H₂O, 2 mg/L CaCl₂.2H₂O, 2 mg/L Na₂MoO₄.2H₂O, 1.9 mg/L CuSO₄.5H₂O, 0.5 mg/L H₃BO₃)) with carbenicillin (100 μg/ml) and spectinomycin (50 μg/ml). Cells were harvested from LB seed cultures and used to inoculate shake flask cultures containing 25 ml M9f to an OD₆₀₀ of 0.1. Cultures were incubated at 37 C, 260 rpm until an OD₆₀₀ of 0.4 was reached, at which point 1.0 mM IPTG and 100 ng/ml ahydrotetracycline (aTc) were added to each culture to induce expression of YbhGFSR and the alkane pathway (adm-aar). After induction with IPTG and aTc, all cultures were returned to 37° C., 260 rpm and incubated for a total of 24 hours.

All flasks were sampled at 24 hours for hydrocarbon detection by GC-FID and to determine culture density. 2 OD-ml of cells from each flask culture were extracted with acetone containing 25 μg/ml butylated hydroxytoluene (BHT) and 25 μg/ml eicosane (ABE) by resuspension of the de-wetted cell pellet in 1 ml ABE, vortexing for 30 seconds, and centrifugation at 15,000 rpm for 4 minutes. For detection of extracellular hydrocarbons, 500 μl of cell-free supernatant of each culture was extracted with 1 ml EBE (ethyl acetate+25 μg/ml butylated hydroxytoluene (BHT) and 25 μg/ml eicosane (ABE)) by vortexing for 30 seconds, and centrifugation at 15,000 rpm for 2 minutes. Results showed that disruption of LPS in an E. coli alkanogen (JCC1999/pJB1440/pJB1932) improves hydrocarbon efflux in comparison with the E. coli alkanogen possessing a wild type (undisrupted) LPS layer (JCC1880/pJB1440/pJB1932) (Table 10A). At least 50% secretion was observed in JC1999, the alkane-producing strain comprising a genetic disruption of its LPS layer. The observed improvement in percent of total n-alkanes and n-alkenes secreted is at least 10 fold greater in a strain comprising a genetic disruption of its LPS layer than an otherwise identical strain with an undisrupted LPS layer.

TABLE 10A

| | | Total | Extracellular | % |
|---|---|---|---|---|
| | | alk(a/e)nes | alk(a/e)nes | Alk(a/e)nes |
| strain | OD₆₀₀ | (mg · l⁻¹) | (mg · l⁻¹) | secreted |
| JCC1880 | 6.6 | 17.9 | 0.8 | 4.5 |
| JCC1999 | 7.1 | 13.2 | 7.0 | 53 |

In addition to ADP-heptose:LPS heptosyl transferase I, other genes and their corresponding enzymes involved in LPS layer synthesis or maintenance can be knocked out, mutated, or otherwise attenuated to achieve a similar effect (i.e., increased secretion of alkanes and alkenes relative to the parent strain). Exemplary genes are listed in Table 10B. In certain embodiments, where the alkane producing strain is other than E. coli, homologs of these genes can be easily identified, then knocked out or mutated Likewise, in microbes where other membrane layers in addition to the LPS can be disrupted (e.g., the S layer and/or glycocalyx of cyanobacteria), genes involved in the biosynthesis and maintenance of those layers can identified, then knocked out or mutated to diminish their activity, disrupt the layer of interest, and improve the efflux of hydrocarbons (alkanes, alkenes, etc.) produced by the modified microbe. Exemplary genes involved in the synthesis of the S layer and glycocalyx of cyanobacteria are presented in Table 10C.

TABLE 10B

| | E. coli | |
|---|---|---|
| Enzyme | gene | EC # |
| ADP-heptose: LPS heptosyl transferase I | rfaC | 2.4.—.— |
| ADP-heptose: LPS heptosyltransferase II | rfaF | 2.—.—.— |
| lipopolysaccharide glucosyltransferase I | rfaG | 2.4.1.58 |
| lipopolysaccharide core heptose (I) kinase | rfaP | 2.7.1.— |
| lipopolysaccharide core heptosyl transferase III | rfaQ | 2.4.—.— |
| lipopolysaccharide core heptose (II) kinase | rfaY | 2.7.1.— |
| UDP-D-galactose: (glucosyl)lipopolysaccharide- | rfaB | 2.4.1.44 |
| 1,6-D-galactosyltransferase | | |
| UDP-D-glucose: (glucosyl)LPS α-1,3- | rfaI | 2.4.1.44 |
| glucosyltransferase | | |
| UDP-glucose: (glucosyl)LPS α-1,2- | rfaJ | 2.4.1.58 |
| glucosyltransferase | | |
| heptosyl transferase IV | rfaK | 2.4.—.— |

TABLE 10C

| Gene | Putative function | Genome annotation | Accession Number |
|---|---|---|---|
| SYNPCC7002_A0418 | Glycocalyx synthesis | ABC transporter, ATP- | YP_001733684.1 |
| | | binding protein | |
| SYNPCC7002_A0419 | Glycocalyx synthesis | hypothetical protein | YP_001733685.1 |
| SYNPCC7002_A0420 | Glycocalyx synthesis | hypothetical protein | YP_001733686.1 |
| SYNPCC7002_A0421 | Glycocalyx synthesis | ABC-type transport | YP_001733687.1 |
| | | protein | |
| SYNPCC7002_A0782 | S-layer synthesis | hypothetical protein | YP_001734043.1 |
| SYNPCC7002_A1034 | S-layer synthesis | hypothetical protein | YP_001734292.1 |
| SYNPCC7002_A1214 | Glycocalyx synthesis | UDP-N- | YP_001734468.1 |
| | | acetylglucosamine 2- | |
| | | epimerase | |
| SYNPCC7002_A1423 | Glycocalyx synthesis | glycosyl transferase group | YP_001734670.1 |
| | | 2 family protein | |
| SYNPCC7002_A1500 | Glycocalyx synthesis | hypothetical protein | YP_001734747.1 |
| SYNPCC7002_A1501 | Glycocalyx synthesis | polysaccharide export | YP_001734748.1 |
| | | periplasmic protein | |
| SYNPCC7002_A1634 | S-layer synthesis | S-layer like protein | YP_001734880.1 |
| SYNPCC7002_A1901 | Glycocalyx synthesis | exoD, exopolysaccharide | YP_001735144.1 |
| | | synthesis protein | |
| SYNPCC7002_A2118 | Glycocalyx synthesis | cellulose synthase | YP_001735355.1 |
| | | catalytic subunit | |
| SYNPCC7002_A2340 | Glycocalyx synthesis | UDP-glucose | YP_001735573.1 |
| | | dehydrogenase | |
| SYNPCC7002_A2451 | Glycocalyx synthesis | polysaccharide | YP_001735684.1 |
| | | biosynthesis export | |
| | | protein | |
| SYNPCC7002_A2605 | S-layer synthesis | surface layer protein-like | YP_001735837.1 |
| | | protein | |
| SYNPCC7002_A2813 | S-layer synthesis | S-layer like protein; porin | YP_001736037.1 |
| SYNPCC7002_G0011 | Glycocalyx synthesis | outer membrane protein | YP_001733120.1 |
| SYNPCC7002_G0012 | Glycocalyx synthesis | ATPase, P-type | YP_001733121.1 |
| | | (transporting), HAD | |
| | | superfamily, subfamily | |
| | | IC | |
| SYNPCC7002_G0013 | Glycocalyx synthesis | ExoD family | YP_001733122.1 |
| | | exopolysaccharide | |
| | | synthesis protein | |

### Example 6

### Increased Alkanes Efflux in Photosynthetic Microbes Expressing Recombinant accADBC

This Example shows that the recombinant expression of an acetyl-CoA carboxylase operon leads to increased alkanes secretion by alkane-producing photosynthetic microbes.

Materials and Methods. Construction of the promoter-accADBC expression plasmid. Construction of pJB525: pJB373 plasmid was designed as an empty vector for recombination into Synechococcus sp. PCC 7002 to remove the native Type II restriction enzyme (SYNPCC7002_A0358). Two regions of homology, the Upstream Homology Region (UHR) and the Downstream Homology Region (DHR) were designed to flank the construct. These 750 bp regions of homology correspond to positions 377235-377984 and 381566-382315 (Genbank Accession NC.sub.—005025) for UHR and DHR, respectively. The aadA promoter and gene sequence were designed to confer spectinomycin and streptomycin resistance to the integrated construct. Downstream of the UHR region restriction endonuclease recognition sites were inserted for NotI, NdeI and EcoRI, as well as the sites for BamHI, XhoI, SpeI and PacI. Following the EcoRI site, the natural terminator from the alcohol dehydrogenase gene from Zymomonas mobilis (adhII) terminator was included. Convenient XbaI restriction sites flank the UHR and the DHR allowing cleavage of the DNA intended for recombination from the rest of the vector. pJB373 was constructed by contract synthesis from DNA2.0 (Menlo Park, Calif.). To construct pJB525, the aadA promoter and gene in pJB373 were replaced with the npt promoter and gene using PacI and AscI, thus conferring kanamycin resistance to the integrated construct.

Construction of pJB1623-1626: The E. coli accADBC genes (Genbank AAC73296.1, AAC75376.1, AAC76287.1, AAC76288.1) were codon optimized for E coli and obtained by contract synthesis from DNA 2.0 (Menlo Park, Calif.) as 2 cassettes: accAD and accBC. These cassettes were subcloned using EcoRI and XhoI to make pJB431. lacI-P(trc) was cloned upstream of accADBC with NotI and NdeI to make pJB504. To construct the base transformation plasmid, pJB540, P(trc)-accADBC was cloned into the NotI and EcoRI sites of pJB525. A promoterless cassette was engineered by removing the lacI-P(trc) cassette from pJB540 with NotI and NdeI, blunting the ends with Klenow, and self-ligating to make pJB1623. The DNA sequences of P(psaA) and the ammonia-repressible nitrate reductase promoters, P(nir07) and P(nir09), were obtained from Genbank, and cloned between NotI and NdeI sites immediately upstream of accADBC in pJB540 to make pJB1624, 1625, and 1626, respectively. Final transformation constructs are listed in Table 11. All restriction and ligation enzymes were obtained from New England Biolabs (Ipswich, Mass.). pJB1623-1626 constructs were transformed into NEB 5-α competent E. coli (High Efficiency) (New England Biolabs: Ipswich, Mass.).

TABLE 11

| Plasmid name | Expression cassette |
|---|---|
| pJB1623 | Promoterless_accADBC_kan^{R} |
| pJB1624 | P(psaA)_accADBC_kan^{R} |
| pJB1625 | P(nir07)_accADBC_kan^{R} |
| pJB1626 | P(nir09)_accADBC_kan^{R} |

Plasmid transformation into JCC2055. The constructs as described above were integrated onto the genome of JCC2055 (JCC138 pAQ3::P(nir07)_adm_aar_spec^{R}), which is maintained at approximately 7 copies per cell. The following protocol was used for integrating the DNA cassettes. Genomic DNA was isolated from strains containing the ΔA0358::accADBC insert using Epicentre Masterpure DNA purification kit (Madison, Wis.). JCC2055 was grown in an incubated shaker flask at 37° C. at 1% CO₂ to an OD₇₃₀ of 0.6 in A⁺ medium supplemented with 200 μg/mL spectinomycin. 1000 μL of culture was added to a microcentrifuge tube with 5 μg of genomic DNA. Cells were incubated in the dark for one hour at 37° C. The entire volume of cells was plated on A⁺ plates with 1.5% agar and grown at 37° C. in an illuminated incubator (40-60 μE/m2/s PAR, measured with a LI-250A light meter (LI-COR)) for approximately 24 hours. 50 μg/mL of kanamycin was introduced to the plates by placing the stock solution of antibiotic under the agar, and allowing it to diffuse up through the agar. After further incubation, resistant colonies became visible in 6 days. One colony from each plate was restreaked onto A⁺ plates with 1.5% agar supplemented with 6 mM urea and 200 μg/mL spectinomycin and 50 μg/mL of kanamycin. Colonies were designated as JCC3198-3201 and are listed in Table 12.

Measurement of increased alkane production in cells and in media. Colonies of JCC138, JCC2055, JCC3198, JCC3199, JCC3200, and JCC3201 were inoculated into 5 ml of A+ media containing 3 mM urea, 200 μg/ml spectinomycin, and 50 μg/ml kanamycin as necessary. This culture was incubated at 37° C. with 1% CO₂ in light (40-50 μE/m2/s PAR, measured with a LI-250A light meter (LI-COR)). Strains were subcultured to a starting OD₇₃₀ of 0.5 in 5 ml of JB2.1 media containing 3 mM urea, 200 μg/ml spectinomycin, and 50 μg/ml kanamycin as necessary and cultured in standard glass test tubes for 3 days at 37° C. with 1% CO₂ in light (40-50 μE/m2/s PAR, measured with a LI-250A light meter (LI-COR)).

2 OD-ml of cells from each tube culture were extracted with acetone containing 50.4 g/mL butylated hydroxytoluene (BHT) and 51 μg/ml eicosane (ABE) by resuspension of the cell pellet in 1 ml ABE, vortexing for 30 seconds, and centrifugation at 15,000 rpm for 4 minutes. To measure alkanes present in the media 1 mL of cell culture was centrifuged at 15,000 rpm for 3 minutes. 500 μL was moved to a fresh tube and phase partitioned with 1 mL of ethyl acetate containing 25.3 μg/mL butylated hydroxytoluene (BHT) and 25.11 μg/ml eicosane (EBE). 600 ul of the organic layer was then analyzed for alkanes by GC-FID.

The data is shown in Table 13. The results show that expression of accADBC in alkane-producing microbes results in increased n-alkane secretion levels. The amount of n-alkane secretion observed is greater than 15% in some cases, and generally between 1% and 20%. In strains where the recombinant acetyl-CoA carboxylase genes are functionally linked to a promoter, the percent secretion observed is between 2-fold and 90-fold greater than that observed when culturing otherwise identical strains lacking the recombinant genes encoding acetyl-CoA carboxylase.

TABLE 12
Genotypes of strains with recombinant accADBC

| Strain name | Genotype |
|---|---|
| JCC3198 | JCC138 pAQ3::P(nir07)_adm_aar_spec^{R} |
| | ΔA0358::promoterless-accADBC_kan^{R} |
| JCC3199 | JCC138 pAQ3::P(nir07)_adm_aar_spec^{R} |
| | ΔA0358::P(psaA)-accADBC_kan^{R} |
| JCC3200 | JCC138 pAQ3::P(nir07)_adm_aar_spec^{R} |
| | ΔA0358::P(nir07)-accADBC_kan^{R} |
| JCC3201 | JCC138 pAQ3::P(nir07)_adm_aar_spec^{R} |
| | ΔA0358::P(nir09)-accADBC_kan^{R} |

TABLE 13
Alkane production and efflux by various strains

| | | Cellular + | | |
|---|---|---|---|---|
| | | media | In media | % alkanes |
| Strain | OD₇₃₀ | (mg/L) | (mg/L) | secreted |
| JCC2055 | 10.23 ± 0.15 | 73.70 ± 2.95 | 0.16 ± 0.16 | 0.21 ± 0.21 |
| JCC3198 | 9.15 ± 0.17 | 66.10 ± 1.40 | 0.62 ± 0.21 | 0.92 ± 0.30 |
| JCC3199 | 9.55 ± 0.05 | 79.58 ± 2.00 | 0.88 ± 0.01 | 1.10 ± 0.02 |
| JCC3200 | 10.20 ± 0.04 | 75.04 ± 0.49 | 2.09 ± 0.15 | 2.71 ± 0.17 |
| JCC3201 | 4.25 ± 0.09 | 25.00 ± 0.96 | 6.21 ± 0.37 | 19.93 ± 1.55 |

### Example 7

### Increased Extracellular Alkanes in JCC2055 Strains Expressing YbhGFSR and A0585ProNterm_TolC

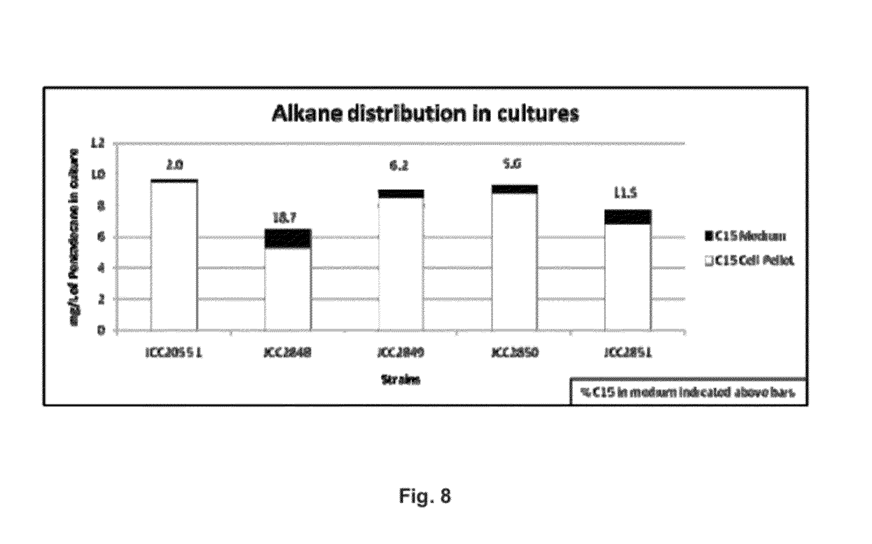

Cultures from single colonies of JCC2055 bearing a kanR marker at the A2208 locus, JCC2848, JCC2849, JCC2850 and JCC2851 (Table 14) were used to inoculate 30 ml of JB 2.1 medium (Patent Application WO/2011/017565) containing 3 mM urea to a starting OD₇₃₀=0.2. Five ml of dodecane containing 25 mg/L butylated hydroxytoluene and 25 mg/L eicosane (DBE solution) was overlayed on top of the cultures. The cultures were incubated in 125 ml flasks in a Multitron II (Infors) shaking incubator (37° C., 150 rpm, 2% CO₂/air, continuous light) for 4-7 days. At the end of the experiments, water was added to compensate for evaporation loss (based on measured mass loss of flasks from beginning to end of experiment assuming no dodecane evaporated) and 50 μl of culture was removed for OD₇₃₀s determination. 500 μl of the cultures was removed and cell pellets obtained through centrifugation for quantification of cell-associated alkanes. The supernatants were discarded and the cells resuspended in 1 ml of milli-Q water and transferred to a new microcentrifuge tube to remove contaminating DBE solution. The cells were pelleted twice more and the supernatants discarded after each spin to remove residual water. The cell pellets were vortexed for 20 seconds in 500 μl of acetone (Acros Organics 326570010) containing 25 mg/L butylated hydroxytoluene and 25 mg/L eicosane (ABE solution). The cellular debris was pelleted by centrifugation and the acetone supernatants were analyzed for the presence of 1-alkenes. The remaining culture containing the dodecane overlay was pelleted by centrifugation and samples of the DBE were removed for quantification of medium-associated alkanes. Both ABE and DBE samples were submitted for quantification of pentadecane by GC/FID. The cell pellet and medium associated pentadecane concentration for each strain and flask were then normalized to the internal standard (eicosane) and reported as mg/L of culture. The strains bearing the transporter complex show an increased percentage of secreted pentadecane in the medium when compared to the control strain which produced a similar titre of pentadecane (FIG. 8). The percentage of alkanes secreted by engineered photosynthetic microbes comprising a recombinant YbhGFSR efflux pump and recombinant OMP is at least two fold higher than that secreted by an otherwise identical strain lacking these recombinant proteins. In certain cases, the percentage of secreted alkanes is increased at least three, four or five fold in the engineered strains comprising the recombinant efflux pump/OMP relative to otherwise identical strains lacking the pump. Alkane secretion levels greater than 5%, greater than 10%, greater than 15% and/or between 5 and 20% and/or between 10 and 20% were observed in this experiment in strains comprising recombinant efflux pump/OMP proteins.

TABLE 14
Table 14: Joule Culture Collection (JCC) numbers of the
JCC2055-derived strains described in Table 15 that were
investigated for the production of pentadecane.

| | A0585_ProNTerm_TolC | P1-P2 | ybhGFSR (driven |
|---|---|---|---|
| Strain | (driven by P1 promoter) | promoters | by P2 promoter) |
| JCC2055- 1* | — | — | — |
| JCC2848 | A0585_ProNTerm_TolC | P(aphII)- | ybhGFSR (driven |
| | (driven by P1 promoter) | P(aphII) | by P2 promoter) |
| JCC2849 | A0585_ProNTerm_TolC | P(aphII)- | ybhGFSR (driven |
| | (driven by P1 promoter) | P(psaA) | by P2 promoter) |
| JCC2850 | A0585_ProNTerm_TolC | P(psaA)- | ybhGFSR (driven |
| | (driven by P1 promoter) | P(tsr2142) | by P2 promoter) |
| JCC2851 | A0585_ProNTerm_TolC | P(nir09)- | ybhGFSR (driven |
| | (driven by P1 promoter) | P(nir07) | by P2 promoter) |

*The strain bears the same marker (kanR) at the amt1-downstream targeted locus described in Table 15.

### Example 8

### YbhGFSR OMP Constructs

JCC2055 is JCC138 (Synechococcus sp. PCC 7002) bearing on the endogenous high-copy plasmid pAQ3 a nitrate-inducible/urea-repressible promoter, P(nir07), a synthetic fragment derived from the nirA promoter of Synechococcus elongatus PCC 7942, directing the transcription of a codon- and restriction-site-optimized synthetic adm-aar operon encoding the alkanal deformylative monooxygenase (Adm; cce_{—}0778) and acyl-acyl-carrier-protein (acyl-ACP) reductase (Aar; cce_{—}1430) proteins from Cyanothece ATCC 51142. The adm-aar operon in JCC2055 is linked to a downstream spectinomycin-resistance marker cassette (aadA), and the strain is fully segregated as determined by PCR. JCC2055 was generated by transforming JCC138 with plasmid pJB1331, a synthetic double-crossover recombination vector bearing upstream and downstream homology regions flanking the heterologous P(nir07)-adm-aar/aadA cassette, targeting said cassette to the intergenic region between the convergently transcribed genes SYNPCC7002_C0006 and SYNPCC7002_C0007 on pAQ3. The DNA sequence of pJB1331 is shown in SEQ ID NO:52.

The sequential enzymatic activities of Aar and Adm convert endogenous hexadecyl-ACP into n-pentadecane via a hexadecanal intermediate in JCC2055. This strain typically generates, after depletion of urea in a mixed nitrate/urea culture medium during photoautotrophic growth, approximately 2% of dry cell weight as n-alkanes, >95% of which comprises n-pentadecane. Wild-type JCC138 makes no detectable n-alkane. Typically, >95% of the n-alkane synthesized by JC2055 are found to be cell-associated, almost certainly being located within the cytosol, i.e., <5% of the n-alkane is found to be growth-medium-associated in this strain.

To make JCC2055 competent to efflux intracellular n-alkane and/or n-alkenes into the growth medium, this strain has been transformed with a panel of DNA constructs (assembled from component fragments in E. coli using standard cloning techniques involving restriction digestion and ligation operation) designed to chromosomally integrate genes encoding an energy-driven tripartite n-alkane efflux pump complex. Tripartite efflux pumps are found in Gram-negative prokaryotes, and are thus called because they comprise proteinaceous components in the inner membrane, in the periplasmic space, and in the outer membrane—all of which interact together to form a functional extrusion pump. Tripartite pumps are energetically driven by either the proton-motive force across the inner membrane or by the ATP hydrolytic activity associated with the cytosolic moiety of the inner membrane component, and catalyze the active efflux of substrates from either the periplasmic space and/or cytosol beyond the outer membrane. The tripartite efflux pump selected for expression in JCC2055, the TolC-YbhGFSR complex, and homologous variants thereof, is of the ATP-hydrolytic variety, its subunits being encoded by the ybhG-ybhF-ybhS-ybhR (ybhGFSR) operon and tolC gene of Escherichia coli K-12, or homologous operons and genes, respectively, thereof. ybhG encodes the periplasmic membrane fusion protein subunit(s), ybhF the cytosolically located ATP-hydrolyzing subunit(s) of the inner membrane component encoded by the paralogous integral membrane proteins encoded by ybhS and ybhR, and tolC the outer membrane protein (OMP—when genic, referred to as omp) subunit(s) known to partner with many different periplasmic/inner membrane efflux pumps in E. coli.

One class of efflux pump constructs integrated into JCC2055 consist of an omp transcriptional unit, P1-omp, adjacent to, and divergently transcribed from, a ybhGFSR operonic transcriptional unit, P2-ybhGFSR, wherein P1 and P2 indicate specific promoters independently driving transcription of omp and ybhGFSR, respectively, the P1-P2 unit being referred to as the divergent promoter. Note that, in this context, P1 and P2 promoters are defined so as to include not only the promoter region itself, but also any and all additional downstream sequence up to the first base pair of the start codon of the associated ORF. Also note that, in this context, omp typically refers to one of a multitude of possible variants of the OMP pump component, and ybhGFSR typically refers to one of a multitude of possible variant YbhG/YbhF/YbhS/YbhR complements. Associated with these divergently transcribed omp-P1-P2-ybhGFSR constructs is an antibiotic-resistance cassette, different from aadA, to permit selection of transformants. Flanking the omp-P1-P2-ybhGFSR/marker cassette are upstream and downstream homology regions used for recombinationally integrating linked constructs into the JCC2055 chromosome. In some omp-P1-P2-ybhGFSR efflux pump constructs, the encoded OMP is E. coli TolC, or a homolog thereof. In other omp-P1-P2-ybhGFSR efflux pump constructs, the encoded OMP is either the TolC homolog of JCC138, SYNPCC7002_A0585 or the TolC homolog of Synechococcus elongatus PCC 7942, Synpcc7942_{—}1761. In yet other omp-P1-P2-ybhGFSR efflux pump constructs, the encoded YbhG is one of several different homologous variants with specifically modified coiled-coil regions designed to promote functional interaction between the YbhGFSR component and either SYNPCC7002_A0585 or E. coli TolC, or a homolog thereof, encoded by the partner omp gene. The second class of efflux pump constructs integrated into JCC2055 consists of a P2-ybhGFSR transcriptional unit integrated at one locus (linked to a unique antibiotic-resistance marker) of the JCC2055 chromosome and a P1-omp transcriptional unit at another, separate, locus of the JCC2055 chromosome (also linked to a unique antibiotic-resistance marker); in some cases, P1-omp corresponds to the wild-type SYNPCC7002_A0585 locus, i.e., native promoter plus native coding sequence.

One set of 14 divergent omp-P1-P2-ybhGFSR efflux pump constructs was integrated into JCC2055 immediately downstream of the amt1 open reading frame (SYNPCC7002_A2208)—referred to as the amt1-downstream locus. This was achieved by using a double-crossover recombination vector bearing upstream and downstream homology regions flanking the heterologous omp-P1-P2-ybhGFSR cassette, targeting said cassette to this region between base pairs 2,299,863 and 2,299,864 of the JCC138 chromosome (NCBI accession #NC_{—}010475). Homology regions and omp-P1-P2-ybhGFSR cassette were harbored on an E. coli vector backbone derived from pJ208 (DNA2.0; Menlo Park, Calif.). The sequence of the homology regions and vector backbone, minus the omp-P1-P2-ybhGFSR cassette, whose insertion site is indicated by a dash, is shown in SEQ ID NO:55.

The omp gene for all 14 amt1-downstream-targeted divergent omp-P1-P2-ybhGFSR pump constructs was either the native tolC gene from E. coli K-12 substr. MG1655 (E. coli MG1655; NCBI accession #NC_{—}000913), or one of two derivatives of this gene modified in the 5′ region. The three E. coli tolC variants differ in their encoded cleavable N-terminal signal sequence: either (1) the natural E. coli signal sequence of TolC, (2) the predicted signal sequence of the JCC138 TolC homolog SYNPCC7002_A0585 (A0585), or (3) the contiguous sequence encompassing both the predicted signal sequence and proline-rich N-terminal region of SYNPCC7002_A0585 (A0585_ProNterm), was employed. Only one ybhGFSR operon was used for all 14 amt1-downstream-targeted divergent tolC-P1-P2-ybhGFSR pump constructs: the native ybhGFSR operon from E. coli MG1655 (the native ybhG start codon being changed from GTG to ATG). Five different variants of the P1-P2 divergent promoter were employed for the 14 constructs, component P1 and P2 promoters being selected from a panel of constitutive (P(aphII), P(psaA), P(tsr2142), and P(ompR)) or nitrate-inducible/urea-repressible promoters (P(nir09) and P(nir07)) active in JCC138. For all amt1-downstream-targeted tolC-P1-P2-ybhGFSR pump constructs, the marker used to select for JCC2055 transformants was a kanamycin-resistance (kan) cassette located between P1 and P2, bearing its own promoter, transcribed in the same direction as P2, and rho-independent transcriptional terminator. The structures of these 14 amt1-downstream-targeted tolC-P1-P2-ybhGFSR pump constructs are summarized in the Table 15; associated DNA and protein sequences are indicated in SEQ ID NOs:56-75. The DNA sequences of each of the 14 fully assembled, chromosomally integrated constructs can be generated by concatenating, in the following order, (1) the appropriate tolC variant DNA sequence in reverse complementary orientation with respect to the indicated DNA sequence, (2) the appropriate P1-P2 divergent promoter (containing the internal kan marker) in the orientation corresponding to the indicated DNA sequence, and (3) the native E. coli ybhGFSR DNA sequence in the orientation corresponding to the indicated DNA sequence, and then situating the resulting tripartite sequence concatamer between the flanking invariant homology region/bidirectional terminator DNA sequences of the amt1-downstream homologous recombination vector (i.e., at the site of the dash in vector backbone of SEQ ID NO:55).

TABLE 15
Summary of the 14 amt1-downstream-targeted divergent omp-P1-
P2-ybhGFSR efflux pump constructs transformed into JCC2055.

| Base | omp-P1-P2-ybhGFSR | omp | P1-P2 divergent | ybhGFSR |
|---|---|---|---|---|
| strain | integration locus | (driven by promoter P1) | promoter | (driven by promoter P2) |
| JCC2055 | Between base pairs | A0585_ProNterm_tolC | P(aphII)-P(aphII) | ybhG-ybhF-ybhS-ybhR |
| | 2,299,863 and 2,299,864 | | P(aphII)-P(psaA) | |
| | of the JCC138 | | P(psaA)-P(tsr2142) | |
| | chromosome (see text) | | P(nir09)-P(nir07) | |
| | | A0585_tolC | P(aphII)-P(aphII) | |
| | | | P(aphII)-P(psaA) | |
| | | | P(psaA)-P(tsr2142) | |
| | | | P(tsr2142)-P(ompR) | |
| | | | P(nir09)-P(nir07) | |
| | | tolC | P(aphII)-P(aphII) | |
| | | | P(aphII)-P(psaA) | |
| | | | P(psaA)-P(tsr2142) | |
| | | | P(tsr2142)-P(ompR) | |
| | | | P(nir09)-P(nir07) | |

The DNA sequences of the indicated omp genes, P1-P2 promoters, and ybhGFSR operon are detailed below.

In addition to the 14 divergent omp-P1-P2-ybhGFSR pump constructs derived from native E. coli genomic DNA discussed above (Table 15), another, larger set of divergent omp-P1-P2-ybhGFSR pump constructs derived from mostly synthetic DNA fragments (DNA2.0; Menlo Park, Calif.) was assembled and transformed into JCC2055. This latter set of synthetic omp-P1-P2-ybhGFSR constructs was integrated into JCC2055 such that the SYNPCC7002_A0358 open reading frame and associated upstream sequence (referred to as the ΔA0358 locus) were deletionally replaced with said constructs. This was achieved by using a double-crossover recombination vector bearing upstream and downstream homology regions flanking the heterologous omp-P1-P2-ybhGFSR, targeting said cassette to this region, replacing base pairs 377,985 to 381,565 of the JCC138 chromosome (NCBI accession #NC_{—}010475). Homology regions and omp-P1-P2-ybhGFSR cassette were harbored on an E. coli vector backbone derived from pJ201 (DNA2.0; Menlo Park, Calif.). The sequence of the homology regions and vector backbone, minus the omp-P1-P2-ybhGFSR cassette, whose insertion site is indicated by a dash, is provided in SEQ ID NO:76. Note that, in contrast to the amt1-downstream-targted omp-P1-P2-ybhGFSR pump constructs (Table 15) that featured a kan marker situated between promoters P1 and P2, the ΔA0358-targted omp-P1-P2-ybhGFSR pump constructs possess a gentamycin-resistance (aacC1) transformant selection marker situated downstream of, and transcribed in the same direction as, the ybhGFSR operon.

Four omp gene variants used for the ΔA0358-targeted divergent omp-P1-P2-ybhGFSR pump constructs were either a restriction- and codon-optimized version of the E. coli MG1655 tolC, tolC_opt, or one of three derivatives of this gene modified in the 5′ region. The four codon-optimized tolC variants differ in their encoded cleavable (codon-optimized) N-terminal signal sequence: either (1) the predicted signal sequence of SYNPCC7002_A0585 (A0585), (2) the predicted signal sequence of the JCC138 OMP85/BamA homolog SYNPCC7002_A0318 (A0318), (3) the contiguous sequence encompassing both the predicted signal sequence and proline-rich N-terminal region of SYNPCC7002_A0585 (A0585_ProNterm), was employed, or (4) the contiguous sequence encompassing both the signal sequence and proline-rich N-terminal region of SYNPCC7002_A0318 (A0318_ProNterm), was used. Two additional omp gene variants used for the ΔA0358-targeted divergent omp-P1-P2-ybhGFSR pump constructs, both restriction- and codon-optimized: (1) the SYNPCC7002_A0585 ORF with its two putative 24 amino acid encoded membrane-fusion-protein-interacting loop regions replaced with the corresponding regions of E. coli TolC, denoted as hybrid_A0585, and (2) the Synpcc7942_{—}1761 ORF, corresponding to the TolC homolog in Synechococcus elongatus PCC 7942, with its two putative 24 amino acid encoded membrane-fusion-protein-interacting loop regions replaced with the corresponding regions of E. coli TolC, denoted as hybrid_{—}1761. The loop regions in question are those located between α-helices H3 and H4 and between α-helices H7 and H8 of E. coli TolC, using the nomenclature and X-ray crystallographic information of Koronakis V et al. (2000). Crystal structure of the bacterial membrane protein TolC central to multidrug efflux and protein export. Nature 405:914-919. Accompanying the six aforementioned omp gene variants, four ybhG gene variants were used for the ΔA0358-targeted divergent omp-P1-P2-ybhGFSR pump constructs, all derived from a restriction- and codon-optimized version of E. coli ybhG, ybhG_opt, but differing in their encoded (codon-optimized) N-terminal region: either (1) the predicted signal sequence of E. coli YbhG, (2) the signal sequence of E. coli TorA, a protein exported into the periplasm via the twin-arginine transport (TAT) system (TorA), (3) the predicted signal sequence of the JCC138 N-acetylmuramyl-L-alanine amidase SYNPCC7002_A0578 (A0578), or (4) the predicted signal sequence of the JCC138 OMP85/BamA homolog SYNPCC7002_A0318 (A0318), was employed. Accompanying the six omp variants and four ybhG_opt variants, three variants of the ybhS-ybhR suboperonic pair were used, all derived from restriction- and codon-optimized gene sequences encoding E. coli ybhS and ybhR, ybhS_opt and ybhR_opt, respectively, but differing in their encoded, augmented (codon-optimized) N-terminal regions: either (1) no additional N-terminal sequences were added to the encoded YbhS and YbhR proteins (i.e., they both had the native amino acids sequences), or, either (2) a 97 amino acid pseudo-leader sequence (PLS) derived from the predicted transmembraneous region encoded within the s110041 open reading frame of Synechocystis sp. PCC 6803 (s110041_Nin_PLS) replacing the N-terminal methionine of both YbhS and YbhR, or (3) a 116 amino acid PLS derived from the predicted transmembraneous region encoded within the slr1044 open reading frame of Synechocystis sp. PCC 6803 (slr1044_Nin_PLS) replacing the N-terminal methionine of both YbhS and YbhR, was used. PLS regions were added in an effort to potentially bias localization of YbhS and YbhR to the plasma membrane, rather than to the thylakoid membrane. The YbhF component of the ΔA0358-targeted divergent omp-P1-P2-ybhGFSR pump constructs was an invariant restriction- and codon-optimized version of E. coli ybhF, ybhF_opt. 22 different variants of the P1-P2 divergent promoter were employed for the each ΔA0358-targeted omp-P1-P2-ybhGFSR construct, some component P1 and P2 promoters being selected from a panel of promoters known to be constitutively active in JCC138, and others being selected as naturally occurring P1-P2 divergent promoters (of unknown activity with respect to JCC138) in non-JCC138 cyanobacterial genomes. Each of these 22 P1-P2 divergent promoters was designed with symmetric terminal NdeI sites such that, during construct assembly in E. coli via NdeI digestion and ligation, it could insert between the omp gene and ybhGFSR operon in either orientation (i.e., complementary or reverse complementary) thereby generating 44 possible divergent promoter sequences driving a given omp-ybhGFSR base construct. The structures of the omp-ybhGFSR constructs integrated at the ΔA0358 locus are summarized in Table 16; associated DNA and protein sequences are provided in SEQ ID NOs:77-88. The DNA sequences of each of the fully assembled, chromosomally integrated constructs can be generated by concatenating, in the following order, (1) the appropriate omp variant DNA sequence in reverse complementary orientation with respect to the indicated DNA sequence, (2) the appropriate P1-P2 divergent promoter in either complementary or reverse complementary orientation with respect to the indicated DNA sequence, (3) the appropriate ybhG variant in the orientation corresponding to the indicated DNA sequence, and (4) the appropriate ybhFSR variant DNA sequence in the orientation corresponding to the indicated DNA sequence, and then situating the resulting tetrapartite sequence concatamer between the flanking invariant homology region/bidirectional terminator DNA sequences of the ΔA0358 homologous recombination vector (SEQ ID NO:76) (i.e., at the site of the dash in the vector backbone in SEQ ID NO:76). Note that ΔA0358-targeted omp-P1-P2-ybhGFSR constructs were combinatorially assembled to generate, at least theoretically, all 3,168 possible combinations of 6 omp variants, 4 ybhG_opt variants, 3 ybhS_opt-ybhR_opt operon variants, and 44 divergent P1-P2 promoters.

TABLE 16

Summary of the ΔA0358-targeted divergent omp-P1-P2-ybhGFSR efflux pump
constructs transformed into JCC2055. The DNA sequences of the
indicated omp genes, P1-P2 promoters, ybhG genes, and ybhFSR
sub-operons are detailed below.

| | omp-P1-P2- | | |
|---|---|---|---|
| | ybhGFSR | | |
| Base | integration | omp | P1-P2 divergent promoter |
| strain | locus | (driven by promoter P1) | (either orientation) |
| JCC2055 | Replacing base | A0585_tolC_opt, | P(aphII)-P(psaA)_v1, |
| | pairs 377,985 to | A0318_tolC_opt, | P(aphII)-P(EM7), |
| | 381,565 of the | A0585_ProNterm_tolC_opt, | P(psaA)-P(EM7), |
| | JCC138 | A0318_ProNterm_tolC_opt, | P(cpcC)-P(EM7), |
| | chromosome | hybrid_A0585, | P(aphII)-P(psaA)_v2, |
| | (see text)¹ | hybrid_1761 | P(psaA)-P(tsr2412), |
| | | | P(tsr2412)-P(ompR), |
| | | | P(aphII)-P(aphII), |
| | | | cce_0538-cce_0539, |
| | | | cce_3068-cce_3069, |
| | | | all2487-alr2488, |
| | | | all1697-alr1698, |
| | | | all0307-alr0308, |
| | | | Synpcc7942_0945- |
| | | | Synpcc7942_0946, |
| | | | Synpcc7942_0012- |
| | | | Synocc7942_0013, |
| | | | sll1837-slr1912, |
| | | | sll0586-slr0623, |
| | | | tll1506-tlr1507, |
| | | | tll0460-tlr0461, |
| | | | cce_1144-cce_1145, |
| | | | cce_2528-cce_2529, |
| | | | all4289-alr4290 |

| | ybhG | ybhFSR |
|---|---|---|
| Base | (driven by | (operonic with ybhG; driven |
| strain | promoter P2) | by P2) |
| JCC2055 | ybhG_opt, | ybhF_opt-ybhS_opt-ybhR_opt, |
| | torA_ybhG_opt, | ybhF_opt- |
| | A0578_ybhG_opt, | sll0041_Nin_PLS_ybhS_opt- |
| | A0318_ybhG_opt | sll0041_Nin_PLS_ybhR_opt, |
| | | ybhF_opt- |
| | | slr1044_Nin_PLS_ybhS_opt- |
| | | slr1044_Nin_PLS_ybhR_opt |

¹The sequence 5′-ACTGCCCTCGATCTGTA between the yhdN/rplQ transcriptional terminator and the 3′ end of omp gene is absent in constructs containing hybrid_A0585 and hybrid_1761.

The 22 divergent promoter sequences used for the ΔA0358-targeted omp-P1-P2-ybhGFSR constructs are shown in Table 17.

TABLE 17

Summary of the 22 divergent promoters used for ΔA0358-targeted
divergent omp-P1-P2-ybhGFSR efflux pump constructs transformed
into JCC2055.

| | |
|---|---|
| Divergent P1-P2 promoter | Sequence (flanked by symmetric, terminal half-NdeI sites) |

| | |
|---|---|
| P(aphII)-P(psaA)_v1 | ATGAAAATCCTCCTAAGAAATTATGTAAGCAGACAGTTTTATTGTTCATGATGAT |
| (SEQ ID NO: 89) | ATATTTTTATCTTGTGCAATGTAACATCAGAGATTTTGAGACACAACGTGGCTTT |
| | CCCCCCCCCCCCCTGTGGAAGTACATACGTGTTGCCTGGCTTTTACGAGATCGTA |
| | AGCGTTTTACGATGTCTTTGTCGCCTTATATTGCCCTTCAAGAGTTTGCAACATT |
| | AGAACTTTGGAGGAGGTGCTACAATTTTGATGACGACACTGATGCGGCATTGGAT |
| | CTTATCCGCCCCTATATTATGCATTTATACCCCCACAATCATGTCAAGAATTCAA |
| | GCATCTTAAATAATGTTAATTATCGGCAAAGTCTGTGCTCCCCTTCTATAATGCT |
| | GAATTGAGCATTCGCCTCCTGAACGGTCTTTATTCTTCCATTGTGGGTCTTTAGA |
| | TTCACGATTCTTCACAATCATTGATCTAAAGATCTTTCTTAGGAGGATTTTCAT |
| P(aphII)-P(EM7) | ATGAAAATCCTCCTAAGAAATTATGTAAGCAGACAGTTTTATTGTTCATGATGAT |
| (SEQ ID NO: 90) | ATATTTTTATCTTGTGCAATGTAACATCAGAGATTTTGAGACACAACGTGGCTTT |
| | CCCCCCCCCCCCCTGCCACACGTTTTGTTCGCAGCAGGAGTTACGGTCGGGTTTG |
| | GAACGTAGCGCAGCGCAGGCGAAATTTTCTCTGCACATCTATGCGTCCGCATTAG |
| | GATGGATGCGCAAGTACCCCAAAATTATGTTAAATCAACACTTTACGTAGTAGGT |
| | GATACGGGAGCTGCCAGCTATACTAATGATCCACTATCTTGACTAGCAATTTCAT |
| | AGAGAAAACTCTCCGGGTCATGCACTCAAAAACCCTTTATACGCTCACCTGCGTC |
| | TCATGTTTTGGTCCAATCGAAGAACGGCTCCCATAACGGGAATGTTGACAATTAA |
| | TCATCGGCATAGTATATCGGCATAGTATAATACGTTTCTTAGGAGGATTTTCAT |
| P(psaA)-P(EM7) | ATGAAAATCCTCCTAAGAAAGATCTTTAGATCAATGATTGTGAAGAATCGTGAAT |
| (SEQ ID NO: 91) | CTAAAGACCCACAATGGAAGAATAAAGACCGTTCAGGAGGCGAATGCTCAATTCA |
| | GCATTATAGAAGGGGAGCACAGACTTTGCCGATAATTAACATTATTTAAGATGCT |
| | TGAATTCTTGACATGATTGTGGGGGTATAAATGCATAATATAGGGGCCACCATCA |
| | TGTTATGTCCCCAGAGACAGTGGTTTTGTGTGGATTACCAGTGACACGAGTCGGG |
| | CGTTCAAACTAGCCGCCGTAATATAGTACGTATCAGTTCATTGCGAGAGCTTTGG |
| | TGAGGATCGCATGGCTCCGAAGCTCGGGAACGACAGGCCACGGGTTACCCGCTTC |
| | GGCCTAGTATAAGAGTCCGTACTGAGTCCTTATGGCAGGCAGTGTTGACAATTAA |
| | TCATCGGCATAGTATATCGGCATAGTATAATACGTTTCTTAGGAGGATTTTCAT |
| P(cpcC)-P(EM7) | ATGAAAATCCTCCTAAGAAAGAGGGTACAAACAAGCCCGGTGTTGTAAACAAAGG |
| (SEQ ID NO: 92) | GTCAGCCCAACGCCGACAACATCTGCTTACCTCACCGGGCAACGAAGGGAAACGC |
| | CTATTATAAGAATAATGCTTGAATCTCTCCTATTAGCCTCCGCCAGCTTCGGTAG |
| | TCTTACTCATGGGTGCGGCCTCGTCTAACAGTTGGCGAGGGCATCGCCACTACCA |
| | TGCTGTGCGGTGAGCCCACTAACACGTTAAAGCACGAACTACGTAGACGAGAGAT |
| | TCCACCTTCATGCTAGATAGATGTGATCGGCGCTAGTTCTCAGACCATGCGCACC |
| | CAGCAGATACACCACTCCAGGGACTCCCTATTGGTCGTTCGGAATAAGACGCTAT |
| | TGAGGTCCACCTGGCTAGACCAGTCTGCTTCACAATCAAGTATGTTGACAATTAA |
| | TCATCGGCATAGTATATCGGCATAGTATAATACGTTTCTTAGGAGGATTTTCAT |
| P(aphII)-P(psaA)_v2¹ | ATGATCACTTGTATTACTGTTTATGTAAGCAGACAGTTTTATTGTTCATGATGAT |
| (SEQ ID NO: 93) | ATATTTTTATCTTGTGCAATGTAACATCAGAGATTTTGAGACACAACGTGGCTTT |
| | CCCCCCCCCCCCCTTAATTAATTGGCGCGCCGAGCATCTCTTCGAAGTATTCCAG |
| | GCATCAAATAAAACGAAAGGCTCAGTCGAAAGACTGGGCCTTTCGTTTTATCTGT |
| | TGTTTGTCGGTGAACGCTCTCTACTAGAGTCACACTGGCTCACCTTCGGGTGGGC |
| | CTTTCTGCGTTTATAAAGCTTGCCCCTATATTATGCATTTATACCCCCACAATCA |
| | TGTCAAGAATTCAAGCATCTTAAATAATGTTAATTATCGGCAAAGTCTGTGCTCC |
| | CCTTCTATAATGCTGAATTGAGCATTCGCCTCCTGAACGGTCTTTATTCTTCCAT |
| | TGTGGGTCTTTAGATTCACGATTCTTCACAATCATTGATCTAAGGATCTTTGTAG |
| | ATTCTCTGTACAT |
| P(psaA)-P(tsr2412)¹ | ATGATCAGAGAATCTACAAAGATCCTTAGATCAATGATTGTGAAGAATCGTGAAT |
| (SEQ ID NO: 94) | CTAAAGACCCACAATGGAAGAATAAAGACCGTTCAGGAGGCGAATGCTCAATTCA |
| | GCATTATAGAAGGGGAGCACAGACTTTGCCGATAATTAACATTATTTAAGATGCT |
| | TGAATTCTTGACATGATTGTGGGGGTATAAATGCATAATATAGGGGCTTAATTAA |
| | TTGGCGCGCCGAGCATCTCTTCGAAGTATTCCAGGCATCAAATAAAACGAAAGGC |
| | TCAGTCGAAAGACTGGGCCTTTCGTTTTATCTGTTGTTTGTCGGTGAACGCTCTC |
| | TACTAGAGTCACACTGGCTCACCTTCGGGTGGGCCTTTCTGCGTTTATAAAGCTT |
| | CCAAGGTGGCTACTTCAACGATAGCTTAAACTTCGCTGCTCCAGCGAGGGGATTT |
| | CACTGGTTTGAATGCTTCAATGCTTGCCAAAAGAGTGCTACTGGAACTTACAAGA |
| | GTGACCCTGCGTCAGGGGAGCTAGCACTCAAAAAAGACTCCTCCTGTACAT |
| P(tsr2412)-P(ompR)¹ | ATGATCAGGAGGAGTCTTTTTTGAGTGCTAGCTCCCCTGACGCAGGGTCACTCTT |
| (SEQ ID NO: 95) | GTAAGTTCCAGTAGCACTCTTTTGGCAAGCATTGAAGCATTCAAACCAGTGAAAT |
| | CCCCTCGCTGGAGCAGCGAAGTTTAAGCTATCGTTGAAGTAGCCACCTTGGTTAA |
| | TTAATTGGCGCGCCGAGCATCTCTTCGAAGTATTCCAGGCATCAAATAAAACGAA |
| | AGGCTCAGTCGAAAGACTGGGCCTTTCGTTTTATCTGTTGTTTGTCGGTGAACGC |
| | TCTCTACTAGAGTCACACTGGCTCACCTTCGGGTGGGCCTTTCTGCGTTTATAAA |
| | GCTTTAGTACAAAAAGACGATTAACCCCATGGGTAAAAGCAGGGGAGCCACTAAA |
| | GTTCACAGGTTTACACCGAATTTTCCATTTGAAAAGTAGTAAATCATACAGAAAA |
| | CAATCATGTAAAAATTGAATACTCTAATGGTTTGATGTCCGAAAAAGTCTAGTTT |
| | CTTCTATTCTTCGACCAAATCTATGGCAGGGCACTATCACAGAGCTGGCTTAATA |
| | ATTTGGGAGAAATGGGTGGGGGCGGACTTTCGTAGAACAATGTAGATTAAAGTAC |
| | TGTACAT |
| P(aphII)-P(aphII)¹ | ATGATCACTTGTATTACTGTTTATGTAAGCAGACAGTTTTATTGTTCATGATGAT |
| (SEQ ID NO: 96) | ATATTTTTATCTTGTGCAATGTAACATCAGAGATTTTGAGACACAACGTGGCTTT |
| | CCCCCCCCCCCCCTTAATTAATTGGCGCGCCGAGCATCTCTTCGAAGTATTCCAG |
| | GCATCAAATAAAACGAAAGGCTCAGTCGAAAGACTGGGCCTTTCGTTTTATCTGT |
| | TGTTTGTCGGTGAACGCTCTCTACTAGAGTCACACTGGCTCACCTTCGGGTGGGC |
| | CTTTCTGCGTTTATAAAGCTTGGGGGGGGGGGGGAAAGCCACGTTGTGTCTCAAA |
| | ATCTCTGATGTTACATTGCACAAGATAAAAATATATCATCATGAACAATAAAACT |
| | GTCTGCTTACATAAACAGTAATACAAGTGTACAT |
| cce_0538-cce_0539² | ATGTGACTTAACTCCTGATTGAACATCAATATATTTTTTTATGGTTGCTTATTTT |
| (SEQ ID NO: 97) | TAATAACTTTTTTCTAAAAATAAAATTAAGTTTTATAAAGAATGATTAAAAGAAT |
| | TACAAAATATAAACATAATCTTCACATAAAAATCTTTACATAAAGCGTAATTCTA |
| | CTAACGACAGAAACAGGGTGCCTTATGTTAGCCTATAGTTAGATTTAGTCCATAT |
| | AAACAATTTAGATTCAGAATTGATTCCCTGTTTCAATATTTCCTATCCTTACCAT |
| | CAATTGTATTAAATATAGGTAGCAT |
| cce_3068-cce_3069² | ATGAGAGAGTTATCCTGAATCAAAATTTCTTTGAAAAAAAAAGAGAAGGAAAAAA |
| (SEQ ID NO: 98) | AAGATATTTTTAACAACAATGTTTGAAATTAATATCAGTTCATCTATTTTGATTA |
| | GAAGTTGACAATAGTTTGCAATTACAAAAAAAGATGGACGTTTGGTTGATTTTTA |
| | GCTATTCTTGAAGTAGAAAGAAATATTCTAAGAATAAAGTATAGCTTAAGAATTT |
| | TATTGGGTTAGGTAAACTGACAT |
| all2487-alr2488² | ATGAATTTTCCCTAAGTTATAGTGAACTTTTTTCTTGTTTATTAAAACAAAAAAT |
| (SEQ ID NO: 99) | TTGCATTTTGAAAACTGTATTTATCCCTTTTCACAAAATATTAATAATACGTAAA |
| | TTCTCTCAAAGGTTTCCATACAAAAAACCCAGAGTTTCTACTGAGTTAATTAACC |
| | ATGACGACATAAATATTTAGTGTCAATCTTCCGATTGAGTATCAGCTTGATAAAC |
| | TAGGAGCTAAGTTCCCTCATCAGCAATTTCTCAGGAAAACAT |
| all1697-alr1698² | ATGTGGATATGTCCTGATATTTGCACTCAACAGCTAAAAATATATTTACAATTCA |
| (SEQ ID NO: 100) | TTGAGAATTGCTATACAATTTTATTCTGATAAGAAGGGGAGTAGCTGCTGGCAAA |
| | AGCCAGTACATCTGAATCAACATACTGGCGATGAGCCTGGTTCAGGTGACAACTA |
| | GAAAATATTTGGAAGCGAGACCTTCACTAAGTTCACATTTAAGATGTGGCTTGGT |
| | GGGGTCTTTTGGCATTCATCAAGCTTCACATCGGTAAACATTTTTCAGGAGCTTG |
| | AGCAT |
| all0307-alr0308² | ATGCTGTAATCCTTACACAAAGAGTGAAAAATCCTATGAGTGTTGTCTATCGTTG |
| (SEQ ID NO: 101) | GCTACAACTACTTTAATTTTGCAACACCAAAATCACGTTTATAGTGTTTTCTAGT |
| | CTGCTGGCGTGCCAATTTATCTGCGTCCATCTGGGGTTAAGTGTTTCTTGTTCTC |
| | ATTTACTGCGTCGTGCGTATCTGTCGGGAGTTGTCATGTCAGTGGTTTTTGACCT |
| | GGTTTAATGCTCTATCCCCTTGTGGTGTATTTTTAGATGGCTATCACTATATGAC |
| | GTTTTCATCGCCATCCCATAGAAACTTTTACTCAGAGAAACTTTGTTTTATGTTC |
| | GACTGTAGGCGATGATTTCCGGTCGGTAGCAGACGGAGGCTGCGTTAATGCCAAT |
| | ACTCAGCATACGAAACTCTGGCAATTATGGAAAATAATATATGTAAGTCGAGTAT |
| | CGTAAGACTCACTTGATTTCCTCATTTCCTCTAGGAACAT |
| Synpcc7942_0945- | ATGAGAACTAGCACCTAGATTGGAGGAGATTACAGTCATGGACAAATTCTGCGAT |
| Synpcc7942_0946² | CGGACTTGAGGACTATCGTTACTGTAGCGTCAAGGCAACGAGAAACAAGAGGTAC |
| (SEQ ID NO: 102) | TGTTTTGCTCAAAAGCTGATTGAACGCTCACTCCTTGATCACTGTGCTAACTGGC |
| | TCTTGCTCTGAATGTTACTGAGCATTTCTAAACCCAGAAGCCAATAGAAACGGGT |
| | GATATATCTAAAGCTGTTGAAAACAGCATTGTTCATTGGCAGCCCTAGAGTCAGC |
| | GAGACAGTGCTTCGTAGCTGCTCAGCTAGATTCTGTCCGGCTGAGTTCATTGTCT |
| | GACCCAAGCTCAATTTCCCTTTGCCCTAAGGACTGGTGGCCAT |
| Synpcc7942_0012- | ATGAACCAATCCTTATGGTCATGGGGCTCCAAATCTTCAGCTGGTTTTACCCAGT |
| Synpcc7942_0013² | GAGTTTGAAGCAAGGATCTTTTAGTTTACCGAAAAATGAGGCTCAGCGATCGCAG |
| (SEQ ID NO: 103) | CAAGTTCTTGCCGACTGAGGAGGCGATCGCGGCAGCAGTGTTTGCCCGAGGTGGT |
| | CAAAGGAGCAGTTTTGGTAAAAGTCTAAAGGAAATATAAAGACTGCTGCCTTGCG |
| | GGACGAGCAATGGACTTCTCTACCCTAGGGAAAACTGATTTAGAAGTGAACTAAT |
| | CGCATAGATGATTTAATGCGTACCTTCTTTTCCACTAACTACTATTGGAATTAAA |
| | GGACACTTAAATTTAGGAATCGACAT |
| sll1837-slr1912² | ATGAACTCCTCAAACCACAGAAATTGTTAACGCCAATCTTACTAGAACTAGGCTG |
| (SEQ ID NO: 104) | GCTTTGCCCACGGCCAGGGATGGGCTTACCCTGGGGATAAATAGTTTTTTGGTAT |
| | TAAACTAAACAGGCCGTAACGGACAATACGGAAATTGTCGCTCCCAAAACACAAA |
| | ATAGTCAGCACATCGACATAATTGACGGCGATCGCCTAAATTACTAGAGTTGAGG |
| | CCAGTTTTGCCGTTGCCTTTTTTTCTTTTGTGTGAGGAGTCCAT |
| sll0586-slr0623² | ATGTTTGACCAACCTTTATCTCTGGATTTCACTGGAAAATGGATCTAATCACCCC |
| (SEQ ID NO: 105) | AAAAATCCCTTTAAAAAACTTAACAAATACGGAACTCCCCACCGGCAAAAACCCT |
| | ATGCCCCCCGTCCCAACCTGTACAATGAAGAGGGCGGAGACGTAAGTTTCCGTTC |
| | ACTCCTCACACCACACTCCGCCTGGATGATGTTCGGGCGGTTTCTTCTTATCTGC |
| | TCCCCAGGGGGAAAAGTGTGACGCCAACTGTGACAAAAGATGAATAAATTCTAAG |
| | TTTCACGATATTTTTCCATACAGGGGTCAACAATTGGTTATGGTAGTATTCTAAT |
| | CAGCCCATCACGAGGTTTAGAAGGATTTCCCAT |
| tll1506-tlr1507² | ATGCGTTGTTCCTCTTTAACAGTGACTGTGCCGAATAGAGCAATCTCTACGGGCA |
| (SEQ ID NO: 106) | ACCTTTGCAATGGGTAGTGTGAACGCTACGATTCCCCGCAAATGGGGCAAAATTG |
| | AGCAGTGCAAAACTCAGCGAGATGATGCAACCATCCGCAAGCCTGTGATATTGTC |
| | GTAGGTCTTATGCTTAGGATCAGCTTAGTTGATACCCAATGCAATAACTGTTGCT |
| | TTGGAGATTCTTAATTATTCTATAGGTTTGGGTTATCAATCTTTAGAGTTGTTTA |
| | TAGGTTTCTAATTAGAGGTGTACAACTATAGTCTCCCTTCTATTCAACAGGCACT |
| | GATGATTGCCTGAAATCAATTTAATGGTCCTCATGGGGGGCGATCGCTCTATTGT |
| | TTTTGAAAAAAAGGGGGTGGAATTCAT |
| tll0460-tlr0461² | ATGTGTTTCTATCCTCACACCATAACTCCCGCGTAGGGAATGACTAACCCTACAG |
| (SEQ ID NO: 107) | CCACTGAGAGTCTGTGATTCAATGTATATCACTCTATGTTCAGTCCTAGGGTCAA |
| | CATTCGGTTCTTGGTAAAACCTGCTAGAGTGGCACTACAGCCCTTTCCAAGATAT |
| | ACAGTCCATCCAGGGGAGGTCTTTCTTCCCCAGAGGGCCTCTGGCGGTTTTGAGC |
| | GGGTTTCATTTCCGTAAAAAGGGCGGTAGATTGACTGTGGTTGCCCTCTTTCTGA |
| | ACGGGGCAAGGCCATTTTTGTTGGTGTGAGGTCGAGGGTCAT |
| cce_1144-cce_1145² | ATGTAATAATAACCCTGAAAGTAACCCTAAGTCTGATGATCAAGTTTCGCTATCC |
| (SEQ ID NO: 108) | TTAAAAAATTCTCAATTTGGTCAAATTAAGGAAAGTGGAAGTAGAATTAGAGTAG |
| | TAGATCCTAAAGATACCACATTTGAAAGGTATGATGGTGATCCACCTGCACAACG |
| | TTAATTGTAAGCTAATGGTTATTGATTTTAAAAGTTGGGTTTTCTTTTACCCCAA |
| | CTTTTAGTCAACTTTAATAATACGATAAAACATTGCAAAATACTAATATGATTTT |
| | TAAAATTTAGGTTTCCATA |
| cce_2528-cce_2529² | ATGTTATTGAAGACCTTTTATAATATAAAAATTACCATACTTGTGAGATACAAAA |
| (SEQ ID NO: 109) | GTGATCTCGAAGAGATCCGCTTCGCGGTGCGCTTTGAGGCAGAGAGAGGTGTTAG |
| | GTTTACCTTATGAGTCCGAGAAACCCTATATAAATCCTATTATCATAATATCAAC |
| | TAAACTTGTGAGTTATCAATGTCTGGAAAAAGAGGCGATCGCTGATCATGGATCA |
| | TGGTCAAACTTATAGTAATCTAACATTAAGGCTCATTACTTTCATTATAATTCCA |
| | TGTTAAGTTTAAGGGTAACAT |
| all4289-alr4290² | ATGAATATCTTGGCCTGTGAGTTCTTCCCTTTTAAGAGTCTGCCACCTGAATAGG |
| (SEQ ID NO: 110) | ATGTCTTGCAAGCTCAAGATTAGTTAGTTAACCGTTGACAGTTAACGGTTAACTA |
| | AGTCCAATGTCAAGATTTCTGAGAAAAGTTGTGTCAGATTGTAAAATTTCTGATA |
| | TTCATAGTATTTAATAGGTTCGTGTTTAATGGTTGATTCACATTGGATGGATTAA |
| | GCAAAAGCCGAACTAATATGGTAAGTTAAGAATCATTAAGTTACCACACGCTAGG |
| | TGACTAGCTGATGGTGCGTGTAAAGACATAACTCTGAGAAAAGCCAATTTAACTA |
| | ATTGGTAGCCTCTCAGGAACTCAGAAGTTTTAAGACAACTGAGAATGTCAAAAAA |
| | AACGTTATTTCCTCGCGGTAGTTGCCAAAAGTTGGGAAACCCAGCTAAAGCACTG |
| | CTTAAAGACGTTGCAATTTTTAGTAAAAGAGGATTTTAGTCAT |

¹These divergent promoters contain an internal copy of the rho-independent transcriptional terminator BBa_B0015 (Registry of Standard Biological Parts; http://partsregistry.org/).
²These divergent promoters were derived by PCR amplification from natural cyanobacterial genomic DNA templates; the other sequences were synthesized (DNA2.0; Menlo Park, CA).

In addition to the amt1-downstream-targeted (Table 15) and ΔA0358-targeted (Table 16) divergent omp-P1-P2-ybhGFSR pump constructs discussed above, another set of non-divergent JCC2055 transformants was generated bearing an invariant P(tsr2412)-ybhGFSR transcriptional unit (expressing the native E. coli ybhGFSR operon) integrated at the amt1-downstream locus, and, in addition, one of each of 31 different P1-omp constructs integrated, separately, at the ΔA0358 locus. The DNA sequence corresponding to the integrated P(tsr2412)-ybhGFSR construct corresponds to the tolC-P(psaA)-kan-P(tsr2142)-ybhG-ybhF-ybhS-ybhR assembly described in Table 15, except that the DNA sequence between the amt1-downstream upstream homology region and the 5′ end of the kan cassette, i.e., that encompassing the P(psaA)-tolC unit as well as 100 bp downstream of it, was entirely deleted. The JCC2055-derived base strain bearing this kan-linked P(tsr2412)-ybhGFSR transcriptional unit was JCC2522. The DNA sequence corresponding to the base plasmid used to transform JCC2522 with the 31 P1-omp constructs corresponds to the sequence detailed above covering the ΔA0358-targeted homology regions and associated vector backbone, except that the approximately 70 bp between the ΔA0358 upstream homology region and the Tn10 bidirectional terminator (itself upstream of the gentamycin-resistance cassette), has been replaced by the rho-independent transcriptional terminator BBa_B0015 (Registry of Standard Biological Parts; http://partsregistry.org/), downstream of which is a P1-omp DNA sequence, transcribed in the same direction as the gentamycin-resistance marker (and also in the same direction as the “forward direction” of the BBa_B0015 terminator). The structures of the 31 P1-omp constructs transformed into JCC2522 are shown in Table 17; they encompass hybrid_A0585, hybrid_{—}1761, 12 derivatives of tolC_opt variously modified in their 5′ (i.e., encoded N-terminal) and 3′ regions i.e., encoded C-terminal), and three P1 promoter variants. The N-terminal tolC_opt variants employed have been previously discussed. The three different C-terminal tolC_opt variants differ in their encoded (non-cleaved) carboxyl terminal sequences: either (1) the native E. coli TolC terminal sequence was used, (2) it was replaced by the corresponding C-terminal residues of SYNPCC7002_A0585 (A0585C), or (3) it was replaced by the corresponding C-terminal residues of SYNPCC7002_A0318 (A0318C). The rationale for the using the C-terminal modifications was that C-terminal residues are known to be important for proper insertion of certain OMPs into the outer membrane (Robert V et al. (2006). Assembly Factor Omp85 Recognizes Its Outer Membrane Protein Substrates by a Species-Specific C-Terminal Motif. PLoS Biol 4:e377). The DNA sequences of each of the 31 fully assembled, chromosomally integrated P1-omp constructs can be generated by concatenating, in the following order, (1) the appropriate P1 promoter in the orientation corresponding to the indicated DNA sequence and (2) the appropriate omp DNA sequence in the orientation corresponding to the indicated DNA sequence, and then situating the resulting bipartite sequence concatamer between the flanking invariant homology region/bidirectional terminator DNA sequences of the ΔA0358-downstream homologous recombination vector—minus the aforementioned 70 bp between the ΔA0358 upstream homology region and the Tn10 bidirectional terminator—as was described for the constructs described in Table 16.

TABLE 18
Summary of the 31 ΔA0358-targeted P1-omp efflux OMP pump constructs
transformed into JCC2522, a derivative of JCC2055 bearing a P(tsr2412)-
ybhGFSR transcriptional unit integrated at the amt1-downstream locus.

| | P1-omp | Promoter | omp |
|---|---|---|---|
| Base strain | integration locus | P1 | (driven by promoter P1) |
| JCC2522 | Replacing base pairs 377,985 to | P(aphII) | A0585_tolC_opt |
| | 381,565 of the JCC138 chromosome | | A0585_tolC_opt_A0585C |
| | (see text) | | A0318_ProNTerm_tolC_opt |
| | | | A0318_ProNTerm_tolC_opt_A0585C |
| | | | A0585_ProNTerm_tolC_opt |
| | | | A0585_ProNTerm_tolC_opt_A0318C |
| | | | hybrid_A0585 |
| | | | hybrid_1761 |
| | | P(psaA) | A0585_tolC_opt |
| | | | A0585_tolC_opt_A0318C |
| | | | A0585_tolC_opt_A0585C |
| | | | A0318_tolC_opt |
| | | | A0585_ProNTerm_tolC_opt |
| | | | A0585_ProNTerm_tolC_opt_A0318C |
| | | | A0318_ProNTerm_tolC_opt |
| | | | A0318_ProNTerm_tolC_opt_A0318C |
| | | | A0318_ProNTerm_tolC_opt_A0585C |
| | | | hybrid_A0585 |
| | | | hybrid_1761 |
| | | P(tsr2142) | A0585_tolC_opt |
| | | | A0585_tolC_opt_A0318C |
| | | | A0585_tolC_opt_A0585C |
| | | | A0318_tolC_opt |
| | | | A0585_ProNTerm_tolC_opt |
| | | | A0585_ProNTerm_tolC_opt_A0318C |
| | | | A0585_ProNTerm_tolC_opt_A0585C |
| | | | A0318_ProNTerm_tolC_opt |
| | | | A0318_ProNTerm_tolC_opt_A0318C |
| | | | A0318_ProNTerm_tolC_opt_A0585C |
| | | | hybrid_A0585 |
| | | | hybrid_1761 |

The DNA sequences of the indicated P1 promoters and omp genes are detailed below.

In addition to the amt1-downstream-targeted (Table 15) and ΔA0358-targeted (Table 16) divergent omp-P1-P2-ybhGFSR pump constructs and to the split amd-downstream-/ΔA0358-targeted omplybhGFSR pump constructs (Table 18) discussed above, yet another set of JCC2055 transformants was generated bearing a panel of internally modified ybhG variants, generally expressed divergently with respect to an upstream omp variant, at the ΔA0358 locus. The rationale underlying the design of said ybhG variants was to engineer YbhGFSR transporter complexes to become able to functionally interact with the endogenous TolC-homologous OMP of JCC138, SYNPCC7002_A0585. Accordingly, amino acid sequence alignments were performed of E. coli MacA (NCBI accession #NP_{—}415399.4), E. coli AcrA (NCBI accession #NP_{—}414996.1), E. coli YbhG, and SYNPCC7002_A1723 (NCBI accession #YP_{—}001734968.1), a distant homolog of YbhG found in JCC138 which is believed to dock with SYNPCC7002_A0585. The α-helix hairpin and binding tip regions of MacA and AcrA (Kim H-M et al. (2010). Functional relationships between the AcrA hairpin tip region and the TolC aperture region for the formation of the bacterial tripartite pump AcrAB-TolC. J. Bacteriol. 192:4498-4503) were used to identify the corresponding regions in YbhG and SYNPCC7002_A1723. Chimeric YbhG proteins were designed to replace the binding tip, and the coiled-coil heptads flanking said binding tip, with the corresponding sequences of SYNPCC7002_A 1723 (YbhG_opt_hp1), or to replace the entire hairpin and binding tip of YbhG with those of SYNPCC7002_A1723 (YbhG_opt_hp2), or to replace the binding tip sequence of YbhG with that of SYNPCC7002_A1723 (YbhG_opt_hp4). As part of this strategy, a YbhG chimera was designed to contain the SYNPCC7002_A1723 hairpin and retain the binding tip and flanking coiled-coil heptads of YbhG (YbhG_opt_hp3); this YbhG variant may allow the YbhGFSR complex to span the periplasm and peptidoglycan of JCC138 to successfully dock with heterologously expressed E. coli TolC, or homologs thereof. The structures of the omp-ybhGFSR constructs transformed into JCC2055 are shown in Table 19. The DNA sequences of each of the fully assembled, chromosomally integrated efflux pump constructs can be generated by concatenating, in the following order, (1) the appropriate omp variant DNA sequence in reverse complementary orientation with respect to the indicated DNA sequence, (2) the appropriate P1-P2 divergent promoter in either complementary or reverse complementary orientation with respect to the indicated DNA sequence, (3) the appropriate ybhG hairpin variant in the orientation corresponding to the indicated DNA sequence, and (4) the appropriate ybhFSR variant DNA sequence in the orientation corresponding to the indicated DNA sequence, and then situating the resulting tetrapartite sequence concatamer between the flanking invariant homology region/bidirectional terminator DNA sequences of the ΔA0358 homologous recombination vector (SEQ ID NO:76). Note that ΔA0358-targeted omp-ybhGFSR constructs were designed to be able to be combinatorially assembled to generate, at least theoretically, all 14,784 possible combinations of 2 omp variants, 12 ybhG_opt variants (_hp1, _hp2, _hp4), 4 ybhS_opt-ybhR_opt operon variants, and 44 divergent P1-P2 promoters plus 15 omp variants, 4 ybhG_opt variants (_hp3), 4 ybhS_opt-ybhR_opt operon variants, and 44 divergent P1-P2 promoters.

TABLE 19
Table 19 Summary of the ΔA0358-targeted divergent omp-P1-P2-ybhGFSR efflux pump constructs transformed into JCC2055.

| | omp-P1- | | | | |
|---|---|---|---|---|---|
| | P2- | | | | |
| | ybhGFSR | | | | |
| | inte- | | P1-P2 divergent | ybhG | ybhFSR |
| Base | gration | omp | promoter | (driven by | (operonic with ybhG; |
| strain | locus | (driven by promoter P1) | (either orientation) | promoter P2) | driven by P2) |
| JCC2055 | Replacing | none¹, | P(aphII)-P(psaA)_v1, | ybhG_opt_hp1, | ybhF-ybhS-ybhR |
| | base pairs | SYNPCC7002_A0585 | P(aphII)-P(EM7), | ybhG_opt_hp2, | ybhF_opt-ybhS_opt-ybhR_opt, |
| | 377,985 to | | P(psaA)-P(EM7), | ybhG_opt_hp4, | ybhF_opt- |
| | 381,565 of | | P(cpcC)-P(EM7), | torA_ybhG_opt_hp1, | sll0041_Nin_PLS_ybhS_opt- |
| | the | | P(aphII)-P(psaA)_v2, | torA_ybhG_opt_hp2, | sll0041_Nin_PLS_ybhR_opt, |
| | JCC138 | | P(psaA)-P(tsr2412), | torA_ybhG_opt_hp4, | ybhF_opt- |
| | chro- | | P(tsr2412)-P(ompR), | A0318_ybhG_opt_hp1, | slr1044_Nin_PLS_ybhS_opt- |
| | mosome | | P(aphII)-P(aphII), | A0318_ybhG_opt_hp2, | slr1044_Nin_PLS_ybhR_opt |
| | (see text) | | cce_0538-cce_0539, | A0318_ybhG_opt_hp4, | |
| | | | cce_3068-cce_3069, | A0578_ybhG_opt_hp1, | |
| | | | all2487-alr2488, | A0578_ybhG_opt_hp2, | |
| | | | all1697-alr1698, | A0578_ybhG_opt_hp4 | |
| | | hybrid_A0585, | all0307-alr0308, | ybhG_opt_hp3, | |
| | | hybrid_1761, | Synpcc7942_0945- | torA_ybhG_opt_hp3, | |
| | | tolC | Synpcc7942_0946, | A0318_ybhG_opt_hp3, | |
| | | A0585_tolC, | Synpcc7942_0012- | A0578_ybhG_opt_hp3 | |
| | | A0585_tolC_opt, | Synpcc7942_0013, | | |
| | | A0585_tolC_opt_A0318C, | sll1837-slr1912, | | |
| | | A0585_tolC_opt_A0585C, | sll0586-slr0623, | | |
| | | A0585_ProNterm_tolC, | tll1506-tlr1507, | | |
| | | A0585_ProNTerm_tolC_opt, | tll0460-tlr0461, | | |
| | | A0585_ProNTerm_tolC_opt_A0318C, | cce_1144-cce_1145, | | |
| | | A0585_ProNTerm_tolC_opt_A0585C, | cce_2528-cce_2529, | | |
| | | A0318_tolC_opt, | all4289-alr4290 | | |
| | | A0318_ProNTerm_tolC_opt, | | | |
| | | A0318_ProNTerm_tolC_opt_A0318C, | | | |
| | | A0318_ProNTerm_tolC_opt_A0585C | | | |

¹Indicates that no omp was included in the omp-P1-P2-ybhGFSR construct. In this case, the OMP is provided by the native expression of the endogenous SYNPCC7002_A0585 gene.
The DNA sequences of the indicated omp genes, P1-P2 promoters, ybhG genes, and ybhFSR sub-operons are detailed below. Note that YbhG derivatives encoded by ybhG variants of hairpin (hp) subtype _hp1, _hp2, and _hp4, are designed to interact with SYNPCC7002_A0585, whereas those encoded by subtype _hp3 are designed to interact with E. coli TolC derivatives.

### Example 9

### Functional Combinations of ABC Efflux Pump Proteins for Expression in Cyanobacteria

Table 20 indicates all possible functional combinations of the OMP, YbhG, YbhF, YbhS, and YbhR proteins to be expressed in JCC2055. The appropriate combinations of OMP, YbhG, YbhF, YbhS, and YbhR are designed to lead to the formation of functional ABC efflux pumps capable of catalyzing efflux of intracellular n-pentandecane.

TABLE 20
Table 20. Protein sequences forming functional OMP-YbhGFSR ABC efflux pump variants.

| OMP variant | YbhG variant | YbhF | YbhS/YbhR variants |
|---|---|---|---|
| SYNPCC7002_A0585 | YbhG_hp1, | YbhF | YbhS/YbhR, |
| | YbhG_hp2, | | sll0041_Nin_PLS_YbhS/sll0041_Nin_PLS_YbhR, |
| | YbhG_hp4, | | slr1044_Nin_PLS_YbhS/slr1044_Nin_PLS_YbhR |
| | TorA_YbhG_hp1, | | |
| | TorA_YbhG_hp2, | | |
| | TorA_YbhG_hp4, | | |
| | A0318_YbhG_hp1, | | |
| | A0318_YbhG_hp2, | | |
| | A0318_YbhG_hp4, | | |
| | A0578_YbhG_hp1, | | |
| | A0578_YbhG_hp2, | | |
| | A0578_YbhG_hp4 | | |
| hybrid_A0585, | YbhG, | | |
| hybrid_1761, | TorA_YbhG, | | |
| TolC, | A0578_YbhG, | | |
| A0585_TolC, | A0318_YbhG, | | |
| A0585_TolC_A0318C, | YbhG_hp3, | | |
| A0585_TolC_A0585C, | TorA_YbhG_hp3, | | |
| A0585_ProNterm_TolC, | A0318_YbhG_hp3, | | |
| A0585_ProNTerm_TolC_A0318C, | A0578_YbhG_hp3 | | |
| A0585_ProNTerm_TolC_A0585C, | | | |
| A0318_TolC, | | | |
| A0318_ProNTerm_TolC, | | | |
| A0318_ProNTerm_TolC_A0318C, | | | |
| A0318_ProNTerm_TolC_A0585C | | | |

“Set 1” OMP and YbhG variants are listed in the two upper left boxes, respectively; “Set 2” OMP and YbhG variants are listed in the two lower left boxes, respectively.

There are two main efflux pump protein complement sets with respect to the OMP involved. In the first set (Set 1), SYNPCC7002_A0585 (NCBI Accession #YP_{—}001733848.1; encoded naturally by JCC138) is the single OMP variant, to be paired with one of 12 possible YbhG variants: YbhG_hp1, YbhG_hp2, YbhG_hp4, TorA_YbhG_hp1, TorA_YbhG_hp2, TorA_YbhG_hp4, A0318_YbhG_hp1, A0318_YbhG_hp2, A0318_YbhG_hp4, A0578_YbhG_hp1, A0578_YbhG_hp2, or A0578_YbhG_hp4.

In the second said set (Set 2), one of 13 possible OMP variants (hybrid_A0585, hybrid_{—}1761, TolC, A0585_TolC, A0585_TolC_A0318C, A0585_TolC_A0585C, A0585_ProNterm_TolC, A0585_ProNTerm_TolC_A0318C, A0585_ProNTerm_TolC_A0585C, A0318_TolC, A0318_ProNTerm_TolC, A0318_ProNTerm_TolC_A0318C, or A0318_ProNTerm_TolC_A0585C) is to be paired with one of 8 possible YbhG variants: YbhG, TorA_YbhG, A0578_YbhG, A0318_YbhG, YbhG_hp3, TorA_YbhG_hp3, A0318_YbhG_hp3, or A0578_YbhG_hp3.

Any given OMP/YbhG variant pair within each of the said sets can be functionally paired with YbhF—only one variant thereof, corresponding to the wild-type E. coli sequence—and one of three possible YbhS/YbhR paralog pairs: wild-type YbhS plus wild-type YbhR, s110041_Nin_PLS_YbhS plus s110041_Nin_PLS_YbhR, or slr1044_Nin_PLS_YbhS plus slr1044_Nin_PLS_YbhR.

The OMP and YbhG protein sequences associated with Set 1 are provided in SEQ ID NOs:174-186. Note that the TorA, A0318, and A0578 prefixes indicate differences only in the cleavable N-terminal signal sequence relative to the native YbhG signal sequence; other than this signal sequence difference, all mature YbhG variants of the same hairpin subtype, e.g., YbhG_hp1, TorA_YbhG_hp1, A0318_YbhG_hp1, and A0578_YbhG_hp1, are of identical protein sequence. Also note that all mature YbhG variants of the hairpin subtypes _hp1 and _hp4 are >95% identical at the amino acid level. But note that all mature YbhG variants of the hairpin subtype _hp2 are <60% identical at the amino acid level to those of either subtypes _hp1 or _hp4.

The OMP and YbhG protein sequences associated with Set 2 are provided in SEQ ID NOs:187-207. Note that A0585_TolC, A0585_TolC_A0318C, A0585_TolC_A0585C, A0585_ProNterm_TolC, A0585_ProNTerm_TolC_A0318C, A0585_ProNTerm_TolC_A0585C, A0318_TolC, A0318_ProNTerm_TolC, A0318_ProNTerm_TolC_A0318C, and A0318_ProNTerm_TolC_A0585C all contain >95% of the entire mature (i.e., post signal sequence cleavage) TolC. Note, however, that neither Hybrid_A0585 nor Hybrid_{—}1761 bears more than 35% identity at the amino acid level to TolC. Also, note that Hybrid_A0585 and Hybrid_{—}1761 are only 42% identical at the amino acid level. With respect to the YbhG variants of Set 2, as with Set 1, the TorA, A0318, and A0578 prefixes indicate differences only in the cleavable N-terminal signal sequence relative to the native YbhG signal sequence; other than this signal sequence difference YbhG, TorA_YbhG, A0578_YbhG, and A0318_YbhG are of identical mature protein sequence. But note that mature YbhG and mature YbhG variants of the hairpin subtype _hp3 bear significant alignment-based discontiguity to one another at the amino acid level.

The YbhF and YbhS/YbhR protein sequences associated with both Set 1 and Set 2 are are provided in SEQ ID NOs:208-214. Note both s110041_Nin_PLS_YbhS and slr1044_Nin_PLS_YbhS contain the entire YbhS sequence, excluding its N-terminal methionine, and that both s110041_Nin_PLS_YbhR and slr1044_Nin_PLS_YbhR contain the entire YbhR sequence, excluding its N-terminal methionine.

### Informal Sequence Listing

### SEQ ID NO:19

ybhG

GTGATGAAAAAACCTGTCGTGATCGGATTGGCGGTAGTGGTACTTGCCGC
CGTGGTTGCCGGAGGCTACTGGTGGTATCAAAGCCGCCAGGATAACGGCC
TGACGCTGTATGGCAACGTGGATATTCGTACGGTAAATCTTAGTTTCCGT
GTTGGGGGGCGCGTTGAATCGCTGGCGGTGGACGAAGGTGATGCTATCAA
AGCGGGCCAGGTGCTGGGCGAACTGGATCACAAGCCGTATGAGATTGCCC
TGATGCAGGCGAAAGCGGGTGTTTCGGTGGCACAGGCGCAGTATGACCTG
ATGCTTGCCGGGTATCGCAATGAAGAAATCGCTCAGGCCGCCGCAGCGGT
GAAACAGGCGCAAGCCGCCTATGACTATGCGCAGAACTTCTATAACCGCC
AGCAAGGGTTGTGGAAAAGCCGCACTATTTCGGCAAATGACCTGGAAAAT
GCCCGCTCCTCGCGCGACCAGGCGCAGGCAACGCTGAAATCAGCACAGGA
TAAATTGCGTCAGTACCGTTCCGGTAACCGTGAACAGGACATCGCTCAGG
CGAAAGCCAGCCTCGAACAGGCGCAGGCGCAACTGGCGCAGGCGGAGTTG
AATTTACAGGACTCAACGTTGATAGCCCCGTCTGATGGCACGCTGTTAAC
GCGCGCGGTGGAGCCAGGCACGGTCCTCAATGAAGGTGGCACGGTGTTTA
CCGTTTCACTAACGCGTCCGGTGTGGGTGCGCGCTTATGTTGATGAACGT
AATCTTGACCAGGCCCAGCCGGGGCGCAAAGTGCTGCTTTATACCGATGG
TCGCCCGGACAAGCCGTATCACGGGCAGATTGGTTTCGTTTCGCCGACTG
CTGAATTTACCCCGAAAACCGTCGAAACGCCGGATCTGCGTACCGACCTC
GTCTATCGCCTGCGTATTGTGGTGACCGACGCCGATGATGCGTTACGCCA
GGGAATGCCAGTGACGGTACAATTCGGTGACGAGGCAGGACATGAATGA

### SEQ ID NO:20

ybhF

ATGAATGATGCCGTTATCACGCTGAACGGCCTGGAAAAACGCTTTCCGGG
CATGGACAAGCCCGCCGTCGCGCCGCTCGATTGTACCATTCACGCCGGTT
ATGTGACGGGGTTGGTGGGGCCGGACGGTGCAGGTAAAACCACGCTGATG
CGGATGTTGGCGGGATTACTGAAACCCGACAGCGGCAGTGCCACGGTGAT
TGGCTTTGATCCGATCAAAAACGACGGCGCGCTGCACGCCGTGCTCGGTT
ATATGCCGCAGAAATTTGGTCTGTATGAAGATCTCACGGTGATGGAGAAC
CTCAATCTGTACGCGGATTTGCGCAGCGTCACCGGCGAGGCACGTAAGCA
AACTTTTGCTCGCCTGCTGGAGTTTACGTCTCTTGGGCCGTTTACCGGAC
GCCTGGCGGGCAAGCTCTCCGGTGGGATGAAACAAAAACTCGGTCTGGCC
TGTACCCTGGTGGGCGAACCGAAAGTGTTGCTGCTCGATGAACCCGGCGT
CGGCGTTGACCCTATCTCACGGCGCGAACTGTGGCAGATGGTGCATGAGC
TGGCGGGCGAAGGGATGTTAATCCTCTGGAGTACCTCGTATCTCGACGAA
GCCGAGCAGTGCCGTGACGTGTTACTGATGAACGAAGGCGAGTTGCTGTA
TCAGGGAGAACCAAAAGCCCTGACACAAACCATGGCCGGACGCAGCTTTC
TGATGACCAGTCCACACGAGGGCAACCGCAAACTGTTGCAACGCGCCTTG
AAACTGCCGCAGGTCAGCGACGGCATGATTCAGGGGAAATCGGTACGTCT
GATCCTCAAAAAAGAGGCCACACCAGACGATATTCGCCATGCCGACGGGA
TGCCGGAAATCAACATCAACGAAACTACGCCGCGTTTTGAAGATGCGTTT
ATTGATTTGCTGGGCGGTGCCGGAACCTCGGAATCGCCGCTGGGCGCAAT
ATTACATACGGTAGAAGGCACACCCGGCGAGACGGTGATCGAAGCGAAAG
AACTGACCAAGAAATTTGGGGATTTTGCCGCCACCGATCACGTCAACTTT
GCCGTTAAACGTGGGGAGATTTTTGGTTTGCTGGGGCCAAACGGCGCGGG
TAAATCGACCACCTTTAAGATGATGTGCGGTTTGCTGGTGCCGACTTCCG
GCCAGGCGCTGGTGCTGGGGATGGATCTGAAAGAGAGTTCCGGTAAAGCG
CGCCAGCATCTCGGCTATATGGCGCAAAAATTTTCGCTCTACGGTAACCT
GACGGTCGAACAGAATTTACGCTTTTTCTCTGGTGTGTATGGCTTACGCG
GTCGGGCGCAGAACGAAAAAATCTCCCGCATGAGCGAGGCGTTCGGCCTG
AAAAGTATCGCCTCCCACGCCACCGATGAACTGCCATTAGGTTTTAAACA
GCGGCTGGCGCTGGCCTGTTCGCTGATGCATGAACCGGACATTCTGTTTC
TCGACGAACCGACTTCCGGCGTTGACCCCCTCACCCGCCGTGAATTTTGG
CTGCACATCAACAGCATGGTAGAGAAAGGCGTCACGGTGATGGTCACCAC
CCACTTTATGGATGAAGCGGAATATTGCGACCGCATCGGCCTGGTGTACC
GCGGGAAATTAATCGCCAGCGGCACGCCGGACGATTTGAAAGCACAGTCG
GCTAACGATGAGCAACCCGATCCCACCATGGAGCAAGCCTTTATTCAGTT
GATCCACGACTGGGATAAGGAGCATAGCAATGAGTAA

### SEQ ID NO:21

ybhS

ATGAGTAACCCGATCCTGTCCTGGCGTCGCGTACGGGCGCTGTGCGTTAA
AGAGACGCGGCAGATCGTTCGCGATCCGAGTAGCTGGCTGATTGCGGTAG
TGATCCCGCTGCTACTGCTGTTTATTTTTGGTTACGGCATTAACCTCGAC
TCCAGCAAGCTGCGGGTCGGGATTTTACTGGAACAGCGTAGCGAAGCGGC
GCTGGATTTCACCCACACCATGACCGGTTCGCCCTACATCGACGCCACCA
TCAGCGATAACCGTCAGGAACTGATCGCCAAAATGCAGGCGGGGAAAATT
CGCGGTCTGGTGGTTATTCCGGTGGATTTTGCGGAACAGATGGAGCGCGC
CAACGCCACCGCACCGATTCAGGTGATCACCGACGGCAGTGAGCCGAATA
CCGCTAACTTTGTACAGGGGTATGTCGAAGGGATCTGGCAGATCTGGCAA
ATGCAGCGAGCGGAGGACAACGGGCAGACTTTTGAACCGCTTATTGATGT
ACAAACCCGCTACTGGTTTAACCCGGCGGCGATTAGCCAGCACTTCATTA
TCCCCGGTGCGGTGACCATTATCATGACGGTCATCGGCGCGATTCTCACC
TCGCTGGTGGTGGCGCGAGAATGGGAACGCGGCACCATGGAGGCTCTGCT
CTCTACGGAGATTACCCGCACGGAACTGCTGCTGTGTAAGCTGATCCCTT
ATTACTTTCTCGGGATGCTGGCGATGTTGCTGTGTATGCTGGTGTCAGTG
TTTATTCTCGGCGTGCCGTATCGCGGGTCGCTGCTGATTCTGTTTTTTAT
CTCCAGCCTGTTTTTACTCAGTACCCTGGGGATGGGGCTGCTGATTTCCA
CGATTACCCGCAACCAGTTCAATGCCGCTCAGGTCGCCCTGAACGCCGCT
TTTCTGCCGTCGATTATGCTTTCCGGCTTTATTTTTCAGATCGACAGTAT
GCCCGCGGTGATCCGCGCGGTGACGTACATTATTCCCGCTCGTTATTTCG
TCAGCACCCTGCAAAGCCTGTTCCTCGCCGGGAATATTCCAGTGGTGCTG
GTGGTAAACGTGCTGTTTTTGATCGCTTCGGCGGTGATGTTTATCGGCCT
GACGTGGCTGAAAACCAAACGTCGGCTGGATTAG

### SEQ ID NO:22

ybhR

ATGTTTCATCGCTTATGGACGTTAATCCGCAAAGAGTTGCAGTCGTTGCT
GCGCGAACCGCAAACCCGCGCGATTCTGATTTTACCCGTGCTAATTCAGG
TGATCCTGTTCCCGTTCGCCGCCACGCTGGAAGTGACTAACGCCACCATC
GCCATCTACGATGAAGATAACGGCGAGCATTCGGTGGAGCTGACCCAACG
TTTTGCCCGCGCCAGCGCCTTTACTCATGTGCTGCTGCTGAAAAGCCCAC
AGGAGATCCGCCCAACCATCGACACACAAAAGGCGTTACTACTGGTGCGT
TTCCCGGCTGACTTCTCGCGCAAACTGGATACCTTCCAGACCGCGCCTTT
GCAGTTGATCCTCGACGGGCGTAACTCCAACAGTGCGCAAATTGCCGCCA
ACTACCTGCAACAGATCGTCAAAAATTATCAGCAGGAGCTGCTGGAAGGA
AAACCGAAACCTAACAACAGCGAGCTGGTGGTACGCAACTGGTATAACCC
GAATCTCGACTACAAATGGTTTGTGGTGCCGTCACTGATCGCCATGATCA
CCACTATCGGCGTAATGATCGTCACTTCACTTTCCGTCGCCCGCGAACGT
GAACAAGGTACGCTCGATCAGCTACTGGTTTCGCCGCTCACCACCTGGCA
GATCTTCATCGGCAAAGCCGTACCGGCGTTAATTGTCGCCACCTTCCAGG
CCACCATTGTGCTGGCGATTGGTATCTGGGCGTATCAAATCCCCTTCGCC
GGATCGCTGGCGCTGTTCTACTTTACGATGGTGATTTATGGTTTATCGCT
GGTGGGATTCGGTCTGTTGATTTCATCACTCTGTTCAACACAACAGCAGG
CGTTTATCGGCGTGTTTGTCTTTATGATGCCCGCCATTCTCCTTTCCGGT
TACGTTTCTCCGGTGGAAAACATGCCGGTATGGCTGCAAAACCTGACGTG
GATTAACCCTATTCGCCACTTTACGGACATTACCAAGCAGATTTATTTGA
AGGATGCGAGTCTGGATATTGTGTGGAATAGTTTGTGGCCGCTACTGGTG
ATAACGGCCACGACAGGGTCAGCGGCGTACGCGATGTTTAGACGTAAGGT
GATGTAA

### SEQ ID NO:23

tolC

ATGAAGAAATTGCTCCCCATTCTTATCGGCCTGAGCCTTTCTGGGTTCAG
TTCGTTGAGCCAGGCCGAGAACCTGATGCAAGTTTATCAGCAAGCACGCC
TTAGTAACCCGGAATTGCGTAAGTCTGCCGCCGATCGTGATGCTGCCTTT
GAAAAAATTAATGAAGCGCGCAGTCCATTACTGCCACAGCTAGGTTTAGG
TGCAGATTACACCTATAGCAACGGCTACCGCGACGCGAACGGCATCAACT
CTAACGCGACCAGTGCGTCCTTGCAGTTAACTCAATCCATTTTTGATATG
TCGAAATGGCGTGCGTTAACGCTGCAGGAAAAAGCAGCAGGGATTCAGGA
CGTCACGTATCAGACCGATCAGCAAACCTTGATCCTCAACACCGCGACCG
CTTATTTCAACGTGTTGAATGCTATTGACGTTCTTTCCTATACACAGGCA
CAAAAAGAAGCGATCTACCGTCAATTAGATCAAACCACCCAACGTTTTAA
CGTGGGCCTGGTAGCGATCACCGACGTGCAGAACGCCCGCGCACAGTACG
ATACCGTGCTGGCGAACGAAGTGACCGCACGTAATAACCTTGATAACGCG
GTAGAGCAGCTGCGCCAGATCACCGGTAACTACTATCCGGAACTGGCTGC
GCTGAATGTCGAAAACTTTAAAACCGACAAACCACAGCCGGTTAACGCGC
TGCTGAAAGAAGCCGAAAAACGCAACCTGTCGCTGTTACAGGCACGCTTG
AGCCAGGACCTGGCGCGCGAGCAAATTCGCCAGGCGCAGGATGGTCACTT
ACCGACTCTGGATTTAACGGCTTCTACCGGGATTTCTGACACCTCTTATA
GCGGTTCGAAAACCCGTGGTGCCGCTGGTACCCAGTATGACGATAGCAAT
ATGGGCCAGAACAAAGTTGGCCTGAGCTTCTCGCTGCCGATTTATCAGGG
CGGAATGGTTAACTCGCAGGTGAAACAGGCACAGTACAACTTTGTCGGTG
CCAGCGAGCAACTGGAAAGTGCCCATCGTAGCGTCGTGCAGACCGTGCGT
TCCTCCTTCAACAACATTAATGCATCTATCAGTAGCATTAACGCCTACAA
ACAAGCCGTAGTTTCCGCTCAAAGCTCATTAGACGCGATGGAAGCGGGCT
ACTCGGTCGGTACGCGTACCATTGTTGATGTGTTGGATGCGACCACCACG
TTGTACAACGCCAAGCAAGAGCTGGCGAATGCGCGTTATAACTACCTGAT
TAATCAGCTGAATATTAAGTCAGCTCTGGGTACGTTGAACGAGCAGGATC
TGCTGGCACTGAACAATGCGCTGAGCAAACCGGTTTCCACTAATCCGGAA
AACGTTGCACCGCAAACGCCGGAACAGAATGCTATTGCTGATGGTTATGC
GCCTGATAGCCCGGCACCAGTCGTTCAGCAAACATCCGCACGCACTACCA
CCAGTAACGGTCATAACCCTTTCCGTAACTGA

### SEQ ID NO:24

yhiI

ATGGATAAGAGTAAGCGCCATCTGGCGTGGTGGGTTGTCGGGTTACTGGC
GGTGGCGGCTATCGTGGCGTGGTGGCTGTTGCGCCCGGCAGGTGTGCCGG
AAGGCTTTGCTGTCAGTAATGGGCGCATTGAAGCGACGGAAGTGGATATT
GCCAGCAAAATTGCCGGGCGTATCGACACCATTCTGGTGAAAGAAGGCAA
GTTTGTTCGCGAAGGTGAAGTGCTGGCGAAGATGGATACTCGCGTGTTGC
AGGAACAGCGACTGGAAGCCATCGCGCAAATCAAAGAGGCACAAAGCGCC
GTTGCTGCCGCGCAGGCTTTGCTGGAGCAACGACAAAGCGAAACTCGTGC
CGCACAGTCGCTGGTTAATCAACGCCAGGCAGAACTGGACTCCGTAGCAA
AACGTCATACGCGTTCCCGTTCACTGGCCCAACGAGGGGCTATTTCTGCG
CAACAGCTGGATGACGATCGCGCCGCCGCTGAGAGCGCCCGAGCTGCGCT
GGAATCGGCGAAAGCTCAGGTATCGGCTTCTAAAGCGGCTATAGAAGCGG
CACGCACCAATATCATTCAGGCGCAAACCCGCGTCGAAGCGGCACAAGCC
ACTGAACGGCGCATTGCCGCAGATATCGATGACAGCGAACTGAAAGCCCC
GCGTGACGGACGCGTGCAGTATCGGGTTGCCGAGCCAGGCGAAGTGCTGG
CGGCAGGCGGTCGGGTGCTGAATATGGTCGATCTCAGCGACGTCTATATG
ACTTTCTTCCTGCCAACCGAACAGGCGGGCACGCTGAAACTGGGCGGTGA
AGCCCGGCTGATCCTCGATGCCGCGCCAGATCTGCGTATTCCTGCAACCA
TCAGTTTTGTCGCCAGTGTCGCCCAGTTCACGCCAAAAACCGTCGAAACC
AGCGATGAACGGCTGAAACTGATGTTCCGCGTCAAAGCGCGTATCCCACC
GGAATTACTCCAGCAGCATCTGGAATATGTCAAAACCGGTTTGCCGGGCG
TAGCGTGGGTGCGGGTGAATGAAGAACTTCCGTGGCCTGACGACCTCGTG
GTGAGGTTGCCGCAATGA

### SEQ ID NO:25

rbbA

ATGACGCATCTGGAACTGGTTCCCGTCCCGCCTGTCGCGCAACTGGCGGG
CGTGAGCCAGCATTATGGAAAAACCGTTGCGCTGAACAATATCACTCTCG
ATATTCCGGCCCGCTGTATGGTCGGGCTGATTGGCCCGGACGGCGTCGGG
AAGTCGAGCTTGTTGTCGTTGATTTCCGGTGCCCGCGTCATTGAACAGGG
CAATGTGATGGTGCTGGGCGGCGATATGCGCGACCCGAAGCATCGCCGCG
ACGTCTGCCCGCGCATCGCCTGGATGCCGCAGGGGCTGGGCAAAAACCTC
TACCACACCTTGTCGGTGTATGAAAACGTCGATTTTTTCGCTCGCCTGTT
CGGTCACGACAAAGCGGAGCGGGAAGTGCGAATCAATGAGCTGCTGACCA
GCACCGGGTTAGCACCGTTTCGCGATCGTCCGGCAGGGAAACTCTCCGGC
GGGATGAAGCAAAAACTTGGGCTGTGCTGCGCGTTAATCCACGACCCGGA
ACTGTTGATCCTTGATGAGCCAACAACGGGGGTTGACCCGCTCTCCCGCT
CCCAGTTCTGGGATCTGATCGACAGTATTCGCCAGCGGCAGAGCAATATG
AGCGTGCTGGTCGCCACCGCCTATATGGAAGAGGCCGAACGCTTCGACTG
GCTGGTAGCGATGAATGCCGGAGAAGTGCTGGCAACTGGCAGCGCCGAAG
AGCTACGGCAGCAAACGCAAAGCGCTACGCTGGAAGAAGCATTTATAAAT
CTGTTACCGCAAGCGCAACGCCAGGCGCATCAGGCGGTAGTGATCCCACC
GTATCAACCTGAAAACGCAGAGATTGCCATCGAAGCGCGCGATCTGACCA
TGCGTTTTGGTTCCTTCGTTGCCGTTGATCACGTTAATTTCCGCATTCCA
CGCGGGGAGATTTTTGGTTTTCTTGGTTCGAACGGCTGCGGTAAATCCAC
CACCATGAAAATGCTCACCGGACTGCTGCCCGCCAGCGAAGGTGAGGCGT
GGCTGTTCGGGCAACCGGTTGATCCAAAAGATATCGATACCCGCCGTCGG
GTGGGCTATATGTCGCAGGCGTTTTCGCTCTATAACGAACTCACCGTGCG
GCAAAACCTTGAGTTACATGCCCGTTTGTTTCACATCCCGGAAGCGGAAA
TTCCCGCAAGAGTGGCTGAAATGAGCGAGCGTTTTAAGCTCAACGACGTT
GAAGATATTCTGCCGGAGTCATTGCCGCTCGGCATTCGCCAGCGGCTTTC
GCTGGCGGTGGCGGTGATTCATCGCCCGGAGATGTTAATCCTCGATGAGC
CTACTTCTGGTGTCGATCCGGTGGCGAGGGATATGTTCTGGCAGTTGATG
GTCGATCTCTCGCGCCAGGACAAAGTGACTATCTTCATCTCCACCCACTT
TATGAACGAAGCGGAACGTTGCGACCGCATCTCACTGATGCACGCCGGAA
AAGTGCTTGCCAGCGGTACACCGCAGGAACTGGTTGAGAAACGCGGAGCC
GCCAGTCTGGAAGAGGCATTTATCGCCTATTTGCAGGAAGCGGCAGGGCA
GAGCAACGAAGCCGAAGCGCCGCCCGTGGTACACGACACCACCCACGCGC
CGCGTCAGGGATTTAGCCTGCGCCGTCTGTTTAGCTACAGCCGCCGCGAA
GCGCTGGAACTGCGACGCGATCCAGTACGTTCGACGCTGGCGCTGATGGG
AACGGTGATCCTGATGCTGATAATGGGTTACGGCATCAGTATGGATGTGG
AAAACCTGCGCTTTGCGGTGCTCGACCGCGACCAGACCGTCAGTAGCCAG
GCGTGGACACTCAACCTCTCCGGTTCCCGTTACTTTATCGAACAGCCGCC
GCTCACCAGTTATGACGAGCTTGATCGTCGGATGCGTGCGGGCGATATCA
CGGTGGCGATTGAGATCCCGCCCAATTTCGGGCGCGATATCGCGCGTGGT
ACGCCTGTGGAACTCGGCGTCTGGATCGACGGAGCGATGCCGAGCCGTGC
TGAAACGGTAAAAGGTTACGTGCAGGCCATGCACCAGAGCTGGTTACAGG
ATGTGGCGAGCCGACAATCGACACCCGCCAGCCAAAGCGGGCTGATGAAT
ATTGAGACGCGCTATCGCTATAACCCGGACGTAAAAAGCCTGCCAGCGAT
TGTTCCGGCGGTGATCCCGCTTCTGCTGATGATGATCCCGTCAATGCTAA
GCGCCCTTAGCGTGGTGCGGGAAAAAGAGCTTGGGTCGATTATCAACCTT
TACGTGACCCCCACCACGCGTAGTGAATTTTTGCTTGGTAAACAGTTGCC
ATACATCGCGCTGGGGATGCTGAACTTTTTCCTGCTCTGCGGCCTGTCGG
TGTTTGTGTTTGGCGTACCGCATAAAGGCAGTTTCCTGACGCTCACCCTG
GCGGCGCTGCTGTATATCATCATTGCCACCGGAATGGGGCTGCTGATCTC
CACCTTTATGAAAAGCCAGATTGCCGCCATTTTCGGAACGGCGATTATCA
CGTTGATCCCGGCGACACAGTTTTCCGGGATGATCGATCCGGTAGCTTCG
CTGGAAGGGCCTGGACGTTGGATCGGCGAGGTTTACCCGACCAGTCATTT
TCTGACTATCGCCCGCGGGACGTTCTCGAAAGCGCTGGATCTGACTGATT
TGTGGCAACTTTTTATCCCGTTACTGATAGCCATCCCGCTGGTGATGGGC
TTAAGTATCCTGCTGCTGAAAAAACAGGAGGGATGA

### SEQ ID NO:26

yhhJ

ATGCGCCATTTACGCAATATTTTTAATCTGGGTATCAAAGAGTTGCGCAG
TCTGCTCGGTGATAAAGCGATGCTGACGCTGATTGTCTTCTCGTTTACGG
TGTCGGTGTATTCGTCAGCGACCGTTACGCCAGGATCGTTGAACCTCGCG
CCGATCGCCATTGCCGATATGGATCAATCGCAGTTATCGAACCGGATCGT
TAACAGCTTCTATCGTCCGTGGTTTTTGCCACCGGAGATGATCACCGCCG
ATGAGATGGATGCCGGACTGGACGCCGGACGCTATACCTTCGCGATAAAT
ATTCCGCCTAATTTTCAGCGTGATGTCCTCGCCGGACGCCAGCCGGATAT
TCAGGTGAACGTCGATGCCACGCGCATGAGCCAGGCATTTACCGGCAATG
GGTATATCCAGAATATTATCAACGGTGAAGTGAACAGCTTTGTCGCGCGC
TACCGTGATAACAGCGAACCGTTGGTATCGCTGGAAACCCGGATGCGCTT
TAACCCGAACCTCGATCCCGCGTGGTTTGGCGGGGTGATGGCGATCATCA
ACAACATTACCATGCTGGCGATTGTATTGACCGGATCGGCGCTGATCCGC
GAGCGTGAACACGGCACGGTGGAACACTTACTGGTGATGCCGATAACGCC
GTTTGAGATCATGATGGCGAAGATCTGGTCGATGGGGCTGGTGGTGCTGG
TGGTATCGGGATTATCGCTGGTGCTGATGGTGAAAGGTGTACTGGGCGTA
CCGATTGAAGGCTCGATCCCGCTGTTTATGCTGGGCGTGGCGCTCAGTCT
GTTTGCCACCACGTCAATCGGCATTTTTATGGGGACGATAGCGCGTTCAA
TGCCGCAACTGGGGCTGCTGGTGATTCTGGTGCTGCTGCCGCTGCAAATG
CTTTCCGGTGGTTCCACGCCGCGCGAAAGTATGCCGCAGATGGTGCAGGA
CATTATGCTGACCATGCCGACGACACACTTTGTTAGCCTCGCGCAGGCCA
TCCTCTACCGGGGTGCCGGATTCGAAATCGTCTGGCCGCAGTTTCTGACG
CTGATGGCAATTGGCGGCGCATTTTTCACCATTGCGCTGCTGCGATTCAG
GAAGACGATTGGGACAATGGCGTAA

### SEQ ID NO:27

### YbhG

MMKKPVVIGLAVVVLAAVVAGGYWWYQSRQDNGLTLYGNVDIRTVNLSFR
VGGRVESLAVDEGDAIKAGQVLGELDHKPYEIALMQAKAGVSVAQAQYDL
MLAGYRNEEIAQAAAAVKQAQAAYDYAQNFYNRQQGLWKSRTISANDLEN
ARSSRDQAQATLKSAQDKLRQYRSGNREQDIAQAKASLEQAQAQLAQAEL
NLQDSTLIAPSDGTLLTRAVEPGTVLNEGGTVFTVSLTRPVWVRAYVDER
NLDQAQPGRKVLLYTDGRPDKPYHGQIGFVSPTAEFTPKTVETPDLRTDL
VYRLRIVVTDADDALRQGMPVTVQFGDEAGHE

### SEQ ID NO:28

### YbhF

MNDAVITLNGLEKRFPGMDKPAVAPLDCTIHAGYVTGLVGPDGAGKTTLM
RMLAGLLKPDSGSATVIGFDPIKNDGALHAVLGYMPQKFGLYEDLTVMEN
LNLYADLRSVTGEARKQTFARLLEFTSLGPFTGRLAGKLSGGMKQKLGLA
CTLVGEPKVLLLDEPGVGVDPISRRELWQMVHELAGEGMLILWSTSYLDE
AEQCRDVLLMNEGELLYQGEPKALTQTMAGRSFLMTSPHEGNRKLLQRAL
KLPQVSDGMIQGKSVRLILKKEATPDDIRHADGMPEININETTPRFEDAF
IDLLGGAGTSESPLGAILHTVEGTPGETVIEAKELTKKFGDFAATDHVNF
AVKRGEIFGLLGPNGAGKSTTFKMMCGLLVPTSGQALVLGMDLKESSGKA
RQHLGYMAQKFSLYGNLTVEQNLRFFSGVYGLRGRAQNEKISRMSEAFGL
KSIASHATDELPLGFKQRLALACSLMHEPDILFLDEPTSGVDPLTRREFW
LHINSMVEKGVTVMVTTHFMDEAEYCDRIGLVYRGKLIASGTPDDLKAQS
ANDEQPDPTMEQAFIQLIHDWDKEHSNE

### SEQ ID NO:29

### YbhS

MSNPILSWRRVRALCVKETRQIVRDPSSWLIAVVIPLLLLFIFGYGINLD
SSKLRVGILLEQRSEAALDFTHTMTGSPYIDATISDNRQELIAKMQAGKI
RGLVVIPVDFAEQMERANATAPIQVITDGSEPNTANFVQGYVEGIWQIWQ
MQRAEDNGQTFEPLIDVQTRYWFNPAAISQHFIIPGAVTIIMTVIGAILT
SLVVAREWERGTMEALLSTEITRTELLLCKLIPYYFLGMLAMLLCMLVSV
FILGVPYRGSLLILFFISSLFLLSTLGMGLLISTITRNQFNAAQVALNAA
FLPSIMLSGFIFQIDSMPAVIRAVTYIIPARYFVSTLQSLFLAGNIPVVL
VVNVLFLIASAVMFIGLTWLKTKRRLD

### SEQ ID NO:30

### YbhR

MFHRLWTLIRKELQSLLREPQTRAILILPVLIQVILFPFAATLEVTNATI
AIYDEDNGEHSVELTQRFARASAFTHVLLLKSPQEIRPTIDTQKALLLVR
FPADFSRKLDTFQTAPLQLILDGRNSNSAQIAANYLQQIVKNYQQELLEG
KPKPNNSELVVRNWYNPNLDYKWFVVPSLIAMITTIGVMIVTSLSVARER
EQGTLDQLLVSPLTTWQIFIGKAVPALIVATFQATIVLAIGIWAYQIPFA
GSLALFYFTMVIYGLSLVGFGLLISSLCSTQQQAFIGVFVFMMPAILLSG
YVSPVENMPVWLQNLTWINPIRHFTDITKQIYLKDASLDIVWNSLWPLLV
ITATTGSAAYAMFRRKVM

### SEQ ID NO:31

### TolC

MKKLLPILIGLSLSGFSSLSQAENLMQVYQQARLSNPELRKSAADRDAAF
EKINEARSPLLPQLGLGADYTYSNGYRDANGINSNATSASLQLTQSIFDM
SKWRALTLQEKAAGIQDVTYQTDQQTLILNTATAYFNVLNAIDVLSYTQA
QKEAIYRQLDQTTQRFNVGLVAITDVQNARAQYDTVLANEVTARNNLDNA
VEQLRQITGNYYPELAALNVENFKTDKPQPVNALLKEAEKRNLSLLQARL
SQDLAREQIRQAQDGHLPTLDLTASTGISDTSYSGSKTRGAAGTQYDDSN
MGQNKVGLSFSLPIYQGGMVNSQVKQAQYNFVGASEQLESAHRSVVQTVR
SSFNNINASISSINAYKQAVVSAQSSLDAAGYSVGTRTIVDVLDATTTLY
NAKQELANARYNYLINQLNKSALGTLNEQDLLALNNALSKPVSTNPENVA
PQTPEQNAIADGYAPDSPAPVVQQTSARTTTSNGHNPFRN

### SEQ ID NO:32

### YhiI

MDKSKRHLAWWVVGLLAVAAIVAWWLLRPAGVPEGFAVSNGRIEATEVDI
ASKIAGRIDTILVKEGKFVREGEVLAKMDTRVLQEQRLEAIAQIKEAQSA
VAAAQALLEQRQSETRAAQSLVNQRQAELDSVAKRHTRSRSLAQRGAISA
QQLDDDRAAAESARAALESAKAQVSASKAAIEAARTNIIQAQTRVEAAQA
TERRIAADIDDSELKAPRDGRVQYRVAEPGEVLAAGGRVLNMVDLSDVYM
TFFLPTEQAGTLKLGGEARLILDAAPDLRIPATISFVASVAQFTPKTVET
SDERLKLMFRVKARIPPELLQQHLEYVKTGLPGVAWVRVNEELPWPDDLV
VRLPQ

### SEQ ID NO:33

### RbbA

MTHLELVPVPPVAQLAGVSQHYGKTVALNNITLDIPARCMVGLIGPDGVG
KSSLLSLISGARVIEQGNVMVLGGDMRDPKHRRDVCPRIAWMPQGLGKNL
YHTLSVYENVDFFARLFGHDKAEREVRINELLTSTGLAPFRDRPAGKLSG
GMKQKLGLCCALIHDPELLILDEPTTGVDPLSRSQFWDLIDSIRQRQSNM
SVLVATAYMEEAERFDWLVAMNAGEVLATGSAEELRQQTQSATLEEAFIN
LLPQAQRQAHQAVVIPPYQPENAEIAIEARDLTMRFGSFVAVDHVNFRIP
RGEIFGFLGSNGCGKSTTMKMLTGLLPASEGEAWLFGQPVDPKDIDTRRR
VGYMSQAFSLYNELTVRQNLELHARLFHIPEAEIPARVAEMSERFKLNDV
EDILPESLPLGIRQRLSLAVAVIHRPEMLILDEPTSGVDPVARDMFWQLM
VDLSRQDKVTIFISTHFMNEAERCDRISLMHAGKVLASGTPQELVEKRGA
ASLEEAFIAYLQEAAGQSNEAEAPPVVHDTTHAPRQGFSLRRLFSYSRRE
ALELRRDPVRSTLALMGTVILMLIMGYGISMDVENLRFAVLDRDQTVSSQ
AWTLNLSGSRYFIEQPPLTSYDELDRRMRAGDITVAIEIPPNFGRDIARG
TPVELGVWIDGAMPSRAETVKGYVQAMHQSWLQDVASRQSTPASQSGLMN
IETRYRYNPDVKSLPAIVPAVIPLLLMMIPSMLSALSVVREKELGSIINL
YVTPTTRSEFLLGKQLPYIALGMLNFFLLCGLSVFVFGVPHKGSFLTLTL
AALLYIIIATGMGLLISTFMKSQIAAIFGTAIITLIPATQFSGMIDPVAS
LEGPGRWIGEVYPTSHFLTIARGTFSKALDLTDLWQLFIPLLIAIPLVMG
LSILLLKKQEG

### SEQ ID NO:34

### YhhJ

MRHLRNIFNLGIKELRSLLGDKAMLTLIVFSFTVSVYSSATVTPGSLNLA
PIAIADMDQSQLSNRIVNSFYRPWFLPPEMITADEMDAGLDAGRYTFAIN
IPPNFQRDVLAGRQPDIQVNVDATRMSQAFTGNGYIQNIINGEVNSFVAR
YRDNSEPLVSLETRMRFNPNLDPAWFGGVMAIINNITMLAIVLTGSALIR
EREHGTVEHLLVMPITPFEIMMAKIWSMGLVVLVVSGLSLVLMVKGVLGV
PIEGSIPLFMLGVALSLFATTSIGIFMGTIARSMPQLGLLVILVLLPLQM
LSGGSTPRESMPQMVQDIMLTMPTTHFVSLAQAILYRGAGFEIVWPQFLT
LMAIGGAFFTIALLRFRKTIGTMA

### SEQ ID NO:35

pJB1440 Sequence

CTCATGACCAAAATCCCTTAACGTGAGTTACGCGCGCGTCGTTCCACTGAGCGTCAGACCCCGTAGAAAA
GATCAAAGGATCTTCTTGAGATCCTTTTTTTCTGCGCGTAATCTGCTGCTTGCAAACAAAAAAACCACCG
CTACCAGCGGTGGTTTGTTTGCCGGATCAAGAGCTACCAACTCTTTTTCCGAAGGTAACTGGCTTCAGCA
GAGCGCAGATACCAAATACTGTTCTTCTAGTGTAGCCGTAGTTAGCCCACCACTTCAAGAACTCTGTAGC
ACCGCCTACATACCTCGCTCTGCTAATCCTGTTACCAGTGGCTGCTGCCAGTGGCGATAAGTCGTGTCTT
ACCGGGTTGGACTCAAGACGATAGTTACCGGATAAGGCGCAGCGGTCGGGCTGAACGGGGGGTTCGTGCA
CACAGCCCAGCTTGGAGCGAACGACCTACACCGAACTGAGATACCTACAGCGTGAGCTATGAGAAAGCGC
CACGCTTCCCGAAGGGAGAAAGGCGGACAGGTATCCGGTAAGCGGCAGGGTCGGAACAGGAGAGCGCACG
AGGGAGCTTCCAGGGGGAAACGCCTGGTATCTTTATAGTCCTGTCGGGTTTCGCCACCTCTGACTTGAGC
GTCGATTTTTGTGATGCTCGTCAGGGGGGCGGAGCCTATGGAAAAACGCCAGCAACGCGGCCTTTTTACG
GTTCCTGGCCTTTTGCTGGCCTTTTGCTCACATGTTCTTTCCTGCGTTATCCCCTGATTCTGTGGATAAC
CGTATTACCGCCTTTGAGTGAGCTGATACCGCTCGCCGCAGCCGAACGACCGAGCGCAGCGAGTCAGTGA
GCGAGGAAGCGGAAGGCGAGAGTAGGGAACTGCCAGGCATCAAACTAAGCAGAAGGCCCCTGACGGATGG
CCTTTTTGCGTTTCTACAAACTCTTTCTGTGTTGTAAAACGACGGCCAGTCTTAAGCTCGGGCCCC

[symbol]

[symbol]

[symbol]

GGCGCCTGTCACTTTGCTTGATATATGAGA
ATTATTTAACCTTATAAATGAGAAAAAAGCAACGCACTTTAAATAAGATACGTTGCTTTTTCGATTGATG
AACACCTATAATTAAACTATTCATCTATTATTTATGATTTTTTGTATATACAATATTTCTAGTTTGTTAA
AGAGAATTAAGAAAATAAATCTCGAAAATAATAAAGGGAAAATCAGTTTTTGATATCAAAATTATACATG
TCAACGATAATACAAAATATAATACAAACTATAAGATGTTATCAGTATTTATTATGCATTTAGAATAAAT
TTTGTGTCGCCCTTAATTGTGAGCGGATAACAATTACGAGCTTCATGCACAGTGAAATCATGAAAAATTT
ATTTGCTTTGTGAGCGGATAACAATTATAATATGTGGAATTGTGAGCGCTCACAATTCCACAACGGTTTC
CCTCTAGAAATAATTTTGTTTAACTTTTAGGAGGTAAAACAT

[symbol]

[symbol]

[symbol]

[symbol]

[symbol]

[symbol]

[symbol]

[symbol]

[symbol]

[symbol]

[symbol]

tccaggaaatctga

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

gaattcAAAacgtttcaattggctaataggatccTAGACGTC
gcTAAtacggccggccacccttttttaggtagcGCTAGCatagggcccTAACTCGAGCCCCAAGGGCGAC
ACCCCAT

[symbol]

[symbol]

[symbol]

CAAGGGGTGTTATGAGCCATATTCAGGT
ATAAATGGGCTCGCGATAATGTTCAGAATTGGTTAATTGGTTGTAACACTGACCCCTATTTGTTTATTTT
TCTAAATACATTCAAATATGTATCCGCTCATGAGACAATAACCCTGATAAATGCTTCAATAATATTGAAA
AAGGAAGAAT

[symbol]

[symbol]

[symbol]

[symbol]

[symbol]

[symbol]

[symbol]

[symbol]

[symbol]

[symbol]

[symbol]

[symbol]

[symbol]

GCGGCGCGCCATCGAATGGCGCAAAACCTTTCGCGGTAT
GGCATGATAGCGCCCGGAAGAGAGTCAATTCAGGGTGGTGAAT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

pUC ori—1^{st} underlined sequence
rpn txn terminator—1^{st} italicized sequence
bla txn terminator—2^{nd} underlined sequence
T5 promoter—1^{St} double-underlined sequence
adm_PCC7942—1^{st} italicized and underlined sequence
aar_PCC7942—2^{nd} italicized and underlined sequence
rrnB1-B2 T1 txn terminator 2^{nd} italicized sequence
bla—3^{rd} italicized and underlined sequence
lacI—4^{th} italicized and underlined sequence

### SEQ ID NO:36

Kanamycin promoter and gene coding sequence

CTGTCAAACATGAGAATTAATTCCGGGGATCCGTCGACCTGCAGTTCGAAGTTCCTATTCTCTAGAAAGT
ATAGGAACTTCAGAGCGCTTTTGAAGCTCACGCTGCCGCAAGCACTCAGGGCGCAAGGGCTGCTAAAGGA
AGCGGAACACGTAGAAAGCCAGTCCGCAGAAACGGTGCTGACCCCGGATGAATGTCAGCTACTGGGCTAT
CTGGACAAGGGAAAACGCAAGCGCAAAGAGAAAGCAGGTAGCTTGCAGTGGGCTTACATGGCGATAGCTA
GACTGGGCGGTTTTATGGACAGCAAGCGAA

[symbol]

[symbol]

[symbol]

ATGATTGAACAAGATGGATTGCACGCAGGTTCTCCGGCCGCT
TGGGTGGAGAGGCTATTCGGCTATGACTGGGCACAACAGACAATCGGCTGCTCTGATGCCGCCGTGTTCC
GGCTGTCAGCGCAGGGGCGCCCGGTTCTTTTTGTCAAGACCGACCTGTCCGGTGCCCTGAATGAACTGCA
GGACGAGGCAGCGCGGCTATCGTGGCTGGCCACGACGGGCGTTCCTTGCGCAGCTGTGCTCGACGTTGTC
ACTGAAGCGGGAAGGGACTGGCTGCTATTGGGCGAAGTGCCGGGGCAGGATCTCCTGTCATCTCACCTTG
CTCCTGCCGAGAAAGTATCCATCATGGCTGATGCAATGCGGCGGCTGCATACGCTTGATCCGGCTACCTG
CCCATTCGACCACCAAGCGAAACATCGCATCGAGCGAGCACGTACTCGGATGGAAGCCGGTCTTGTCGAT
CAGGATGATCTGGACGAAGAGCATCAGGGGCTCGCGCCAGCCGAACTGTTCGCCAGGCTCAAGGCGCGCA
TGCCCGACGGCGAGGATCTCGTCGTGACCCATGGCGATGCCTGCTTGCCGAATATCATGGTGGAAAATGG
CCGCTTTTCTGGATTCATCGACTGTGGCCGGCTGGGTGTGGCGGACCGCTATCAGGACATAGCGTTGGCT
CTCCCGATTCGCAGCGCATCGCCTTCTATCGCCTTCTTGACGAGTTCTTCTAATAAGGGGATCTTGAAGT
TCCTATTCCGAAGTTCCTATTCTCTAGAAAGTATAGGAACTTCGAAGCAGCTCCAGCCTACAC

Kanamycin promoter region—italicized
Kan^{R} marker—underlined

### SEQ ID NO:39

tetR_P_{Ltet01}-ybhGFSR DNA sequence (start codon of ybhG changed from native ‘GTG’ sequence to ‘ATG’)
The nucleotide sequence for:
tetR is in bold
P_{Ltet01} is lower-case
ybhGFSR is underlined

TTAAGACCCACTTTCACATTTAAGTTGTTTTTCTAATCCGCAAATGATCA
ATTCAAGGCCGAATAAGAAGGCTGGCTCTGCACCTTGGTGTTCAAATAAT
TCGATAGCTTGTCGTAATAATGCTGGCATACTATCAGTAGTAGGTGTTTC
CCTTTCTTCTTTAGCGACTTGATGCTCTTGATCTTCCAATACGCAACCTA
AAGTAAAATGCCCCACAGCGCTGAGTGCATATAATGCATTCTCTAGTGAA
AAACCTTGTTGGCATAAAAAGGCTAATTGATTTTCGAGAGTTTCATACTG
TTTTTCTGTAGGCCGTGTACCTAAATGTACTTTTGCTCCATCGCGATGAC
TTAGTAAAGCACATCTAAAACTTTTAGCGTTATTACGTAAAAAATCTTGC
CAGCTTTCCCCTTCTAAAGGGCAAAAGTGAGTATGGTGCCTATCTAACAT
CTCAATGGCTAAGGCGTCGAGCAAAGCCCGCTTATTTTTTACATGCCAAT
ACAATGTAGGCTGCTCTACACCCAGCTTCTGGGCGAGTTTACGGGTTTTT
AAACCTTCGATTCCGACCTCATTAAGCAGCTCTAATGCGCTGTTAATCAC
TTTACTTTTATCTAATCTGGACATCATTTGGTTTTCCTCCAGCAAAATGT
ACAGCAACCATTATCACCGCCAGAGGTAAAATAGTCAACACGCACGGTGT
TAGAGCTCtccctatcagtgatagagattgacatccctatcagtgataga
gatactgagcacatcagcaggacgcactgacccAATTCATTAAAGAGGAG
AAAGGTCATATGATGAAAAAACCTGTCGTGATCGGATTGGCGGTAGTGGT
ACTTGCCGCCGTGGTTGCCGGAGGCTACTGGTGGTATCAAAGCCGCCAGG
ATAACGGCCTGACGCTGTATGGCAACGTGGATATTCGTACGGTAAATCTT
AGTTTCCGTGTTGGGGGGCGCGTTGAATCGCTGGCGGTGGACGAAGGTGA
TGCTATCAAAGCGGGCCAGGTGCTGGGCGAACTGGATCACAAGCCGTATG
AGATTGCCCTGATGCAGGCGAAAGCGGGTGTTTCGGTGGCACAGGCGCAG
TATGACCTGATGCTTGCCGGGTATCGCAATGAAGAAATCGCTCAGGCCGC
CGCAGCGGTGAAACAGGCGCAAGCCGCCTATGACTATGCGCAGAACTTCT
ATAACCGCCAGCAAGGGTTGTGGAAAAGCCGCACTATTTCGGCAAATGAC
CTGGAAAATGCCCGCTCCTCGCGCGACCAGGCGCAGGCAACGCTGAAATC
AGCACAGGATAAATTGCGTCAGTACCGTTCCGGTAACCGTGAACAGGACA
TCGCTCAGGCGAAAGCCAGCCTCGAACAGGCGCAGGCGCAACTGGCGCAG
GCGGAGTTGAATTTACAGGACTCAACGTTGATAGCCCCGTCTGATGGCAC
GCTGTTAACGCGCGCGGTGGAGCCAGGCACGGTCCTCAATGAAGGTGGCA
CGGTGTTTACCGTTTCACTAACGCGTCCGGTGTGGGTGCGCGCTTATGTT
GATGAACGTAATCTTGACCAGGCCCAGCCGGGGCGCAAAGTGCTGCTTTA
TACCGATGGTCGCCCGGACAAGCCGTATCACGGGCAGATTGGTTTCGTTT
CGCCGACTGCTGAATTTACCCCGAAAACCGTCGAAACGCCGGATCTGCGT
ACCGACCTCGTCTATCGCCTGCGTATTGTGGTGACCGACGCCGATGATGC
GTTACGCCAGGGAATGCCAGTGACGGTACAATTCGGTGACGAGGCAGGAC
ATGAATGATGCCGTTATCACGCTGAACGGCCTGGAAAAACGCTTTCCGGG
CATGGACAAGCCCGCCGTCGCGCCGCTCGATTGTACCATTCACGCCGGTT
ATGTGACGGGGTTGGTGGGGCCGGACGGTGCAGGTAAAACCACGCTGATG
CGGATGTTGGCGGGATTACTGAAACCCGACAGCGGCAGTGCCACGGTGAT
TGGCTTTGATCCGATCAAAAACGACGGCGCGCTGCACGCCGTGCTCGGTT
ATATGCCGCAGAAATTTGGTCTGTATGAAGATCTCACGGTGATGGAGAAC
CTCAATCTGTACGCGGATTTGCGCAGCGTCACCGGCGAGGCACGTAAGCA
AACTTTTGCTCGCCTGCTGGAGTTTACGTCTCTTGGGCCGTTTACCGGAC
GCCTGGCGGGCAAGCTCTCCGGTGGGATGAAACAAAAACTCGGTCTGGCC
TGTACCCTGGTGGGCGAACCGAAAGTGTTGCTGCTCGATGAACCCGGCGT
CGGCGTTGACCCTATCTCACGGCGCGAACTGTGGCAGATGGTGCATGAGC
TGGCGGGCGAAGGGATGTTAATCCTCTGGAGTACCTCGTATCTCGACGAA
GCCGAGCAGTGCCGTGACGTGTTACTGATGAACGAAGGCGAGTTGCTGTA
TCAGGGAGAACCAAAAGCCCTGACACAAACCATGGCCGGACGCAGCTTTC
TGATGACCAGTCCACACGAGGGCAACCGCAAACTGTTGCAACGCGCCTTG
AAACTGCCGCAGGTCAGCGACGGCATGATTCAGGGGAAATCGGTACGTCT
GATCCTCAAAAAAGAGGCCACACCAGACGATATTCGCCATGCCGACGGGA
TGCCGGAAATCAACATCAACGAAACTACGCCGCGTTTTGAAGATGCGTTT
ATTGATTTGCTGGGCGGTGCCGGAACCTCGGAATCGCCGCTGGGCGCAAT
ATTACATACGGTAGAAGGCACACCCGGCGAGACGGTGATCGAAGCGAAAG
AACTGACCAAGAAATTTGGGGATTTTGCCGCCACCGATCACGTCAACTTT
GCCGTTAAACGTGGGGAGATTTTTGGTTTGCTGGGGCCAAACGGCGCGGG
TAAATCGACCACCTTTAAGATGATGTGCGGTTTGCTGGTGCCGACTTCCG
GCCAGGCGCTGGTGCTGGGGATGGATCTGAAAGAGAGTTCCGGTAAAGCG
CGCCAGCATCTCGGCTATATGGCGCAAAAATTTTCGCTCTACGGTAACCT
GACGGTCGAACAGAATTTACGCTTTTTCTCTGGTGTGTATGGCTTACGCG
GTCGGGCGCAGAACGAAAAAATCTCCCGCATGAGCGAGGCGTTCGGCCTG
AAAAGTATCGCCTCCCACGCCACCGATGAACTGCCATTAGGTTTTAAACA
GCGGCTGGCGCTGGCCTGTTCGCTGATGCATGAACCGGACATTCTGTTTC
TCGACGAACCGACTTCCGGCGTTGACCCCCTCACCCGCCGTGAATTTTGG
CTGCACATCAACAGCATGGTAGAGAAAGGCGTCACGGTGATGGTCACCAC
CCACTTTATGGATGAAGCGGAATATTGCGACCGCATCGGCCTGGTGTACC
GCGGGAAATTAATCGCCAGCGGCACGCCGGACGATTTGAAAGCACAGTCG
GCTAACGATGAGCAACCCGATCCCACCATGGAGCAAGCCTTTATTCAGTT
GATCCACGACTGGGATAAGGAGCATAGCAATGAGTAACCCGATCCTGTCC
TGGCGTCGCGTACGGGCGCTGTGCGTTAAAGAGACGCGGCAGATCGTTCG
CGATCCGAGTAGCTGGCTGATTGCGGTAGTGATCCCGCTGCTACTGCTGT
TTATTTTTGGTTACGGCATTAACCTCGACTCCAGCAAGCTGCGGGTCGGG
ATTTTACTGGAACAGCGTAGCGAAGCGGCGCTGGATTTCACCCACACCAT
GACCGGTTCGCCCTACATCGACGCCACCATCAGCGATAACCGTCAGGAAC
TGATCGCCAAAATGCAGGCGGGGAAAATTCGCGGTCTGGTGGTTATTCCG
GTGGATTTTGCGGAACAGATGGAGCGCGCCAACGCCACCGCACCGATTCA
GGTGATCACCGACGGCAGTGAGCCGAATACCGCTAACTTTGTACAGGGGT
ATGTCGAAGGGATCTGGCAGATCTGGCAAATGCAGCGAGCGGAGGACAAC
GGGCAGACTTTTGAACCGCTTATTGATGTACAAACCCGCTACTGGTTTAA
CCCGGCGGCGATTAGCCAGCACTTCATTATCCCCGGTGCGGTGACCATTA
TCATGACGGTCATCGGCGCGATTCTCACCTCGCTGGTGGTGGCGCGAGAA
TGGGAACGCGGCACCATGGAGGCTCTGCTCTCTACGGAGATTACCCGCAC
GGAACTGCTGCTGTGTAAGCTGATCCCTTATTACTTTCTCGGGATGCTGG
CGATGTTGCTGTGTATGCTGGTGTCAGTGTTTATTCTCGGCGTGCCGTAT
CGCGGGTCGCTGCTGATTCTGTTTTTTATCTCCAGCCTGTTTTTACTCAG
TACCCTGGGGATGGGGCTGCTGATTTCCACGATTACCCGCAACCAGTTCA
ATGCCGCTCAGGTCGCCCTGAACGCCGCTTTTCTGCCGTCGATTATGCTT
TCCGGCTTTATTTTTCAGATCGACAGTATGCCCGCGGTGATCCGCGCGGT
GACGTACATTATTCCCGCTCGTTATTTCGTCAGCACCCTGCAAAGCCTGT
TCCTCGCCGGGAATATTCCAGTGGTGCTGGTGGTAAACGTGCTGTTTTTG
ATCGCTTCGGCGGTGATGTTTATCGGCCTGACGTGGCTGAAAACCAAACG
TCGGCTGGATTAGGGAGAAGAGCATGTTTCATCGCTTATGGACGTTAATC
CGCAAAGAGTTGCAGTCGTTGCTGCGCGAACCGCAAACCCGCGCGATTCT
GATTTTACCCGTGCTAATTCAGGTGATCCTGTTCCCGTTCGCCGCCACGC
TGGAAGTGACTAACGCCACCATCGCCATCTACGATGAAGATAACGGCGAG
CATTCGGTGGAGCTGACCCAACGTTTTGCCCGCGCCAGCGCCTTTACTCA
TGTGCTGCTGCTGAAAAGCCCACAGGAGATCCGCCCAACCATCGACACAC
AAAAGGCGTTACTACTGGTGCGTTTCCCGGCTGACTTCTCGCGCAAACTG
GATACCTTCCAGACCGCGCCTTTGCAGTTGATCCTCGACGGGCGTAACTC
CAACAGTGCGCAAATTGCCGCCAACTACCTGCAACAGATCGTCAAAAATT
ATCAGCAGGAGCTGCTGGAAGGAAAACCGAAACCTAACAACAGCGAGCTG
GTGGTACGCAACTGGTATAACCCGAATCTCGACTACAAATGGTTTGTGGT
GCCGTCACTGATCGCCATGATCACCACTATCGGCGTAATGATCGTCACTT
CACTTTCCGTCGCCCGCGAACGTGAACAAGGTACGCTCGATCAGCTACTG
GTTTCGCCGCTCACCACCTGGCAGATCTTCATCGGCAAAGCCGTACCGGC
GTTAATTGTCGCCACCTTCCAGGCCACCATTGTGCTGGCGATTGGTATCT
GGGCGTATCAAATCCCCTTCGCCGGATCGCTGGCGCTGTTCTACTTTACG
ATGGTGATTTATGGTTTATCGCTGGTGGGATTCGGTCTGTTGATTTCATC
ACTCTGTTCAACACAACAGCAGGCGTTTATCGGCGTGTTTGTCTTTATGA
TGCCCGCCATTCTCCTTTCCGGTTACGTTTCTCCGGTGGAAAACATGCCG
GTATGGCTGCAAAACCTGACGTGGATTAACCCTATTCGCCACTTTACGGA
CATTACCAAGCAGATTTATTTGAAGGATGCGAGTCTGGATATTGTGTGGA
ATAGTTTGTGGCCGCTACTGGTGATAACGGCCACGACAGGGTCAGCGGCG
TACGCGATGTTTAGACGTAAGGTGATGTAA

### SEQ ID NO:42

DNA sequence of rfaC locus in JCC1880 (ΔfadE)

TGACGCTGCGGAGGGTTATCACCAGAGCTTAATCGACATTACTCCCCAGC
GCGTACTGGAAGAACTCAACGCGCTATTGTTACAAGAGGAAGCCTGACGG
atgCGGGTTTTGATCGTTAAAACATCGTCGATGGGCGATGTTCTCCATAC
GTTGCCCGCACTCACTGATGCCCAGCAGGCAATCCCAGGGATTAAGTTTG
ACTGGGTGGTGGAAGAAGGGTTCGCACAGATTCCTTCCTGGCACGCTGCC
GTTGAGCGAGTTATTCCTGTGGCAATACGTCGCTGGCGTAAAGCCTGGTT
CTCGGCCCCCATAAAAGCGGAACGCAAAGCGTTTCGTGAAGCGCTACAAG
CAGAGAACTATGACGCAGTTATCGACGCTCAGGGGCTGGTAAAAAGCGCG
GCGCTGGTGACGCGTCTGGCGCATGGCGTAAAGCATGGCATGGACTGGCA
AACCGCTCGCGAACCTTTAGCCAGCCTGTTTTACAATCGTAAGCATCATA
TTGCAAAACAGCAGCACGCCGTAGAACGCACCCGCGAACTGTTTGCCAAA
AGTTTGGGCTATAGCAAACCGCAAACCCAGGGCGATTATGCTATCGCACA
GCATTTTCTGACGAACCTGCCTACAGATGCTGGCGAATATGCCGTATTTC
TTCATGCGACGACCCGTGATGATAAACACTGGCCGGAAGAACACTGGCGA
GAATTGATTGGTTTACTGGCTGATTCAGGAATACGGATTAAACTTCCGTG
GGGCGCGCCGCATGAGGAAGAACGGGCGAAACGACTGGCGGAAGGATTTG
CTTATGTTGAAGTATTGCCGAAGATGAGTCTGGAAGGCGTTGCCCGCGTG
CTGGCCGGGGCTAAATTTGTAGTGTCGGTGGATACGGGGTTAAGCCATTT
AACGGCGGCACTGGATAGACCCAATATCACGGTTTATGGACCAACCGATC
CGGGATTAATTGGTGGGTATGGGAAGAATCAGATGGTATGTAGGGCTCCA
AGAGAAAATTTAATTAACCTCAACAGTCAAGCAGTTTTGGAAAAGTTATC
ATCATTAtaaAGGTAAAACATGCTAACATCCTTTAAACTTCATTCATTGA
AACCTTACACTCTGAAATCATCAATGATTTTAGAGATAATAACTTATATA
TTATGTTTTT

rfaC ORF is underlined, H1 and H2 italicized

### SEQ ID NO:43

DNA sequence of rfaC locus in JCC1999 (ΔfadEΔrfaC)

TGACGCTGCGGAGGGTTATCACCAGAGCTTAATCGACATTACTCCCCAGC
GCGTACTGGAAGAACTCAACGCGCTATTGTTACAAGAGGAAGCCTGACGG
gtgtaggctggagctgcttcgaagttcctatactttctagagaataggaa
cttcgaactgcaggtcgacggatccccggaattaattctcatgtttgaca
gAGGTAAAACATGCTAACATCCTTTAAACTTCATTCATTGAAACCTTAC
ACTCTGAAATCATCAATGATTTTAGAGATAATAACTTATATATTATGTT
TTT

H1 and H2 italicized

### SEQ ID NO:44

P(psaA) DNA sequence

GCCCCTATATTATGCATTTATACCCCCACAATCATGTCAAGAATTCAAGC
ATCTTAAATAATGTTAATTATCGGCAAAGTCTGTGCTCCCCTTCTATAAT
GCTGAATTGAGCATTCGCCTCCTGAACGGTCTTTATTCTTCCATTGTGGG
TCTTTAGATTCACGATTCTTCACAATCATTGATCTAAAGATCTTTCTAGA
TTCTCGAGGCA

### SEQ ID NO:45

P(nir07) DNA sequence

GGCCGCTTGTAGCAATTGCTACTAAAAACTGCGATCGCTGCTGAAATGAG
CTGGAATTTTGTCCCTCTCAGCTCAAAAAGTATCAATGATTACTTAATGT
TTGTTCTGCGCAAACTTCTTGCAGAACATGCATGATTTACAAAAAGTTGT
AGTTTCTGTTACCAATTGCGAATCGAGAACTGCCTAATCTGCCGAGTATG
CGATCCTTTAGCAGGAGGAAAACCA

### SEQ ID NO:46

P(nir09) DNA sequence

GCTACTCATTAGTTAAGTGTAATGCAGAAAACGCATATTCTCTATTAAAC
TTACGCATTAATACGAGAATTTTGTAGCTACTTATACTATTTTACCTGAG
ATCCCGACATAACCTTAGAAGTATCGAAATCGTTACATAAACATTCACAC
AAACCACTTGACAAATTTAGCCAATGTAAAAGACTACAGTTTCTCCCCGG
TTTAGTTCTAGAGTTACCTTCAGTGAAACATCGGCGGCGTGTCAGTCATT
GAAGTAGCATAAATCAATTCAAAATACCCTGCGGGAAGGCTGCGCCAACA
AAATTAAATATTTGGTTTTTCACTATTAGAGCATCGATTCATTAATCAAA
AACCTTACCCCCCAGCCCCCTTCCCTTGTAGGGAAGTGGGAGCCAAACTC
CCCTCTCCGCGTCGGAGCGAAAAGTCTGAGCGGAGGTTTCCTCCGAACAG
AACTTTTAAAGAGAGAGGGGTTGGGGGAGAGGTTCTTTCAAGATTACTAA
ATTGCTATCACTAGACCTCGTAGAACTAGCAAAGACTACGGGTGGATTGA
TCTTGAGCAAAAAAACTTTATGAGAACTTTAGCAGGAGGAAAACCA

### SEQ ID NO:47

accA Codon optimized DNA sequence

ATGAGCCTGAATTTCCTGGACTTTGAACAACCTATTGCTGAACTGGAGGC
AAAAATCGATTCCCTGACTGCCGTTAGCCGCCAGGACGAAAAGCTGGATA
TCAACATCGACGAAGAAGTACATCGCCTGCGTGAGAAATCTGTTGAACTG
ACCCGTAAAATCTTCGCCGATCTGGGCGCCTGGCAGATCGCGCAGCTGGC
TCGCCACCCACAACGTCCGTATACCCTGGACTACGTACGTCTGGCTTTCG
ATGAGTTCGACGAGCTGGCGGGCGATCGTGCCTACGCGGACGACAAAGCT
ATCGTGGGCGGTATCGCTCGTCTGGACGGTCGTCCGGTAATGATCATCGG
CCATCAAAAGGGTCGTGAAACCAAAGAGAAAATCCGTCGTAACTTCGGTA
TGCCTGCACCGGAAGGCTATCGTAAAGCCCTGCGTCTGATGCAAATGGCG
GAGCGTTTCAAAATGCCGATTATCACCTTTATCGATACTCCTGGTGCTTA
CCCAGGTGTCGGTGCGGAAGAACGTGGCCAGTCCGAGGCTATCGCCCGTA
ACCTGCGTGAAATGTCCCGCCTGGGTGTCCCGGTTGTTTGCACCGTTATT
GGCGAGGGTGGCTCCGGTGGTGCGCTGGCAATCGGTGTTGGTGACAAAGT
TAACATGCTGCAGTACTCTACCTACAGCGTCATCTCTCCGGAGGGCTGCG
CTTCTATCCTGTGGAAATCCGCTGACAAAGCTCCGCTGGCAGCTGAAGCT
ATGGGCATCATCGCACCGCGCCTGAAAGAGCTGAAACTGATCGACTCTAT
CATCCCTGAGCCGCTGGGTGGTGCTCACCGCAACCCAGAAGCGATGGCAG
CGTCCCTGAAAGCACAACTGCTGGCTGACCTGGCGGATCTGGATGTTCTG
TCTACTGAGGATCTGAAAAATCGTCGTTACCAACGTCTGATGTCCTATGG
TTACGCTTGA

### SEQ ID NO:48

accD Codon optimized DNA sequence

ATGTCGTGGATCGAGCGTATTAAATCTAACATCACCCCAACTCGTAAGGC
ATCCATTCCGGAAGGCGTTTGGACGAAATGTGATTCTTGCGGCCAGGTTC
TGTATCGCGCCGAACTGGAACGTAACCTGGAGGTTTGTCCGAAGTGTGAC
CACCACATGCGTATGACCGCGCGCAATCGTCTGCATAGCCTGCTGGATGA
GGGCAGCCTGGTCGAACTGGGTTCCGAGCTGGAGCCGAAAGATGTTCTGA
AATTCCGTGATTCTAAAAAGTATAAAGACCGTCTGGCGTCTGCTCAAAAG
GAAACCGGCGAGAAGGATGCACTGGTAGTTATGAAAGGCACTCTGTATGG
CATGCCGGTGGTTGCAGCGGCTTTTGAGTTCGCTTTTATGGGCGGTAGCA
TGGGTAGCGTAGTTGGTGCTCGTTTTGTACGTGCGGTGGAACAGGCCCTG
GAGGACAACTGCCCGCTGATCTGCTTCTCCGCTTCTGGCGGTGCGCGTAT
GCAGGAAGCACTGATGTCCCTGATGCAGATGGCTAAAACCTCTGCTGCAC
TGGCGAAAATGCAGGAGCGTGGCCTGCCATACATCTCTGTTCTGACGGA
CCCGACGATGGGTGGTGTTTCCGCTTCTTTCGCGATGCTGGGCGACCT
GAACATTGCCGAACCGAAGGCGCTGATCGGTTTCGCGGGTCCGCGTG
TTATCGAACAGACGGTACGCGAAAAACTGCCGCCAGGTTTCCAACGC
AGCGAGTTTCTGATCGAAAAAGGTGCAATCGACATGATCGTTCGTCGC
CCTGAGATGCGTCTGAAGCTGGCTTCCATCCTGGCGAAACTGATGAAC
CTGCCAGCCCCGAATCCGGAAGCGCCGCGTGAAGGCGTTGTTGTCCCAC
CAGTACCAGACCAGGAACCGGAGGCGTAA

### SEQ ID NO:49

accB Codon optimized DNA sequence

ATGGACATCCGTAAAATCAAGAAACTGATCGAACTGGTTGAGGAGTCTGG
CATCAGCGAGCTGGAGATTTCCGAAGGCGAAGAATCCGTCCGTATCAGCC
GTGCTGCCCCGGCAGCCAGCTTCCCGGTCATGCAACAGGCTTATGCTGCT
CCGATGATGCAGCAACCGGCACAGAGCAACGCTGCGGCTCCGGCGACTGT
TCCGTCTATGGAGGCTCCGGCAGCTGCAGAAATCAGCGGCCACATCGTTC
GTAGCCCTATGGTGGGCACCTTCTACCGTACCCCATCTCCGGACGCGAAA
GCGTTCATCGAAGTAGGCCAGAAAGTCAACGTTGGTGACACCCTGTGTAT
CGTCGAAGCGATGAAAATGATGAACCAAATCGAGGCAGATAAATCCGGCA
CCGTAAAGGCGATCCTGGTTGAATCTGGTCAGCCGGTTGAATTTGATGAA
CCGCTGGTTGTCATCGAATAA

### SEQ ID NO:50

accC Codon optimized DNA sequence

ATGCTGGATAAAATCGTTATTGCTAACCGCGGCGAGATTGCTCTGCGCAT
CCTGCGCGCATGCAAAGAACTGGGTATTAAAACCGTTGCAGTTCATTCTT
CCGCCGATCGCGACCTGAAGCACGTCCTGCTGGCCGATGAAACTGTATGC
ATCGGTCCAGCACCGTCCGTTAAATCCTACCTGAACATTCCGGCGATCAT
CTCTGCCGCGGAAATCACCGGCGCTGTAGCTATCCACCCGGGTTATGGTT
TTCTGTCCGAAAACGCCAACTTTGCGGAGCAGGTTGAGCGCAGCGGCTTT
ATCTTCATCGGTCCGAAGGCTGAAACCATCCGTCTGATGGGCGATAAAGT
GTCCGCTATCGCGGCAATGAAAAAGGCAGGTGTTCCATGCGTTCCGGGCT
CTGACGGCCCGCTGGGCGACGATATGGATAAAAACCGCGCTATCGCAAAA
CGTATCGGTTATCCGGTTATTATCAAGGCATCTGGCGGTGGTGGTGGTCG
TGGTATGCGCGTTGTTCGTGGTGACGCGGAACTGGCTCAGAGCATTAGCA
TGACCCGTGCGGAAGCGAAAGCGGCTTTCTCTAACGATATGGTGTATATG
GAAAAGTACCTGGAGAACCCGCGTCACGTGGAAATTCAGGTGCTGGCTGA
TGGTCAGGGTAACGCTATCTACCTGGCTGAGCGCGATTGCTCTATGCAGC
GTCGTCACCAGAAGGTGGTTGAAGAAGCTCCGGCACCGGGCATCACTCCA
GAGCTGCGTCGCTACATCGGCGAACGTTGTGCGAAAGCCTGCGTGGATAT
CGGTTACCGTGGTGCTGGCACTTTCGAATTTCTGTTTGAAAACGGTGAGT
TCTACTTCATTGAAATGAACACTCGTATCCAGGTTGAACACCCTGTCACC
GAAATGATTACCGGCGTTGACCTGATTAAAGAACAACTGCGTATCGCAGC
GGGTCAGCCGCTGTCTATTAAGCAGGAAGAAGTCCATGTCCGTGGTCACG
CCGTCGAATGCCGTATCAACGCAGAAGACCCGAACACCTTCCTGCCGTCC
CCGGGTAAAATCACTCGCTTTCACGCGCCAGGTGGTTTCGGTGTCCGTTG
GGAGTCCCACATTTATGCTGGTTACACGGTACCGCCGTACTACGACTCCA
TGATCGGTAAACTGATCTGCTATGGCGAAAACCGTGACGTAGCGATCGCG
CGTATGAAGAACGCTCTGCAGGAGCTGATTATTGATGGCATCAAAACCAA
TGTTGACCTGCAGATCCGCATTATGAACGACGAGAACTTCCAGCACGGCG
GCACCAACATCCATTATCTGGAGAAGAAACTGGGTCTGCAGGAAAAATAA

### SEQ ID NO:51

Base vector sequence for pJB1623-1626
EcoRI/NotI-flanked sequence of plasmid pJB525. EcoRI and NotI sites are in lower case, DHR and UHR are in italics (in that order), and the kanamycin cassette coding sequence is underlined

gaattcGGTTTTCCGTCCTGTCTTGATTTTCAAGCAAACAATGCCTCCGA
TTTCTAATCGGAGGCATTTGTTTTTGTTTATTGCAAAAACAAAAAATATT
GTTACAAATTTTTACAGGCTATTAAGCCTACCGTCATAAATAATTTGCCA
TTTACTAGTTTTTAATTAAACCCCTATTTGTTTATTTTTCTAAATACATT
CAAATATGTATCCGCTCATGAGACAATAACCCTGATAAATGCTTCAATAA
TATTGAAAAAGGAAGAGTATGATTGAACAAGATGGCCTGCATGCTGGTTC
TCCGGCTGCTTGGGTGGAACGCCTGTTTGGTTACGACTGGGCTCAGCTGA
CTATTGGCTGTAGCGATGCAGCGGTTTTCCGTCTGTCTGCACAGGGTCGT
CCGGTTCTGTTTGTGAAAACCGACCTGTCCGGCGCACTGAACGAACTGCA
GGACGAAGCGGCCCGTCTGTCCTGGCTCGCGACGACTGGTGTTCCGTGCG
CGGCAGTTCTGGACGTAGTTACTGAAGCCGGTCGCGATTGGCTGCTGCTG
GGTGAAGTTCCGGGTCAGGATCTGCTGAGCAGCCACCTCGCTCCGGCAG
AAAAAGTTTCCATCATGGCGGACGCGATGCGCCGTCTGCACACCCTGGAC
CCGGCAACTTGCCCGTTTGACCATCAGGCTAAACACCGTATTGAACGTGC
ACGCACTCGTATGGAAGCGGGTCTGGTTGATCAGGACGACCTGGATGAAG
AGCACCAGGGCCTCGCACCGGCGGAACTGTTTGCACGTCTGAAAGCCCGC
ATGCCGGACGGCGAAGACCTGGTGGTAACGCATGGCGACGCTTGTCTGCC
AAACATTATGGTGGAAAACGGCCGCTTCTCTGGTTTTATTGACTGTGGCC
GTCTGGGTGTAGCTGATCGCTATCAGGATATCGCCCTCGCTACCCGCGAT
ATTGCAGAAGAACTGGGTGGTGAATGGGCTGACCGTTTCCTGGTGCTGTA
CGGTATCGCAGCGCCGGATTCTCAGCGCATTGCCTTCTACCGTCTGCTGG
ATGAGTTCTTCTAAGGCGCGCCTGATCAGTTGGTGCTGCATTAGCTAAGA
AGGTCAGGAGATATTATTCGACATCTAGCTGACGGCCATTGCGATCATAA
ACGAGGATATCCCACTGGCCATTTTCAGCGGCTTCAAAGGCAATTTTAGA
CCCATCAGCACTAATGGTTGGATTACGCACTTCTTGGTTTAAGTTATCGG
TTAAATTCCGCTTTTGTTCAAACTCGCGATCATAGAGATAAATATCAGAT
TCGCCGCGACGATTGACCGCAAAGACAATGTAGCGACCATCTTCAGAAAC
GGCAGGATGGGAGGCAATTTCATTTAGGGTATTGAGGCCCGGTAACAGAA
TCGTTTGCCTGGTGCTGGTATCAAATAGATAGATATCCTGGGAACCATTG
CGGTCTGAGGCAAAAACGAGGTAGGGTTCGGCGATCGCCGGGTCAAATTC
GAGGGCCCGACTATTTAAACTGCGGCCACCGGGATCAACGGGAAAATTGA
CAATGCGCGGATAACCAACGCAGCTCTGGAGCAGCAAACCGAGGCTACCG
AGGAAAAAACTGCGTAGAAAAGAAACATAGCGCATAGGTCAAAGGGAAAT
CAAAGGGCGGGCGATCGCCAATTTTTCTATAATATTGTCCTAACAGCACA
CTAAAACAGAGCCATGCTAGCAAAAATTTGGAGTGCCACCATTGTCGGGG
TCGATGCCCTCAGGGTCGGGGTGGAAGTGGATATTTCCGGCGGCTTACCG
AAAATGATGGTGGTCGGACTGCGGCCGGCCAAAATGAAGTGAAGTTCCTA
TACTTTCTAGAGAATAGGAACTTCTATAGTGAGTCGAATAAGGGCGACAC
AAAATTTATTCTAAATGCATAATAAATACTGATAACATCTTATAGTTTGT
ATTATATTTTGTATTATCGTTGACATGTATAATTTTGATATCAAAAACTG
ATTTTCCCTTTATTATTTTCGAGATTTATTTTCTTAATTCTCTTTAACAA
ACTAGAAATATTGTATATACAAAAAATCATAAATAATAGATGAATAGTTT
AATTATAGGTGTTCATCAATCGAAAAAGCAACGTATCTTATTTAAAGTGC
GTTGCTTTTTTCTCATTTATAAGGTTAAATAATTCTCATATATCAAGCAA
AGTGACAGGCGCCCTTAAATATTCTGACAAATGCTCTTTCCCTAAACTCC
CCCCATAAAAAAACCCGCCGAAGCGGGTTTTTACGTTATTTGCGGATTAA
CGATTACTCGTTATCAGAACCGCCCAGGGGGCCCGAGCTTAAGACTGGCC
GTCGTTTTACAACACAGAAAGAGTTTGTAGAAACGCAAAAAGGCCATCCG
TCAGGGGCCTTCTGCTTAGTTTGATGCCTGGCAGTTCCCTACTCTCGCCT
TCCGCTTCCTCGCTCACTGACTCGCTGCGCTCGGTCGTTCGGCTGCGGCG
AGCGGTATCAGCTCACTCAAAGGCGGTAATACGGTTATCCACAGAATCAG
GGGATAACGCAGGAAAGAACATGTGAGCAAAAGGCCAGCAAAAGGCCAGG
AACCGTAAAAAGGCCGCGTTGCTGGCGTTTTTCCATAGGCTCCGCCCCCC
TGACGAGCATCACAAAAATCGACGCTCAAGTCAGAGGTGGCGAAACCCGA
CAGGACTATAAAGATACCAGGCGTTTCCCCCTGGAAGCTCCCTCGTGCGC
TCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTT
CGGTGTAGGTCGTTCGCTCCAAGCTGGGCTGTGTGCACGAACCCCCCGTT
CAGCCCGACCGCTGCGCCTTATCCGGTAACTATCGTCTTGAGTCCAACCC
GGTAAGACACGACTTATCGCCACTGGCAGCAGCCACTGGTAACAGGATTA
GCAGAGCGAGGTATGTAGGCGGTGCTACAGAGTTCTTGAAGTGGTGGGCT
AACTACGGCTACACTAGAAGAACAGTATTTGGTATCTGCGCTCTGCTGAA
GCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGC
AGAAAAAAAGGATCTCAAGAAGATCCTTTGATCTTTTCTACGGGGTCTGA
CGCTCAGTGGAACGACGCGCGCGTAACTCACGTTAAGGGATTTTGGTCAT
GAGCTTGCGCCGTCCCGTCAAGTCAGCGTAATGCTCTGCTTTTAGAAAAA
CTCATCGAGCATCAAATGAAACTGCAATTTATTCATATCAGGATTATCAA
TACCATATTTTTGAAAAAGCCGTTTCTGTAATGAAGGAGAAAACTCACCG
AGGCAGTTCCATAGGATGGCAAGATCCTGGTATCGGTCTGCGATTCCGAC
TCGTCCAACATCAATACAACCTATTAATTTCCCCTCGTCAAAAATAAGGT
TATCAAGTGAGAAATCACCATGAGTGACGACTGAATCCGGTGAGAATGGC
AAAAGTTTATGCATTTCTTTCCAGACTTGTTCAACAGGCCAGCCATTACG
CTCGTCATCAAAATCACTCGCATCAACCAAACCGTTATTCATTCGTGATT
GCGCCTGAGCGAGGCGAAATACGCGATCGCTGTTAAAAGGACAATTACAA
ACAGGAATCGAGTGCAACCGGCGCAGGAACACTGCCAGCGCATCAACAAT
ATTTTCACCTGAATCAGGATATTCTTCTAATACCTGGAACGCTGTTTTTC
CGGGGATCGCAGTGGTGAGTAACCATGCATCATCAGGAGTACGGATAAAA
TGCTTGATGGTCGGAAGTGGCATAAATTCCGTCAGCCAGTTTAGTCTGAC
CATCTCATCTGTAACATCATTGGCAACGCTACCTTTGCCATGTTTCAGAA
ACAACTCTGGCGCATCGGGCTTCCCATACAAGCGATAGATTGTCGCACCT
GATTGCCCGACATTATCGCGAGCCCATTTATACCCATATAAATCAGCATC
CATGTTGGAATTTAATCGCGGCCTCGACGTTTCCCGTTGAATATGGCTCA
TATTCTTCCTTTTTCAATATTATTGAAGCATTTATCAGGGTTATTGTCTC
ATGAGCGGATACATATTTGAATGTATTTAGAAAAATAAACAAATAGGGGT
CAGTGTTACAACCAATTAACCAATTCTGAACATTATCGCGAGCCCATTTA
TACCTGAATATGGCTCATAACACCCCTTGTTTGCCTGGCGGCAGTAGCGC
GGTGGTCCCACCTGACCCCATGCCGAACTCAGAAGTGAAACGCCGTAGCG
CCGATGGTAGTGTGGGGACTCCCCATGCGAGAGTAGGGAACTGCCAGGCA
TCAAATAAAACGAAAGGCTCAGTCGAAAGACTGGGCCTTTCGCCCGGGC
TAATTAGGGGGTGTCGCCCTTATTCGACTCTATAGTGAAGTTCCTATTCT
CTAGAAAGTATAGGAACTTCTGAAGTGGGGCCTGCAGGACAACTCGGCTT
CCGAGCTTGGCTCCACCATGGTTATATCTGGAGTAACCAGAATTTCGACA
ACTTCGACGACTATCTCGGTGCTTTTACCTCCAACCAACGCAAAAACATT
AAGCGCGAACGCAAAGCCGTTGACAAAGCAGGTTTATCCCTCAAGATGAT
GACCGGGGACGAAATTCCCGCCCATTACTTCCCACTCATTTATCGTTTCT
ATAGCAGCACCTGCGACAAATTTTTTTGGGGGAGTAAATATCTCCGGAAA
CCCTTTTTTGAAACCCTAGAATCTACCTATCGCCATCGCGTTGTTCTGGC
CGCCGCTTACACGCCAGAAGATGACAAACATCCCGTCGGTTTATCTTTTT
GTATCCGTAAAGATGATTATCTTTATGGTCGTTATTGGGGGGCCTTTGAT
GAATATGACTGTCTCCATTTTGAAGCCTGCTATTACAAACCGATCCAATG
GGCAATCGAGCAGGGAATTACGATGTACGATCCGGGCGCTGGCGGAAAAC
ATAAGCGACGACGTGGTTTCCCGGCAACCCCAAACTATAGCCTCCACCGT
TTTTATCAACCCCGCATGGGCCAAGTTTTAGACGCTTATATTGATGAAAT
TAATGCCATGGAGCAACAGGAAATTGAAGCGATCAATGCGGATATTCCCT
TTAAACGGCAGGAAGTTCAATTGAAAATTTCCTAGCTTCACTAGCCAAAA
GCGCGATCGCCCACCGACCATCCTCCCTTGGGGGAGATgcggccgc

### SEQ ID NO:52

Underlined (2) Upstream, downstream homology regions targeted to the locus between base pairs 7,676 and 7,677 of pAQ3 (NCBI accession #NC_{—}010477)
Italicized P(nir07) promoter
Bold (3) adm, aar, aadA coding open reading frames (ORFs), in that order
Lowercase E. coli vector backbone (DNA2.0; Menlo Park, Calif.)

CGAGCATTTCAACGATGATGAATGGGACGGCGAACCCACTGAACCCGTCG
CCATTGACCCAGAACCGCGCAAAGAACGGGAAAAAATTGATCTCGATCTG
GAGGATGAACCAGAGGAAAACCGCAAACCGCAAAAAATCAAAGTGAAGTT
AGCCGATGGGAAAGAGCGGGAACTCGCCCATACTCAAACCACAACTTTTT
GGGATGCTGATGGTAAACCCATTTCCGCCCAAGAATTTATCGAAAAGCTA
TTTGGCGACCTGCCCGACCTCTTCAAGGATGAAGCCGAACTACGCACCAT
CTGGGGGAAACCCGATACCCGTAAATCGTTCCTGACCGGACTCGCGGAAA
AAGGCTACGGTGACACCCAACTGAAGGCGATCGCACGCATTGCCGAAGCG
GAAAAAAGTGATGTCTATGATGTCCTGACTTGGGTTGCCTACAACACCAA
ACCCATTAGCAGAGAAGAGCGAGTAATTAAGCATCGAGATCTGATTTTCT
CGAAGTACACCGGAAAGCAGCAAGAATTTTTAGATTTTGTCCTAGACCAA
TACATTCGAGAAGGAGTGGAGGAACTTGATCGGGGGAAACTGCCTACCCT
CATCGAAATCAAATACCAAACCGTTAATGAAGGTTTAGTGATCTTGGGTC
AGGATATCGGTCAAGTATTCGCAGATTTTCAGGCGGATTTATATACCGAA
GATGTGGCATAAAAAAGGACGGCGATCGCCGGGGGCGTTGCCTGCCTTGA
GCGGCCGCTTGTAGCAATTGCTACTAAAAACTGCGATCGCTGCTGAAATG
AGCTGGAATTTTGTCCCTCTCAGCTCAAAAAGTATCAATGATTACTTAAT
GTTTGTTCTGCGCAAACTTCTTGCAGAACATGCATGATTTACAAAAAGTT
GTAGTTTCTGTTACCAATTGCGAATCGAGAACTGCCTAATCTGCCGAGTA
TGCGATCCTTTAGCAGGAGGAAAACCATATGCAAGAACTGGCCCTGAGAA
GCGAGCTGGACTTCAATAGCGAAACCTATAAAGATGCGTATAGCCGTATT
AACGCCATTGTGATCGAAGGCGAGCAAGAAGCATACCAAAACTACCTGGA
CATGGCGCAACTGCTGCCGGAGGACGAGGCTGAGCTGATTCGTTTGAGCA
AGATGGAGAACCGTCACAAAAAGGGTTTTCAAGCGTGCGGCAAGAACCTC
AATGTGACTCCGGATATGGATTATGCACAGCAGTTCTTTGCGGAGCTGCA
CGGCAATTTTCAGAAGGCTAAAGCCGAGGGTAAGATTGTTACCTGCCTGC
TCATCCAAAGCCTGATCATCGAGGCGTTTGCGATTGCAGCCTACAACATT
TACATTCCAGTGGCTGATCCGTTTGCACGTAAAATCACCGAGGGTGTCGT
CAAGGATGAGTATACCCACCTGAATTTCGGCGAAGTTTGGTTGAAGGAAC
ATTTTGAAGCAAGCAAGGCGGAGTTGGAGGACGCCAACAAAGAGAACTTA
CCGCTGGTCTGGCAGATGTTGAACCAGGTCGAAAAGGATGCCGAAGTGCT
GGGTATGGAGAAAGAGGCTCTGGTGGAGGACTTTATGATTAGCTATGGTG
AGGCACTGAGCAACATCGGCTTTTCTACGAGAGAAATCATGAAGATGAGC
GCGTACGGTCTGCGTGCAGCATAACTCGAGTATAAGTAGGAGATAAAAAC
ATGTTCGGCTTGATTGGCCACCTGACTAGCCTGGAGCACGCGCACAGCGT
GGCGGATGCGTTTGGCTACGGCCCGTACGCAACCCAGGGTTTAGACCTGT
GGTGTAGCGCACCGCCACAGTTTGTTGAGCACTTTCATGTCACGAGCATT
ACGGGCCAAACGATTGAGGGTAAATACATTGAGAGCGCGTTTTTGCCGGA
GATGTTGATTAAACGTCGTATCAAAGCAGCGATCCGTAAGATTCTGAACG
CGATGGCATTTGCGCAGAAGAACAATTTGAACATTACCGCGCTGGGTGGC
TTCAGCAGCATTATCTTTGAGGAGTTTAATCTGAAGGAGAATCGTCAGGT
TCGCAATGTGAGCTTGGAGTTTGACCGCTTCACCACCGGTAACACCCATA
CTGCTTACATTATCTGCCGTCAAGTCGAACAGGCGAGCGCGAAACTGGGT
ATCGACCTGTCCCAAGCGACCGTGGCGATTTGCGGTGCCACGGGTGATAT
TGGCAGCGCAGTTTGTCGCTGGCTGGATCGCAAAACCGACACCCAAGAGC
TGTTCCTGATTGCGCGCAATAAGGAACGCTTGCAACGTCTGCAAGATGAA
CTGGGTCGCGGCAAGATCATGGGCCTGGAAGAGGCACTGCCGGAAGCAGA
CATTATTGTGTGGGTTGCCTCCATGCCGAAGGGCGTGGAGATTAATGCGG
AAACCCTGAAGAAGCCGTGTCTGATCATTGACGGTGGCTACCCGAAGAAT
CTGGACACGAAAATCAAGCATCCGGACGTGCACATTTTGAAGGGTGGTAT
TGTAGAGCATTCGTTGGACATTGATTGGAAAATCATGGAAACCTGGACGT
TCCGAGCCGTCAAATGTTTGCGTGCTTCGCAGAGGCGATCTTGCTGGAGT
TCGAGCAATGGCACACGAACTTCTCGTGGGGTCGCAATCAAATCACGGTG
ACGAAGATGGAACAGATTGGTGAGGCGAGCGTGAAGCATGGTCTGCAACC
GCTGCTGTCCTGGTAAGAATTCGGTTTTCCGTCCTGTCTTGATTTTCAAG
CAAACAATGCCTCCGATTTCTAATCGGAGGCATTTGTTTTTGTTTATTGC
AAAAACAAAAAATATTGTTACAAATTTTTACAGGCTATTAAGCCTACCGT
CATAAATAATTTGCCATTTACTAGTTTTTAATTAACCAGAACCTTGACCG
AACGCAGCGGTGGTAACGGCGCAGTGGCGGTTTTCATGGCTTGTTATGAC
TGTTTTTTTGGGGTACAGTCTATGCCTCGGGCATCCAAGCAGCAAGCGCG
TTACGCCGTGGGTCGATGTTTGATGTTATGGAGCAGCAACGATGTTACGC
AGCAGGGCAGTCGCCCTAAAACAAAGTTAAACATCATGAGGGAAGCGGTG
ATCGCCGAAGTATCGACTCAACTATCAGAGGTAGTTGGCGTCATCGAGCG
CCATCTCGAACCGACGTTGCTGGCCGTACATTTGTACGGCTCCGCAGTGG
ATGGCGGCCTGAAGCCACACAGTGATATTGATTTGCTGGTTACGGTGACC
GTAAGGCTTGATGAAACAACGCGGCGAGCTTTGATCAACGACCTTTTGGA
AACTTCGGCTTCCCCTGGAGAGAGCGAGATTCTCCGCGCTGTAGAAGTCA
CCATTGTTGTGCACGACGACATCATTCCGTGGCGTTATCCAGCTAAGCGC
GAACTGCAATTTGGAGAATGGCAGCGCAATGACATTCTTGCAGGTATCTT
CGAGCCAGCCACGATCGACATTGATCTGGCTATCTTGCTGACAAAAGCAA
GAGAACATAGCGTTGCCTTGGTAGGTCCAGCGGCGGAGGAACTCTTTGAT
CCGGTTCCTGAACAGGATCTATTTGAGGCGCTAAATGAAACCTTAACGCT
ATGGAACTCGCCGCCCGACTGGGCTGGCGATGAGCGAAATGTAGTGCTTA
CGTTGTCCCGCATTTGGTACAGCGCAGTAACCGGCAAAATCGCGCCGAAG
GATGTCGCTGCCGACTGGGCAATGGAGCGCCTGCCGGCCCAGTATCAGCC
CGTCATACTTGAAGCTAGACAGGCTTATCTTGGACAAGAAGAAGATCGCT
TGGCCTCGCGCGCAGATCAGTTGGAAGAATTTGTCCACTACGTGAAAGGC
GAGATCACCAAGGTAGTCGGCAAATAATGTCTAACAATTCGTTCAAGCCG
ACGCCGCTTCGCGGCGCGGCTTAACTCAAGCGTTAGATGCACTAAGCACA
TAATTGCTCACAGCCAAACTATCAGGTCAAGTCTGCTTTTATTATTTTTA
AGCGTGCATAATAAGCCCTACACAAATTGGGAGATATATCATGAGGCGCG
CCACGAGAAAGAGTTATGACAAATTAAAATTCTGACTCTTAGATTATTTC
CAGAGAGGCTGATTTTCCCAATCTTTGGGAAAGCCTAAGTTTTTAGATTC
TATTTCTGGATACATCTCAAAAGTTCTTTTTAAATGCTGTGCAAAATTAT
GCTCTGGTTTAATTCTGTCTAAGAGATACTGAATACAACATAAGCCAGTG
AAAATTTTACGGCTGTTTCTTTGATTAATATCCTCCAATACTTCTCTAGA
GAGCCATTTTCCTTTTAACCTATCAGGCAATTTAGGTGATTCTCCTAGCT
GTATATTCCAGAGCCTTGAATGATGAGCGCAAATATTTCTAATATGCGAC
AAAGACCGTAACCAAGATATAAAAAACTTGTTAGGTAATTGGAAATGAGT
ATGTATTTTTTGTCGTGTCTTAGATGGTAATAAATTTGTGTACATTCTAG
ATAACTGCCCAAAGGCGATTATCTCCAAAGCCATATATGACGGCGGTAGT
AGAGGATTTGTGTACTTGTTTCGATAATGCCCGATAAATTCTTCTACTTT
TTTAGATTGGCAATATTGAGTAATCGAATCGATTAATTCTTGATGCTTCC
CAGTGTCATAAAATAAACTTTTATTCAGATACCAATGAGGATCATAATCA
TGGGAGTAGTGATAAATCATTTGAGTTCTGACTGCTACTTCTATCGACTC
CGTAGCATTAAAAATAAGCATTCTCAAGGATTTATCAAACTTGTATAGAT
TTggccggcccgtcaaaagggcgacaccccataattagcccgggcgaaag
gcccagtctttcgactgagcctttcgttttatttgatgcctggcagttcc
ctactctcgcatggggagtccccacactaccatcggcgctacggcgtttc
acttctgagttcggcatggggtcaggtgggaccaccgcgctactgccgcc
aggcaaacaaggggtgttatgagccatattcaggtataaatgggctcgcg
ataatgttcagaattggttaattggttgtaacactgacccctatttgttt
atttttctaaatacattcaaatatgtatccgctcatgagacaataaccct
gataaatgcttcaataatattgaaaaaggaagaatatgagtattcaacat
ttccgtgtcgcccttattcccttttttgcggcattttgccttcctgtttt
tgctcacccagaaacgctggtgaaagtaaaagatgctgaagatcagttgg
gtgcacgagtgggttacatcgaactggatctcaacagcggtaagatcctt
gagagttttcgccccgaagaacgttttccaatgatgagcacttttaaagt
tctgctatgtggcgcggtattatcccgtattgacgccgggcaagagcaac
tcggtcgccgcatacactattctcagaatgacttggttgagtactcacca
gtcacagaaaagcatcttacggatggcatgacagtaagagaattatgcag
tgctgccataaccatgagtgataacactgcggccaacttacttctgacaa
cgatcggaggaccgaaggagctaaccgcttttttgcacaacatgggggat
catgtaactcgccttgatcgttgggaaccggagctgaatgaagccatacc
aaacgacgagcgtgacaccacgatgcctgtagcgatggcaacaacgttgc
gcaaactattaactggcgaactacttactctagcttcccggcaacaatta
atagactggatggaggcggataaagttgcaggaccacttctgcgctcggc
ccttccggctggctggtttattgctgataaatccggagccggtgagcgtg
gttctcgcggtatcatcgcagcgctggggccagatggtaagccctcccgt
atcgtagttatctacacgacggggagtcaggcaactatggatgaacgaaa
tagacagatcgctgagataggtgcctcactgattaagcattggtaaaagc
agagcattacgctgacttgacgggacggcgcaagctcatgaccaaaatcc
cttaacgtgagttacgcgcgcgtcgttccactgagcgtcagaccccgtag
aaaagatcaaaggatcttcttgagatcctttttttctgcgcgtaatctgc
tgcttgcaaacaaaaaaaccaccgctaccagcggtggtttgtttgccgga
tcaagagctaccaactctttttccgaaggtaactggcttcagcagagcgc
agataccaaatactgttcttctagtgtagccgtagttagcccaccacttc
aagaactctgtagcaccgcctacatacctcgctctgctaatcctgttacc
agtggctgctgccagtggcgataagtcgtgtcttaccgggttggactcaa
gacgatagttaccggataaggcgcagcggtcgggctgaacggggggttcg
tgcacacagcccagcttggagcgaacgacctacaccgaactgagatacct
acagcgtgagctatgagaaagcgccacgcttcccgaagggagaaaggcgg
acaggtatccggtaagcggcagggtcggaacaggagagcgcacgagggag
cttccagggggaaacgcctggtatctttatagtcctgtcgggtttcgcca
cctctgacttgagcgtcgatttttgtgatgctcgtcaggggggcggagcc
tatggaaaaacgccagcaacgcggcctttttacggttcctggccttttgc
tggccttttgctcacatgttctttcctgcgttatcccctgattctgtgga
taaccgtattaccgcctttgagtgagctgataccgctcgccgcagccgaa
cgaccgagcgcagcgagtcagtgagcgaggaagcggaaggcgagagtagg
gaactgccaggcatcaaactaagcagaaggcccctgacggatggcctttt
tgcgtttctacaaactctttctgtgttgtaaaacgacggccagtcttaag
ctcgggccccctgggcggttctgataacgagtaatcgttaatccgcaaat
aacgtaaaaacccgcttcggcgggtttttttatggggggagtttagggaa
agagcatttgtcagaatatttaagggcgcctgtcactttgcttgatatat
gagaattatttaaccttataaatgagaaaaaagcaacgcactttaaataa
gatacgttgctttttcgattgatgaacacctataattaaactattcatct
attatttatgattttttgtatatacaatatttctagtttgttaaagagaa
ttaagaaaataaatctcgaaaataataaagggaaaatcagtttttgatat
caaaattatacatgtcaacgataatacaaaatataatacaaactataaga
tgttatcagtatttattatgcatttagaataaattttgtgtcgcccttcg
ctgaacctgcagg

### SEQ ID NO:53

Adm amino acid sequence encoded by pJB1331

MQELALRSELDFNSETYKDAYSRINAIVIEGEQEAYQNYLDMAQLLPEDE
AELIRLSKMENRHKKGFQACGKNLNVTPDMDYAQQFFAELHGNFQKAKAE
GKIVTCLLIQSLIIEAFAIAAYNIYIPVADPFARKITEGVVKDEYTHLNF
GEVWLKEHFEASKAELEDANKENLPLVWQMLNQVEKDAEVLGMEKEALVE
DFMISYGEALSNIGFSTREIMKMSAYGLRAA

### SEQ ID NO:54

Aar amino acid sequence encoded by pJB1331

MFGLIGHLTSLEHAHSVADAFGYGPYATQGLDLWCSAPPQFVEHFHVTSI
TGQTIEGKYIESAFLPEMLIKRRIKAAIRKILNAMAFAQKNNLNITALGG
FSSIIFEEFNLKENRQVRNVSLEFDRFTTGNTHTAYIICRQVEQASAKLG
IDLSQATVAICGATGDIGSAVCRWLDRKTDTQELFLIARNKERLQRLQDE
LGRGKIMGLEEALPEADIIVWVASMPKGVEINAETLKKPCLIIDGGYPKN
LDTKIKHPDVHILKGGIVEHSLDIDWKIMETVNMDVPSRQMFACFAEAIL
LEFEQWHTNFSWGRNQITVTKMEQIGEASVKHGLQPLLSW

### SEQ ID NO:55

Underlined (2) Upstream, downstream homology regions targeted to the locus between base pairs 2,299,863 and 2,299,864 of the JCC138 chromosome. The synthetically generated upstream homology region contains three silent single-nucleotide changes, and the downstream homology region, also synthetically generated, two single-nucleotide changes, with respect to the wild-type JCC138 genomic sequence. This was done to eliminate certain natural restriction sites so as to facilitate DNA sequence assembly by restriction digestion/ligation.
Bold (2) Bidirectional rho-independent transcriptional terminators BBa_B0011 (with an A-to-G single-nucleotide change) and BBa_B1002, in that order. Both sequences were derived from the Registry of Standard Biological Parts (http://partsregistry.org/). These sequences were incorporated to transcriptionally insulate the integrated divergent omp-P1-P2-ybhGFSR cassette.
Lowercase E. coli vector backbone (DNA2.0; Menlo Park, Calif.)

GTGGGTGCTGCAGTAGTCGGGCCTCGCCTCGGCAAATACCGTGATGGTCA
AGTCCACGCCATTCCTGGTCACAACATGAGTATTGCGACCTTAGGCTGTC
TAATTCTTTGGATTGGCTGGTTTGGTTTTAACCCCGGTTCTCAATTGGCA
GCAGATGCTGCGGTGCCTTACATCGCAATCACTACAAACCTTTCGGCTGC
AGCTGGGGGAATCACCGCAACCGCAACCTCTTGGATCAAAGATGGGAAGC
CAGACCTGTCTATGATTATTAACGGTATTTTGGCTGGTCTCGTTGGGATT
ACAGCCGGTTGTGATGGCGTCAGTTTCTTTTCTGCTGTGATCATCGGGGC
GATCGCCGGTGTACTCGTCGTCTTCTCTGTGGCCTTCTTCGATGCTATTA
AAATCGATGACCCCGTTGGTGCGACCTCTGTGCACCTCGTCTGCGGTATC
TGGGGAACTCTTGCCGTTGGTCTGTTCAAGATGGATGGGGGTTTATTCAC
TGGCGGTGGCATCCAACAGCTGATTGCCCAAATCGTCGGAATCCTTTCCA
TTGGTGGCTTTACCGTCGCCTTTAGCTTTATTGTTTGGTATGCCCTATCG
GCAGTCCTTGGTGGCATTCGCGTCGAAAAAGACGAGGAACTCCGGGGTCT
CGACATTGGTGAGCACGGCATGGAAGCTTACAGCGGCTTTGTTAAAGAGT
CCGATGTTATCTTCCGAGGGACTGCCACTGGTTCCGAAACCGAAGGATAA
GCGGCCGCGGTACTGCCCTCGATCTGTAAGAGAATATAAAAAGCCAGATT
ATTAATCCGGCTTTTTTGTTATTTCTATACATCTTATATCCGTGGGATCC
-GAGCTCTCAGGTATCCGGTACGCCGCCGCAAAAAACCCCGCTTCGGCGG
GGTTTTTTCGCGGCGCGCCATCCTCCCAGGAAATCCTTAAAACAATCTAA
AGAAATTTTTCCTAACCTTCCTTACCCAAGGGAGGTTTTTTATGTGAGTT
CACATTTTGTTACGTTACCCAATCAATACTTGAGCCGCTCAAAAAGTCTG
ACCTAGAGCAGAAAGTCCCTGAGTATATCGACTCATTAATCCGGTCTTTC
CGCTTGGTTTCTTGAGTTGATTTTCTGCGAAATTTTGGAAATTCAGAGAT
GTAACCTTAGGGGGAGTCCACTTAAAAACGGCTCTGCTCAACCTTGCAAA
TGCCCTACTCTTCTTCTGTCTAGCCCAAGCACTCCCTGAGAAAATTAGCG
GCGATCGCCTATAAACATGAAGTTTTATGACAGATCATTTTACAAGATGT
AATGTTTAAATGCCGGCAGACGTTGTATAACATTTACCTAAGATTAAGAG
TCACTCGCAGTACTCCTTAGAAACCCCATAGGTTCCAAGGAACTAGCATG
AACTTTATCTGGCAACTTTAAGAATCTGAGAAATTCAATGAATGTAAAGT
TTCTTAAATGCCAAGGTGAAAAACAAGCAAAAATAGCTGACACTCTTAAT
TGGCTTTGGGGATTAAGTTTCCAACTCGAAAACAAAACCTTTTATCGACT
CTAGGATTTTGTTTTCAGCAAGAGAGCCCCTCAGCACTTGCTTCACTCTT
GTTAGTAAGCAAACCGCACAAAATAAATCCCACTCATCAAAATATAAGTA
GGAGATAAAAACATGTTTGggccggccaaaagagtcgaataagggcgaca
caaaatttattctaaatgcataataaatactgataacatcttatagtttg
tattatattttgtattatcgttgacatgtataattttgatatcaaaaact
gattttccctttattattttcgagatttattttcttaattctctttaaca
aactagaaatattgtatatacaaaaaatcataaataatagatgaatagtt
taattataggtgttcatcaatcgaaaaagcaacgtatcttatttaaagtg
cgttgcttttttctcatttataaggttaaataattctcatatatcaagca
aagtgacaggcgcccttaaatattctgacaaatgctctttccctaaactc
cccccataaaaaaacccgccgaagcgggtttttacgttatttgcggatta
acgattactcgttatcagaaccgcccagggggcccgagcttaagactggc
cgtcgttttacaacacagaaagagtttgtagaaacgcaaaaaggccatcc
gtcaggggccttctgcttagtttgatgcctggcagttccctactctcgcc
ttccgcttcctcgctcactgactcgctgcgctcggtcgttcggctgcggc
gagcggtatcagctcactcaaaggcggtaatacggttatccacagaatca
ggggataacgcaggaaagaacatgtgagcaaaaggccagcaaaaggccag
gaaccgtaaaaaggccgcgttgctggcgtttttccataggctccgccccc
ctgacgagcatcacaaaaatcgacgctcaagtcagaggtggcgaaacccg
acaggactataaagataccaggcgtttccccctggaagctccctcgtgcg
ctctcctgttccgaccctgccgcttaccggatacctgtccgcctttctcc
cttcgggaagcgtggcgctttctcatagctcacgctgtaggtatctcagt
tcggtgtaggtcgttcgctccaagctgggctgtgtgcacgaaccccccgt
tcagcccgaccgctgcgccttatccggtaactatcgtcttgagtccaacc
cggtaagacacgacttatcgccactggcagcagccactggtaacaggat
tagcagagcgaggtatgtaggcggtgctacagagttcttgaagtggtgg
gctaactacggctacactagaagaacagtatttggtatctgcgctctgc
tgaagccagttaccttcggaaaaagagttggtagctcttgatccggcaa
acaaaccaccgctggtagcggtggtttttttgtttgcaagcagcagatt
acgcgcagaaaaaaaggatctcaagaagatcctttgatcttttctacggg
gtctgacgctcagtggaacgacgcgcgcgtaactcacgttaagggatttt
ggtcatgagcttgcgccgtcccgtcaagtcagcgtaatgctctgcttagg
tggcggtacttgggtcgatatcaaagtgcatcacttcttcccgtatgccc
aactttgtatagagagccactgcgggatcgtcaccgtaatctgcttgcac
gtagatcacataagcaccaagcgcgttggcctcatgcttgaggagattga
tgagcgcggtggcaatgccctgcctccggtgctcgccggagactgcgaga
tcatagatatagatctcactacgcggctgctcaaacttgggcagaacgta
agccgcgagagcgccaacaaccgcttcttggtcgaaggcagcaagcgcga
tgaatgtcttactacggagcaagttcccgaggtaatcggagtccggctga
tgttgggagtaggtggctacgtcaccgaactcacgaccgaaaagatcaag
agcagcccgcatggatttgacttggtcagggccgagcctacatgtgcgaa
tgatgcccatacttgagccacctaactttgttttagggcgactgccctgc
tgcgtaacatcgttgctgctccataacatcaaacatcgacccacggcgta
acgcgcttgctgcttggatgcccgaggcatagactgtacaaaaaaacagt
cataacaagccatgaaaaccgccactgcgccgttaccaccgctgcgttcg
gtcaaggttctggaccagttgcgtgagcgcatttttttttcctcctcggc
gtttacgccccgccctgccactcatcgcagtactgttgtaattcattaag
cattctgccgacatggaagccatcacagacggcatgatgaacctgaatcg
ccagcggcatcagcaccttgtcgccttgcgtataatatttgcccatagtg
aaaacgggggcgaagaagttgtccatattggccacgtttaaatcaaaact
ggtgaaactcacccagggattggcgctgacgaaaaacatattctcaataa
accctttagggaaataggccaggttttcaccgtaacacgccacatcttgc
gaatatatgtgtagaaactgccggaaatcgtcgtgtgcactcatggaaaa
cggtgtaacaagggtgaacactatcccatatcaccagctcaccgtctttc
attgccatacggaactccggatgagcattcatcaggcgggcaagaatgtg
aataaaggccggataaaacttgtgcttatttttctttacggtctttaaaa
aggccgtaatatccagctgaacggtctggttataggtacattgagcaact
gactgaaatgcctcaaaatgttctttacgatgccattgggatatatcaac
ggtggtatatccagtgatttttttctccatttttttttcctcctttagaa
aaactcatcgagcatcaaatgaaactgcaatttattcatatcaggattat
caataccatatttttgaaaaagccgtttctgtaatgaaggagaaaactca
ccgaggcagttccataggatggcaagatcctggtatcggtctgcgattcc
gactcgtccaacatcaatacaacctattaatttcccctcgtcaaaaataa
ggttatcaagtgagaaatcaccatgagtgacgactgaatccggtgagaa
tggcaaaagtttatgcatttctttccagacttgttcaacaggccagccat
tacgctcgtcatcaaaatcactcgcatcaaccaaaccgttattcattcgt
gattgcgcctgagcgaggcgaaatacgcgatcgctgttaaaaggacaatt
acaaacaggtgcacactgccagcgcatcaacaatattttcacctgaatca
ggatattcttctaatacctggaacgctgtttttccggggatcgcagtggt
gagtaaccatgcatcatcaggagtacggataaaatgcttgatggtcggaa
gtggcataaattccgtcagccagtttagtctgaccatctcatctgtaaca
tcattggcaacgctacctttgccatgtttcagaaacaactctggcgcatc
gggcttcccatacaagcgatagattgtcgcacctgattgcccgacattat
cgcgagcccatttatacccatataaatcagcatccatgttggaatttaat
cgcggcctcgacgtttcccgttgaatatggctcatttttttttcctcctt
taccaatgcttaatcagtgaggcacctatctcagcgatctgtctatttcg
ttcatccatagttgcctgactccccgtcgtgtagataactacgatacggg
agggcttaccatctggccccagcgctgcgatgataccgcgagaaccacgc
caccggctccggatttatcagcaataaaccagccagccggaagggccgag
gcagaagtggtcctgcaactttatccgcctccatccagtctattaattgt
tgccgggaagctagagtaagtagttcgccagttaatagtttgcgcaacgt
tgttgccatcgctacaggcatcgtggtgtcacgctcgtcgtttggtatg
gcttcattcagctccggttcccaacgatcaaggcgagttacatgatcccc
catgttgtgcaaaaaagcggttagctccttcggtcctccgatcgttgtca
gaagtaagttggccgcagtgttatcactcatggttatggcagcactgcat
aattctcttactgtcatgccatccgtaagatgcttttctgtgactggtga
gtactcaaccaagtcattctgagaatagtgtatgcggcgaccgagttgct
cttgcccggcgtcaatacgggataataccgctttaaaagtgctcatcatt
ggaaaacgttcttcggggcgaaaactctcaaggatcttaccgctgttgag
atccagttcgatgtaacccactcgtgcacccaactgatcttcagcatctt
ttactttcaccagcgtttctgggtgagcaaaaacaggaaggcaaaatgc
cgcaaaaaagggaataagggcgacacggaaatgttgaatactcatattct
tcctttttcaatattattgaagcatttatcagggttattgtctcatgagc
ggatacatatttgaatgtatttagaaaaataaacaaataggggtcagtgt
tacaaccaattaaccaattctgaacattatcgcgagcccatttatacctg
aatatggctcataacaccccttgtttgcctggcggcagtagcgcggtggt
cccacctgaccccatgccgaactcagaagtgaaacgccgtagcgccgatg
gtagtgtggggactccccatgcgagagtagggaactgccaggcatcaaat
aaaacgaaaggctcagtcgaaagactgggcctttcgcccgggctaattg
aggggtgtcgcccttattcgactcggggcctgcagg

### SEQ ID NO:56

The DNA sequence of A0585_ProNterm_tolC (native E. coli tolC with its encoded signal sequence replaced by the codon-optimized signal sequence and N-terminal proline-rich region of SYNPCC7002_A0585), integrated at the amt1-downstream locus, is:

ATGTTTGCCTTCCGTGACTTCCTGACGTTTAGCACGGGCGGTTTGGTCGT
GTTGAGCGGTGGCGGTGTTGCGATTGCACAAACCACCCCTCCGCAGATCG
CCACTCCGGAGCCGTTTATCGGTCAGACGCCGCAGGCACCGCTGCCACCG
CTGGCTGCGCCGTCCGTTGAAAGCCTGGACACCGCGGCTTTCCTGCCGAG
CCTGGGCGGTCTGTCCCAACCGACCACCCTGGCCGCACTGCCTTTGCCGA
GCCCGGAGTTGAACCTGTCGCCTACGGCGCATCTGGGTACCATCCAGGCG
CCAAGCCCGCTGTTGGCGCAAGTGGATACCACTGCGACCCCGAGCCCGAC
CACCGCGATTGACGTCACCCTGCCGACGGCGGAAACGAATCAAACCATTC
CGCTGGTCCAGCCGCTGCCGCCAGACCGCGTCATCAATGAGGACCTGAAC
CAACTGCTGGAGCCGATTGATAACCCGGCAGTTACGGTGCCGCAGGAAGC
GACCGCCGTTACGACCGATAATGTTGTGGATGAGAACCTGATGCAAGTTT
ATCAGCAAGCACGCCTTAGTAACCCGGAATTGCGTAAGTCTGCCGCCGAT
CGTGATGCTGCCTTTGAAAAAATTAATGAAGCGCGCAGTCCATTACTGCC
ACAGCTAGGTTTAGGTGCAGATTACACCTATAGCAACGGCTACCGCGACG
CGAACGGCATCAACTCTAACGCGACCAGTGCGTCCTTGCAGTTAACTCAA
TCCATTTTTGATATGTCGAAATGGCGTGCGTTAACGCTGCAGGAAAAAGC
AGCAGGGATTCAGGACGTCACGTATCAGACCGATCAGCAAACCTTGATCC
TCAACACCGCGACCGCTTATTTCAACGTGTTGAATGCTATTGACGTTCTT
TCCTATACACAGGCACAAAAAGAAGCGATCTACCGTCAATTAGATCAAAC
CACCCAACGTTTTAACGTGGGCCTGGTAGCGATCACCGACGTGCAGAACG
CCCGCGCACAGTACGATACCGTGCTGGCGAACGAAGTGACCGCACGTAAT
AACCTTGATAACGCGGTAGAGCAGCTGCGCCAGATCACCGGTAACTACTA
TCCGGAACTGGCTGCGCTGAATGTCGAAAACTTTAAAACCGACAAACCAC
AGCCGGTTAACGCGCTGCTGAAAGAAGCCGAAAAACGCAACCTGTCGCTG
TTACAGGCACGCTTGAGCCAGGACCTGGCGCGCGAGCAAATTCGCCAGGC
GCAGGATGGTCACTTACCGACTCTGGATTTAACGGCTTCTACCGGGATTT
CTGACACCTCTTATAGCGGTTCGAAAACCCGTGGTGCCGCTGGTACCCAG
TATGACGATAGCAATATGGGCCAGAACAAAGTTGGCCTGAGCTTCTCGCT
GCCGATTTATCAGGGCGGAATGGTTAACTCGCAGGTGAAACAGGCACAGT
ACAACTTTGTCGGTGCCAGCGAGCAACTGGAAAGTGCCCATCGTAGCGTC
GTGCAGACCGTGCGTTCCTCCTTCAACAACATTAATGCATCTATCAGTAG
CATTAACGCCTACAAACAAGCCGTAGTTTCCGCTCAAAGCTCATTAGACG
CGATGGAAGCGGGCTACTCGGTCGGTACGCGTACCATTGTTGATGTGTTG
GATGCGACCACCACGTTGTACAACGCCAAGCAAGAGCTGGCGAATGCGCG
TTATAACTACCTGATTAATCAGCTGAATATTAAGTCAGCTCTGGGTACGT
TGAACGAGCAGGATCTGCTGGCACTGAACAATGCGCTGAGCAAACCGGTT
TCCACTAATCCGGAAAACGTTGCACCGCAAACGCCGGAACAGAATGCTAT
TGCTGATGGTTATGCGCCTGATAGCCCGGCACCAGTCGTTCAGCAAACAT
CCGCACGCACTACCACCAGTAACGGTCATAACCCTTTCCGTAACTGA

### SEQ ID NO:57

The protein sequence encoded by A0585_ProNterm_tolC (native E. coli tolC with its encoded signal sequence replaced by the codon-optimized signal sequence and N-terminal proline-rich region of SYNPCC7002_A0585), integrated at the amt1-downstream locus, is:

MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDENLMQVYQQARLSNPELRKSAAD
RDAAFEKINEARSPLLPQLGLGADYTYSNGYRDANGINSNATSASLQLTQ
SIFDMSKWRALTLQEKAAGIQDVTYQTDQQTLILNTATAYFNVLNAIDVL
SYTQAQKEAIYRQLDQTTQRFNVGLVAITDVQNARAQYDTVLANEVTARN
NLDNAVEQLRQITGNYYPELAALNVENFKTDKPQPVNALLKEAEKRNLSL
LQARLSQDLAREQIRQAQDGHLPTLDLTASTGISDTSYSGSKTRGAAGTQ
YDDSNMGQNKVGLSFSLPIYQGGMVNSQVKQAQYNFVGASEQLESAHR
SVVQTVRSSFNNINASISSINAYKQAVVSAQSSLDAMEAGYSVGTRTIVD
VLDATTTLYNAKQELANARYNYLINQLNIKSALGTLNEQDLLALNNALSK
PVSTNPENVAPQTPEQNAIADGYAPDSPAPVVQQTSARTTTSNGHNPFRN

### SEQ ID NO:58

The DNA sequence of A0585_tolC (native E. coli tolC with its encoded signal sequence replaced by the codon-optimized signal sequence of SYNPCC7002_A0585), integrated at the amt1-downstream locus, is:

ATGTTTGCCTTTCGTGACTTCTTGACCTTCAGCACCGGTGGCCTGGTTGT
CCTGTCCGGCGGTGGTGTTGCGATTGCGGAGAACCTGATGCAAGTTTATC
AGCAAGCACGCCTTAGTAACCCGGAATTGCGTAAGTCTGCCGCCGATCGT
GATGCTGCCTTTGAAAAAATTAATGAAGCGCGCAGTCCATTACTGCCACA
GCTAGGTTTAGGTGCAGATTACACCTATAGCAACGGCTACCGCGACGCGA
ACGGCATCAACTCTAACGCGACCAGTGCGTCCTTGCAGTTAACTCAATCC
ATTTTTGATATGTCGAAATGGCGTGCGTTAACGCTGCAGGAAAAAGCAGC
AGGGATTCAGGACGTCACGTATCAGACCGATCAGCAAACCTTGATCCTCA
ACACCGCGACCGCTTATTTCAACGTGTTGAATGCTATTGACGTTCTTTCC
TATACACAGGCACAAAAAGAAGCGATCTACCGTCAATTAGATCAAACCAC
CCAACGTTTTAACGTGGGCCTGGTAGCGATCACCGACGTGCAGAACGCCC
GCGCACAGTACGATACCGTGCTGGCGAACGAAGTGACCGCACGTAATAAC
CTTGATAACGCGGTAGAGCAGCTGCGCCAGATCACCGGTAACTACTATCC
GGAACTGGCTGCGCTGAATGTCGAAAACTTTAAAACCGACAAACCACAGC
CGGTTAACGCGCTGCTGAAAGAAGCCGAAAAACGCAACCTGTCGCTGTTA
CAGGCACGCTTGAGCCAGGACCTGGCGCGCGAGCAAATTCGCCAGGCGCA
GGATGGTCACTTACCGACTCTGGATTTAACGGCTTCTACCGGGATTTCTG
ACACCTCTTATAGCGGTTCGAAAACCCGTGGTGCCGCTGGTACCCAGTAT
GACGATAGCAATATGGGCCAGAACAAAGTTGGCCTGAGCTTCTCGCTGCC
GATTTATCAGGGCGGAATGGTTAACTCGCAGGTGAAACAGGCACAGTACA
ACTTTGTCGGTGCCAGCGAGCAACTGGAAAGTGCCCATCGTAGCGTCGTG
CAGACCGTGCGTTCCTCCTTCAACAACATTAATGCATCTATCAGTAGCAT
TAACGCCTACAAACAAGCCGTAGTTTCCGCTCAAAGCTCATTAGACGCGA
TGGAAGCGGGCTACTCGGTCGGTACGCGTACCATTGTTGATGTGTTGGAT
GCGACCACCACGTTGTACAACGCCAAGCAAGAGCTGGCGAATGCGCGTTA
TAACTACCTGATTAATCAGCTGAATATTAAGTCAGCTCTGGGTACGTTGA
ACGAGCAGGATCTGCTGGCACTGAACAATGCGCTGAGCAAACCGGTTTCC
ACTAATCCGGAAAACGTTGCACCGCAAACGCCGGAACAGAATGCTATTGC
TGATGGTTATGCGCCTGATAGCCCGGCACCAGTCGTTCAGCAAACATCCG
CACGCACTACCACCAGTAACGGTCATAACCCTTTCCGTAACTGA

### SEQ ID NO:59

The protein sequence encoded by A0585_tolC (native E. coli tolC with its encoded signal sequence replaced by the codon-optimized signal sequence of SYNPCC7002_A0585), integrated at the amt1-downstream locus, is:

MFAFRDFLTFSTGGLVVLSGGGVAIAENLMQVYQQARLSNPELRKSAADR
DAAFEKINEARSPLLPQLGLGADYTYSNGYRDANGINSNATSASLQLTQS
IFDMSKWRALTLQEKAAGIQDVTYQTDQQTLILNTATAYFNVLNAIDVLS
YTQAQKEAIYRQLDQTTQRFNVGLVAITDVQNARAQYDTVLANEVTARNN
LDNAVEQLRQITGNYYPELAALNVENFKTDKPQPVNALLKEAEKRNLSLL
QARLSQDLAREQIRQAQDGHLPTLDLTASTGISDTSYSGSKTRGAAGTQY
DDSNMGQNKVGLSFSLPIYQGGMVNSQVKQAQYNFVGASEQLESAHRSVV
QTVRSSFNNINASISSINAYKQAVVSAQSSLDAMEAGYSVGTRTIVDVLD
ATTTLYNAKQELANARYNYLINQLNIKSALGTLNEQDLLALNNALSKPVS
TNPENVAPQTPEQNAIADGYAPDSPAPVVQQTSARTTTSNGHNPFRN

### SEQ ID NO:60

The DNA sequence of tolC (native E. coli tolC), integrated at the amt1-downstream locus, is:

ATGAAGAAATTGCTCCCCATTCTTATCGGCCTGAGCCTTTCTGGGTTCAG
TTCGTTGAGCCAGGCCGAGAACCTGATGCAAGTTTATCAGCAAGCACGCC
TTAGTAACCCGGAATTGCGTAAGTCTGCCGCCGATCGTGATGCTGCCTTT
GAAAAAATTAATGAAGCGCGCAGTCCATTACTGCCACAGCTAGGTTTAGG
TGCAGATTACACCTATAGCAACGGCTACCGCGACGCGAACGGCATCAACT
CTAACGCGACCAGTGCGTCCTTGCAGTTAACTCAATCCATTTTTGATATG
TCGAAATGGCGTGCGTTAACGCTGCAGGAAAAAGCAGCAGGGATTCAGGA
CGTCACGTATCAGACCGATCAGCAAACCTTGATCCTCAACACCGCGACCG
CTTATTTCAACGTGTTGAATGCTATTGACGTTCTTTCCTATACACAGGCA
CAAAAAGAAGCGATCTACCGTCAATTAGATCAAACCACCCAACGTTTTAA
CGTGGGCCTGGTAGCGATCACCGACGTGCAGAACGCCCGCGCACAGTACG
ATACCGTGCTGGCGAACGAAGTGACCGCACGTAATAACCTTGATAACGCG
GTAGAGCAGCTGCGCCAGATCACCGGTAACTACTATCCGGAACTGGCTGC
GCTGAATGTCGAAAACTTTAAAACCGACAAACCACAGCCGGTTAACGCGC
TGCTGAAAGAAGCCGAAAAACGCAACCTGTCGCTGTTACAGGCACGCTTG
AGCCAGGACCTGGCGCGCGAGCAAATTCGCCAGGCGCAGGATGGTCACTT
ACCGACTCTGGATTTAACGGCTTCTACCGGGATTTCTGACACCTCTTATA
GCGGTTCGAAAACCCGTGGTGCCGCTGGTACCCAGTATGACGATAGCAAT
ATGGGCCAGAACAAAGTTGGCCTGAGCTTCTCGCTGCCGATTTATCAGGG
CGGAATGGTTAACTCGCAGGTGAAACAGGCACAGTACAACTTTGTCGGTG
CCAGCGAGCAACTGGAAAGTGCCCATCGTAGCGTCGTGCAGACCGTGCGT
TCCTCCTTCAACAACATTAATGCATCTATCAGTAGCATTAACGCCTACAA
ACAAGCCGTAGTTTCCGCTCAAAGCTCATTAGACGCGATGGAAGCGGGCT
ACTCGGTCGGTACGCGTACCATTGTTGATGTGTTGGATGCGACCACCACG
TTGTACAACGCCAAGCAAGAGCTGGCGAATGCGCGTTATAACTACCTGAT
TAATCAGCTGAATATTAAGTCAGCTCTGGGTACGTTGAACGAGCAGGATC
TGCTGGCACTGAACAATGCGCTGAGCAAACCGGTTTCCACTAATCCGGAA
AACGTTGCACCGCAAACGCCGGAACAGAATGCTATTGCTGATGGTTATGC
GCCTGATAGCCCGGCACCAGTCGTTCAGCAAACATCCGCACGCACTACCA
CCAGTAACGGTCATAACCCTTTCCGTAACTGA

### SEQ ID NO:61

The protein sequence encoded by tolC (native E. coli to tolC), integrated at the amt1-downstream locus, is:

MKKLLPILIGLSLSGFSSLSQAENLMQVYQQARLSNPELRKSAADRDAAF
EKINEARSPLLPQLGLGADYTYSNGYRDANGINSNATSASLQLTQSIFDM
SKWRALTLQEKAAGIQDVTYQTDQQTLILNTATAYFNVLNAIDVLSYTQA
QKEAIYRQLDQTTQRFNVGLVAITDVQNARAQYDTVLANEVTARNNLDNA
VEQLRQITGNYYPELAALNVENFKTDKPQPVNALLKEAEKRNLSLLQARL
SQDLAREQIRQAQDGHLPTLDLTASTGISDTSYSGSKTRGAAGTQYDDSN
MGQNKVGLSFSLPIYQGGMVNSQVKQAQYNFVGASEQLESAHRSVVQTVR
SSFNNINASISSINAYKQAVVSAQSSLDAMEAGYSVGTRTIVDVLDATTT
LYNAKQELANARYNYLINQLNIKSALGTLNEQDLLALNNALSKPVSTNPE
NVAPQTPEQNAIADGYAPDSPAPVVQQTSARTTTSNGHNPFRN

### SEQ ID NO:62

The DNA sequence of the P(aphII)-P(aphII) promoter, with the kanamycin-resistance cassette indicated in bold, integrated at the amt1-downstream locus, is:

ATGATCACTTGTATTACTGTTTATGTAAGCAGACAGTTTTATTGTTCATG
ATGATATATTTTTATCTTGTGCAATGTAACATCAGAGATTTTGAGACACA
ACGTGGCTTTCCCCCCCCCCCCCTTAATTAAACCCCTATTTGTTTATTTT
TCTAAATACATTCAAATATGTATCCGCTCATGAGACAATAACCCTGATAA
ATGCTTCAATAATATTGAAAAAGGAAGAGTATGATTGAACAAGATGGCCT
GCATGCTGGTTCTCCGGCTGCTTGGGTGGAACGCCTGTTTGGTTACGACT
GGGCTCAGCTGACTATTGGCTGTAGCGATGCAGCGGTTTTCCGTCTGTCT
GCACAGGGTCGTCCGGTTCTGTTTGTGAAAACCGACCTGTCCGGCGCACT
GAACGAACTGCAGGACGAAGCGGCCCGTCTGTCCTGGCTCGCGACGACTG
GTGTTCCGTGCGCGGCAGTTCTGGACGTAGTTACTGAAGCCGGTCGCGAT
TGGCTGCTGCTGGGTGAAGTTCCGGGTCAGGATCTGCTGAGCAGCCACCT
CGCTCCGGCAGAAAAAGTTTCCATCATGGCGGACGCGATGCGCCGTCTGC
ACACCCTGGACCCGGCAACTTGCCCGTTTGACCATCAGGCTAAACACCGT
ATTGAACGTGCACGCACTCGTATGGAAGCGGGTCTGGTTGATCAGGACGA
CCTGGATGAAGAGCACCAGGGCCTCGCACCGGCGGAACTGTTTGCACGTC
TGAAAGCCCGCATGCCGGACGGCGAAGACCTGGTGGTAACGCATGGCGAC
GCTTGTCTGCCAAACATTATGGTGGAAAACGGCCGCTTCTCTGGTTTTAT
TGACTGTGGCCGTCTGGGTGTAGCTGATCGCTATCAGGATATCGCCCTCG
CTACCCGCGATATTGCAGAAGAACTGGGTGGTGAATGGGCTGACCGTTTC
CTGGTGCTGTACGGTATCGCAGCGCCGGATTCTCAGCGCATTGCCTTCTA
CCGTCTGCTGGATGAGTTCTTCTAAGGCGCGCCGAGCATCTCTTCGAAGT
ATTCCAGGCATCAAATAAAACGAAAGGCTCAGTCGAAAGACTGGGCCTTT
CGTTTTATCTGTTGTTTGTCGGTGAACGCTCTCTACTAGAGTCACACTGG
CTCACCTTCGGGTGGGCCTTTCTGCGTTTATAAAGCTTGGGGGGGGGGGG
GAAAGCCACGTTGTGTCTCAAAATCTCTGATGTTACATTGCACAAGATAA
AAATATATCATCATGAACAATAAAACTGTCTGCTTACATAAACAGTAATA
CAAGTGTACAT

### SEQ ID NO:63

The DNA sequence of the P(aphII)-P(psaA) promoter, with the kanamycin-resistance cassette indicated in bold, integrated at the amt1-downstream locus, is:

ATGATCACTTGTATTACTGTTTATGTAAGCAGACAGTTTTATTGTTCATG
ATGATATATTTTTATCTTGTGCAATGTAACATCAGAGATTTTGAGACACA
ACGTGGCTTTCCCCCCCCCCCCCTTAATTAAACCCCTATTTGTTTATTTT
TCTAAATACATTCAAATATGTATCCGCTCATGAGACAATAACCCTGATAA
ATGCTTCAATAATATTGAAAAAGGAAGAGTATGATTGAACAAGATGGCCT
GCATGCTGGTTCTCCGGCTGCTTGGGTGGAACGCCTGTTTGGTTACGACT
GGGCTCAGCTGACTATTGGCTGTAGCGATGCAGCGGTTTTCCGTCTGTCT
GCACAGGGTCGTCCGGTTCTGTTTGTGAAAACCGACCTGTCCGGCGCACT
GAACGAACTGCAGGACGAAGCGGCCCGTCTGTCCTGGCTCGCGACGACTG
GTGTTCCGTGCGCGGCAGTTCTGGACGTAGTTACTGAAGCCGGTCGCGAT
TGGCTGCTGCTGGGTGAAGTTCCGGGTCAGGATCTGCTGAGCAGCCACCT
CGCTCCGGCAGAAAAAGTTTCCATCATGGCGGACGCGATGCGCCGTCTGC
ACACCCTGGACCCGGCAACTTGCCCGTTTGACCATCAGGCTAAACACCGT
ATTGAACGTGCACGCACTCGTATGGAAGCGGGTCTGGTTGATCAGGACGA
CCTGGATGAAGAGCACCAGGGCCTCGCACCGGCGGAACTGTTTGCACGTC
TGAAAGCCCGCATGCCGGACGGCGAAGACCTGGTGGTAACGCATGGCGAC
GCTTGTCTGCCAAACATTATGGTGGAAAACGGCCGCTTCTCTGGTTTTAT
TGACTGTGGCCGTCTGGGTGTAGCTGATCGCTATCAGGATATCGCCCTCG
CTACCCGCGATATTGCAGAAGAACTGGGTGGTGAATGGGCTGACCGTTTC
CTGGTGCTGTACGGTATCGCAGCGCCGGATTCTCAGCGCATTGCCTTCTA
CCGTCTGCTGGATGAGTTCTTCTAAGGCGCGCCGAGCATCTCTTCGAAGT
ATTCCAGGCATCAAATAAAACGAAAGGCTCAGTCGAAAGACTGGGCCTTT
CGTTTTATCTGTTGTTTGTCGGTGAACGCTCTCTACTAGAGTCACACTGG
CTCACCTTCGGGTGGGCCTTTCTGCGTTTATAAAGCTTGCCCCTATATTA
TGCATTTATACCCCCACAATCATGTCAAGAATTCAAGCATCTTAAATAAT
GTTAATTATCGGCAAAGTCTGTGCTCCCCTTCTATAATGCTGAATTGAGC
ATTCGCCTCCTGAACGGTCTTTATTCTTCCATTGTGGGTCTTTAGATTCA
CGATTCTTCACAATCATTGATCTAAGGATCTTTGTAGATTCTCTGTACAT

### SEQ ID NO:64

The DNA sequence of the P(psaA)-P(tsr2142) promoter, with the kanamycin-resistance cassette indicated in bold, integrated at the amt1-downstream locus, is:

ATGATCAGAGAATCTACAAAGATCCTTAGATCAATGATTGTGAAGAATCG
TGAATCTAAAGACCCACAATGGAAGAATAAAGACCGTTCAGGAGGCGAAT
GCTCAATTCAGCATTATAGAAGGGGAGCACAGACTTTGCCGATAATTAAC
ATTATTTAAGATGCTTGAATTCTTGACATGATTGTGGGGGTATAAATGCA
TAATATAGGGGCTTAATTAAACCCCTATTTGTTTATTTTTCTAAATACAT
TCAAATATGTATCCGCTCATGAGACAATAACCCTGATAAATGCTTCAATA
ATATTGAAAAAGGAAGAGTATGATTGAACAAGATGGCCTGCATGCTGGTT
CTCCGGCTGCTTGGGTGGAACGCCTGTTTGGTTACGACTGGGCTCAGCTG
ACTATTGGCTGTAGCGATGCAGCGGTTTTCCGTCTGTCTGCACAGGGTCG
TCCGGTTCTGTTTGTGAAAACCGACCTGTCCGGCGCACTGAACGAACTGC
AGGACGAAGCGGCCCGTCTGTCCTGGCTCGCGACGACTGGTGTTCCGTGC
GCGGCAGTTCTGGACGTAGTTACTGAAGCCGGTCGCGATTGGCTGCTGCT
GGGTGAAGTTCCGGGTCAGGATCTGCTGAGCAGCCACCTCGCTCCGGCAG
AAAAAGTTTCCATCATGGCGGACGCGATGCGCCGTCTGCACACCCTGGAC
CCGGCAACTTGCCCGTTTGACCATCAGGCTAAACACCGTATTGAACGTGC
ACGCACTCGTATGGAAGCGGGTCTGGTTGATCAGGACGACCTGGATGAAG
AGCACCAGGGCCTCGCACCGGCGGAACTGTTTGCACGTCTGAAAGCCCGC
ATGCCGGACGGCGAAGACCTGGTGGTAACGCATGGCGACGCTTGTCTGCC
AAACATTATGGTGGAAAACGGCCGCTTCTCTGGTTTTATTGACTGTGGCC
GTCTGGGTGTAGCTGATCGCTATCAGGATATCGCCCTCGCTACCCGCGAT
ATTGCAGAAGAACTGGGTGGTGAATGGGCTGACCGTTTCCTGGTGCTGTA
CGGTATCGCAGCGCCGGATTCTCAGCGCATTGCCTTCTACCGTCTGCTGG
ATGAGTTCTTCTAAGGCGCGCCGAGCATCTCTTCGAAGTATTCCAGGCAT
CAAATAAAACGAAAGGCTCAGTCGAAAGACTGGGCCTTTCGTTTTATCTG
TTGTTTGTCGGTGAACGCTCTCTACTAGAGTCACACTGGCTCACCTTCGG
GTGGGCCTTTCTGCGTTTATAAAGCTTCCAAGGTGGCTACTTCAACGATA
GCTTAAACTTCGCTGCTCCAGCGAGGGGATTTCACTGGTTTGAATGCTTC
AATGCTTGCCAAAAGAGTGCTACTGGAACTTACAAGAGTGACCCTGCGTC
AGGGGAGCTAGCACTCAAAAAAGACTCCTCCTGTACAT

### SEQ ID NO:65

The DNA sequence of the P(tsr2142)-P(ompR) promoter, with the kanamycin-resistance cassette indicated in bold, integrated at the amt1-downstream locus, is:

ATGATCAGGAGGAGTCTTTTTTGAGTGCTAGCTCCCCTGACGCAGGGTCA
CTCTTGTAAGTTCCAGTAGCACTCTTTTGGCAAGCATTGAAGCATTCAAA
CCAGTGAAATCCCCTCGCTGGAGCAGCGAAGTTTAAGCTATCGTTGAAGT
AGCCACCTTGGTTAATTAAACCCCTATTTGTTTATTTTTCTAAATACATT
CAAATATGTATCCGCTCATGAGACAATAACCCTGATAAATGCTTCAATAA
TATTGAAAAAGGAAGAGTATGATTGAACAAGATGGCCTGCATGCTGGTTC
TCCGGCTGCTTGGGTGGAACGCCTGTTTGGTTACGACTGGGCTCAGCTGA
CTATTGGCTGTAGCGATGCAGCGGTTTTCCGTCTGTCTGCACAGGGTCGT
CCGGTTCTGTTTGTGAAAACCGACCTGTCCGGCGCACTGAACGAACTGCA
GGACGAAGCGGCCCGTCTGTCCTGGCTCGCGACGACTGGTGTTCCGTGCG
CGGCAGTTCTGGACGTAGTTACTGAAGCCGGTCGCGATTGGCTGCTGCTG
GGTGAAGTTCCGGGTCAGGATCTGCTGAGCAGCCACCTCGCTCCGGCAGA
AAAAGTTTCCATCATGGCGGACGCGATGCGCCGTCTGCACACCCTGGACC
CGGCAACTTGCCCGTTTGACCATCAGGCTAAACACCGTATTGAACGTGCA
CGCACTCGTATGGAAGCGGGTCTGGTTGATCAGGACGACCTGGATGAAGA
GCACCAGGGCCTCGCACCGGCGGAACTGTTTGCACGTCTGAAAGCCCGCA
TGCCGGACGGCGAAGACCTGGTGGTAACGCATGGCGACGCTTGTCTGCCA
AACATTATGGTGGAAAACGGCCGCTTCTCTGGTTTTATTGACTGTGGCCG
TCTGGGTGTAGCTGATCGCTATCAGGATATCGCCCTCGCTACCCGCGATA
TTGCAGAAGAACTGGGTGGTGAATGGGCTGACCGTTTCCTGGTGCTGTAC
GGTATCGCAGCGCCGGATTCTCAGCGCATTGCCTTCTACCGTCTGCTGGA
TGAGTTCTTCTAAGGCGCGCCGAGCATCTCTTCGAAGTATTCCAGGCATC
AAATAAAACGAAAGGCTCAGTCGAAAGACTGGGCCTTTCGTTTTATCTGT
TGTTTGTCGGTGAACGCTCTCTACTAGAGTCACACTGGCTCACCTTCGGG
TGGGCCTTTCTGCGTTTATAAAGCTTTAGTACAAAAAGACGATTAACCCC
ATGGGTAAAAGCAGGGGAGCCACTAAAGTTCACAGGTTTACACCGAATTT
TCCATTTGAAAAGTAGTAAATCATACAGAAAACAATCATGTAAAAATTGA
ATACTCTAATGGTTTGATGTCCGAAAAAGTCTAGTTTCTTCTATTCTTCG
ACCAAATCTATGGCAGGGCACTATCACAGAGCTGGCTTAATAATTTGGGA
GAAATGGGTGGGGGCGGACTTTCGTAGAACAATGTAGATTAAAGTACTGT
ACAT

### SEQ ID NO:66

The DNA sequence of the P(nir09)-P(nir07) promoter, with the kanamycin-resistance cassette indicated in bold, integrated at the amt1-downstream locus, is:

ATGATCATCCTCCTCCTAAAGTTCTCATAAAGTTTTTTTGCTCAAGATCA
ATCCACCCGTAGTCTTTGCTAGTTCTACGAGGTCTAGTGATAGCAATTTA
GTAATCTTGAAAGAACCTCTCCCCCAACCCCTCTCTCTTTAAAAGTTCTG
TTCGGAGGAAACCTCCGCTCAGACTTTTCGCTCCGACGCGGAGAGGGGAG
TTTGGCTCCCACTTCCCTACAAGGGAAGGGGGCTGGGGGGTAAGGTTTTT
GATTAATGAATCGATGCTCTAATAGTGAAAAACCAAATATTTAATTTTGT
TGGCGCAGCCTTCCCGCAGGGTATTTTGAATTGATTTATGCTACTTCAAT
GACTGACACGCCGCCGATGTTTCACTGAAGGTAACTCTAGAACTAAACCG
GGGAGAAACTGTAGTCTTTTACATTGGCTAAATTTGTCAAGTGGTTTGTG
TGAATGTTTATGTAACGATTTCGATACTTCTAAGGTTATGTCGGGATCTC
AGGTAAAATAGTATAAGTAGCTACAAAATTCTCGTATTAATGCGTAAGTT
TAATAGAGAATATGCGTTTTCTGCATTACACTTAACTAATGAGTAGTTAA
TTAAACCCCTATTTGTTTATTTTTCTAAATACATTCAAATATGTATCCGC
TCATGAGACAATAACCCTGATAAATGCTTCAATAATATTGAAAAAGGAAG
AGTATGATTGAACAAGATGGCCTGCATGCTGGTTCTCCGGCTGCTTGGGT
GGAACGCCTGTTTGGTTACGACTGGGCTCAGCTGACTATTGGCTGTAGCG
ATGCAGCGGTTTTCCGTCTGTCTGCACAGGGTCGTCCGGTTCTGTTTGTG
AAAACCGACCTGTCCGGCGCACTGAACGAACTGCAGGACGAAGCGGCCCG
TCTGTCCTGGCTCGCGACGACTGGTGTTCCGTGCGCGGCAGTTCTGGACG
TAGTTACTGAAGCCGGTCGCGATTGGCTGCTGCTGGGTGAAGTTCCGGGT
CAGGATCTGCTGAGCAGCCACCTCGCTCCGGCAGAAAAAGTTTCCATCAT
GGCGGACGCGATGCGCCGTCTGCACACCCTGGACCCGGCAACTTGCCCGT
TTGACCATCAGGCTAAACACCGTATTGAACGTGCACGCACTCGTATGGAA
GCGGGTCTGGTTGATCAGGACGACCTGGATGAAGAGCACCAGGGCCTCGC
ACCGGCGGAACTGTTTGCACGTCTGAAAGCCCGCATGCCGGACGGCGAAG
ACCTGGTGGTAACGCATGGCGACGCTTGTCTGCCAAACATTATGGTGGAA
AACGGCCGCTTCTCTGGTTTTATTGACTGTGGCCGTCTGGGTGTAGCTGA
TCGCTATCAGGATATCGCCCTCGCTACCCGCGATATTGCAGAAGAACTGG
GTGGTGAATGGGCTGACCGTTTCCTGGTGCTGTACGGTATCGCAGCGCCG
GATTCTCAGCGCATTGCCTTCTACCGTCTGCTGGATGAGTTCTTCTAAGG
CGCGCCGAGCATCTCTTCGAAGTATTCCAGGCATCAAATAAAACGAAAGG
CTCAGTCGAAAGACTGGGCCTTTCGTTTTATCTGTTGTTTGTCGGTGAAC
GCTCTCTACTAGAGTCACACTGGCTCACCTTCGGGTGGGCCTTTCTGCGT
TTATAAAGCTTGCTTGTAGCAATTGCTACTAAAAACTGCGATCGCTGCTG
AAATGAGCTGGAATTTTGTCCCTCTCAGCTCAAAAAGTATCAATGATTAC
TTAATGTTTGTTCTGCGCAAACTTCTTGCAGAACATGCATGATTTACAAA
AAGTTGTAGTTTCTGTTACCAATTGCGAATCGAGAACTGCCTAATCTGCC
GAGTATGCGATCCTTTAGCAGGAGGATGTACAT

### SEQ ID NO:67

The DNA sequence of the ybhG-ybhF-ybhS-ybhR operon (native E. coli ybhGFSR operon with overlaps between ybhG and ybhF and also between ybhF and ybhS), integrated at the amt1-downstream locus, is:

ATGATGAAAAAACCTGTCGTGATCGGATTGGCGGTAGTGGTACTTGCCGC
CGTGGTTGCCGGAGGCTACTGGTGGTATCAAAGCCGCCAGGATAACGGCC
TGACGCTGTATGGCAACGTGGATATTCGTACGGTAAATCTTAGTTTCCGT
GTTGGGGGGCGCGTTGAATCGCTGGCGGTGGACGAAGGTGATGCTATCAA
AGCGGGCCAGGTGCTGGGCGAACTGGATCACAAGCCGTATGAGATTGCCC
TGATGCAGGCGAAAGCGGGTGTTTCGGTGGCACAGGCGCAGTATGACCTG
ATGCTTGCCGGGTATCGCAATGAAGAAATCGCTCAGGCCGCCGCAGCGGT
GAAACAGGCGCAAGCCGCCTATGACTATGCGCAGAACTTCTATAACCGCC
AGCAAGGGTTGTGGAAAAGCCGCACTATTTCGGCAAATGACCTGGAAAAT
GCCCGCTCCTCGCGCGACCAGGCGCAGGCAACGCTGAAATCAGCACAGGA
TAAATTGCGTCAGTACCGTTCCGGTAACCGTGAACAGGACATCGCTCAGG
CGAAAGCCAGCCTCGAACAGGCGCAGGCGCAACTGGCGCAGGCGGAGTTG
AATTTACAGGACTCAACGTTGATAGCCCCGTCTGATGGCACGCTGTTAAC
GCGCGCGGTGGAGCCAGGCACGGTCCTCAATGAAGGTGGCACGGTGTTTA
CCGTTTCACTAACGCGTCCGGTGTGGGTGCGCGCTTATGTTGATGAACGT
AATCTTGACCAGGCCCAGCCGGGGCGCAAAGTGCTGCTTTATACCGATGG
TCGCCCGGACAAGCCGTATCACGGGCAGATTGGTTTCGTTTCGCCGACTG
CTGAATTTACCCCGAAAACCGTCGAAACGCCGGATCTGCGTACCGACCTC
GTCTATCGCCTGCGTATTGTGGTGACCGACGCCGATGATGCGTTACGCCA
GGGAATGCCAGTGACGGTACAATTCGGTGACGAGGCAGGACATGAATGAT
GCCGTTATCACGCTGAACGGCCTGGAAAAACGCTTTCCGGGCATGGACAA
GCCCGCCGTCGCGCCGCTCGATTGTACCATTCACGCCGGTTATGTGACGG
GGTTGGTGGGGCCGGACGGTGCAGGTAAAACCACGCTGATGCGGATGTTG
GCGGGATTACTGAAACCCGACAGCGGCAGTGCCACGGTGATTGGCTTTGA
TCCGATCAAAAACGACGGCGCGCTGCACGCCGTGCTCGGTTATATGCCGC
AGAAATTTGGTCTGTATGAAGATCTCACGGTGATGGAGAACCTCAATCTG
TACGCGGATTTGCGCAGCGTCACCGGCGAGGCACGTAAGCAAACTTTTGC
TCGCCTGCTGGAGTTTACGTCTCTTGGGCCGTTTACCGGACGCCTGGCGG
GCAAGCTCTCCGGTGGGATGAAACAAAAACTCGGTCTGGCCTGTACCCTG
GTGGGCGAACCGAAAGTGTTGCTGCTCGATGAACCCGGCGTCGGCGTTGA
CCCTATCTCACGGCGCGAACTGTGGCAGATGGTGCATGAGCTGGCGGGCG
AAGGGATGTTAATCCTCTGGAGTACCTCGTATCTCGACGAAGCCGAGCAG
TGCCGTGACGTGTTACTGATGAACGAAGGCGAGTTGCTGTATCAGGGAGA
ACCAAAAGCCCTGACACAAACCATGGCCGGACGCAGCTTTCTGATGACCA
GTCCACACGAGGGCAACCGCAAACTGTTGCAACGCGCCTTGAAACTGCCG
CAGGTCAGCGACGGCATGATTCAGGGGAAATCGGTACGTCTGATCCTCAA
AAAAGAGGCCACACCAGACGATATTCGCCATGCCGACGGGATGCCGGAAA
TCAACATCAACGAAACTACGCCGCGTTTTGAAGATGCGTTTATTGATTTG
CTGGGCGGTGCCGGAACCTCGGAATCGCCGCTGGGCGCAATATTACATAC
GGTAGAAGGCACACCCGGCGAGACGGTGATCGAAGCGAAAGAACTGACCA
AGAAATTTGGGGATTTTGCCGCCACCGATCACGTCAACTTTGCCGTTAAA
CGTGGGGAGATTTTTGGTTTGCTGGGGCCAAACGGCGCGGGTAAATCGAC
CACCTTTAAGATGATGTGCGGTTTGCTGGTGCCGACTTCCGGCCAGGCGC
TGGTGCTGGGGATGGATCTGAAAGAGAGTTCCGGTAAAGCGCGCCAGCAT
CTCGGCTATATGGCGCAAAAATTTTCGCTCTACGGTAACCTGACGGTCGA
ACAGAATTTACGCTTTTTCTCTGGTGTGTATGGCTTACGCGGTCGGGCGC
AGAACGAAAAAATCTCCCGCATGAGCGAGGCGTTCGGCCTGAAAAGTATC
GCCTCCCACGCCACCGATGAACTGCCATTAGGTTTTAAACAGCGGCTGGC
GCTGGCCTGTTCGCTGATGCATGAACCGGACATTCTGTTTCTCGACGAAC
CGACTTCCGGCGTTGACCCCCTCACCCGCCGTGAATTTTGGCTGCACATC
AACAGCATGGTAGAGAAAGGCGTCACGGTGATGGTCACCACCCACTTTAT
GGATGAAGCGGAATATTGCGACCGCATCGGCCTGGTGTACCGCGGGAAAT
TAATCGCCAGCGGCACGCCGGACGATTTGAAAGCACAGTCGGCTAACGAT
GAGCAACCCGATCCCACCATGGAGCAAGCCTTTATTCAGTTGATCCACGA
CTGGGATAAGGAGCATAGCAATGAGTAACCCGATCCTGTCCTGGCGTCGC
GTACGGGCGCTGTGCGTTAAAGAGACGCGGCAGATCGTTCGCGATCCGAG
TAGCTGGCTGATTGCGGTAGTGATCCCGCTGCTACTGCTGTTTATTTTTG
GTTACGGCATTAACCTCGACTCCAGCAAGCTGCGGGTCGGGATTTTACTG
GAACAGCGTAGCGAAGCGGCGCTGGATTTCACCCACACCATGACCGGTTC
GCCCTACATCGACGCCACCATCAGCGATAACCGTCAGGAACTGATCGCCA
AAATGCAGGCGGGGAAAATTCGCGGTCTGGTGGTTATTCCGGTGGATTTT
GCGGAACAGATGGAGCGCGCCAACGCCACCGCACCGATTCAGGTGATCAC
CGACGGCAGTGAGCCGAATACCGCTAACTTTGTACAGGGGTATGTCGAAG
GGATCTGGCAGATCTGGCAAATGCAGCGAGCGGAGGACAACGGGCAGACT
TTTGAACCGCTTATTGATGTACAAACCCGCTACTGGTTTAACCCGGCGGC
GATTAGCCAGCACTTCATTATCCCCGGTGCGGTGACCATTATCATGACGG
TCATCGGCGCGATTCTCACCTCGCTGGTGGTGGCGCGAGAATGGGAACGC
GGCACCATGGAGGCTCTGCTCTCTACGGAGATTACCCGCACGGAACTGCT
GCTGTGTAAGCTGATCCCTTATTACTTTCTCGGGATGCTGGCGATGTTGC
TGTGTATGCTGGTGTCAGTGTTTATTTCGCTGCTGATTCTGTTTTTTATC
TCCAGCCTGTTTTTACTCAGTACCCTGGGGATGGGGCTGCTGATTTCCAC
GATTACCCGCAACCAGTTCAATGCCGCTCAGGTCGCCCTGAACGCCGCTT
TTCTGCCGTCGATTATGCTTTCCGGCTTTATTTTTCAGATCGACAGTATG
CCCGCGGTGATCCGCGCGGTGACGTACATTATTCCCGCTCGTTATTTCGT
CAGCACCCTGCAAAGCCTGTTCCTCGCCGGGAATATTCCAGTGGTGCTGG
TGGTAAACGTGCTGTTTTTGATCGCTTCGGCGGTGATGTTTATCGGCCTG
ACGTGGCTGAAAACCAAACGTCGGCTGGATTAGGGAGAAGAGCATGTTTC
ATCGCTTATGGACGTTAATCCGCAAAGAGTTGCAGTCGTTGCTGCGCGAA
CCGCAAACCCGCGCGATTCTGATTTTACCCGTGCTAATTCAGGTGATCCT
GTTCCCGTTCGCCGCCACGCTGGAAGTGACTAACGCCACCATCGCCATCT
ACGATGAAGATAACGGCGAGCATTCGGTGGAGCTGACCCAACGTTTTGCC
CGCGCCAGCGCCTTTACTCATGTGCTGCTGCTGAAAAGCCCACAGGAGAT
CCGCCCAACCATCGACACACAAAAGGCGTTACTACTGGTGCGTTTCCCGG
CTGACTTCTCGCGCAAACTGGATACCTTCCAGACCGCGCCTTTGCAGTTG
ATCCTCGACGGGCGTAACTCCAACAGTGCGCAAATTGCCGCCAACTACCT
GCAACAGATCGTCAAAAATTATCAGCAGGAGCTGCTGGAAGGAAAACCGA
AACCTAACAACAGCGAGCTGGTGGTACGCAACTGGTATAACCCGAATCTC
GACTACAAATGGTTTGTGGTGCCGTCACTGATCGCCATGATCACCACTAT
CGGCGTAATGATCGTCACTTCACTTTCCGTCGCCCGCGAACGTGAACAAG
GTACGCTCGATCAGCTACTGGTTTCGCCGCTCACCACCTGGCAGATCTTC
ATCGGCAAAGCCGTACCGGCGTTAATTGTCGCCACCTTCCAGGCCACCAT
TGTGCTGGCGATTGGTATCTGGGCGTATCAAATCCCCTTCGCCGGATCGC
TGGCGCTGTTCTACTTTACGATGGTGATTTATGGTTTATCGCTGGTGGGA
TTCGGTCTGTTGATTTCATCACTCTGTTCAACACAACAGCAGGCGTTTAT
CGGCGTGTTTGTCTTTATGATGCCCGCCATTCTCCTTTCCGGTTACGTTT
CTCCGGTGGAAAACATGCCGGTATGGCTGCAAAACCTGACGTGGATTAAC
CCTATTCGCCACTTTACGGACATTACCAAGCAGATTTATTTGAAGGATGC
GAGTCTGGATATTGTGTGGAATAGTTTGTGGCCGCTACTGGTGATAACGG
CCACGACAGGGTCAGCGGCGTACGCGATGTTTAGACGTAAGGTGATGTAA

### SEQ ID NO:68

The DNA sequence encoding the ybhG ORF in the ybhG-ybhF-ybhS-ybhR operon, integrated at the amt1-downstream locus, is:

ATGATGAAAAAACCTGTCGTGATCGGATTGGCGGTAGTGGTACTTGCCGC
CGTGGTTGCCGGAGGCTACTGGTGGTATCAAAGCCGCCAGGATAACGGCC
TGACGCTGTATGGCAACGTGGATATTCGTACGGTAAATCTTAGTTTCCGT
GTTGGGGGGCGCGTTGAATCGCTGGCGGTGGACGAAGGTGATGCTATCAA
AGCGGGCCAGGTGCTGGGCGAACTGGATCACAAGCCGTATGAGATTGCCC
TGATGCAGGCGAAAGCGGGTGTTTCGGTGGCACAGGCGCAGTATGACCTG
ATGCTTGCCGGGTATCGCAATGAAGAAATCGCTCAGGCCGCCGCAGCGGT
GAAACAGGCGCAAGCCGCCTATGACTATGCGCAGAACTTCTATAACCGCC
AGCAAGGGTTGTGGAAAAGCCGCACTATTTCGGCAAATGACCTGGAAAAT
GCCCGCTCCTCGCGCGACCAGGCGCAGGCAACGCTGAAATCAGCACAGGA
TAAATTGCGTCAGTACCGTTCCGGTAACCGTGAACAGGACATCGCTCAGG
CGAAAGCCAGCCTCGAACAGGCGCAGGCGCAACTGGCGCAGGCGGAGTTG
AATTTACAGGACTCAACGTTGATAGCCCCGTCTGATGGCACGCTGTTAAC
GCGCGCGGTGGAGCCAGGCACGGTCCTCAATGAAGGTGGCACGGTGTTTA
CCGTTTCACTAACGCGTCCGGTGTGGGTGCGCGCTTATGTTGATGAACGT
AATCTTGACCAGGCCCAGCCGGGGCGCAAAGTGCTGCTTTATACCGATGG
TCGCCCGGACAAGCCGTATCACGGGCAGATTGGTTTCGTTTCGCCGACTG
CTGAATTTACCCCGAAAACCGTCGAAACGCCGGATCTGCGTACCGACCTC
GTCTATCGCCTGCGTATTGTGGTGACCGACGCCGATGATGCGTTACGCCA
GGGAATGCCAGTGACGGTACAATTCGGTGACGAGGCAGGACATGAATGA

### SEQ ID NO:69

The protein sequence encoded by ybhG ORF in the ybhG-ybhF-ybhS-ybhR operon, integrated at the amt1-downstream locus, is:

MMKKPVVIGLAVVVLAAVVAGGYWWYQSRQDNGLTLYGNVDIRTVNLSFR
VGGRVESLAVDEGDAIKAGQVLGELDHKPYEIALMQAKAGVSVAQAQYDL
MLAGYRNEEIAQAAAAVKQAQAAYDYAQNFYNRQQGLWKSRTISANDLEN
ARSSRDQAQATLKSAQDKLRQYRSGNREQDIAQAKASLEQAQAQLAQAEL
NLQDSTLIAPSDGTLLTRAVEPGTVLNEGGTVFTVSLTRPVWVRAYVDER
NLDQAQPGRKVLLYTDGRPDKPYHGQIGFVSPTAEFTPKTVETPDLRTDL
VYRLRIVVTDADDALRQGMPVTVQFGDEAGHE

### SEQ ID NO:70

The DNA sequence encoding the ybhF ORF in the ybhG-ybhF-ybhS-ybhR operon, integrated at the amt1-downstream locus, is:

ATGAATGATGCCGTTATCACGCTGAACGGCCTGGAAAAACGCTTTCCGGG
CATGGACAAGCCCGCCGTCGCGCCGCTCGATTGTACCATTCACGCCGGTT
ATGTGACGGGGTTGGTGGGGCCGGACGGTGCAGGTAAAACCACGCTGATG
CGGATGTTGGCGGGATTACTGAAACCCGACAGCGGCAGTGCCACGGTGAT
TGGCTTTGATCCGATCAAAAACGACGGCGCGCTGCACGCCGTGCTCGGTT
ATATGCCGCAGAAATTTGGTCTGTATGAAGATCTCACGGTGATGGAGAAC
CTCAATCTGTACGCGGAATTTGCGCAGCGTCACCGGCGAGGCACGTAAGC
AAACTTTTGCTCGCCTGCTGGAGTTTACGTCTCTTGGGCCGTTTACCGGA
CGCCTGGCGGGCAAGCTCTCCGGTGGGATGAAACAAAAACTCGGTCTGGC
CTGTACCCTGGTGGGCGAACCGAAAGTGTTGCTGCTCGATGAACCCGGCG
TCGGCGTTGACCCTATCTCACGGCGCGAACTGTGGCAGATGGTGCATGAG
CTGGCGGGCGAAGGGATGTTAATCCTCTGGAGTACCTCGTATCTCGACGA
AGCCGAGCAGTGCCGTGACGTGTTACTGATGAACGAAGGCGAGTTGCTGT
ATCAGGGAGAACCAAAAGCCCTGACACAAACCATGGCCGGACGCAGCTTT
CTGATGACCAGTCCACACGAGGGCAACCGCAAACTGTTGCAACGCGCCTT
GAAACTGCCGCAGGTCAGCGACGGCATGATTCAGGGGAAATCGGTACGTC
TGATCCTCAAAAAAGAGGCCACACCAGACGATATTCGCCATGCCGACGGG
ATGCCGGAAATCAACATCAACGAAACTACGCCGCGTTTTGAAGATGCGTT
TATTGATTTGCTGGGCGGTGCCGGAACCTCGGAATCGCCGCTGGGCGCAA
TATTACATACGGTAGAAGGCACACCCGGCGAGACGGTGATCGAAGCGAAA
GAACTGACCAAGAAATTTGGGGATTTTGCCGCCACCGATCACGTCAACTT
TGCCGTTAAACGTGGGGAGATTTTTGGTTTGCTGGGGCCAAACGGCGCGG
GTAAATCGACCACCTTTAAGATGATGTGCGGTTTGCTGGTGCCGACTTCC
GGCCAGGCGCTGGTGCTGGGGATGGATCTGAAAGAGAGTTCCGGTAAAGC
GCGCCAGCATCTCGGCTATATGGCGCAAAAATTTTCGCTCTACGGTAACC
TGACGGTCGAACAGAATTTACGCTTTTTCTCTGGTGTGTATGGCTTACGC
GGTCGGGCGCAGAACGAAAAAATCTCCCGCATGAGCGAGGCGTTCGGCCT
GAAAAGTATCGCCTCCCACGCCACCGATGAACTGCCATTAGGTTTTAAAC
AGCGGCTGGCGCTGGCCTGTTCGCTGATGCATGAACCGGACATTCTGTTT
CTCGACGAACCGACTTCCGGCGTTGACCCCCTCACCCGCCGTGAATTTTG
GCTGCACATCAACAGCATGGTAGAGAAAGGCGTCACGGTGATGGTCACCA
CCCACTTTATGGATGAAGCGGAATATTGCGACCGCATCGGCCTGGTGTAC
CGCGGGAAATTAATCGCCAGCGGCACGCCGGACGATTTGAAAGCACAGTC
GGCTAACGATGAGCAACCCGATCCCACCATGGAGCAAGCCTTTATTCAGT
TGATCCACGACTGGGATAAGGAGCATAGCAATGAGTAA

### SEQ ID NO:71

The protein sequence encoded by ybhF ORF in the ybhG-ybhF-ybhS-ybhR operon, integrated at the amt1-downstream locus, is:

MNDAVITLNGLEKRFPGMDKPAVAPLDCTIHAGYVTGLVGPDGAGKTTLM
RMLAGLLKPDSGSATVIGFDPIKNDGALHAVLGYMPQKFGLYEDLTVMEN
LNLYADLRSVTGEARKQTFARLLEFTSLGPFTGRLAGKLSGGMKQKLGLA
CTLVGEPKVLLLDEPGVGVDPISRRELWQMVHELAGEGMLILWSTSYLDE
AEQCRDVLLMNEGELLYQGEPKALTQTMAGRSFLMTSPHEGNRKLLQRAL
KLPQVSDGMIQGKSVRLILKKEATPDDIRHADGMPEININETTPRFEDAF
IDLLGGAGTSESPLGAILHTVEGTPGETVIEAKELTKKFGDFAATDHVNF
AVKRGEIFGLLGPNGAGKSTTFKMMCGLLVPTSGQALVLGMDLKESSGKA
RQHLGYMAQKFSLYGNLTVEQNLRFFSGVYGLRGRAQNEKISRMSEAFGL
KSIASHATDELPLGFKQRLALACSLMHEPDILFLDEPTSGVDPLTRREFW
LHINSMVEKGVTVMVTTHFMDEAEYCDRIGLVYRGKLIASGTPDDLKAQS
ANDEQPDPTMEQAFIQLIHDWDKEHSNE

### SEQ ID NO:72

The DNA sequence encoding the ybhS ORF in the ybhG-ybhF-ybhS-ybhR operon, integrated at the amt1-downstream locus, is:

ATGAGTAACCCGATCCTGTCCTGGCGTCGCGTACGGGCGCTGTGCGTTAA
AGAGACGCGGCAGATCGTTCGCGATCCGAGTAGCTGGCTGATTGCGGTAG
TGATCCCGCTGCTACTGCTGTTTATTTTTGGTTACGGCATTAACCTCGAC
TCCAGCAAGCTGCGGGTCGGGATTTTACTGGAACAGCGTAGCGAAGCGGC
GCTGGATTTCACCCACACCATGACCGGTTCGCCCTACATCGACGCCACCA
TCAGCGATAACCGTCAGGAACTGATCGCCAAAATGCAGGCGGGGAAAATT
CGCGGTCTGGTGGTTATTCCGGTGGATTTTGCGGAACAGATGGAGCGCGC
CAACGCCACCGCACCGATTCAGGTGATCACCGACGGCAGTGAGCCGAATA
CCGCTAACTTTGTACAGGGGTATGTCGAAGGGATCTGGCAGATCTGGCAA
ATGCAGCGAGCGGAGGACAACGGGCAGACTTTTGAACCGCTTATTGATGT
ACAAACCCGCTACTGGTTTAACCCGGCGGCGATTAGCCAGCACTTCATTA
TCCCCGGTGCGGTGACCATTATCATGACGGTCATCGGCGCGATTCTCACC
TCGCTGGTGGTGGCGCGAGAATGGGAACGCGGCACCATGGAGGCTCTGCT
CTCTACGGAGATTACCCGCACGGAACTGCTGCTGTGTAAGCTGATCCCTT
ATTACTTTCTCGGGATGCTGGCGATGTTGCTGTGTATGCTGGTGTCAGTG
TTTATTCTCGGCGTGCCGTATCGCGGGTCGCTGCTGATTCTGTTTTTTAT
CTCCAGCCTGTTTTTACTCAGTACCCTGGGGATGGGGCTGCTGATTTCCA
CGATTACCCGCAACCAGTTCAATGCCGCTCAGGTCGCCCTGAACGCCGCT
TTTCTGCCGTCGATTATGCTTTCCGGCTTTATTTTTCAGATCGACAGTAT
GCCCGCGGTGATCCGCGCGGTGACGTACATTATTCCCGCTCGTTATTTCG
TCAGCACCCTGCAAAGCCTGTTCCTCGCCGGGAATATTCCAGTGGTGCTG
GTGGTAAACGTGCTGTTTTTGATCGCTTCGGCGGTGATGTTTATCGGCCT
GACGTGGCTGAAAACCAAACGTCGGCTGGATTAG

### SEQ ID NO:73

The protein sequence encoded by ybhS ORF in the ybhG-ybhF-ybhS-ybhR operon, integrated at the amt1-downstream locus, is:

MSNPILSWRRVRALCVKETRQIVRDPSSWLIAVVIPLLLLFIFGYGINLD
SSKLRVGILLEQRSEAALDFTHTMTGSPYIDATISDNRQELIAKMQAGKI
RGLVVIPVDFAEQMERANATAPIQVITDGSEPNTANFVQGYVEGIWQIWQ
MQRAEDNGQTFEPLIDVQTRYWFNPAAISQHFIIPGAVTIIMTVIGAILT
SLVVAREWERGTMEALLSTEITRTELLLCKLIPYYFLGMLAMLLCMLVSV
FILGVPYRGSLLILFFISSLFLLSTLGMGLLISTITRNQFNAAQVALNAA
FLPSIMLSGFIFQIDSMPAVIRAVTYIIPARYFVSTLQSLFLAGNIPVVL
VVNVLFLIASAVMFIGLTWLKTKRRLD

### SEQ ID NO:74

The DNA sequence encoding the ybhR ORF in the ybhG-ybhF-ybhS-ybhR operon, integrated at the amt1-downstream locus, is:

ATGTTTCATCGCTTATGGACGTTAATCCGCAAAGAGTTGCAGTCGTTGCT
GCGCGAACCGCAAACCCGCGCGATTCTGATTTTACCCGTGCTAATTCAGG
TGATCCTGTTCCCGTTCGCCGCCACGCTGGAAGTGACTAACGCCACCATC
GCCATCTACGATGAAGATAACGGCGAGCATTCGGTGGAGCTGACCCAACG
TTTTGCCCGCGCCAGCGCCTTTACTCATGTGCTGCTGCTGAAAAGCCCAC
AGGAGATCCGCCCAACCATCGACACACAAAAGGCGTTACTACTGGTGCGT
TTCCCGGCTGACTTCTCGCGCAAACTGGATACCTTCCAGACCGCGCCTTT
GCAGTTGATCCTCGACGGGCGTAACTCCAACAGTGCGCAAATTGCCGCCA
ACTACCTGCAACAGATCGTCAAAAATTATCAGCAGGAGCTGCTGGAAGGA
AAACCGAAACCTAACAACAGCGAGCTGGTGGTACGCAACTGGTATAACCC
GAATCTCGACTACAAATGGTTTGTGGTGCCGTCACTGATCGCCATGATCA
CCACTATCGGCGTAATGATCGTCACTTCACTTTCCGTCGCCCGCGAACGT
GAACAAGGTACGCTCGATCAGCTACTGGTTTCGCCGCTCACCACCTGGCA
GATCTTCATCGGCAAAGCCGTACCGGCGTTAATTGTCGCCACCTTCCAGG
CCACCATTGTGCTGGCGATTGGTATCTGGGCGTATCAAATCCCCTTCGCC
GGATCGCTGGCGCTGTTCTACTTTACGATGGTGATTTATGGTTTATCGCT
GGTGGGATTCGGTCTGTTGATTTCATCACTCTGTTCAACACAACAGCAGG
CGTTTATCGGCGTGTTTGTCTTTATGATGCCCGCCATTCTCCTTTCCGGT
TACGTTTCTCCGGTGGAAAACATGCCGGTATGGCTGCAAAACCTGACGTG
GATTAACCCTATTCGCCACTTTACGGACATTACCAAGCAGATTTATTTGA
AGGATGCGAGTCTGGATATTGTGTGGAATAGTTTGTGGCCGCTACTGGTG
ATAACGGCCACGACAGGGTCAGCGGCGTACGCGATGTTTAGACGTAAGGT
GATGTAA

### SEQ ID NO:75

The protein sequence encoded by ybhR ORF in the ybhG-ybhF-ybhS-ybhR operon, integrated at the amt1-downstream locus, is

MFHRLWTLIRKELQSLLREPQTRAILILPVLIQVILFPFAATLEVTNATI
AIYDEDNGEHSVELTQRFARASAFTHVLLLKSPQEIRPTIDTQKALLLVR
FPADFSRKLDTFQTAPLQLILDGRNSNSAQIAANYLQQIVKNYQQELLEG
KPKPNNSELVVRNWYNPNLDYKWFVVPSLIAMITTIGVMIVTSLSVARER
EQGTLDQLLVSPLTTWQIFIGKAVPALIVATFQATIVLAIGIWAYQIPFA
GSLALFYFTMVIYGLSLVGFGLLISSLCSTQQQAFIGVFVFMMPAILLSG
YVSPVENMPVWLQNLTWINPIRHFTDITKQIYLKDASLDIVWNSLWPLLV
ITATTGSAAYAMFRRKVM

### SEQ ID NO:76

Underlined (2) Upstream, downstream homology regions deletionally targeted to the locus encompassing base pairs 377,985 to 381,565 of the JCC138 chromosome (NCBI accession #NC_{—}010475).
Bold (2) Bidirectional rho-independent transcriptional terminators, incorporated to transcriptionally insulate the integrated divergent tolC-ybhGFSR cassette. The first terminator sequence was derived from the intergenic region between yhdN and rplQ in E. coli MG1655 (Wright J J et al. (1992). Hypersymmetry in a transcriptional terminator of Escherichia coli confers increased efficiency as well as bidirectionality. EMBO 11:1957-1964). The second terminator sequence was derived from a Tn10 bidirectional terminator (Hillen W and Schollmeier K (1983). Nucleotide sequence of the Tn10 encoded tetracycline resistance gene. Nucleic Acids Res. 11:525-539).
Italics Synthetic gentamycin-resistance cassette, containing promoter plus open reading frame aacC1 plus flanking restriction sites
Lowercase E. coli vector backbone (DNA2.0; Menlo Park, Calif.)

ACAACTCGGCTTCCGAGCTTGGCTCCACCATGGTTATATCTGGAGTAACCAGAATTTCGACAACTTCGACGACTATCTC
GGTGCTTTTACCTCCAACCAACGCAAAAACATTAAGCGCGAACGCAAAGCCGTTGACAAAGCAGGTTTATCCCTCAAGA
TGATGACCGGGGACGAAATTCCCGCCCATTACTTCCCACTCATTTATCGTTTCTATAGCAGCACCTGCGACAAATTTTT
TTGGGGGAGTAAATATCTCCGGAAACCCTTTTTTGAAACCCTAGAATCTACCTATCGCCATCGCGTTGTTCTGGCCGCC
GCTTACACGCCAGAAGATGACAAACATCCCGTCGGTTTATCTTTTTGTATCCGTAAAGATGATTATCTTTATGGTCGTT
ATTGGGGGGCCTTTGATGAATATGACTGTCTCCATTTTGAAGCCTGCTATTACAAACCGATCCAATGGGCAATCGAGCA
GGGAATTACGATGTACGATCCGGGCGCTGGCGGAAAACATAAGCGACGACGTGGTTTCCCGGCAACCCCAAACTATAGC
CTCCACCGTTTTTATCAACCCCGCATGGGCCAAGTTTTAGACGCTTATATTGATGAAATTAATGCCATGGAGCAACAGG
AAATTGAAGCGATCAATGCGGATATTCCCTTTAAACGGCAGGAAGTTCAATTGAAAATTTCCTAGCTTCACTAGCCAAA
AGCGCGATCGCCCACCGACCATCCTCCCTTGGGGGAGATGCGGCCGCAACGTAAAAAAACCCGCCCCGGCGGGTTTTTT
TATACCGGTACTGCCCTCGATCTGTA-GAATTCTGCACGCAGATGTGCCGAAGTAAAAAATGCCCTCTTGGGTTATCAA
GAGGGTCATTATAT [symbol] AATTAACGAATCCATGTGGGAGTTTATTCTTGACACAGATATTTATGATATAATAACTGAGTA
AGCTTAACATAAGGAGGAAAAACTAATGTTACGCAGCAGCAACGATGTTACGCAGCAGGGCAGTCGCCCTAAAACAAAG
TTAGGTGGCTCAAGTATGGGCATCATTCGCACATGTAGGCTCGGCCCTGACCAAGTCAAATCCATGCGGGCTGCTCTTG
ATCTTTTCGGTCGTGAGTTCGGAGACGTAGCCACCTACTCCCAACATCAGCCGGACTCCGATTACCTCGGGAACTTGCT
CCGTAGTAAGACATTCATCGCGCTTGCTGCCTTCGACCAAGAAGCGGTTGTTGGCGCTCTCGCGGCTTACGTTCTGCCC
AAGTTTGAGCAGCCGCGTAGTGAGATCTATATCTATGATCTCGCAGTCTCCGGCGAGCACCGGAGGCAGGGCATTGCCA
CCGCGCTCATCAATCTCCTCAAGCATGAGGCCAACGCGCTTGGTGCTTATGTGATCTACGTGCAAGCAGATTACGGTGA
CGATCCCGCAGTGGCTCTCTATACAAAGTTGGGCATACGGGAAGAAGTGATGCACTTTGATATCGACCCAAGTACCGCC
ACCTAGGCGCGCCTGATCAGTTGGTGCTGCATTAGCTAAGAAGGTCAGGAGATATTATTCGACATCTAGCTGACGGCCA
TTGCGATCATAAACGAGGATATCCCACTGGCCATTTTCAGCGGCTTCAAAGGCAATTTTAGACCCATCAGCACTAATGG
TTGGATTACGCACTTCTTGGTTTAAGTTATCGGTTAAATTCCGCTTTTGTTCAAACTCGCGATCATAGAGATAAATATC
AGATTCGCCGCGACGATTGACCGCAAAGACAATGTAGCGACCATCTTCAGAAACGGCAGGATGGGAGGCAATTTCATTT
AGGGTATTGAGGCCCGGTAACAGAATCGTTTGCCTGGTGCTGGTATCAAATAGATAGATATCCTGGGAACCATTGCGGT
CTGAGGCAAAAACGAGGTAGGGTTCGGCGATCGCCGGGTCAAATTCGAGGGCCCGACTATTTAAACTGCGGCCACCGGG
ATCAACGGGAAAATTGACAATGCGCGGATAACCAACGCAGCTCTGGAGCAGCAAACCGAGGCTACCGAGGAAAAAACTG
CGTAGAAAAGAAACATAGCGCATAGGTCAAAGGGAAATCAAAGGGCGGGCGATCGCCAATTTTTCTATAATATTGTCCT
AACAGCACACTAAAACAGAGCCATGCTAGCAAAAATTTGGAGTGCCACCATTGTCGGGGTCGATGCCCTCAGGGTCGGG
GTGGAAGTGGATATTTCCGGCGGCTTACCGAAAATGATGGTGGTCGGACTGCggccggccaaaatgaagtgaagttcct
atactttctagagaataggaacttctatagtgagtcgaataagggcgacacaaaatttattctaaatgcataataaata
ctgataacatcttatagtttgtattatattttgtattatcgttgacatgtataattttgatatcaaaaactgattttcc
ctttattattttcgagatttattttcttaattctctttaacaaactagaaatattgtatatacaaaaaatcataaataa
tagatgaatagtttaattataggtgttcatcaatcgaaaaagcaacgtatcttatttaaagtgcgttgcttttttctca
tttataaggttaaataattctcatatatcaagcaaagtgacaggcgcccttaaatattctgacaaatgctctttcccta
aactccccccataaaaaaacccgccgaagcgggtttttacgttatttgcggattaacgattactcgttatcagaaccgc
ccagggggcccgagcttaagactggccgtcgttttacaacacagaaagagtttgtagaaacgcaaaaaggccatccgtc
aggggccttctgcttagtttgatgcctggcagttccctactctcgccttccgcttcctcgctcactgactcgctgcgct
cggtcgttcggctgcggcgagcggtatcagctcactcaaaggcggtaatacggttatccacagaatcaggggataacgc
aggaaagaacatgtgagcaaaaggccagcaaaaggccaggaaccgtaaaaaggccgcgttgctggcgtttttccatagg
ctccgcccccctgacgagcatcacaaaaatcgacgctcaagtcagaggtggcgaaacccgacaggactataaagatacc
aggcgtttccccctggaagctccctcgtgcgctctcctgttccgaccctgccgcttaccggatacctgtccgcctttct
cccttcgggaagcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtccaacccggtaa
gacacgacttatcgccactggcagcagccactggtaacaggattagcagagcgaggtatgtaggcggtgctacagagtt
cttgaagtggtgggctaactacggctacactagaagaacagtatttggtatctgcgctctgctgaagccagttaccttc
ggaaaaagagttggtagctcttgatccggcaaacaaaccaccgctggtagcggtggtttttttgtttgcaagcagcaga
ttacgcgcagaaaaaaaggatctcaagaagatcctttgatcttttctacggggtctgacgctcagtggaacgacgcgcg
cgtaactcacgttaagggattttggtcatgagcttgcgccgtcccgtcaagtcagcgtaatgctctgcttttagaaaaa
ctcatcgagcatcaaatgaaactgcaatttattcatatcaggattatcaataccatatttttgaaaaagccgtttctgt
aatgaaggagaaaactcaccgaggcagttccataggatggcaagatcctggtatcggtctgcgattccgactcgtccaa
catcaatacaacctattaatttcccctcgtcaaaaataaggttatcaagtgagaaatcaccatgagtgacgactgaatc
cggtgagaatggcaaaagtttatgcatttctttccagacttgttcaacaggccagccattacgctcgtcatcaaaatca
ctcgcatcaaccaaaccgttattcattcgtgattgcgcctgagcgaggcgaaatacgcgatcgctgttaaaaggacaat
tacaaacaggaatcgagtgcaaccggcgcaggaacactgccagcgcatcaacaatattttcacctgaatcaggatattc
ttctaatacctggaacgctgtttttccggggatcgcagtggtgagtaaccatgcatcatcaggagtacggataaaatgc
ttgatggtcggaagtggcataaattccgtcagccagtttagtctgaccatctcatctgtaacatcattggcaacgctac
ctttgccatgtttcagaaacaactctggcgcatcgggcttcccatacaagcgatagattgtcgcacctgattgcccgac
attatcgcgagcccatttatacccatataaatcagcatccatgttggaatttaatcgcggcctcgacgtttcccgttga
atatggctcatattcttcctttttcaatattattgaagcatttatcagggttattgtctcatgagcggatacatatttg
aatgtatttagaaaaataaacaaataggggtcagtgttacaaccaattaaccaattctgaacattatcgcgagcccatt
tatacctgaatatggctcataacaccccttgtttgcctggcggcagtagcgcggtggtcccacctgaccccatgccgaa
ctcagaagtgaaacgccgtagcgccgatggtagtgtggggactccccatgcgagagtagggaactgccaggcatcaaat
aaaacgaaaggctcagtcgaaagactgggcctttcgcccgggctaattagggggtgtcgcccttattcgactctatagt
gaagttcctattctctagaaagtataggaacttctgaagtggggcctgcagg

### SEQ ID NO:77

The DNA sequence encoding A0585_tolC_opt, integrated at the ΔA0358 locus, is:

ATGTTTGCCTTTCGTGACTTCTTGACCTTCAGCACCGGTGGCCTGGTTGT
CCTGTCCGGCGGTGGTGTTGCGATTGCGGAGAATTTGATGCAGGTTTACC
AGCAGGCGCGTCTGTCCAATCCGGAGCTGCGTAAAAGCGCTGCCGACCGT
GATGCCGCGTTTGAGAAGATTAACGAAGCCCGCAGCCCGCTGCTGCCGCA
GCTGGGTTTGGGCGCTGACTACACCTACTCCAACGGCTATCGTGACGCCA
ACGGTATCAATAGCAATGCGACCAGCGCCAGCCTGCAACTGACCCAAAGC
ATTTTTGATATGAGCAAATGGCGCGCTCTGACCCTGCAAGAGAAAGCGGC
AGGTATCCAGGATGTGACCTACCAAACGGACCAGCAGACCCTGATCTTGA
ACACGGCGACCGCGTATTTCAATGTTTTGAACGCAATCGATGTCCTGAGC
TATACCCAGGCCCAGAAGGAAGCGATTTATCGTCAGTTGGATCAGACCAC
CCAGCGCTTCAATGTGGGTCTGGTGGCGATTACGGATGTTCAAAATGCGC
GTGCGCAATACGATACTGTTTTGGCAAACGAAGTGACGGCGCGTAACAAT
CTGGATAATGCCGTTGAACAGCTGCGTCAAATCACGGGCAACTACTATCC
GGAACTGGCAGCACTGAACGTTGAGAATTTCAAGACGGATAAGCCGCAAC
CTGTGAACGCGCTGCTGAAAGAGGCGGAAAAGCGCAATCTGAGCCTGCTG
CAAGCCCGTCTGAGCCAAGACCTGGCGCGTGAGCAGATTCGTCAGGCACA
AGATGGCCACCTGCCAACCCTGGACTTGACGGCATCCACGGGTATCTCGG
ACACCAGCTACTCCGGTAGCAAGACTCGCGGTGCAGCAGGTACGCAGTAT
GACGACTCTAACATGGGTCAAAACAAAGTCGGCCTGTCTTTCAGCCTGCC
GATCTACCAAGGTGGCATGGTTAATTCTCAAGTTAAACAGGCGCAATACA
ACTTTGTCGGCGCGAGCGAACAGCTGGAGAGCGCTCACCGTAGCGTGGTC
CAGACCGTCCGTTCTTCTTTTAACAACATTAACGCGAGCATCAGCAGCAT
TAACGCATACAAACAAGCGGTGGTGAGCGCGCAATCGAGCCTGGACGCAA
TGGAGGCGGGTTACAGCGTCGGTACGCGCACCATTGTCGACGTGCTGGAT
GCAACTACCACCCTGTATAATGCAAAGCAAGAACTGGCAAATGCGCGCTA
CAACTATCTGATTAACCAGCTGAATATCAAATCCGCGCTGGGCACGCTGA
ACGAGCAGGATCTGCTGGCATTGAACAACGCGCTGAGCAAGCCGGTAAGC
ACGAATCCGGAGAACGTCGCCCCACAAACCCCGGAACAGAATGCTATCGC
GGACGGCTATGCCCCGGACAGCCCGGCTCCGGTTGTGCAGCAGACTAGCG
CTCGCACCACCACCAGCAATGGTCATAATCCGTTCCGTAATTAA

### SEQ ID NO:78

The protein sequence encoded by A0585_tolC_opt, integrated at the ΔA0358 is:

MFAFRDFLTFSTGGLVVLSGGGVAIAENLMQVYQQARLSNPELRKSAADR
DAAFEKINEARSPLLPQLGLGADYTYSNGYRDANGINSNATSASLQLTQS
IFDMSKWRALTLQEKAAGIQDVTYQTDQQTLILNTATAYFNVLNAIDVLS
YTQAQKEAIYRQLDQTTQRFNVGLVAITDVQNARAQYDTVLANEVTARNN
LDNAVEQLRQITGNYYPELAALNVENFKTDKPQPVNALLKEAEKRNLSLL
QARLSQDLAREQIRQAQDGHLPTLDLTASTGISDTSYSGSKTRGAAGTQY
DDSNMGQNKVGLSFSLPIYQGGMVNSQVKQAQYNFVGASEQLESAHRSVV
QTVRSSFNNINASISSINAYKQAVVSAQSSLDAMEAGYSVGTRTIVDVLD
ATTTLYNAKQELANARYNYLINQLNIKSALGTLNEQDLLALNNALSKPVS
TNPENVAPQTPEQNAIADGYAPDSPAPVVQQTSARTTTSNGHNPFRN

### SEQ ID NO:79

The DNA sequence encoding A0318_tolC_opt, integrated at the ΔA0358 locus, is:

ATGCAGAAACAACAAAATCTGGACTACTTTAGCCCGCAGGCCCTGGCCCT
GTGGGCTGCGATTGCGAGCTTGGGTGTTATGTCCCCTGCGCATGCGGAGA
ATTTGATGCAGGTTTACCAGCAGGCGCGTCTGTCCAATCCGGAGCTGCGT
AAAAGCGCTGCCGACCGTGATGCCGCGTTTGAGAAGATTAACGAAGCCCG
CAGCCCGCTGCTGCCGCAGCTGGGTTTGGGCGCTGACTACACCTACTCCA
ACGGCTATCGTGACGCCAACGGTATCAATAGCAATGCGACCAGCGCCAGC
CTGCAACTGACCCAAAGCATTTTTGATATGAGCAAATGGCGCGCTCTGAC
CCTGCAAGAGAAAGCGGCAGGTATCCAGGATGTGACCTACCAAACGGACC
AGCAGACCCTGATCTTGAACACGGCGACCGCGTATTTCAATGTTTTGAAC
GCAATCGATGTCCTGAGCTATACCCAGGCCCAGAAGGAAGCGATTTATCG
TCAGTTGGATCAGACCACCCAGCGCTTCAATGTGGGTCTGGTGGCGATTA
CGGATGTTCAAAATGCGCGTGCGCAATACGATACTGTTTTGGCAAACGAA
GTGACGGCGCGTAACAATCTGGATAATGCCGTTGAACAGCTGCGTCAAAT
CACGGGCAACTACTATCCGGAACTGGCAGCACTGAACGTTGAGAATTTCA
AGACGGATAAGCCGCAACCTGTGAACGCGCTGCTGAAAGAGGCGGAAAAG
CGCAATCTGAGCCTGCTGCAAGCCCGTCTGAGCCAAGACCTGGCGCGTGA
GCAGATTCGTCAGGCACAAGATGGCCACCTGCCAACCCTGGACTTGACGG
CATCCACGGGTATCTCGGACACCAGCTACTCCGGTAGCAAGACTCGCGGT
GCAGCAGGTACGCAGTATGACGACTCTAACATGGGTCAAAACAAAGTCGG
CCTGTCTTTCAGCCTGCCGATCTACCAAGGTGGCATGGTTAATTCTCAAG
TTAAACAGGCGCAATACAACTTTGTCGGCGCGAGCGAACAGCTGGAGAGC
GCTCACCGTAGCGTGGTCCAGACCGTCCGTTCTTCTTTTAACAACATTAA
CGCGAGCATCAGCAGCATTAACGCATACAAACAAGCGGTGGTGAGCGCGC
AATCGAGCCTGGACGCAATGGAGGCGGGTTACAGCGTCGGTACGCGCACC
ATTGTCGACGTGCTGGATGCAACTACCACCCTGTATAATGCAAAGCAAGA
ACTGGCAAATGCGCGCTACAACTATCTGATTAACCAGCTGAATATCAAAT
CCGCGCTGGGCACGCTGAACGAGCAGGATCTGCTGGCATTGAACAACGCG
CTGAGCAAGCCGGTAAGCACGAATCCGGAGAACGTCGCCCCACAAACCCC
GGAACAGAATGCTATCGCGGACGGCTATGCCCCGGACAGCCCGGCTCCGG
TTGTGCAGCAGACTAGCGCTCGCACCACCACCAGCAATGGTCATAATCCG
TTCCGTAATTAA

### SEQ ID NO:80

The protein sequence encoded by A0318_tolC_opt, integrated at the ΔA0358 is:

MQKQQNLDYFSPQALALWAAIASLGVMSPAHAENLMQVYQQARLSNPELR
KSAADRDAAFEKINEARSPLLPQLGLGADYTYSNGYRDANGINSNATSAS
LQLTQSIFDMSKWRALTLQEKAAGIQDVTYQTDQQTLILNTATAYFNVLN
AIDVLSYTQAQKEAIYRQLDQTTQRFNVGLVAITDVQNARAQYDTVLANE
VTARNNLDNAVEQLRQITGNYYPELAALNVENFKTDKPQPVNALLKEAEK
RNLSLLQARLSQDLAREQIRQAQDGHLPTLDLTASTGISDTSYSGSKTRG
AAGTQYDDSNMGQNKVGLSFSLPIYQGGMVNSQVKQAQYNFVGASEQLES
AHRSVVQTVRSSFNNINASISSINAYKQAVVSAQSSLDAMEAGYSVGTRT
IVDVLDATTTLYNAKQELANARYNYLINQLNIKSALGTLNEQDLLALNNA
LSKPVSTNPENVAPQTPEQNAIADGYAPDSPAPVVQQTSARTTTSNGHNP
FRN

### SEQ ID NO:81

The DNA sequence encoding A0585_ProNterm_tolC_opt, integrated at the ΔA0358 locus, is:

ATGTTTGCCTTCCGTGACTTCCTGACGTTTAGCACGGGCGGTTTGGTCGT
GTTGAGCGGTGGCGGTGTTGCGATTGCACAAACCACCCCTCCGCAGATCG
CCACTCCGGAGCCGTTTATCGGTCAGACGCCGCAGGCACCGCTGCCACCG
CTGGCTGCGCCGTCCGTTGAAAGCCTGGACACCGCGGCTTTCCTGCCGAG
CCTGGGCGGTCTGTCCCAACCGACCACCCTGGCCGCACTGCCTTTGCCGA
GCCCGGAGTTGAACCTGTCGCCTACGGCGCATCTGGGTACCATCCAGGCG
CCAAGCCCGCTGTTGGCGCAAGTGGATACCACTGCGACCCCGAGCCCGAC
CACCGCGATTGACGTCACCCTGCCGACGGCGGAAACGAATCAAACCATTC
CGCTGGTCCAGCCGCTGCCGCCAGACCGCGTCATCAATGAGGACCTGAAC
CAACTGCTGGAGCCGATTGATAACCCGGCAGTTACGGTGCCGCAGGAAGC
GACCGCCGTTACGACCGATAATGTTGTGGATGAGAATTTGATGCAGGTTT
ACCAGCAGGCGCGTCTGTCCAATCCGGAGCTGCGTAAAAGCGCTGCCGAC
CGTGATGCCGCGTTTGAGAAGATTAACGAAGCCCGCAGCCCGCTGCTGCC
GCAGCTGGGTTTGGGCGCTGACTACACCTACTCCAACGGCTATCGTGACG
CCAACGGTATCAATAGCAATGCGACCAGCGCCAGCCTGCAACTGACCCAA
AGCATTTTTGATATGAGCAAATGGCGCGCTCTGACCCTGCAAGAGAAAGC
GGCAGGTATCCAGGATGTGACCTACCAAACGGACCAGCAGACCCTGATCT
TGAACACGGCGACCGCGTATTTCAATGTTTTGAACGCAATCGATGTCCTG
AGCTATACCCAGGCCCAGAAGGAAGCGATTTATCGTCAGTTGGATCAGAC
CACCCAGCGCTTCAATGTGGGTCTGGTGGCGATTACGGATGTTCAAAATG
CGCGTGCGCAATACGATACTGTTTTGGCAAACGAAGTGACGGCGCGTAAC
AATCTGGATAATGCCGTTGAACAGCTGCGTCAAATCACGGGCAACTACTA
TCCGGAACTGGCAGCACTGAACGTTGAGAATTTCAAGACGGATAAGCCGC
AACCTGTGAACGCGCTGCTGAAAGAGGCGGAAAAGCGCAATCTGAGCCTG
CTGCAAGCCCGTCTGAGCCAAGACCTGGCGCGTGAGCAGATTCGTCAGGC
ACAAGATGGCCACCTGCCAACCCTGGACTTGACGGCATCCACGGGTATCT
CGGACACCAGCTACTCCGGTAGCAAGACTCGCGGTGCAGCAGGTACGCAG
TATGACGACTCTAACATGGGTCAAAACAAAGTCGGCCTGTCTTTCAGCCT
GCCGATCTACCAAGGTGGCATGGTTAATTCTCAAGTTAAACAGGCGCAAT
ACAACTTTGTCGGCGCGAGCGAACAGCTGGAGAGCGCTCACCGTAGCGTG
GTCCAGACCGTCCGTTCTTCTTTTAACACATTACGCGAGCATCAGCAGCA
TTAACGCATACAACAGCGGTGGTGAGCGCGCAATCGAGCCTGGACGCAAT
GGAGGCGGGTTACAGCGTCGGTACGCGCACCATTGTCGACGTGCTGGATG
CAACTACCACCCTGTATAATGCAAAGCAAGAACTGGCAAATGCGCGCTAC
AACTATCTGATTAACCAGCTGAATATCAAATCCGCGCTGGGCACGCTGAA
CGAGCAGGATCTGCTGGCATTGAACAACGCGCTGAGCAAGCCGGTAAGCA
CGAATCCGGAGAACGTCGCCCCACAAACCCCGGAACAGAATGCTATCGCG
GACGGCTATGCCCCGGACAGCCCGGCTCCGGTTGTGCAGCAGACTAGCGC
TCGCACCACCACCAGCAATGGTCATAATCCGTTCCGTAATTAA

### SEQ ID NO:82

The protein sequence encoded by A0585_ProNterm_tolC_opt, integrated at the ΔA0358 is:

MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDENLMQVYQQARLSNPELRKSAAD
RDAAFEKINEARSPLLPQLGLGADYTYSNGYRDANGINSNATSASLQLTQ
SIFDMSKWRALTLQEKAAGIQDVTYQTDQQTLILNTATAYFNVLNAIDVL
SYTQAQKEAIYRQLDQTTQRFNVGLVAITDVQNARAQYDTVLANEVTARN
NLDNAVEQLRQITGNYYPELAALNVENFKTDKPQPVNALLKEAEKRNLSL
LQARLSQDLAREQIRQAQDGHLPTLDLTASTGISDTSYSGSKTRGAAGTQ
YDDSNMGQNKVGLSFSLPIYQGGMVNSQVKQAQYNFVGASEQLESAHRSV
VQTVRSSFNNINASISSINAYKQAVVSAQSSLDAMEAGYSVGTRTIVDVL
DATTTLYNAKQELANARYNYLINQLNIKSALGTLNEQDLLALNNALSKPV
STNPENVAPQTPEQNAIADGYAPDSPAPVVQQTSARTTTSNGHNPFRN

### SEQ ID NO:83

The DNA sequence encoding A0318_ProNterm_tolC_opt, integrated at the ΔA0358 locus, is:

ATGCAAAAACAACAGAATCTGGACTACTTTAGCCCGCAGGCGTTGGCACT
GTGGGCGGCTATTGCTTCCCTGGGTGTTATGAGCCCGGCACACGCGGAGC
CGCGTAGCGAGGGCAGCCATTCTGATCCGCTGGTTCCGACCGCGACGCAG
GTCGTGGTTCCGGCGCTGCCGGTGGAGGACGTTGCGCCGACCGCCGCACC
GGCATCGCAGACCCCGGCTCCTCAGAGCGAAAACTTGGCGCAATCCAGCA
CCCAAGCCGTCACGAGCCCTGTGGCGCAGGCGCAGGAAGCCCCGCAAGAC
AGCAATCTGCCGCAACTGTATGCCCAGCAGCAAGGTAACCCAAATGCCCA
ACAGGCGAACCCGGAGAATTTGATGCAGGTTTACCAGCAGGCGCGTCTGT
CCAATCCGGAGCTGCGTAAAAGCGCTGCCGACCGTGATGCCGCGTTTGAG
AAGATTAACGAAGCCCGCAGCCCGCTGCTGCCGCAGCTGGGTTTGGGCGC
TGACTACACCTACTCCAACGGCTATCGTGACGCCAACGGTATCAATAGCA
ATGCGACCAGCGCCAGCCTGCAACTGACCCAAAGCATTTTTGATATGAGC
AAATGGCGCGCTCTGACCCTGCAAGAGAAAGCGGCAGGTATCCAGGATGT
GACCTACCAAACGGACCAGCAGACCCTGATCTTGAACACGGCGACCGCGT
ATTTCAATGTTTTGAACGCAATCGATGTCCTGAGCTATACCCAGGCCCAG
AAGGAAGCGATTTATCGTCAGTTGGATCAGACCACCCAGCGCTTCAATGT
GGGTCTGGTGGCGATTACGGATGTTCAAAATGCGCGTGCGCAATACGATA
CTGTTTTGGCAAACGAAGTGACGGCGCGTAACAATCTGGATAATGCCGTT
GAACAGCTGCGTCAAATCACGGGCAACTACTATCCGGAACTGGCAGCACT
GAACGTTGAGAATTTCAAGACGGATAAGCCGCAACCTGTGAACGCGCTGC
TGAAAGAGGCGGAAAAGCGCAATCTGAGCCTGCTGCAAGCCCGTCTGAGC
CAAGACCTGGCGCGTGAGCAGATTCGTCAGGCACAAGATGGCCACCTGCC
AACCCTGGACTTGACGGCATCCACGGGTATCTCGGACACCAGCTACTCCG
GTAGCAAGACTCGCGGTGCAGCAGGTACGCAGTATGACGACTCTAACATG
GGTCAAAACAAAGTCGGCCTGTCTTTCAGCCTGCCGATCTACCAAGGTGG
CATGGTTAATTCTCAAGTTAAACAGGCGCAATACAACTTTGTCGGCGCGA
GCGAACAGCTGGAGAGCGCTCACCGTAGCGTGGTCCAGACCGTCCGTTCT
TCTTTTAACAACATTAACGCGAGCATCAGCAGCATTAACGCATACAAACA
AGCGGTGGTGAGCGCGCAATCGAGCCTGGACGCAATGGAGGCGGGTTACA
GCGTCGGTACGCGCACCATTGTCGACGTGCTGGATGCAACTACCACCCTG
TATAATGCAAAGCAAGAACTGGCAAATGCGCGCTACAACTATCTGATTAA
CCAGCTGAATATCAAATCCGCGCTGGGCACGCTGAACGAGCAGGATCTGC
TGGCATTGAACAACGCGCTGAGCAAGCCGGTAAGCACGAATCCGGAGAAC
GTCGCCCCACAAACCCCGGAACAGAATGCTATCGCGGACGGCTATGCCCC
GGACAGCCCGGCTCCGGTTGTGCAGCAGACTAGCGCTCGCACCACCACCA
GCAATGGTCATAATCCGTTCCGTAATTAA

### SEQ ID NO:84

The protein sequence encoded by A0318_ProNterm_tolC_opt, integrated at the ΔA0358 is:

MQKQQNLDYFSPQALALWAAIASLGVMSPAHAEPRSEGSHSDPLVPTATQ
VVVPALPVEDVAPTAAPASQTPAPQSENLAQSSTQAVTSPVAQAQEAPQD
SNLPQLYAQQQGNPNAQQANPENLMQVYQQARLSNPELRKSAADRDAAFE
KINEARSPLLPQLGLGADYTYSNGYRDANGINSNATSASLQLTQSIFDMS
KWRALTLQEKAAGIQDVTYQTDQQTLILNTATAYFNVLNAIDVLSYTQAQ
KEAIYRQLDQTTQRFNVGLVAITDVQNARAQYDTVLANEVTARNNLDNAV
EQLRQITGNYYPELAALNVENFKTDKPQPVNALLKEAEKRNLSLLQARLS
QDLAREQIRQAQDGHLPTLDLTASTGISDTSYSGSKTRGAAGTQYDDSNM
GQNKVGLSFSLPIYQGGMVNSQVKQAQYNFVGASEQLESAHRSVVQTVRS
SFNNINASISSINAYKQAVVSAQSSLDAMEAGYSVGTRTIVDVLDATTTL
YNAKQELANARYNYLINQLNIKSALGTLNEQDLLALNNALSKPVSTNPEN
VAPQTPEQNAIADGYAPDSPAPVVQQTSARTTTSNGHNPFRN

### SEQ ID NO:85

The DNA sequence encoding hybrid_A0585, integrated at the ΔA0358 locus, is:

ATGTTCGCTTTTCGCGACTTTCTGACCTTTTCGACTGGCGGCCTGGTCGT
TCTGTCCGGTGGCGGTGTTGCGATTGCGCAGACCACCCCTCCGCAGATCG
CGACCCCGGAACCGTTTATCGGTCAGACGCCGCAAGCCCCGCTGCCTCCG
CTGGCCGCTCCGAGCGTTGAGAGCCTGGATACCGCGGCTTTCTTGCCGTC
GCTGGGCGGTCTGAGCCAACCGACCACGCTGGCAGCACTGCCGCTGCCGA
GCCCAGAGCTGAATCTGTCCCCGACCGCCCACCTGGGTACGATCCAAGCC
CCGAGCCCGTTGCTGGCGCAAGTGGATACCACCGCTACGCCGAGCCCGAC
GACCGCCATTGATGTGACTTTGCCGACCGCGGAAACGAATCAAACGATTC
CGCTGGTTCAACCGCTGCCGCCTGATCGTGTGATTAACGAAGATCTGAAC
CAGCTGCTGGAACCGATCGACAATCCGGCGGTCACCGTCCCGCAAGAGGC
AACCGCGGTGACCACCGATAATGTGGTTGACCTGACGCTCGAGGAAACGA
TCCGCCTGGCACTGGAGCGCAACGAAACCTTGCAAGAGGCGCGTCTGAAC
TATGACCGCAGCGAGGAGCTGGTGCGTGAGGCGATTGCGGCTGAGTACCC
GAATTTGTCGAACCAGGTCGACATTACCCGTACTGACAGCGCGAACGGTG
AGCTGCAAGCTCGTCGTCTGGGTGGTGACAATAATGCCACCACCGCCATC
AATGGTCGCCTGGAAGTGAGCTACGACATCTATACCGGCGGTCGCCGTAG
CGCGCAGATTGAGGCGGCACAGACCCAGCTGCAAATTGCCGAGCTGGATA
TCGAACGCCTGACCGAGGAGACTCGTCTGGCTGCGGCGGTGAATTACTAT
AATCTGCAATCTGCGGACGCGCAGGTTGTTATTGAACAGAGCTCAGTTTT
TGATGCAACCCAGCAACTGGATCAAACTACTCAGCGTTTCAACGTGGGTC
TGGTGGCAATTACGGACGTTCAGAACGCGCGTGCAGAGCTGGCTAGCGCC
CAACAGCGTCTGACGCGCGCTGAAGCCACCCAGCGCACGGCACGTCGTCA
ACTGGCGCAGTTGCTGAGCTTGGAGCCGACCATCGACCCGCGCACGGCCG
ACGAGATCAACCTGGCGGGTCGTTGGGAGATCAGCCTGGAGGAAACCATT
GTTCTGGCCTTGCAGAATCGTCAAGAACTGCGTCAACAGCTGCTGCAACG
TGAGGTGGATGGCTACCAGGAGCGCATCGCGTTGGCGGCAGTCCGCCCAC
TGGTGAGCGTCTTTGCGAATTATGACGTCCTGGAGGTATTTGACGATAGC
TTGGGCCCAGCGGATGGTTTGACTGTCGGTGCTCGTATGCGTTGGAACTT
CTTCGACGGCGGTGCTGCGGCAGCGCGTGCCAACCAGGAACAAGTGGATC
AGGCCATCGCGGAGAATCGCTTTGCAAACCAACGCAACCAGATTCGTCTG
GCAGTCGAAACCGCATATTACGACTTCGAAGCGAGCGAACAGAACATTAC
CACGGCCGCAGCGGCCGTAACGTTAGCAGAAGAAAGCCTGGACGCGATGG
AGGCTGGTTACTCCGTTGGTACCCGCACTATCGTTGATGTCCTGGATGCG
ACGACGGGCCTGAATACGGCCCGGGGTAACTACCTGCAAGCGGTTACCGA
TTACAACCGTGCGTTCGCGCAGCTGAAGCGTGAAGTTGGCCTGGGCGACG
CCGTCATTGCGCCTGCGGCTCCGTAA

### SEQ ID NO:86

The protein sequence encoded by hybrid_A0585, integrated at the ΔA0358 is:

MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDLTLEETIRLALERNETLQEARLN
YDRSEELVREAIAAEYPNLSNQVDITRTDSANGELQARRLGGDNNATTAI
NGRLEVSYDIYTGGRRSAQIEAAQTQLQIAELDIERLTEETRLAAAVNYY
NLQSADAQVVIEQSSVFDATQQLDQTTQRFNVGLVAITDVQNARAELASA
QQRLTRAEATQRTARRQLAQLLSLEPTIDPRTADEINLAGRWEISLEETI
VLALQNRQELRQQLLQREVDGYQERIALAAVRPLVSVFANYDVLEVFDDS
LGPADGLTVGARMRWNFFDGGAAAARANQEQVDQAIAENRFANQRNQIRL
AVETAYYDFEASEQNITTAAAAVTLAEESLDAMEAGYSVGTRTIVDVLDA
TTGLNTARGNYLQAVTDYNRAFAQLKREVGLGDAVIAPAAP

### SEQ ID NO:87

The DNA sequence encoding hybrid_{—}1761, integrated at the ΔA0358 locus, is:

ATGGCGGCCTTCTTGTACCGCCTGAGCTTCCTGAGCGCGCTGGCAATCGC
GGCTCACGGCGTTACCCCACCGACCGCCATCGCTGAGCTCGCGGAGGCGA
CCACCGCAGAACCAACCCCGACCGTCGCCCAAGCTACGACCCCACCGGCT
ACCACGCCGACGACCACCCCGGCTCCTGGCCCGGTCAAAGAAGTCGTGCC
GGACGCGAATCTGCTGAAGGAGCTGCAAGCCAACCCGAACCCGTTCCAGC
TGCCGAACCAGCCGAATCAGGTGAAAACCGAGGCCCTGCAACCGTTGACC
CTCGAGCAGGCTCTGAATCTGGCGCGTTTGAATAACCCGCAGATTCAGGT
GCGTCAGCTGCAAGTTCAGCAACGCCAGGCGGCATTGCGTGGTACGGAAG
CAGCCCTGTACCCTACTCTGGGCCTGCAAGGTACGGCAGGCTATCAGCAA
AACGGCACGCGCTTGAACGTGACCGAGGGTACCCCGACGCAGCCGACCGG
CAGCTCCCTGTTCACGACCCTGGGTGAGAGCAGCATCGGCGCAACCCTGA
ACCTGAATTACACGATTTTTGATTTCGTCCGTGGTGCACAACTGGCGGCC
AGCCGTGACCAGGTGACGCAGGCGGAATTGGATCTGGAGGCGGCACTGGA
GGACCTGCAACTGACTGTTTCGGAAGCGTACTATCGTTTGCAGAATGCGG
ATCAATTGGTCCGCATCGCTCGCGAGTCTGTCGTCGCGTCCGAGCGTCAG
TTGGATCAGACCACCCAACGCTTTAATGTTGGCCTGGTGGCGATCACGGA
TGTGCAAAATGCCCGTGCCCAGCTGGCACAAGACCAGCAGAATCTGGTCG
ACTCGATCGGTAACCAGGACAAGGCGCGTCGCGCGCTGGTTCAGGCACTG
AACCTGCCGCAGAATGTTAATGTCCTGACCGCTGATCCGGTTGAACTGGC
TGCGCCGTGGAATCTGAGCCTGGATGAGTCTATTGTTCTGGCTTTCCAGA
ACCGTCCGGAGCTGGAGCGCGAGGTGTTGCAACGTAACATTAGCTATAAC
CAAGCGCAAGCAGCTCGCGGTCAAGTTCTGCCGCAGCTGGGTCTGCAAGC
GAGCTACGGCGTCAACGGTGCCATCAATTCTAATCTGCGTAGCGGTAGCC
AAGCGCTGACCTTCCCGAGCCCGACTCTGACGAACACGAGCAGCTATAAC
TACTCCATTGGTCTGGTTTTGAATGTGCCGCTGTTTGACGGCGGTCTGGC
GAACGCGAACGCACAGCAACAGGAATTGAACGGTCAGATTGCTGAACAAA
ACTTTGTGCTGACCCGCAATCAGATTCGTACGGACGTCGAGACTGCCTTT
TACGACCTGCAAACCAATCTGGCAAATATCGGTACCACCCGTAAAGCGGT
GGAACAAGCTCGTGAAAGCCTGGACGCGATGGAAGCGGGTTATAGCGTGG
GTACCCGTACCATTGTTGACGTTCTGGATGCCACGACGGATCTGACCCGT
GCAGAGGCGAATGCGCTGAATGCCATCACCGCGTATAACCTGGCACTGGC
GCGTATTAAGCGCGCAGTGAGCAACGTTAACAACCTGGCGCGTGCGGGTG
GCTAA

### SEQ ID NO:88

The protein sequence encoded by hybrid_{—}1761, integrated at the ΔA0358 is:

MAAFLYRLSFLSALAIAAHGVTPPTAIAELAEATTAEPTPTVAQATTPPA
TTPTTTPAPGPVKEVVPDANLLKELQANPNPFQLPNQPNQVKTEALQPLT
LEQALNLARLNNPQIQVRQLQVQQRQAALRGTEAALYPTLGLQGTAGYQQ
NGTRLNVTEGTPTQPTGSSLFTTLGESSIGATLNLNYTIFDFVRGAQLAA
SRDQVTQAELDLEAALEDLQLTVSEAYYRLQNADQLVRIARESVVASERQ
LDQTTQRFNVGLVAITDVQNARAQLAQDQQNLVDSIGNQDKARRALVQAL
NLPQNVNVLTADPVELAAPWNLSLDESIVLAFQNRPELEREVLQRNISYN
QAQAARGQVLPQLGLQASYGVNGAINSNLRSGSQALTFPSPTLTNTSSYN
YSIGLVLNVPLFDGGLANANAQQQELNGQIAEQNFVLTRNQIRTDVETAF
YDLQTNLANIGTTRKAVEQARESLDAMEAGYSVGTRTIVDVLDATTDLTR
AEANALNAITAYNLALARIKRAVSNVNNLARAGG

### SEQ ID NO:111

The DNA sequence encoding ybhG_opt, integrated at the ΔA0358 locus, is:

ATGATGAAAAAGCCGGTTGTTATTGGCCTGGCGGTTGTCGTGTTGGCAGC
CGTGGTCGCGGGTGGTTACTGGTGGTATCAGAGCCGCCAAGATAACGGTC
TGACTCTGTACGGTAATGTTGATATCCGCACGGTGAACCTGAGCTTCCGT
GTCGGTGGTCGTGTAGAGTCTCTGGCTGTCGACGAGGGCGATGCGATCAA
GGCGGGTCAGGTGTTGGGCGAGTTGGACCATAAACCGTATGAAATCGCCC
TGATGCAAGCAAAGGCGGGTGTCAGCGTGGCCCAGGCGCAATACGACCTG
ATGCTGGCAGGTTACCGTAATGAGGAGATTGCCCAGGCAGCAGCGGCGGT
GAAGCAGGCCCAAGCGGCATACGATTATGCGCAAAACTTTTACAACCGTC
AGCAAGGTCTGTGGAAAAGCCGTACGATCTCCGCGAATGACTTGGAAAAC
GCCCGTAGCAGCCGCGACCAAGCGCAGGCTACGCTGAAAAGCGCGCAGGA
CAAACTGCGCCAGTACCGTTCTGGCAATCGCGAACAAGACATTGCACAGG
CTAAAGCCAGCCTGGAGCAAGCGCAAGCCCAACTGGCACAGGCGGAACTG
AACTTGCAGGACTCGACCCTGATTGCGCCGAGCGACGGTACCCTGCTGAC
CCGTGCTGTCGAACCAGGCACCGTTCTGAATGAAGGTGGCACCGTTTTTA
CCGTGAGCCTGACCCGTCCGGTGTGGGTCCGCGCTTATGTTGACGAACGC
AATCTGGATCAGGCGCAGCCGGGTCGTAAGGTTCTGCTGTATACCGATGG
TCGTCCGGATAAGCCGTACCACGGCCAAATTGGCTTTGTTTCCCCTACGG
CAGAGTTCACCCCGAAAACGGTCGAGACTCCGGATTTGCGTACCGATCTG
GTTTATCGCCTGCGTATCGTGGTTACCGATGCGGACGATGCGCTGCGTCA
GGGTATGCCGGTGACGGTCCAATTCGGCGACGAGGCAGGCCACGAGTAA

### SEQ ID NO:112

The DNA sequence encoding torA_ybhG_opt, integrated at the ΔA0358 locus, is:

ATGAACAATAACGACTTGTTTCAGGCAAGCCGCCGTCGCTTCCTGGCGCA
GCTGGGTGGCCTGACGGTGGCAGGCATGCTGGGTCCGAGCTTGCTGACCC
CGCGTCGTGCCACCGCGGGTGGTTACTGGTGGTATCAGAGCCGCCAAGAT
AACGGTCTGACTCTGTACGGTAATGTTGATATCCGCACGGTGAACCTGAG
CTTCCGTGTCGGTGGTCGTGTAGAGTCTCTGGCTGTCGACGAGGGCGATG
CGATCAAGGCGGGTCAGGTGTTGGGCGAGTTGGACCATAAACCGTATGAA
ATCGCCCTGATGCAAGCAAAGGCGGGTGTCAGCGTGGCCCAGGCGCAATA
CGACCTGATGCTGGCAGGTTACCGTAATGAGGAGATTGCCCAGGCAGCAG
CGGCGGTGAAGCAGGCCCAAGCGGCATACGATTATGCGCAAAACTTTTAC
AACCGTCAGCAAGGTCTGTGGAAAAGCCGTACGATCTCCGCGAATGACTT
GGAAAACGCCCGTAGCAGCCGCGACCAAGCGCAGGCTACGCTGAAAAGCG
CGCAGGACAAACTGCGCCAGTACCGTTCTGGCAATCGCGAACAAGACATT
GCACAGGCTAAAGCCAGCCTGGAGCAAGCGCAAGCCCAACTGGCACAGGC
GGAACTGAACTTGCAGGACTCGACCCTGATTGCGCCGAGCGACGGTACCC
TGCTGACCCGTGCTGTCGAACCAGGCACCGTTCTGAATGAAGGTGGCACC
GTTTTTACCGTGAGCCTGACCCGTCCGGTGTGGGTCCGCGCTTATGTTGA
CGAACGCAATCTGGATCAGGCGCAGCCGGGTCGTAAGGTTCTGCTGTATA
CCGATGGTCGTCCGGATAAGCCGTACCACGGCCAAATTGGCTTTGTTTCC
CCTACGGCAGAGTTCACCCCGAAAACGGTCGAGACTCCGGATTTGCGTAC
CGATCTGGTTTATCGCCTGCGTATCGTGGTTACCGATGCGGACGATGCGC
TGCGTCAGGGTATGCCGGTGACGGTCCAATTCGGCGACGAGGCAGGCCAC
GAGTAA

### SEQ ID NO:113

The protein sequence encoded by torA_ybhG_opt, integrated at the ΔA0358 is:

MNNNDLFQASRRRFLAQLGGLTVAGMLGPSLLTPRRATAGGYWWYQSRQD
NGLTLYGNVDIRTVNLSFRVGGRVESLAVDEGDAIKAGQVLGELDHKPYE
IALMQAKAGVSVAQAQYDLMLAGYRNEEIAQAAAAVKQAQAAYDYAQNFY
NRQQGLWKSRTISANDLENARSSRDQAQATLKSAQDKLRQYRSGNREQDI
AQAKASLEQAQAQLAQAELNLQDSTLTAPSDGTLLTRAVEPGTVLNEGGT
VFTVSLTRPVWVRAYVDERNLDQAQPGRKVLLYTDGRPDKPYHGQIGFVS
PTAEFTPKTVETPDLRTDLVYRLRIVVTDADDALRQGMPVTVQFGDEA
GHE

### SEQ ID NO:114

The DNA sequence encoding A0578_ybhG_opt, integrated at the ΔA0358 locus, is:

ATGCGTTTCTTTTGGTTCTTTCTGACGCTGCTGACCTTGAGCACCTGGCA
ACTGCCGGCGTGGGCAGGTGGTTACTGGTGGTATCAGAGCCGCCAAGATA
ACGGTCTGACTCTGTACGGTAATGTTGATATCCGCACGGTGAACCTGAGC
TTCCGTGTCGGTGGTCGTGTAGAGTCTCTGGCTGTCGACGAGGGCGATGC
GATCAAGGCGGGTCAGGTGTTGGGCGAGTTGGACCATAAACCGTATGAAA
TCGCCCTGATGCAAGCAAAGGCGGGTGTCAGCGTGGCCCAGGCGCAATAC
GACCTGATGCTGGCAGGTTACCGTAATGAGGAGATTGCCCAGGCAGCAGC
GGCGGTGAAGCAGGCCCAAGCGGCATACGATTATGCGCAAAACTTTTACA
ACCGTCAGCAAGGTCTGTGGAAAAGCCGTACGATCTCCGCGAATGACTTG
GAAAACGCCCGTAGCAGCCGCGACCAAGCGCAGGCTACGCTGAAAAGCGC
GCAGGACAAACTGCGCCAGTACCGTTCTGGCAATCGCGAACAAGACATTG
CACAGGCTAAAGCCAGCCTGGAGCAAGCGCAAGCCCAACTGGCACAGGCG
GAACTGAACTTGCAGGACTCGACCCTGATTGCGCCGAGCGACGGTACCCT
GCTGACCCGTGCTGTCGAACCAGGCACCGTTCTGAATGAAGGTGGCACCG
TTTTTACCGTGAGCCTGACCCGTCCGGTGTGGGTCCGCGCTTATGTTGAC
GAACGCAATCTGGATCAGGCGCAGCCGGGTCGTAAGGTTCTGCTGTATAC
CGATGGTCGTCCGGATAAGCCGTACCACGGCCAAATTGGCTTTGTTTCCC
CTACGGCAGAGTTCACCCCGAAAACGGTCGAGACTCCGGATTTGCGTACC
GATCTGGTTTATCGCCTGCGTATCGTGGTTACCGATGCGGACGATGCGCT
GCGTCAGGGTATGCCGGTGACGGTCCAATTCGGCGACGAGGCAGGCCACG
AGTAA

### SEQ ID NO:115

The protein sequence encoded by A0578_ybhG_opt, integrated at the ΔA0358 is:

MRFFWFFLTLLTLSTWQLPAWAGGYWWYQSRQDNGLTLYGNVDIRTVNLS
FRVGGRVESLAVDEGDAIKAGQVLGELDHKPYEIALMQAKAGVSVAQAQY
DLMLAGYRNEEIAQAAAAVKQAQAAYDYAQNFYNRQQGLWKSRTISANDL
ENARSSRDQAQATLKSAQDKLRQYRSGNREQDIAQAKASLEQAQAQLAQA
ELNLQDSTLIAPSDGTLLTRAVEPGTVLNEGGTVFTVSLTRPVWVRAYVD
ERNLDQAQPGRKVLLYTDGRPDKPYHGQIGFVSPTAEFTPKTVETPDLRT
DLVYRLRIVVTDADDALRQGMPVTVQFGDEAGHE

### SEQ ID NO:116

The DNA sequence encoding A0318_ybhG_opt, integrated at the ΔA0358 locus, is:

ATGCAGAAACAACAAAATCTGGACTACTTTAGCCCGCAGGCCCTGGCCCT
GTGGGCTGCGATTGCGAGCTTGGGTGTTATGTCCCCTGCGCATGCGGGTG
GTTACTGGTGGTATCAGAGCCGCCAAGATAACGGTCTGACTCTGTACGGT
AATGTTGATATCCGCACGGTGAACCTGAGCTTCCGTGTCGGTGGTCGTGT
AGAGTCTCTGGCTGTCGACGAGGGCGATGCGATCAAGGCGGGTCAGGTGT
TGGGCGAGTTGGACCATAAACCGTATGAAATCGCCCTGATGCAAGCAAAG
GCGGGTGTCAGCGTGGCCCAGGCGCAATACGACCTGATGCTGGCAGGTTA
CCGTAATGAGGAGATTGCCCAGGCAGCAGCGGCGGTGAAGCAGGCCCAAG
CGGCATACGATTATGCGCAAAACTTTTACAACCGTCAGCAAGGTCTGTGG
AAAAGCCGTACGATCTCCGCGAATGACTTGGAAAACGCCCGTAGCAGCCG
CGACCAAGCGCAGGCTACGCTGAAAAGCGCGCAGGACAAACTGCGCCAGT
ACCGTTCTGGCAATCGCGAACAAGACATTGCACAGGCTAAAGCCAGCCTG
GAGCAAGCGCAAGCCCAACTGGCACAGGCGGAACTGAACTTGCAGGACTC
GACCCTGATTGCGCCGAGCGACGGTACCCTGCTGACCCGTGCTGTCGAAC
CAGGCACCGTTCTGAATGAAGGTGGCACCGTTTTTACCGTGAGCCTGACC
CGTCCGGTGTGGGTCCGCGCTTATGTTGACGAACGCAATCTGGATCAGGC
GCAGCCGGGTCGTAAGGTTCTGCTGTATACCGATGGTCGTCCGGATAAGC
CGTACCACGGCCAAATTGGCTTTGTTTCCCCTACGGCAGAGTTCACCCCG
AAAACGGTCGAGACTCCGGATTTGCGTACCGATCTGGTTTATCGCCTGCG
TATCGTGGTTACCGATGCGGACGATGCGCTGCGTCAGGGTATGCCGGTGA
CGGTCCAATTCGGCGACGAGGCAGGCCACGAGTAA

### SEQ ID NO:117

The protein sequence encoded by A0318_ybhG_opt, integrated at the ΔA0358 is:

MQKQQNLDYFSPQALALWAAIASLGVMSPAHAGGYWWYQSRQDNGLTLYG
NVDIRTVNLSFRVGGRVESLAVDEGDAIKAGQVLGELDHKPYEIALMQAK
AGVSVAQAQYDLMLAGYRNEEIAQAAAAVKQAQAAYDYAQNFYNRQQGLW
KSRTISANDLENARSSRDQAQATLKSAQDKLRQYRSGNREQDIAQAKASL
EQAQAQLAQAELNLQDSTLIAPSDGTLLTRAVEPGTVLNEGGTVFTVSLT
RPVWVRAYVDERNLDQAQPGRKVLLYTDGRPDKPYHGQIGFVSPTAEFTP
KTVETPDLRTDLVYRLRIVVTDADDALRQGMPVTVQFGDEAGHE

### SEQ ID NO:118

The DNA sequence encoding the ybhF_opt-ybhS_opt-ybhR_opt operon integrated at the ΔA0358 locus is below, lower case sequence representing intergenic sequence, and upper case sequence indicating the three consecutive, non-overlapping open reading frames:

caattgtatataaactgcagtataagtaggaggtaaaatcATGAACGACG
CAGTAATCACCCTGAACGGCCTGGAAAAACGCTTCCCGGGCATGGACAAA
CCGGCTGTTGCTCCATTGGACTGTACCATCCACGCCGGTTACGTGACGGG
TCTGGTTGGTCCGGATGGTGCGGGCAAAACCACCTTGATGCGTATGCTGG
CGGGTCTGCTGAAGCCGGACAGCGGCTCCGCGACCGTTATCGGTTTTGAC
CCGATTAAGAATGACGGTGCATTGCACGCGGTTTTGGGCTACATGCCGCA
GAAATTCGGCCTGTACGAAGATCTGACCGTCATGGAAAATCTGAATCTGT
ATGCTGATTTGCGCTCTGTTACGGGTGAGGCGCGTAAACAAACCTTTGCG
CGTTTGCTGGAATTTACCTCTCTGGGCCCGTTTACGGGTCGTCTGGCGGG
TAAGCTGAGCGGTGGTATGAAGCAGAAACTGGGTTTGGCATGCACCCTGG
TGGGCGAGCCGAAAGTCCTGCTGCTGGATGAGCCGGGTGTGGGCGTCGAT
CCGATTAGCCGTCGTGAGCTGTGGCAGATGGTCCACGAACTGGCTGGCGA
AGGCATGTTGATCCTGTGGAGCACCAGCTATCTGGATGAAGCGGAGCAGT
GCCGTGATGTTCTGTTGATGAATGAGGGCGAGCTGCTGTACCAAGGCGAA
CCAAAAGCGCTGACCCAAACGATGGCGGGTCGCAGCTTCCTGATGACCAG
CCCGCATGAGGGCAACCGTAAACTGCTGCAACGCGCATTGAAACTGCCGC
AAGTCAGCGACGGCATGATTCAGGGCAAATCCGTTCGTCTGATTCTGAAG
AAAGAGGCAACCCCGGACGACATTCGTCATGCAGATGGCATGCCTGAAAT
CAATATCAACGAAACGACCCCGCGTTTCGAGGATGCCTTCATCGATCTGC
TGGGTGGTGCCGGTACCTCTGAGAGCCCGCTGGGCGCAATCCTGCATACC
GTGGAAGGTACTCCGGGTGAGACTGTTATTGAAGCGAAGGAGCTGACGAA
AAAGTTCGGTGACTTTGCCGCGACCGATCACGTGAATTTCGCGGTCAAAC
GTGGTGAGATCTTCGGCCTGCTGGGTCCTAACGGTGCAGGTAAATCCACC
ACTTTTAAGATGATGTGTGGTCTGTTGGTGCCAACGAGCGGTCAGGCGCT
GGTCCTGGGTATGGACCTGAAGGAAAGCAGCGGCAAAGCTCGCCAACACC
TGGGTTACATGGCACAAAAGTTTTCTCTGTACGGCAATTTGACGGTGGAG
CAGAACTTGCGCTTTTTCAGCGGTGTGTATGGTCTGCGTGGTCGCGCCCA
AAATGAAAAGATTAGCCGCATGAGCGAAGCGTTCGGTCTGAAAAGCATCG
CGAGCCACGCAACCGACGAGTTGCCGCTGGGTTTCAAACAACGCCTGGCG
CTGGCCTGTAGCCTGATGCACGAGCCGGATATTCTGTTTCTGGACGAGCC
GACCAGCGGTGTCGATCCGCTGACGCGTCGTGAGTTCTGGCTGCACATTA
ACAGCATGGTCGAAAAGGGCGTTACCGTGATGGTTACTACGCATTTCATG
GACGAAGCCGAGTATTGCGATCGTATCGGCCTGGTGTATCGTGGCAAGTT
GATTGCGTCCGGTACGCCGGATGATCTGAAGGCACAGTCGGCGAACGACG
AGCAGCCGGACCCGACGATGGAACAGGCCTTTATCCAGCTGATTCACGAC
TGGGACAAGGAGCATAGCAACGAGTAAggatcctcaagtaggaggtacta
gtaATGAGCAATCCAATCCTGAGCTGGCGTCGCGTCCGTGCACTGTGCGT
GAAAGAAACTCGCCAAATCGTCCGCGACCCGAGCTCCTGGCTGATCGCCG
TTGTGATTCCGCTGCTGCTGTTGTTCATCTTCGGCTATGGTATCAACCTG
GATAGCAGCAAACTGCGCGTCGGTATTCTGCTGGAGCAGCGTAGCGAAGC
TGCCCTGGACTTCACCCACACCATGACGGGCTCCCCGTATATCGACGCTA
CCATTTCTGATAATCGTCAGGAACTGATTGCGAAGATGCAAGCGGGCAAG
ATTCGCGGTCTGGTTGTTATTCCGGTTGACTTCGCAGAGCAAATGGAGCG
TGCCAATGCGACCGCCCCAATTCAGGTGATTACCGACGGTAGCGAACCGA
ATACCGCGAACTTTGTTCAAGGTTACGTAGAAGGTATTTGGCAAATCTGG
CAGATGCAACGTGCAGAGGACAACGGTCAGACCTTCGAACCGCTGATTGA
TGTGCAGACCCGTTACTGGTTTAACCCTGCGGCCATTAGCCAACATTTCA
TCATCCCGGGTGCCGTCACCATCATTATGACGGTTATCGGCGCGATTCTG
ACGAGCTTGGTTGTGGCGCGTGAATGGGAGCGTGGTACGATGGAGGCATT
GCTGAGCACGGAGATCACCCGTACCGAGTTGCTGTTGTGCAAGCTGATTC
CGTACTATTTCCTGGGCATGCTGGCGATGCTGCTGTGTATGTTGGTCAGC
GTGTTCATCCTGGGCGTGCCGTATCGTGGTAGCCTGCTGATCTTGTTCTT
TATCTCTAGCTTGTTTCTGCTGTCTACCCTGGGTATGGGTCTGCTGATTA
GCACCATCACGCGCAACCAGTTTAACGCAGCACAGGTCGCGCTGAACGCG
GCGTTTCTGCCGAGCATCATGCTGAGCGGTTTTATCTTTCAGATTGATTC
CATGCCGGCTGTTATCCGTGCGGTCACTTACATTATTCCTGCGCGCTACT
TCGTGTCGACGTTGCAAAGCCTGTTCCTGGCAGGCAATATTCCGGTCGTG
CTGGTGGTTAATGTTCTGTTCCTGATTGCATCCGCGGTTATGTTTATCGG
CCTGACGTGGCTGAAAACCAAACGCCGTCTGGATTAActcgagactcata
ggaggacatctagATGTTTCATAGATTATGGACACTAATCAGAAAAGAAC
TGCAATCCCTGCTGCGTGAACCTCAGACGCGTGCGATCCTGATCTTGCCG
GTGCTGATTCAGGTCATCCTGTTCCCGTTTGCCGCTACCTTGGAAGTCAC
GAATGCCACTATTGCGATCTACGACGAGGATAACGGTGAACACAGCGTCG
AGCTGACCCAGCGTTTCGCGCGTGCCTCTGCTTTTACCCACGTGCTGTTG
CTGAAAAGCCCGCAGGAAATTCGCCCGACGATTGATACGCAAAAGGCGCT
GCTGCTGGTTCGCTTTCCGGCCGACTTTAGCCGTAAGCTGGACACCTTTC
AGACCGCACCTCTGCAACTGATCCTGGATGGCCGCAACTCGAATAGCGCG
CAGATTGCTGCGAATTACCTGCAACAAATTGTGAAAAACTATCAGCAAGA
GCTGCTGGAGGGTAAACCGAAGCCAAATAACTCCGAGCTGGTTGTCCGTA
ACTGGTATAATCCGAATTTGGACTATAAGTGGTTCGTGGTTCCGAGCCTG
ATTGCGATGATTACCACCATTGGTGTGATGATTGTTACCAGCTTGAGCGT
TGCACGTGAACGTGAGCAAGGTACGCTGGATCAACTGCTGGTTTCTCCGC
TGACCACCTGGCAGATTTTCATCGGTAAAGCTGTTCCGGCGTTGATCGTA
GCGACCTTTCAGGCGACCATCGTGCTGGCAATCGGTATCTGGGCGTACCA
GATCCCGTTCGCCGGCAGCCTGGCGCTGTTCTACTTCACGATGGTGATTT
ATGGTCTGAGCCTGGTCGGCTTCGGTCTGCTGATTAGCAGCCTGTGCAGC
ACCCAGCAACAGGCCTTCATTGGCGTGTTCGTGTTTATGATGCCGGCAAT
CTTGCTGTCGGGCTACGTCAGCCCAGTCGAGAATATGCCGGTTTGGTTGC
AAAACCTGACGTGGATCAACCCGATCCGTCATTTTACGGACATCACGAAG
CAGATTTATCTGAAAGATGCAAGCCTGGACATTGTTTGGAACTCCCTGTG
GCCGCTGCTGGTCATCACCGCAACTACCGGCAGCGCGGCATACGCTATGT
TCCGCCGCAAGGTTATGTAA

### SEQ ID NO:119

The DNA sequence encoding the ybhF_opt-s110041_Nin_PLS_ybhS_opt-s110041_Nin_PLS_ybhR_opt operon integrated at the ΔA0358 locus is below, lower case sequence representing intergenic sequence, and upper case sequence indicating the three consecutive, non-overlapping open reading frames:

caattgtatataaactgcagtataagtaggaggtaaaatcATGAACGACG
CAGTAATCACCCTGAACGGCCTGGAAAAACGCTTCCCGGGCATGGACAAA
CCGGCTGTTGCTCCATTGGACTGTACCATCCACGCCGGTTACGTGACGGG
TCTGGTTGGTCCGGATGGTGCGGGCAAAACCACCTTGATGCGTATGCTGG
CGGGTCTGCTGAAGCCGGACAGCGGCTCCGCGACCGTTATCGGTTTTGAC
CCGATTAAGAATGACGGTGCATTGCACGCGGTTTTGGGCTACATGCCGCA
GAAATTCGGCCTGTACGAAGATCTGACCGTCATGGAAAATCTGAATCTGT
ATGCTGATTTGCGCTCTGTTACGGGTGAGGCGCGTAAACAAACCTTTGCG
CGTTTGCTGGAATTTACCTCTCTGGGCCCGTTTACGGGTCGTCTGGCGGG
TAAGCTGAGCGGTGGTATGAAGCAGAAACTGGGTTTGGCATGCACCCTGG
TGGGCGAGCCGAAAGTCCTGCTGCTGGATGAGCCGGGTGTGGGCGTCGAT
CCGATTAGCCGTCGTGAGCTGTGGCAGATGGTCCACGAACTGGCTGGCGA
AGGCATGTTGATCCTGTGGAGCACCAGCTATCTGGATGAAGCGGAGCAGT
GCCGTGATGTTCTGTTGATGAATGAGGGCGAGCTGCTGTACCAAGGCGAA
CCAAAAGCGCTGACCCAAACGATGGCGGGTCGCAGCTTCCTGATGACCAG
CCCGCATGAGGGCAACCGTAAACTGCTGCAACGCGCATTGAAACTGCCGC
AAGTCAGCGACGGCATGATTCAGGGCAAATCCGTTCGTCTGATTCTGAAG
AAAGAGGCAACCCCGGACGACATTCGTCATGCAGATGGCATGCCTGAAAT
CAATATCAACGAAACGACCCCGCGTTTCGAGGATGCCTTCATCGATCTGC
TGGGTGGTGCCGGTACCTCTGAGAGCCCGCTGGGCGCAATCCTGCATACC
GTGGAAGGTACTCCGGGTGAGACTGTTATTGAAGCGAAGGAGCTGACGAA
AAAGTTCGGTGACTTTGCCGCGACCGATCACGTGAATTTCGCGGTCAAAC
GTGGTGAGATCTTCGGCCTGCTGGGTCCTAACGGTGCAGGTAAATCCACC
ACTTTTAAGATGATGTGTGGTCTGTTGGTGCCAACGAGCGGTCAGGCGCT
GGTCCTGGGTATGGACCTGAAGGAAAGCAGCGGCAAAGCTCGCCAACACC
TGGGTTACATGGCACAAAAGTTTTCTCTGTACGGCAATTTGACGGTGGAG
CAGAACTTGCGCTTTTTCAGCGGTGTGTATGGTCTGCGTGGTCGCGCCCA
AAATGAAAAGATTAGCCGCATGAGCGAAGCGTTCGGTCTGAAAAGCATCG
CGAGCCACGCAACCGACGAGTTGCCGCTGGGTTTCAAACAACGCCTGGCG
CTGGCCTGTAGCCTGATGCACGAGCCGGATATTCTGTTTCTGGACGAGCC
GACCAGCGGTGTCGATCCGCTGACGCGTCGTGAGTTCTGGCTGCACATTA
ACAGCATGGTCGAAAAGGGCGTTACCGTGATGGTTACTACGCATTTCATG
GACGAAGCCGAGTATTGCGATCGTATCGGCCTGGTGTATCGTGGCAAGTT
GATTGCGTCCGGTACGCCGGATGATCTGAAGGCACAGTCGGCGAACGACG
AGCAGCCGGACCCGACGATGGAACAGGCCTTTATCCAGCTGATTCACGAC
TGGGACAAGGAGCATAGCAACGAGTAAggatcctcaagtaggaggtacta
gtaATGCAAGCACCAACGCAAAGCGGCGGTCTGAGCCTGAGAAACAAAGC
GGTCCTGATTGCACTGCTGATCGGCCTGATTCCGGCAGGCGTTATTGGTG
GTTTGAATCTGAGCAGCGTTGATCGTCTGCCGGTCCCTCAAACCGAGCAG
CAGGTCAAAGATAGCACCACCAAGCAGATTCGTGACCAGATTCTGATCGG
TCTGCTGGTGACCGCAGTGGGTGCAGCGTTCGTCGCGTATTGGATGGTTG
GTGAGAACACCAAAGCGCAAACCGCGCTGGCGCTGAAGGCTAAGTCCAAT
CCGATTCTGAGCTGGCGCCGTGTACGCGCGCTGTGTGTGAAGGAAACCCG
TCAGATTGTGCGTGATCCGAGCTCGTGGCTGATTGCGGTCGTCATCCCGT
TGTTGCTGCTGTTCATTTTTGGCTACGGTATCAACCTGGATAGCAGCAAA
TTGCGCGTTGGTATTTTGCTGGAGCAGCGTAGCGAAGCGGCGCTGGATTT
TACCCATACCATGACGGGCAGCCCGTACATTGACGCCACCATTAGCGACA
ATCGTCAGGAACTGATTGCGAAGATGCAAGCCGGTAAGATCCGTGGCCTG
GTTGTGATCCCGGTCGACTTTGCGGAGCAAATGGAGCGCGCGAATGCGAC
CGCACCGATCCAAGTCATCACGGACGGCAGCGAGCCGAACACCGCTAACT
TCGTTCAGGGTTATGTCGAGGGTATCTGGCAAATTTGGCAGATGCAACGT
GCGGAGGATAATGGCCAGACCTTCGAACCGCTGATCGACGTTCAGACTCG
TTACTGGTTCAATCCAGCCGCTATCAGCCAGCACTTCATCATTCCGGGTG
CGGTTACGATCATTATGACGGTAATCGGTGCGATTCTGACGTCCCTGGTT
GTCGCCCGTGAGTGGGAACGTGGTACGATGGAGGCACTGCTGTCTACCGA
AATTACGCGTACGGAACTGTTGCTGTGCAAATTGATCCCGTACTACTTCC
TGGGTATGTTGGCCATGCTGCTGTGCATGCTGGTGAGCGTGTTCATCCTG
GGTGTGCCGTATCGTGGTTCTCTGCTGATCCTGTTTTTCATCTCTAGCCT
GTTTTTGCTGTCCACTCTGGGCATGGGCCTGCTGATTAGCACTATCACCC
GCAACCAGTTTAATGCGGCCCAGGTGGCCCTGAACGCAGCATTTTTGCCG
AGCATCATGCTGTCCGGTTTCATCTTTCAAATTGATAGCATGCCGGCAGT
GATCCGCGCTGTTACCTATATCATTCCTGCTCGTTACTTCGTTAGCACGC
TGCAATCGCTGTTCTTGGCGGGCAACATTCCGGTCGTGCTGGTTGTTAAC
GTGCTGTTTCTGATTGCCAGCGCTGTGATGTTTATTGGCCTGACCTGGCT
GAAAACGAAACGCCGCCTGGACTAActcgagactcataggaggacatcta
gATGCAAGCACCAACCCAATCCGGCGGCCTGAGCCTGCGCAACAAAGCGG
TTCTGATCGCGTTGCTGATTGGTCTGATTCCGGCAGGTGTGATTGGTGGC
CTGAATCTGTCTAGCGTGGATCGCCTGCCGGTGCCGCAGACTGAACAGCA
GGTGAAGGACTCCACGACCAAGCAAATTCGTGACCAGATTCTGATTGGCC
TGTTGGTTACTGCCGTGGGTGCGGCATTTGTCGCGTATTGGATGGTTGGT
GAAAATACCAAAGCGCAAACCGCGCTGGCTCTGAAGGCGAAATTTCATCG
TCTGTGGACCCTGATCCGTAAGGAGCTGCAAAGCCTGTTGCGTGAGCCGC
AGACCCGTGCTATTCTGATTCTGCCGGTCTTGATCCAAGTGATCCTGTTC
CCGTTTGCCGCTACCCTGGAAGTGACGAATGCCACGATTGCCATTTACGA
TGAGGACAATGGTGAGCACTCCGTTGAACTGACCCAACGTTTTGCACGTG
CGTCCGCTTTCACCCATGTGCTGCTGTTGAAATCTCCGCAGGAGATTCGT
CCGACCATTGATACGCAGAAGGCGCTGCTGCTGGTGCGCTTTCCTGCTGA
CTTCAGCCGTAAGCTGGACACCTTCCAGACCGCGCCATTGCAGCTGATCC
TGGATGGCCGCAATTCTAATAGCGCACAGATCGCCGCAAACTATCTGCAA
CAGATTGTGAAAAACTACCAGCAAGAACTGCTGGAGGGTAAACCGAAACC
GAACAATAGCGAACTGGTCGTCCGTAACTGGTATAACCCGAACCTGGACT
ACAAATGGTTCGTTGTCCCGAGCCTGATCGCGATGATTACCACCATCGGC
GTTATGATCGTCACCAGCCTGAGCGTAGCACGTGAGCGCGAGCAAGGCAC
CCTGGATCAACTGTTGGTGAGCCCTCTGACTACGTGGCAGATCTTCATCG
GTAAGGCGGTTCCGGCACTGATCGTCGCCACGTTCCAGGCGACCATCGTT
TTGGCAATCGGTATTTGGGCGTATCAAATCCCGTTCGCGGGTAGCCTGGC
CCTGTTTTACTTCACGATGGTTATCTACGGCTTGAGCCTGGTTGGCTTCG
GTTTGCTGATTAGCAGCCTGTGCAGCACCCAGCAACAGGCGTTTATCGGT
GTTTTTGTGTTTATGATGCCGGCGATTCTGCTGAGCGGTTACGTCAGCCC
GGTCGAGAACATGCCGGTGTGGCTGCAAAACCTGACGTGGATCAATCCGA
TCCGCCACTTCACGGATATTACCAAGCAGATCTACCTGAAAGACGCGAGC
CTGGACATTGTCTGGAACAGCTTGTGGCCGTTGCTGGTTATCACCGCGAC
GACGGGTTCGGCAGCGTATGCCATGTTCCGCCGTAAGGTAATGTAA

### SEQ ID NO:120

The protein sequence encoded by the ybhS_opt ORF in the ybhF_opt-s110041_Nin_PLS_ybhS_opt-s110041_Nin_PLS_ybhR_opt operon, integrated at the ΔA0358 locus, is:

MQAPTQSGGLSLRNKAVLIALLIGLIPAGVIGGLNLSSVDRLPVPQTEQQ
VKDSTTKQIRDQILIGLLVTAVGAAFVAYWMVGENTKAQTALALKAKSNP
ILSWRRVRALCVKETRQIVRDPSSWLIAVVIPLLLLFIFGYGINLDSSKL
RVGILLEQRSEAALDFTHTMTGSPYIDATISDNRQELIAKMQAGKIRGLV
VIPVDFAEQMERANATAPIQVITDGSEPNTANFVQGYVEGIWQIWQMQRA
EDNGQTFEPLIDVQTRYWFNPAAISQHFIIPGAVTIIMTVIGAILTSLVV
AREWERGTMEALLSTEITRTELLLCKLIPYYFLGMLAMLLCMLVSVFILG
VPYRGSLLILFFISSLFLLSTLGMGLLISTITRNQFNAAQVALNAAFLPS
IMLSGFIFQIDSMPAVIRAVTYIIPARYFVSTLQSLFLAGNIPVVLVVNV
LFLIASAVMFIGLTWLKTKRRLD

### SEQ ID NO:121

The protein sequence encoded by the ybhR_opt ORF in the ybhF_opt-s110041_Nin_PLS_ybhS_opt-s110041_Nin_PLS_ybhR_opt operon, integrated at the ΔA0358 locus, is:

MQAPTQSGGLSLRNKAVLIALLIGLIPAGVIGGLNLSSVDRLPVPQTEQQ
VKDSTTKQIRDQILIGLLVTAVGAAFVAYWMVGENTKAQTALALKAKFHR
LWTLIRKELQSLLREPQTRAILILPVLIQVILFPFAATLEVTNATIAIYD
EDNGEHSVELTQRFARASAFTHVLLLKSPQEIRPTIDTQKALLLVRFPAD
FSRKLDTFQTAPLQLILDGRNSNSAQIAANYLQQIVKNYQQELLEGKPKP
NNSELVVRNWYNPNLDYKWFVVPSLIAMITTIGVMIVTSLSVAREREQGT
LDQLLVSPLTTWQIFIGKAVPALIVATFQATIVLAIGIWAYQIPFAGSLA
LFYFTMVIYGLSLVGFGLLISSLCSTQQQAFIGVFVFMMPAILLSGYVSP
VENMPVWLQNLTWINPIRHFTDITKQIYLKDASLDIVWNSLWPLLVITAT
TGSAAYAMFRRKVM

### SEQ ID NO:122

The DNA sequence encoding the ybhF_opt-slr1044_Nin_PLS_ybhS_opt-slr1044_Nin_PLS_ybhR_opt operon integrated at the ΔA0358 locus is below, lower case sequence representing intergenic sequence, and upper case sequence indicating the three consecutive, non-overlapping open reading frames:

caattgtatataaactgcagtataagtaggaggtaaaatcATGAACGACG
CAGTAATCACCCTGAACGGCCTGGAAAAACGCTTCCCGGGCATGGACAAA
CCGGCTGTTGCTCCATTGGACTGTACCATCCACGCCGGTTACGTGACGGG
TCTGGTTGGTCCGGATGGTGCGGGCAAAACCACCTTGATGCGTATGCTGG
CGGGTCTGCTGAAGCCGGACAGCGGCTCCGCGACCGTTATCGGTTTTGAC
CCGATTAAGAATGACGGTGCATTGCACGCGGTTTTGGGCTACATGCCGCA
GAAATTCGGCCTGTACGAAGATCTGACCGTCATGGAAAATCTGAATCTGT
ATGCTGATTTGCGCTCTGTTACGGGTGAGGCGCGTAAACAAACCTTTGCG
CGTTTGCTGGAATTTACCTCTCTGGGCCCGTTTACGGGTCGTCTGGCGGG
TAAGCTGAGCGGTGGTATGAAGCAGAAACTGGGTTTGGCATGCACCCTGG
TGGGCGAGCCGAAAGTCCTGCTGCTGGATGAGCCGGGTGTGGGCGTCGAT
CCGATTAGCCGTCGTGAGCTGTGGCAGATGGTCCACGAACTGGCTGGCGA
AGGCATGTTGATCCTGTGGAGCACCAGCTATCTGGATGAAGCGGAGCAGT
GCCGTGATGTTCTGTTGATGAATGAGGGCGAGCTGCTGTACCAAGGCGAA
CCAAAAGCGCTGACCCAAACGATGGCGGGTCGCAGCTTCCTGATGACCAG
CCCGCATGAGGGCAACCGTAAACTGCTGCAACGCGCATTGAAACTGCCGC
AAGTCAGCGACGGCATGATTCAGGGCAAATCCGTTCGTCTGATTCTGAAG
AAAGAGGCAACCCCGGACGACATTCGTCATGCAGATGGCATGCCTGAAAT
CAATATCAACGAAACGACCCCGCGTTTCGAGGATGCCTTCATCGATCTGC
TGGGTGGTGCCGGTACCTCTGAGAGCCCGCTGGGCGCAATCCTGCATACC
GTGGAAGGTACTCCGGGTGAGACTGTTATTGAAGCGAAGGAGCTGACGAA
AAAGTTCGGTGACTTTGCCGCGACCGATCACGTGAATTTCGCGGTCAAAC
GTGGTGAGATCTTCGGCCTGCTGGGTCCTAACGGTGCAGGTAAATCCACC
ACTTTTAAGATGATGTGTGGTCTGTTGGTGCCAACGAGCGGTCAGGCGCT
GGTCCTGGGTATGGACCTGAAGGAAAGCAGCGGCAAAGCTCGCCAACACC
TGGGTTACATGGCACAAAAGTTTTCTCTGTACGGCAATTTGACGGTGGAG
CAGAACTTGCGCTTTTTCAGCGGTGTGTATGGTCTGCGTGGTCGCGCCCA
AAATGAAAAGATTAGCCGCATGAGCGAAGCGTTCGGTCTGAAAAGCATCG
CGAGCCACGCAACCGACGAGTTGCCGCTGGGTTTCAAACAACGCCTGGCG
CTGGCCTGTAGCCTGATGCACGAGCCGGATATTCTGTTTCTGGACGAGCC
GACCAGCGGTGTCGATCCGCTGACGCGTCGTGAGTTCTGGCTGCACATTA
ACAGCATGGTCGAAAAGGGCGTTACCGTGATGGTTACTACGCATTTCATG
GACGAAGCCGAGTATTGCGATCGTATCGGCCTGGTGTATCGTGGCAAGTT
GATTGCGTCCGGTACGCCGGATGATCTGAAGGCACAGTCGGCGAACGACG
AGCAGCCGGACCCGACGATGGAACAGGCCTTTATCCAGCTGATTCACGAC
TGGGACAAGGAGCATAGCAACGAGTAAggatcctcaagtaggaggtacta
gtAATGTTCTTAGGATGGTTCACCAACGCATCGCTGTTCCGCAAGCAAAT
CTATATGGCGATTGCGAGCGGTGTTTTTAGCGGCTTTGCTGTTCTGGTGC
TGGGCAGCATTGTGGGTCTGGGTGGTACCCCTAAGGACGTTCCGGCACCG
AGCGGTGAAACCACCACCGAAGCACCGGCAGAAGGTGCACCAGCGGAAGG
CCAAGCTCCGAGCCAGACCCCGGAAGAGGAACCGGGCAAACCGAGCCTGC
TGAACCTGGCGTTCCTGACGGCCATTGCTACGGCGATTGGTGTCTTTCTG
ATTAACCGCTTGCTGATGCAGCAAATCAAAAGCATCATTGACGACCTGCA
AAGCAATCCGATCCTGAGCTGGCGCCGTGTTCGTGCCCTGTGCGTGAAGG
AAACCCGTCAGATTGTGCGTGATCCGAGCTCTTGGCTGATCGCGGTCGTC
ATTCCTCTGCTGCTGCTGTTCATTTTCGGTTATGGTATTAACCTGGATAG
CAGCAAACTGCGTGTTGGTATTCTGCTGGAACAGCGTAGCGAGGCGGCGT
TGGATTTTACCCATACCATGACGGGTTCCCCGTACATTGACGCGACCATC
AGCGATAACCGCCAGGAGCTGATCGCAAAGATGCAGGCCGGCAAAATTCG
TGGCCTGGTGGTGATTCCGGTTGACTTCGCGGAGCAGATGGAGCGCGCAA
ACGCAACCGCACCGATTCAAGTGATTACCGATGGTTCCGAACCGAATACG
GCAAATTTCGTGCAAGGCTATGTGGAGGGTATCTGGCAAATTTGGCAGAT
GCAACGCGCGGAGGATAATGGCCAGACCTTTGAACCGCTGATCGACGTCC
AAACTCGTTACTGGTTTAATCCAGCGGCCATCAGCCAACACTTTATCATT
CCGGGTGCGGTCACCATCATTATGACGGTCATTGGCGCTATCCTGACCTC
TTTGGTAGTCGCCCGTGAGTGGGAGCGTGGTACGATGGAGGCGCTGCTGA
GCACGGAGATCACTCGTACGGAATTGCTGCTGTGCAAACTGATCCCGTAC
TACTTCCTGGGTATGCTGGCGATGCTGTTGTGTATGCTGGTCAGCGTTTT
CATTCTGGGTGTGCCATACCGCGGCAGCTTGTTGATTCTGTTCTTCATCT
CCTCGTTGTTTCTGCTGTCTACCCTGGGCATGGGTCTGCTGATTAGCACG
ATCACCCGCAATCAGTTCAACGCGGCTCAGGTCGCGCTGAATGCCGCCTT
CCTGCCGAGCATCATGCTGAGCGGCTTTATCTTTCAGATCGATTCGATGC
CGGCTGTTATTCGTGCCGTTACGTATATCATCCCGGCACGTTACTTCGTT
TCCACCTTGCAGAGCCTGTTTTTGGCCGGTAACATCCCGGTGGTGCTGGT
TGTTAATGTCTTGTTCCTGATCGCGTCCGCGGTTATGTTTATCGGTCTGA
CTTGGCTGAAAACGAAGCGTCGTCTGGACTAActcgagactcataggagg
acatctagATGTTTTTAGGCTGGTTCACCAATGCCTCGTTATTTCGCAAA
CAGATCTACATGGCCATTGCGAGCGGTGTTTTCTCCGGTTTCGCGGTGCT
GGTTCTGGGTTCCATCGTTGGTCTGGGCGGTACCCCGAAGGACGTCCCTG
CACCGTCTGGCGAAACGACCACGGAGGCACCGGCGGAAGGTGCTCCGGCG
GAGGGCCAAGCGCCGAGCCAGACCCCGGAGGAAGAACCGGGCAAGCCGAG
CTTGTTGAATCTGGCCTTCTTGACCGCTATCGCCACCGCGATCGGTGTCT
TTCTGATTAACCGTCTGCTGATGCAGCAAATCAAGAGCATCATTGACGAT
TTGCAATTTCATCGCCTGTGGACGCTGATTCGTAAGGAGCTGCAAAGCCT
GCTGCGCGAACCACAAACCCGTGCCATTCTGATTCTGCCGGTGCTGATCC
AGGTTATTCTGTTCCCGTTCGCAGCGACCCTGGAGGTGACGAACGCCACC
ATTGCCATCTATGACGAGGATAACGGCGAGCACAGCGTGGAGCTGACCCA
GCGTTTCGCTCGTGCAAGCGCGTTTACGCACGTTCTGCTGCTGAAAAGCC
CGCAGGAGATCCGTCCGACCATTGACACTCAGAAAGCGCTGCTGCTGGTT
CGCTTTCCTGCGGATTTTAGCCGTAAACTGGACACCTTCCAGACGGCACC
GCTGCAACTGATTCTGGATGGTCGTAACAGCAACAGCGCGCAGATTGCGG
CCAACTACCTGCAACAGATTGTTAAGAACTATCAGCAAGAATTGTTGGAG
GGCAAACCGAAGCCGAATAACAGCGAACTGGTCGTGCGTAATTGGTACAA
TCCGAATCTGGACTACAAGTGGTTCGTGGTTCCGAGCCTGATCGCGATGA
TTACCACCATTGGCGTAATGATCGTTACTTCCCTGAGCGTGGCACGCGAA
CGTGAACAAGGTACGCTGGACCAGTTGCTGGTCAGCCCGTTGACCACCTG
GCAGATCTTCATCGGTAAAGCAGTTCCAGCACTGATCGTTGCGACTTTCC
AGGCAACCATCGTGCTGGCCATCGGTATTTGGGCGTACCAGATTCCGTTT
GCGGGTAGCCTGGCTCTGTTTTACTTCACTATGGTCATTTATGGCCTGTC
TTTGGTTGGTTTTGGTTTGCTGATCTCTTCCCTGTGCAGCACCCAGCAAC
AAGCGTTCATTGGTGTCTTTGTGTTTATGATGCCAGCAATTCTGCTGAGC
GGCTATGTGAGCCCGGTCGAGAACATGCCGGTCTGGCTGCAAAATCTGAC
GTGGATCAATCCGATCCGTCATTTCACGGATATTACCAAACAAATCTACC
TGAAGGATGCTAGCCTGGATATCGTGTGGAACAGCTTGTGGCCGCTGCTG
GTCATTACGGCAACCACGGGTTCTGCGGCGTATGCGATGTTCCGTCGCAA
AGTGATGTAA

### SEQ ID NO:123

The protein sequence encoded by the ybhS_opt ORF in the ybhF_opt-slr1044_Nin_PLS_ybhS_opt-slr1044_Nin_PLS_ybhR_opt operon, integrated at the ΔA0358 locus, is:

MFLGWFTNASLFRKQIYMAIASGVFSGFAVLVLGSIVGLGGTPKDVPAPS
GETTTEAPAEGAPAEGQAPSQTPEEEPGKPSLLNLAFLTAIATAIGVFLI
NRLLMQQIKSIIDDLQSNPILSWRRVRALCVKETRQIVRDPSSWLIAVVI
PLLLLFIFGYGINLDSSKLRVGILLEQRSEAALDFTHTMTGSPYIDATIS
DNRQELIAKMQAGKIRGLVVIPVDFAEQMERANATAPIQVITDGSEPNTA
NFVQGYVEGIWQIWQMQRAEDNGQTFEPLIDVQTRYWFNPAAISQHFIIP
GAVTIIMTVIGAILTSLVVAREWERGTMEALLSTEITRTELLLCKLIPYY
FLGMLAMLLCMLVSVFILGVPYRGSLLILFFISSLFLLSTLGMGLLISTI
TRNQFNAAQVALNAAFLPSIMLSGFIFQIDSMPAVIRAVTYIIPARYFVS
TLQSLFLAGNIPVVLVVNVLFLIASAVMFIGLTWLKTKRRLD

### SEQ ID NO:124

The protein sequence encoded by the ybhR_opt ORF in the ybhF_opt-slr1044_Nin_PLS_ybhS_opt-slr1044_Nin_PLS_ybhR_opt operon, integrated at the ΔA0358 locus, is:

MFLGWFTNASLFRKQIYMAIASGVFSGFAVLVLGSIVGLGGTPKDVPAPS
GETTTEAPAEGAPAEGQAPSQTPEEEPGKPSLLNLAFLTAIATAIGVFLI
NRLLMQQIKSIIDDLQFHRLWTLIRKELQSLLREPQTRAILILPVLIQVI
LFPFAATLEVTNATIAIYDEDNGEHSVELTQRFARASAFTHVLLLKSPQE
IRPTIDTQKALLLVRFPADFSRKLDTFQTAPLQLILDGRNSNSAQIAANY
LQQIVKNYQQELLEGKPKPNNSELVVRNWYNPNLDYKWFVVPSLIAMITT
IGVMIVTSLSVAREREQGTLDQLLVSPLTTWQIFIGKAVPALIVATFQAT
IVLAIGIWAYQIPFAGSLALFYFTMVIYGLSLVGFGLLISSLCSTQQQAF
IGVFVFMMPAILLSGYVSPVENMPVWLQNLTWINPIRHFTDITKQIYLKD
ASLDIVWNSLWPLLVITATTGSAAYAMFRRKVM

### SEQ ID NO:125

The DNA sequence of the P(aphII) promoter, integrated at the ΔA0358-downstream locus in JCC2522, is:

GGGGGGGGGGGGGAAAGCCACGTTGTGTCTCAAAATCTCTGATGTTACAT
TGCACAAGATAAAAATATATCATCATGAACAATAAAACTGTCTGCTTACA
TAAACAGTAATACAAGTGTACAT

### SEQ ID NO:126

The DNA sequence of the P(psaA) promoter, integrated at the ΔA0358-downstream locus in JCC2522, is:

GCCCCTATATTATGCATTTATACCCCCACAATCATGTCAAGAATTCAAGC
ATCTTAAATAATGTTAATTATCGGCAAAGTCTGTGCTCCCCTTCTATAAT
GCTGAATTGAGCATTCGCCTCCTGAACGGTCTTTATTCTTCCATTGTGGG
TCTTTAGATTCACGATTCTTCACAATCATTGATCTAAGGATCTTTGTAGA
TTCTCTGTACAT

### SEQ ID NO:127

The DNA sequence of the P(tsr2142) promoter, integrated at the ΔA0358-downstream locus in JCC2522, is:

CCAAGGTGGCTACTTCAACGATAGCTTAAACTTCGCTGCTCCAGCGAGGG
GATTTCACTGGTTTGAATGCTTCAATGCTTGCCAAAAGAGTGCTACTGGA
ACTTACAAGAGTGACCCTGCGTCAGGGGAGCTAGCACTCAAAAAAGACTC
CTCCTGTACAT

### SEQ ID NO:128

The DNA sequence encoding A0318_ProNTerm_tolC_opt_A0318C, integrated at the ΔA0358-downstream locus in JCC2522, is:

ATGCAAAAACAACAGAATCTGGACTACTTTAGCCCGCAGGCGTTGGCACT
GTGGGCGGCTATTGCTTCCCTGGGTGTTATGAGCCCGGCACACGCGGAGC
CGCGTAGCGAGGGCAGCCATTCTGATCCGCTGGTTCCGACCGCGACGCAG
GTCGTGGTTCCGGCGCTGCCGGTGGAGGACGTTGCGCCGACCGCCGCACC
GGCATCGCAGACCCCGGCTCCTCAGAGCGAAAACTTGGCGCAATCCAGCA
CCCAAGCCGTCACGAGCCCTGTGGCGCAGGCGCAGGAAGCCCCGCAAGAC
AGCAATCTGCCGCAACTGTATGCCCAGCAGCAAGGTAACCCAAATGCCCA
ACAGGCGAACCCGGAGAATTTGATGCAGGTTTACCAGCAGGCGCGTCTGT
CCAATCCGGAGCTGCGTAAAAGCGCTGCCGACCGTGATGCCGCGTTTGAG
AAGATTAACGAAGCCCGCAGCCCGCTGCTGCCGCAGCTGGGTTTGGGCGC
TGACTACACCTACTCCAACGGCTATCGTGACGCCAACGGTATCAATAGCA
ATGCGACCAGCGCCAGCCTGCAACTGACCCAAAGCATTTTTGATATGAGC
AAATGGCGCGCTCTGACCCTGCAAGAGAAAGCGGCAGGTATCCAGGATGT
GACCTACCAAACGGACCAGCAGACCCTGATCTTGAACACGGCGACCGCGT
ATTTCAATGTTTTGAACGCAATCGATGTCCTGAGCTATACCCAGGCCCAG
AAGGAAGCGATTTATCGTCAGTTGGATCAGACCACCCAGCGCTTCAATGT
GGGTCTGGTGGCGATTACGGATGTTCAAAATGCGCGTGCGCAATACGATA
CTGTTTTGGCAAACGAAGTGACGGCGCGTAACAATCTGGATAATGCCGTT
GAACAGCTGCGTCAAATCACGGGCAACTACTATCCGGAACTGGCAGCACT
GAACGTTGAGAATTTCAAGACGGATAAGCCGCAACCTGTGAACGCGCTGC
TGAAAGAGGCGGAAAAGCGCAATCTGAGCCTGCTGCAAGCCCGTCTGAGC
CAAGACCTGGCGCGTGAGCAGATTCGTCAGGCACAAGATGGCCACCTGCC
AACCCTGGACTTGACGGCATCCACGGGTATCTCGGACACCAGCTACTCCG
GTAGCAAGACTCGCGGTGCAGCAGGTACGCAGTATGACGACTCTAACATG
GGTCAAAACAAAGTCGGCCTGTCTTTCAGCCTGCCGATCTACCAAGGTGG
CATGGTTAATTCTCAAGTTAAACAGGCGCAATACAACTTTGTCGGCGCGA
GCGAACAGCTGGAGAGCGCTCACCGTAGCGTGGTCCAGACCGTCCGTTCT
TCTTTTAACAACATTAACGCGAGCATCAGCAGCATTAACGCATACAAACA
AGCGGTGGTGAGCGCGCAATCGAGCCTGGACGCAATGGAGGCGGGTTACA
GCGTCGGTACGCGCACCATTGTCGACGTGCTGGATGCAACTACCACCCTG
TATAATGCAAAGCAAGAACTGGCAAATGCGCGCTACAACTATCTGATTAA
CCAGCTGAATATCAAATCCGCGCTGGGCACGCTGAACGAGCAGGATCTGC
TGGCATTGAACAACGCGCTGAGCAAGCCGGTAAGCACGAATCCGGAGAAC
GTCGCCCCACAAACCCCGGAACAGAATGCTATCGCGGACGGCTATGCCCC
GGACAGCCCGGCTCCGGTTGTGCAGCAGACTAGCGCTCGCACCACCACCA
GCAATGGTCATAATCCGTTCCGTAATCGTATTCACTTTGGTATTGGTGAG
CGTTTCTAA

### SEQ ID NO:129

The protein sequence encoded by A0318_ProNTerm_tolC_opt_A0318C, integrated at the ΔA0358-downstream locus in JCC2522, is:

MQKQQNLDYFSPQALALWAAIASLGVMSPAHAEPRSEGSHSDPLVPTATQ
VVVPALPVEDVAPTAAPASQTPAPQSENLAQSSTQAVTSPVAQAQEAPQD
SNLPQLYAQQQGNPNAQQANPENLMQVYQQARLSNPELRKSAADRDAAFE
KINEARSPLLPQLGLGADYTYSNGYRDANGINSNATSASLQLTQSIFDMS
KWRALTLQEKAAGIQDVTYQTDQQTLILNTATAYFNVLNAIDVLSYTQAQ
KEAIYRQLDQTTQRFNVGLVAITDVQNARAQYDTVLANEVTARNNLDNAV
EQLRQITGNYYPELAALNVENFKTDKPQPVNALLKEAEKRNLSLLQARLS
QDLAREQIRQAQDGHLPTLDLTASTGISDTSYSGSKTRGAAGTQYDDSNM
GQNKVGLSFSLPIYQGGMVNSQVKQAQYNFVGASEQLESAHRSVVQTVRS
SFNNINASISSINAYKQAVVSAQSSLDAMEAGYSVGTRTIVDVLDATTTL
YNAKQELANARYNYLINQLNIKSALGTLNEQDLLALNNALSKPVSTNPEN
VAPQTPEQNAIADGYAPDSPAPVVQQTSARTTTSNGHNPFRNRIHFGIGE
RF

### SEQ ID NO:130

The DNA sequence encoding A0318_ProNTerm_tolC_opt_A0585C, integrated at the ΔA0358-downstream locus in JCC2522, is:

ATGCAAAAACAACAGAATCTGGACTACTTTAGCCCGCAGGCGTTGGCACT
GTGGGCGGCTATTGCTTCCCTGGGTGTTATGAGCCCGGCACACGCGGAGC
CGCGTAGCGAGGGCAGCCATTCTGATCCGCTGGTTCCGACCGCGACGCAG
GTCGTGGTTCCGGCGCTGCCGGTGGAGGACGTTGCGCCGACCGCCGCACC
GGCATCGCAGACCCCGGCTCCTCAGAGCGAAAACTTGGCGCAATCCAGCA
CCCAAGCCGTCACGAGCCCTGTGGCGCAGGCGCAGGAAGCCCCGCAAGAC
AGCAATCTGCCGCAACTGTATGCCCAGCAGCAAGGTAACCCAAATGCCCA
ACAGGCGAACCCGGAGAATTTGATGCAGGTTTACCAGCAGGCGCGTCTGT
CCAATCCGGAGCTGCGTAAAAGCGCTGCCGACCGTGATGCCGCGTTTGAG
AAGATTAACGAAGCCCGCAGCCCGCTGCTGCCGCAGCTGGGTTTGGGCGC
TGACTACACCTACTCCAACGGCTATCGTGACGCCAACGGTATCAATAGCA
ATGCGACCAGCGCCAGCCTGCAACTGACCCAAAGCATTTTTGATATGAGC
AAATGGCGCGCTCTGACCCTGCAAGAGAAAGCGGCAGGTATCCAGGATGT
GACCTACCAAACGGACCAGCAGACCCTGATCTTGAACACGGCGACCGCGT
ATTTCAATGTTTTGAACGCAATCGATGTCCTGAGCTATACCCAGGCCCAG
AAGGAAGCGATTTATCGTCAGTTGGATCAGACCACCCAGCGCTTCAATGT
GGGTCTGGTGGCGATTACGGATGTTCAAAATGCGCGTGCGCAATACGATA
CTGTTTTGGCAAACGAAGTGACGGCGCGTAACAATCTGGATAATGCCGTT
GAACAGCTGCGTCAAATCACGGGCAACTACTATCCGGAACTGGCAGCACT
GAACGTTGAGAATTTCAAGACGGATAAGCCGCAACCTGTGAACGCGCTGC
TGAAAGAGGCGGAAAAGCGCAATCTGAGCCTGCTGCAAGCCCGTCTGAGC
CAAGACCTGGCGCGTGAGCAGATTCGTCAGGCACAAGATGGCCACCTGCC
AACCCTGGACTTGACGGCATCCACGGGTATCTCGGACACCAGCTACTCCG
GTAGCAAGACTCGCGGTGCAGCAGGTACGCAGTATGACGACTCTAACATG
GGTCAAAACAAAGTCGGCCTGTCTTTCAGCCTGCCGATCTACCAAGGTGG
CATGGTTAATTCTCAAGTTAAACAGGCGCAATACAACTTTGTCGGCGCGA
GCGAACAGCTGGAGAGCGCTCACCGTAGCGTGGTCCAGACCGTCCGTTCT
TCTTTTAACAACATTAACGCGAGCATCAGCAGCATTAACGCATACAAACA
AGCGGTGGTGAGCGCGCAATCGAGCCTGGACGCAATGGAGGCGGGTTACA
GCGTCGGTACGCGCACCATTGTCGACGTGCTGGATGCAACTACCACCCTG
TATAATGCAAAGCAAGAACTGGCAAATGCGCGCTACAACTATCTGATTAA
CCAGCTGAATATCAAATCCGCGCTGGGCACGCTGAACGAGCAGGATCTGC
TGGCATTGAACAACGCGCTGAGCAAGCCGGTAAGCACGAATCCGGAGAAC
GTCGCCCCACAAACCCCGGAACAGAATGCTATCGCGGACGGCTATGCCCC
GGACAGCCCGGCTCCGGTTGTGCAGCAGACTAGCGCTCGCACCACCACCA
GCAATGGTCATAATCCGTTCCGTAATGGGGATGCGGTGATTGCCCCGGCG
GCTCCCTAA

### SEQ ID NO:131

The protein sequence encoded by A0318_ProNTerm_tolC_opt_A0585C, integrated at the ΔA0358-downstream locus in JCC2522, is:

MQKQQNLDYFSPQALALWAAIASLGVMSPAHAEPRSEGSHSDPLVPTATQ
VVVPALPVEDVAPTAAPASQTPAPQSENLAQSSTQAVTSPVAQAQEAPQD
SNLPQLYAQQQGNPNAQQANPENLMQVYQQARLSNPELRKSAADRDAAFE
KINEARSPLLPQLGLGADYTYSNGYRDANGINSNATSASLQLTQSIFDMS
KWRALTLQEKAAGIQDVTYQTDQQTLILNTATAYFNVLNAIDVLSYTQAQ
KEAIYRQLDQTTQRFNVGLVAITDVQNARAQYDTVLANEVTARNNLDNAV
EQLRQITGNYYPELAALNVENFKTDKPQPVNALLKEAEKRNLSLLQARLS
QDLAREQIRQAQDGHLPTLDLTASTGISDTSYSGSKTRGAAGTQYDDSNM
GQNKVGLSFSLPIYQGGMVNSQVKQAQYNFVGASEQLESAHRSVVQTVRS
SFNNINASISSINAYKQAVVSAQSSLDAMEAGYSVGTRTIVDVLDATTTL
YNAKQELANARYNYLINQLNIKSALGTLNEQDLLALNNALSKPVSTNPEN
VAPQTPEQNAIADGYAPDSPAPVVQQTSARTTTSNGHNPFRNGDAVIAPA
AP

### SEQ ID NO:132

The DNA sequence encoding A0585_tolC_opt_A0318C, integrated at the ΔA0358-downstream locus in JCC2522, is:

ATGTTTGCCTTTCGTGACTTCTTGACCTTCAGCACCGGTGGCCTGGTTGT
CCTGTCCGGCGGTGGTGTTGCGATTGCGGAGAATTTGATGCAGGTTTACC
AGCAGGCGCGTCTGTCCAATCCGGAGCTGCGTAAAAGCGCTGCCGACCGT
GATGCCGCGTTTGAGAAGATTAACGAAGCCCGCAGCCCGCTGCTGCCGCA
GCTGGGTTTGGGCGCTGACTACACCTACTCCAACGGCTATCGTGACGCCA
ACGGTATCAATAGCAATGCGACCAGCGCCAGCCTGCAACTGACCCAAAGC
ATTTTTGATATGAGCAAATGGCGCGCTCTGACCCTGCAAGAGAAAGCGGC
AGGTATCCAGGATGTGACCTACCAAACGGACCAGCAGACCCTGATCTTGA
ACACGGCGACCGCGTATTTCAATGTTTTGAACGCAATCGATGTCCTGAGC
TATACCCAGGCCCAGAAGGAAGCGATTTATCGTCAGTTGGATCAGACCAC
CCAGCGCTTCAATGTGGGTCTGGTGGCGATTACGGATGTTCAAAATGCGC
GTGCGCAATACGATACTGTTTTGGCAAACGAAGTGACGGCGCGTAACAAT
CTGGATAATGCCGTTGAACAGCTGCGTCAAATCACGGGCAACTACTATCC
GGAACTGGCAGCACTGAACGTTGAGAATTTCAAGACGGATAAGCCGCAAC
CTGTGAACGCGCTGCTGAAAGAGGCGGAAAAGCGCAATCTGAGCCTGCTG
CAAGCCCGTCTGAGCCAAGACCTGGCGCGTGAGCAGATTCGTCAGGCACA
AGATGGCCACCTGCCAACCCTGGACTTGACGGCATCCACGGGTATCTCGG
ACACCAGCTACTCCGGTAGCAAGACTCGCGGTGCAGCAGGTACGCAGTAT
GACGACTCTAACATGGGTCAAAACAAAGTCGGCCTGTCTTTCAGCCTGCC
GATCTACCAAGGTGGCATGGTTAATTCTCAAGTTAAACAGGCGCAATACA
ACTTTGTCGGCGCGAGCGAACAGCTGGAGAGCGCTCACCGTAGCGTGGTC
CAGACCGTCCGTTCTTCTTTTAACAACATTAACGCGAGCATCAGCAGCAT
TAACGCATACAAACAAGCGGTGGTGAGCGCGCAATCGAGCCTGGACGCAA
TGGAGGCGGGTTACAGCGTCGGTACGCGCACCATTGTCGACGTGCTGGAT
GCAACTACCACCCTGTATAATGCAAAGCAAGAACTGGCAAATGCGCGCTA
CAACTATCTGATTAACCAGCTGAATATCAAATCCGCGCTGGGCACGCTGA
ACGAGCAGGATCTGCTGGCATTGAACAACGCGCTGAGCAAGCCGGTAAGC
ACGAATCCGGAGAACGTCGCCCCACAAACCCCGGAACAGAATGCTATCGC
GGACGGCTATGCCCCGGACAGCCCGGCTCCGGTTGTGCAGCAGACTAGCG
CTCGCACCACCACCAGCAATGGTCATAATCCGTTCCGTAATCGTATTCAC
TTTGGTATTGGTGAGCGTTTCTAA

### SEQ ID NO:133

The protein sequence encoded by A0585_tolC_opt_A0318C, integrated at the ΔA0358-downstream locus in JCC2522, is:

MFAFRDFLTFSTGGLVVLSGGGVAIAENLMQVYQQARLSNPELRKSAADR
DAAFEKINEARSPLLPQLGLGADYTYSNGYRDANGINSNATSASLQLTQS
IFDMSKWRALTLQEKAAGIQDVTYQTDQQTLILNTATAYFNVLNAIDVLS
YTQAQKEAIYRQLDQTTQRFNVGLVAITDVQNARAQYDTVLANEVTARNN
LDNAVEQLRQITGNYYPELAALNVENFKTDKPQPVNALLKEAEKRNLSLL
QARLSQDLAREQIRQAQDGHLPTLDLTASTGISDTSYSGSKTRGAAGTQY
DDSNMGQNKVGLSFSLPIYQGGMVNSQVKQAQYNFVGASEQLESAHRSVV
QTVRSSFNNINASISSINAYKQAVVSAQSSLDAMEAGYSVGTRTIVDVLD
ATTTLYNAKQELANARYNYLINQLNIKSALGTLNEQDLLALNNALSKPVS
TNPENVAPQTPEQNAIADGYAPDSPAPVVQQTSARTTTSNGHNPFRNRIH
FGIGERF

### SEQ ID NO:134

The DNA sequence encoding A0585_tolC_opt_A0585C, integrated at the ΔA0358-downstream locus in JCC2522, is:

ATGTTTGCCTTTCGTGACTTCTTGACCTTCAGCACCGGTGGCCTGGTTGT
CCTGTCCGGCGGTGGTGTTGCGATTGCGGAGAATTTGATGCAGGTTTACC
AGCAGGCGCGTCTGTCCAATCCGGAGCTGCGTAAAAGCGCTGCCGACCGT
GATGCCGCGTTTGAGAAGATTAACGAAGCCCGCAGCCCGCTGCTGCCGCA
GCTGGGTTTGGGCGCTGACTACACCTACTCCAACGGCTATCGTGACGCCA
ACGGTATCAATAGCAATGCGACCAGCGCCAGCCTGCAACTGACCCAAAGC
ATTTTTGATATGAGCAAATGGCGCGCTCTGACCCTGCAAGAGAAAGCGGC
AGGTATCCAGGATGTGACCTACCAAACGGACCAGCAGACCCTGATCTTGA
ACACGGCGACCGCGTATTTCAATGTTTTGAACGCAATCGATGTCCTGAGC
TATACCCAGGCCCAGAAGGAAGCGATTTATCGTCAGTTGGATCAGACCAC
CCAGCGCTTCAATGTGGGTCTGGTGGCGATTACGGATGTTCAAAATGCGC
GTGCGCAATACGATACTGTTTTGGCAAACGAAGTGACGGCGCGTAACAAT
CTGGATAATGCCGTTGAACAGCTGCGTCAAATCACGGGCAACTACTATCC
GGAACTGGCAGCACTGAACGTTGAGAATTTCAAGACGGATAAGCCGCAAC
CTGTGAACGCGCTGCTGAAAGAGGCGGAAAAGCGCAATCTGAGCCTGCTG
CAAGCCCGTCTGAGCCAAGACCTGGCGCGTGAGCAGATTCGTCAGGCACA
AGATGGCCACCTGCCAACCCTGGACTTGACGGCATCCACGGGTATCTCGG
ACACCAGCTACTCCGGTAGCAAGACTCGCGGTGCAGCAGGTACGCAGTAT
GACGACTCTAACATGGGTCAAAACAAAGTCGGCCTGTCTTTCAGCCTGCC
GATCTACCAAGGTGGCATGGTTAATTCTCAAGTTAAACAGGCGCAATACA
ACTTTGTCGGCGCGAGCGAACAGCTGGAGAGCGCTCACCGTAGCGTGGTC
CAGACCGTCCGTTCTTCTTTTAACAACATTAACGCGAGCATCAGCAGCAT
TAACGCATACAAACAAGCGGTGGTGAGCGCGCAATCGAGCCTGGACGCAA
TGGAGGCGGGTTACAGCGTCGGTACGCGCACCATTGTCGACGTGCTGGAT
GCAACTACCACCCTGTATAATGCAAAGCAAGAACTGGCAAATGCGCGCTA
CAACTATCTGATTAACCAGCTGAATATCAAATCCGCGCTGGGCACGCTGA
ACGAGCAGGATCTGCTGGCATTGAACAACGCGCTGAGCAAGCCGGTAAGC
ACGAATCCGGAGAACGTCGCCCCACAAACCCCGGAACAGAATGCTATCGC
GGACGGCTATGCCCCGGACAGCCCGGCTCCGGTTGTGCAGCAGACTAGCG
CTCGCACCACCACCAGCAATGGTCATAATCCGTTCCGTAATGGGGATGCG
GTGATTGCCCCGGCGGCTCCCTAA

### SEQ ID NO:135

The protein sequence encoded by A0585_tolC_opt_A0585C, integrated at the ΔA0358-downstream locus in JCC2522, is:

MFAFRDFLTFSTGGLVVLSGGGVAIAENLMQVYQQARLSNPELRKSAADR
DAAFEKINEARSPLLPQLGLGADYTYSNGYRDANGINSNATSASLQLTQS
IFDMSKWRALTLQEKAAGIQDVTYQTDQQTLILNTATAYFNVLNAIDVLS
YTQAQKEAIYRQLDQTTQRFNVGLVAITDVQNARAQYDTVLANEVTARNN
LDNAVEQLRQITGNYYPELAALNVENFKTDKPQPVNALLKEAEKRNLSLL
QARLSQDLAREQIRQAQDGHLPTLDLTASTGISDTSYSGSKTRGAAGTQY
DDSNMGQNKVGLSFSLPIYQGGMVNSQVKQAQYNFVGASEQLESAHRSVV
QTVRSSFNNINASISSINAYKQAVVSAQSSLDAMEAGYSVGTRTIVDVLD
ATTTLYNAKQELANARYNYLINQLNIKSALGTLNEQDLLALNNALSKPVS
TNPENVAPQTPEQNAIADGYAPDSPAPVVQQTSARTTTSNGHNPFRNGDA
VIAPAAP

The DNA sequence encoding, and the protein sequence encoded by, A0585_ProNTerm_tolC_opt, integrated at the ΔA0358-downstream locus in JCC2522 are identical to the A0585_ProNTerm_tolC_opt sequences discussed in, and associated with, Table 16.

### SEQ ID NO:136

The DNA sequence encoding A0585_ProNTerm_tolC_opt_A0318C, integrated at the ΔA0358-downstream locus in JCC2522, is:

ATGTTTGCCTTCCGTGACTTCCTGACGTTTAGCACGGGCGGTTTGGTCGT
GTTGAGCGGTGGCGGTGTTGCGATTGCACAAACCACCCCTCCGCAGATCG
CCACTCCGGAGCCGTTTATCGGTCAGACGCCGCAGGCACCGCTGCCACCG
CTGGCTGCGCCGTCCGTTGAAAGCCTGGACACCGCGGCTTTCCTGCCGAG
CCTGGGCGGTCTGTCCCAACCGACCACCCTGGCCGCACTGCCTTTGCCGA
GCCCGGAGTTGAACCTGTCGCCTACGGCGCATCTGGGTACCATCCAGGCG
CCAAGCCCGCTGTTGGCGCAAGTGGATACCACTGCGACCCCGAGCCCGAC
CACCGCGATTGACGTCACCCTGCCGACGGCGGAAACGAATCAAACCATTC
CGCTGGTCCAGCCGCTGCCGCCAGACCGCGTCATCAATGAGGACCTGAAC
CAACTGCTGGAGCCGATTGATAACCCGGCAGTTACGGTGCCGCAGGAAGC
GACCGCCGTTACGACCGATAATGTTGTGGATGAGAATTTGATGCAGGTTT
ACCAGCAGGCGCGTCTGTCCAATCCGGAGCTGCGTAAAAGCGCTGCCGAC
CGTGATGCCGCGTTTGAGAAGATTAACGAAGCCCGCAGCCCGCTGCTGCC
GCAGCTGGGTTTGGGCGCTGACTACACCTACTCCAACGGCTATCGTGACG
CCAACGGTATCAATAGCAATGCGACCAGCGCCAGCCTGCAACTGACCCAA
AGCATTTTTGATATGAGCAAATGGCGCGCTCTGACCCTGCAAGAGAAAGC
GGCAGGTATCCAGGATGTGACCTACCAAACGGACCAGCAGACCCTGATCT
TGAACACGGCGACCGCGTATTTCAATGTTTTGAACGCAATCGATGTCCTG
AGCTATACCCAGGCCCAGAAGGAAGCGATTTATCGTCAGTTGGATCAGAC
CACCCAGCGCTTCAATGTGGGTCTGGTGGCGATTACGGATGTTCAAAATG
CGCGTGCGCAATACGATACTGTTTTGGCAAACGAAGTGACGGCGCGTAAC
AATCTGGATAATGCCGTTGAACAGCTGCGTCAAATCACGGGCAACTACTA
TCCGGAACTGGCAGCACTGAACGTTGAGAATTTCAAGACGGATAAGCCGC
AACCTGTGAACGCGCTGCTGAAAGAGGCGGAAAAGCGCAATCTGAGCCTG
CTGCAAGCCCGTCTGAGCCAAGACCTGGCGCGTGAGCAGATTCGTCAGGC
ACAAGATGGCCACCTGCCAACCCTGGACTTGACGGCATCCACGGGTATCT
CGGACACCAGCTACTCCGGTAGCAAGACTCGCGGTGCAGCAGGTACGCAG
TATGACGACTCTAACATGGGTCAAAACAAAGTCGGCCTGTCTTTCAGCCT
GCCGATCTACCAAGGTGGCATGGTTAATTCTCAAGTTAAACAGGCGCAAT
ACAACTTTGTCGGCGCGAGCGAACAGCTGGAGAGCGCTCACCGTAGCGTG
GTCCAGACCGTCCGTTCTTCTTTTAACAACATTAACGCGAGCATCAGCAG
CATTAACGCATACAAACAAGCGGTGGTGAGCGCGCAATCGAGCCTGGACG
CAATGGAGGCGGGTTACAGCGTCGGTACGCGCACCATTGTCGACGTGCTG
GATGCAACTACCACCCTGTATAATGCAAAGCAAGAACTGGCAAATGCGCG
CTACAACTATCTGATTAACCAGCTGAATATCAAATCCGCGCTGGGCACGC
TGAACGAGCAGGATCTGCTGGCATTGAACAACGCGCTGAGCAAGCCGGTA
AGCACGAATCCGGAGAACGTCGCCCCACAAACCCCGGAACAGAATGCTAT
CGCGGACGGCTATGCCCCGGACAGCCCGGCTCCGGTTGTGCAGCAGACTA
GCGCTCGCACCACCACCAGCAATGGTCATAATCCGTTCCGTAATCGTATT
CACTTTGGTATTGGTGAGCGTTTCTAA

### SEQ ID NO:137

The protein sequence encoded by A0585_ProNTerm_tolC_opt_A0318C, integrated at the ΔA0358-downstream locus in JCC2522, is:

MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDENLMQVYQQARLSNPELRKSAAD
RDAAFEKINEARSPLLPQLGLGADYTYSNGYRDANGINSNATSASLQLTQ
SIFDMSKWRALTLQEKAAGIQDVTYQTDQQTLILNTATAYFNVLNAIDVL
SYTQAQKEAIYRQLDQTTQRFNVGLVAITDVQNARAQYDTVLANEVTARN
NLDNAVEQLRQITGNYYPELAALNVENFKTDKPQPVNALLKEAEKRNLSL
LQARLSQDLAREQIRQAQDGHLPTLDLTASTGISDTSYSGSKTRGAAGTQ
YDDSNMGQNKVGLSFSLPIYQGGMVNSQVKQAQYNFVGASEQLESAHRSV
VQTVRSSFNNINASISSINAYKQAVVSAQSSLDAMEAGYSVGTRTIVDVL
DATTTLYNAKQELANARYNYLINQLNIKSALGTLNEQDLLALNNALSKPV
STNPENVAPQTPEQNAIADGYAPDSPAPVVQQTSARTTTSNGHNPFRNRI
HFGIGERF

### SEQ ID NO:138

The DNA sequence encoding A0585_ProNTerm_tolC_opt_A0585C, integrated at the ΔA0358-downstream locus in JCC2522, is:

ATGTTTGCCTTCCGTGACTTCCTGACGTTTAGCACGGGCGGTTTGGTCGT
GTTGAGCGGTGGCGGTGTTGCGATTGCACAAACCACCCCTCCGCAGATCG
CCACTCCGGAGCCGTTTATCGGTCAGACGCCGCAGGCACCGCTGCCACCG
CTGGCTGCGCCGTCCGTTGAAAGCCTGGACACCGCGGCTTTCCTGCCGAG
CCTGGGCGGTCTGTCCCAACCGACCACCCTGGCCGCACTGCCTTTGCCGA
GCCCGGAGTTGAACCTGTCGCCTACGGCGCATCTGGGTACCATCCAGGCG
CCAAGCCCGCTGTTGGCGCAAGTGGATACCACTGCGACCCCGAGCCCGAC
CACCGCGATTGACGTCACCCTGCCGACGGCGGAAACGAATCAAACCATTC
CGCTGGTCCAGCCGCTGCCGCCAGACCGCGTCATCAATGAGGACCTGAAC
CAACTGCTGGAGCCGATTGATAACCCGGCAGTTACGGTGCCGCAGGAAGC
GACCGCCGTTACGACCGATAATGTTGTGGATGAGAATTTGATGCAGGTTT
ACCAGCAGGCGCGTCTGTCCAATCCGGAGCTGCGTAAAAGCGCTGCCGAC
CGTGATGCCGCGTTTGAGAAGATTAACGAAGCCCGCAGCCCGCTGCTGCC
GCAGCTGGGTTTGGGCGCTGACTACACCTACTCCAACGGCTATCGTGACG
CCAACGGTATCAATAGCAATGCGACCAGCGCCAGCCTGCAACTGACCCAA
AGCATTTTTGATATGAGCAAATGGCGCGCTCTGACCCTGCAAGAGAAAGC
GGCAGGTATCCAGGATGTGACCTACCAAACGGACCAGCAGACCCTGATCT
TGAACACGGCGACCGCGTATTTCAATGTTTTGAACGCAATCGATGTCCTG
AGCTATACCCAGGCCCAGAAGGAAGCGATTTATCGTCAGTTGGATCAGAC
CACCCAGCGCTTCAATGTGGGTCTGGTGGCGATTACGGATGTTCAAAATG
CGCGTGCGCAATACGATACTGTTTTGGCAAACGAAGTGACGGCGCGTAAC
AATCTGGATAATGCCGTTGAACAGCTGCGTCAAATCACGGGCAACTACTA
TCCGGAACTGGCAGCACTGAACGTTGAGAATTTCAAGACGGATAAGCCGC
AACCTGTGAACGCGCTGCTGAAAGAGGCGGAAAAGCGCAATCTGAGCCTG
CTGCAAGCCCGTCTGAGCCAAGACCTGGCGCGTGAGCAGATTCGTCAGGC
ACAAGATGGCCACCTGCCAACCCTGGACTTGACGGCATCCACGGGTATCT
CGGACACCAGCTACTCCGGTAGCAAGACTCGCGGTGCAGCAGGTACGCAG
TATGACGACTCTAACATGGGTCAAAACAAAGTCGGCCTGTCTTTCAGCCT
GCCGATCTACCAAGGTGGCATGGTTAATTCTCAAGTTAAACAGGCGCAAT
ACAACTTTGTCGGCGCGAGCGAACAGCTGGAGAGCGCTCACCGTAGCGTG
GTCCAGACCGTCCGTTCTTCTTTTAACAACATTAACGCGAGCATCAGCAG
CATTAACGCATACAAACAAGCGGTGGTGAGCGCGCAATCGAGCCTGGACG
CAATGGAGGCGGGTTACAGCGTCGGTACGCGCACCATTGTCGACGTGCTG
GATGCAACTACCACCCTGTATAATGCAAAGCAAGAACTGGCAAATGCGCG
CTACAACTATCTGATTAACCAGCTGAATATCAAATCCGCGCTGGGCACGC
TGAACGAGCAGGATCTGCTGGCATTGAACAACGCGCTGAGCAAGCCGGTA
AGCACGAATCCGGAGAACGTCGCCCCACAAACCCCGGAACAGAATGCTAT
CGCGGACGGCTATGCCCCGGACAGCCCGGCTCCGGTTGTGCAGCAGACTA
GCGCTCGCACCACCACCAGCAATGGTCATAATCCGTTCCGTAATGGGGAT
GCGGTGATTGCCCCGGCGGCTCCCTAA

### SEQ ID NO:139

The protein sequence encoded by A0585_ProNTerm_tolC_opt_A0585C, integrated at the ΔA0358-downstream locus in JCC2522, is:

MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDENLMQVYQQARLSNPELRKSAAD
RDAAFEKINEARSPLLPQLGLGADYTYSNGYRDANGINSNATSASLQLTQ
SIFDMSKWRALTLQEKAAGIQDVTYQTDQQTLILNTATAYFNVLNAIDVL
SYTQAQKEAIYRQLDQTTQRFNVGLVAITDVQNARAQYDTVLANEVTARN
NLDNAVEQLRQITGNYYPELAALNVENFKTDKPQPVNALLKEAEKRNLSL
LQARLSQDLAREQIRQAQDGHLPTLDLTASTGISDTSYSGSKTRGAAGTQ
YDDSNMGQNKVGLSFSLPIYQGGMVNSQVKQAQYNFVGASEQLESAHRSV
VQTVRSSFNNINASISSINAYKQAVVSAQSSLDAMEAGYSVGTRTIVDVL
DATTTLYNAKQELANARYNYLINQLNIKSALGTLNEQDLLALNNALSKPV
STNPENVAPQTPEQNAIADGYAPDSPAPVVQQTSARTTTSNGHNPFRNGD
AVIAPAAP

The DNA sequence encoding, and the protein sequence encoded by, hybrid_A0585, integrated at the ΔA0358-downstream locus in JCC2522 are identical to the hybrid_A0585 sequences discussed in, and associated with, Table 16.

The DNA sequence encoding, and the protein sequence encoded by, hybrid_{—}1761, integrated at the ΔA0358-downstream locus in JCC2522 are identical to the hybrid_{—}1761 sequences discussed in, and associated with, Table 16.

The DNA sequences encoding, and the protein sequences encoded by, all omp variants, other than SYNPCC7002_A0585, integrated at the ΔA0358-downstream locus in JCC2055 with the ybhG-hairpin panel have been indicated in the respectively named sequences associated with Table 15 and Table 16.

### SEQ ID NO:140

The DNA sequences encoding SYNPCC7002_A0585, the wild-type JCC138 ORF of the same name, is integrated at the ΔA0358-downstream locus in JCC2055 with the ybhG-hairpin panel, is:

ATGTTCGCTTTTCGAGATTTTCTTACTTTCAGTACCGGTGGCCTTGTGGT
TCTCTCTGGTGGTGGGGTGGCGATCGCCCAAACAACCCCGCCGCAAATCG
CTACTCCAGAACCTTTCATCGGCCAGACCCCCCAGGCGCCATTGCCACCA
TTGGCCGCTCCTAGCGTTGAATCCCTCGATACAGCAGCCTTTTTACCGAG
TCTCGGTGGTCTCAGCCAACCCACAACCCTGGCCGCTTTACCTCTACCTT
CCCCAGAGCTCAATTTATCCCCGACTGCCCACCTCGGCACAATTCAAGCT
CCCTCGCCGCTCCTTGCCCAGGTAGATACAACGGCGACCCCCTCCCCAAC
AACCGCCATTGATGTGACCCTGCCCACCGCAGAGACAAACCAGACGATTC
CCCTTGTGCAACCCTTACCGCCGGATCGGGTGATTAATGAAGATCTAAAT
CAGCTCCTAGAGCCCATCGATAATCCGGCAGTGACAGTCCCCCAGGAGGC
CACGGCGGTGACGACTGACAATGTTGTTGACCTCACCCTAGAAGAAACGA
TTCGTCTGGCCCTAGAGCGCAATGAAACGCTCCAGGAAGCCCGTCTGAAC
TACGACCGATCAGAGGAACTGGTGCGAGAGGCGATCGCCGCCGAATACCC
AAATCTCAGCAACCAGGTTGACATTACCCGCACCGATAGCGCCAACGGAG
AACTCCAGGCCCGACGGCTGGGGGGAGACAACAATGCCACCACAGCGATC
AATGGTCGTCTCGAAGTCAGCTATGACATCTATACCGGGGGGCGTCGCTC
TGCCCAAATTGAAGCAGCCCAGACCCAATTGCAAATTGCTGAACTAGACA
TCGAGCGCCTCACCGAAGAAACTCGTCTAGCCGCTGCGGTGAACTATTAC
AATCTCCAGAGTGCCGACGCCCAGGTGGTTATCGAGCAAAGTTCGGTGTT
TGATGCCACCCAGAGTTTACGGGATGCCACCCTACTAGAACAGGCAGGCT
TGGGCACAAAATTTGATGTGTTGCGGGCCGAGGTCGAACTCGCTAGTGCC
CAACAGCGGCTCACCAGGGCTGAAGCCACCCAAAGAACCGCCCGGCGTCA
ACTGGCTCAACTGCTGAGTTTGGAACCGACCATCGATCCCCGCACCGCCG
ATGAGATTAACCTCGCTGGAAGATGGGAAATTTCTTTAGAAGAAACCATT
GTCCTGGCATTGCAAAACCGCCAAGAATTGCGCCAGCAGCTCCTCCAGCG
GGAAGTTGATGGTTACCAGGAACGGATTGCATTGGCTGCCGTTCGACCTT
TAGTCAGCGTTTTTGCGAATTATGATGTCTTGGAAGTGTTTGATGATAGC
CTTGGCCCCGCCGATGGGTTAACGGTTGGGGCCCGGATGCGTTGGAATTT
CTTTGATGGGGGTGCAGCGGCCGCCCGGGCAAATCAAGAGCAAGTTGATC
AGGCGATCGCCGAAAATCGTTTTGCTAACCAAAGAAACCAAATTCGCCTG
GCGGTGGAAACGGCCTACTATGACTTTGAAGCCAGCGAACAAAACATCAC
GACGGCAGCCGCCGCAGTCACTTTAGCAGAAGAAAGTTTACGCCTGGCTC
GTCTGCGCTTTAATGCAGGGGTCGGCACCCAAACCGATGTAATCTCTGCC
CAAACGGGTCTGAATACGGCCCGGGGGAACTATCTTCAGGCAGTCACCGA
TTACAATCGTGCCTTTGCCCAACTGAAACGGGAAGTCGGTTTAGGGGATG
CGGTGATTGCCCCGGCGGCTCCCTAG

### SEQ ID NO:141

The protein sequence encoded by SYNPCC7002_A0585, the wild-type JCC138 ORF of the same name, is integrated at the ΔA0358-downstream locus in JCC2055 with the ybhG-hairpin panel, is:

MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDLTLEETIRLALERNETLQEARLN
YDRSEELVREAIAAEYPNLSNQVDITRTDSANGELQARRLGGDNNATTAI
NGRLEVSYDIYTGGRRSAQIEAAQTQLQIAELDIERLTEETRLAAAVNYY
NLQSADAQVVIEQSSVFDATQSLRDATLLEQAGLGTKFDVLRAEVELASA
QQRLTRAEATQRTARRQLAQLLSLEPTIDPRTADEINLAGRWEISLEETI
VLALQNRQELRQQLLQREVDGYQERIALAAVRPLVSVFANYDVLEVFDDS
LGPADGLTVGARMRWNFFDGGAAAARANQEQVDQAIAENRFANQRNQIRL
AVETAYYDFEASEQNITTAAAAVTLAEESLRLARLRFNAGVGTQTDVISA
QTGLNTARGNYLQAVTDYNRAFAQLKREVGLGDAVIAPAAP

The DNA sequences of all 22 either-orientation promoters have been indicated in the respectively named sequences associated with Table 16.

### SEQ ID NO:142

The DNA sequence encoding ybhG_opt_hp1, integrated as part of the ybhGFSR operon at the ΔA0358-downstream locus in JCC2055, is:

ATGATGAAAAAGCCGGTTGTTATCGGTTTGGCGGTGGTGGTTCTGGCAGC
AGTCGTTGCGGGTGGCTACTGGTGGTATCAAAGCCGCCAGGATAACGGTT
TGACCCTGTATGGCAATGTTGATATTCGCACCGTCAACCTGTCGTTCCGC
GTGGGTGGCCGTGTGGAGAGCCTGGCCGTGGATGAAGGCGATGCGATCAA
AGCAGGTCAGGTCCTAGGTGAGCTGGATCACAAACCATACGAAATCGCCC
TGATGCAAGCCAAAGCGGGTGTTAGCGTGGCACAAGCGCAGTACGATCTG
ATGTTGGCGGGTTACCGCAATGAAGAGATTGCGCAGGCGGCAGCGGCGGT
GAAACAAGCGCAAGCGGCGTATGACCTGGCTAAGGCCGACGGCGACCGTT
TCCAAGAGCTGTATGCAAGCGGTGTGGTTAGCAAGCAACGTCTGGAGCAG
GCGCAGACCAGCCGTGATCAGGCACAGGCCACGCTGAAGAGCGCGCAGGA
TAAGCTGCGCCAATATCGTAGCGGCAATCGTGAACAAGACATTGCACAGG
CTAAGGCATCTCTGGAACAGGCCCAAGCTCAACTGGCCCAGGCGGAACTG
AACCTGCAGGACTCCACTCTGATCGCACCTTCTGACGGTACTTTGCTGAC
GCGTGCGGTTGAACCGGGTACCGTGCTGAATGAGGGCGGTACGGTTTTCA
CGGTCAGCCTGACGCGTCCGGTCTGGGTTCGTGCCTACGTCGATGAGCGT
AACCTGGACCAGGCGCAACCAGGCCGTAAGGTTCTGCTGTATACCGACGG
TCGCCCGGATAAACCTTACCACGGTCAAATTGGCTTTGTTTCCCCGACGG
CTGAGTTTACCCCGAAAACCGTCGAAACGCCGGACCTGCGTACCGACCTG
GTCTACCGTCTGCGCATCGTCGTGACCGACGCGGATGACGCATTGCGTCA
GGGCATGCCGGTGACCGTGCAGTTCGGCGACGAGGCTGGTCATGAGTAA

### SEQ ID NO:143

The protein sequence encoded by ybhG_opt_hp1, integrated as part of the ybhGFSR operon at the ΔA0358-downstream locus in JCC2055, is:

MMKKPVVIGLAVVVLAAVVAGGYWWYQSRQDNGLTLYGNVDIRTVNLSFR
VGGRVESLAVDEGDAIKAGQVLGELDHKPYEIALMQAKAGVSVAQAQYDL
MLAGYRNEEIAQAAAAVKQAQAAYDLAKADGDRFQELYASGVVSKQRLEQ
AQTSRDQAQATLKSAQDKLRQYRSGNREQDIAQAKASLEQAQAQLAQAEL
NLQDSTLIAPSDGTLLTRAVEPGTVLNEGGTVFTVSLTRPVWVRAYVDER
NLDQAQPGRKVLLYTDGRPDKPYHGQIGFVSPTAEFTPKTVETPDLRTDL
VYRLRIVVTDADDALRQGMPVTVQFGDEAGHE

### SEQ ID NO:144

The DNA sequence encoding ybhG_opt_hp2, integrated as part of the ybhGFSR operon at the ΔA0358-downstream locus in JCC2055, is:

ATGATGAAAAAGCCGGTTGTTATCGGTTTGGCGGTGGTGGTTCTGGCAGC
AGTCGTTGCGGGTGGCTACTGGTGGTATCAAAGCCGCCAGGATAACGGTT
TGACCCTGTATGGCAATGTTGATATTCGCACCGTCAACCTGTCGTTCCGC
GTGGGTGGCCGTGTGGAGAGCCTGGCCGTGGATGAAGGCGATGCGATCAA
AGCAGGTCAGGTCCTAGGTGAGCTGGATAGCGCCGAACTGCAGGCATCCC
TGGATGGTGCACAAGCCCGTATCAATGCGGCGCAGCAGCAGGTTAATCAA
GCACAGCTGCAAATCACCGTGATTGAAAACCAGATTACCGAGGCACAGCT
GACCCAACGCCAAGCACAGGATGACACTGCCGGTCGCGTTAATGCGGCAC
AAGCGAACGTGGCGGCAGCCAAGGCGCAACTGGCCCAGGCGCAAGCGCAG
GTCAAGCAGCTGGAAGCAGAGCTGGCCCTGGCGAAGGCAGACGGTGACCG
TTTCCAAGAACTGTACGCGAGCGGTGTGGTGAGCAAACAGCGTCTGGAGC
AAGCTCAAACCCAATATCTGAGCACGAAAGAGAATCTGGATGCTCGTCGC
GCGGTTGTTGCGGCAGCTGCGGAGCAAGTGAAAACCGCGGAGGGTAACCT
GACGCAAACTCAGGCGTCCCAGTTCAACCCAGACATTCAGTACCTGAGCA
CCAAAGAAAATCTGGACGCACGTCGTGCTGTCGTCGCTGCCGCTGCAGAA
CAAGTTAAGACCGCCGAGGGTAACTTGACTCAGACCCAAGCGAGCCAATT
CAACCCGGACATTCGTGCAGTTCAAGTGCAGCGCCTGCAAACGCAACTGG
TCCAGGCGCAGGCCCAGCTGTCTGCGGCGCAAGCACAAGTTCAGAATGCT
CAGGCCAACTATAACGAGATCGCGGCGAACCTGCAGGACTCCACTCTGAT
CGCACCTTCTGACGGTACTTTGCTGACGCGTGCGGTTGAACCGGGTACCG
TGCTGAATGAGGGCGGTACGGTTTTCACGGTCAGCCTGACGCGTCCGGTC
TGGGTTCGTGCCTACGTCGATGAGCGTAACCTGGACCAGGCGCAACCAGG
CCGTAAGGTTCTGCTGTATACCGACGGTCGCCCGGATAAACCTTACCACG
GTCAAATTGGCTTTGTTTCCCCGACGGCTGAGTTTACCCCGAAAACCGTC
GAAACGCCGGACCTGCGTACCGACCTGGTCTACCGTCTGCGCATCGTCGT
GACCGACGCGGATGACGCATTGCGTCAGGGCATGCCGGTGACCGTGCAGT
TCGGCGACGAGGCTGGTCATGAGTAA

### SEQ ID NO:145

The protein sequence encoded by ybhG_opt_hp2, integrated as part of the ybhGFSR operon at the ΔA0358-downstream locus in JCC2055, is:

MMKKPVVIGLAVVVLAAVVAGGYWWYQSRQDNGLTLYGNVDIRTVNLSFR
VGGRVESLAVDEGDAIKAGQVLGELDSAELQASLDGAQARINAAQQQVNQ
AQLQITVIENQITEAQLTQRQAQDDTAGRVNAAQANVAAAKAQLAQAQAQ
VKQLEAELALAKADGDRFQELYASGVVSKQRLEQAQTQYLSTKENLDARR
AVVAAAAEQVKTAEGNLTQTQASQFNPDIQYLSTKENLDARRAVVAAAAE
QVKTAEGNLTQTQASQFNPDIRAVQVQRLQTQLVQAQAQLSAAQAQVQNA
QANYNEIAANLQDSTLIAPSDGTLLTRAVEPGTVLNEGGTVFTVSLTRPV
WVRAYVDERNLDQAQPGRKVLLYTDGRPDKPYHGQIGFVSPTAEFTPKTV
ETPDLRTDLVYRLRIVVTDADDALRQGMPVTVQFGDEAGHE

### SEQ ID NO:146

The DNA sequence encoding ybhG_opt_hp3, integrated as part of the ybhGFSR operon at the ΔA0358-downstream locus in JCC2055, is:

ATGATGAAAAAGCCGGTTGTTATCGGTTTGGCGGTGGTGGTTCTGGCAGC
AGTCGTTGCGGGTGGCTACTGGTGGTATCAAAGCCGCCAGGATAACGGTT
TGACCCTGTATGGCAATGTTGATATTCGCACCGTCAACCTGTCGTTCCGC
GTGGGTGGCCGTGTGGAGAGCCTGGCCGTGGATGAAGGCGATGCGATCAA
AGCAGGTCAGGTCCTAGGTGAGCTGGATAGCGCCGAACTGCAGGCATCCC
TGGATGGTGCACAAGCCCGTATCAATGCGGCGCAGCAGCAGGTTAATCAA
GCACAGCTGCAAATCACCGTGATTGAAAACCAGATTACCGAGGCACAGCT
GACCCAACGCCAAGCACAGGATGACACTGCCGGTCGCGTTAATGCGGCAC
AAGCGAACGTGGCGGCAGCCAAGGCGCAACTGGCCCAGGCGCAAGCGCAG
GTCAAGCAGCTGGAAGCAGAGCTGGCCTATGCGCAAAACTTTTACAATCG
CCAGCAAGGTTTGTGGAAGAGCCGTACGATTAGCGCAAACGATCTGGAAA
ATGCGCGTTCTCAATATCTGAGCACGAAAGAGAATCTGGATGCTCGTCGC
GCGGTTGTTGCGGCAGCTGCGGAGCAAGTGAAAACCGCGGAGGGTAACCT
GACGCAAACTCAGGCGTCCCAGTTCAACCCAGACATTCAGTACCTGAGCA
CCAAAGAAAATCTGGACGCACGTCGTGCTGTCGTCGCTGCCGCTGCAGAA
CAAGTTAAGACCGCCGAGGGTAACTTGACTCAGACCCAAGCGAGCCAATT
CAACCCGGACATTCGTGCAGTTCAAGTGCAGCGCCTGCAAACGCAACTGG
TCCAGGCGCAGGCCCAGCTGTCTGCGGCGCAAGCACAAGTTCAGAATGCT
CAGGCCAACTATAACGAGATCGCGGCGAACCTGCAGGACTCCACTCTGAT
CGCACCTTCTGACGGTACTTTGCTGACGCGTGCGGTTGAACCGGGTACCG
TGCTGAATGAGGGCGGTACGGTTTTCACGGTCAGCCTGACGCGTCCGGTC
TGGGTTCGTGCCTACGTCGATGAGCGTAACCTGGACCAGGCGCAACCAGG
CCGTAAGGTTCTGCTGTATACCGACGGTCGCCCGGATAAACCTTACCACG
GTCAAATTGGCTTTGTTTCCCCGACGGCTGAGTTTACCCCGAAAACCGTC
GAAACGCCGGACCTGCGTACCGACCTGGTCTACCGTCTGCGCATCGTCGT
GACCGACGCGGATGACGCATTGCGTCAGGGCATGCCGGTGACCGTGCAGT
TCGGCGACGAGGCTGGTCATGAGTAA

### SEQ ID NO:147

The protein sequence encoded by ybhG_opt_hp3, integrated as part of the ybhGFSR operon at the ΔA0358-downstream locus in JCC2055, is:

MMKKPVVIGLAVVVLAAVVAGGYWWYQSRQDNGLTLYGNVDIRTVNLSFR
VGGRVESLAVDEGDAIKAGQVLGELDSAELQASLDGAQARINAAQQQVNQ
AQLQITVIENQITEAQLTQRQAQDDTAGRVNAAQANVAAAKAQLAQAQAQ
VKQLEAELAYAQNFYNRQQGLWKSRTISANDLENARSQYLSTKENLDARR
AVVAAAAEQVKTAEGNLTQTQASQFNPDIQYLSTKENLDARRAVVAAAAE
QVKTAEGNLTQTQASQFNPDIRAVQVQRLQTQLVQAQAQLSAAQAQVQNA
QANYNEIAANLQDSTLIAPSDGTLLTRAVEPGTVLNEGGTVFTVSLTRPV
WVRAYVDERNLDQAQPGRKVLLYTDGRPDKPYHGQIGFVSPTAEFTPKTV
ETPDLRTDLVYRLRIVVTDADDALRQGMPVTVQFGDEAGHE

### SEQ ID NO:148

The DNA sequence encoding ybhG_opt_hp4, integrated as part of the ybhGFSR operon at the ΔA0358-downstream locus in JCC2055, is:

ATGATGAAAAAGCCGGTTGTTATCGGTTTGGCGGTGGTGGTTCTGGCAGC
AGTCGTTGCGGGTGGCTACTGGTGGTATCAAAGCCGCCAGGATAACGGTT
TGACCCTGTATGGCAATGTTGATATTCGCACCGTCAACCTGTCGTTCCGC
GTGGGTGGCCGTGTGGAGAGCCTGGCCGTGGATGAAGGCGATGCGATCAA
AGCAGGTCAGGTCCTAGGTGAGCTGGATCACAAACCATACGAAATCGCCC
TGATGCAAGCCAAAGCGGGTGTTAGCGTGGCACAAGCGCAGTACGATCTG
ATGTTGGCGGGTTACCGCAATGAAGAGATTGCGCAGGCGGCAGCGGCGGT
GAAACAAGCGCAAGCGGCGTATGACTATGCGCAAAACTTTTACAATCGTT
TCCAAGAGCTGTATGCAAGCGGTGTGGTTAGCAAGCAAGATCTGGAAAAT
GCGCGTTCTAGCCGTGATCAGGCACAGGCCACGCTGAAGAGCGCGCAGGA
TAAGCTGCGCCAATATCGTAGCGGCAATCGTGAACAAGACATTGCACAGG
CTAAGGCATCTCTGGAACAGGCCCAAGCTCAACTGGCCCAGGCGGAACTG
AACCTGCAGGACTCCACTCTGATCGCACCTTCTGACGGTACTTTGCTGAC
GCGTGCGGTTGAACCGGGTACCGTGCTGAATGAGGGCGGTACGGTTTTCA
CGGTCAGCCTGACGCGTCCGGTCTGGGTTCGTGCCTACGTCGATGAGCGT
AACCTGGACCAGGCGCAACCAGGCCGTAAGGTTCTGCTGTATACCGACGG
TCGCCCGGATAAACCTTACCACGGTCAAATTGGCTTTGTTTCCCCGACGG
CTGAGTTTACCCCGAAAACCGTCGAAACGCCGGACCTGCGTACCGACCTG
GTCTACCGTCTGCGCATCGTCGTGACCGACGCGGATGACGCATTGCGTCA
GGGCATGCCGGTGACCGTGCAGTTCGGCGACGAGGCTGGTCATGAGTAA

### SEQ ID NO:149

The protein sequence encoded by ybhG_opt_hp4, integrated as part of the ybhGFSR operon at the ΔA0358-downstream locus in JCC2055, is:

MMKKPVVIGLAVVVLAAVVAGGYWWYQSRQDNGLTLYGNVDIRTVNLSFR
VGGRVESLAVDEGDAIKAGQVLGELDHKPYEIALMQAKAGVSVAQAQYDL
MLAGYRNEETAQAAAAVKQAQAAYDYAQNFYNRFQELYASGVVSKQDLEN
ARSSRDQAQATLKSAQDKLRQYRSGNREQDIAQAKASLEQAQAQLAQAEL
NLQDSTLIAPSDGTLLTRAVEPGTVLNEGGTVFTVSLTRPVWVRAYVDER
NLDQAQPGRKVLLYTDGRPDKPYHGQIGFVSPTAEFTPKTVETPDLRTDL
VYRLRIVVTDADDALRQGMPVTVQFGDEAGHE

### SEQ ID NO:150

The DNA sequence encoding torA_ybhG_opt_hp1, integrated as part of the ybhGFSR operon at the ΔA0358-downstream locus in JCC2055, is:

ATGAACAACAACGATCTGTTTCAAGCAAGCCGCCGTCGCTTTCTGGCGCA
GCTGGGCGGCTTGACCGTCGCTGGCATGCTGGGTCCGAGCCTGCTGACGC
CACGCCGTGCAACCGCTGGTGGCTACTGGTGGTATCAAAGCCGCCAGGAT
AACGGTTTGACCCTGTATGGCAATGTTGATATTCGCACCGTCAACCTGTC
GTTCCGCGTGGGTGGCCGTGTGGAGAGCCTGGCCGTGGATGAAGGCGATG
CGATCAAAGCAGGTCAGGTCCTAGGTGAGCTGGATCACAAACCATACGAA
ATCGCCCTGATGCAAGCCAAAGCGGGTGTTAGCGTGGCACAAGCGCAGTA
CGATCTGATGTTGGCGGGTTACCGCAATGAAGAGATTGCGCAGGCGGCAG
CGGCGGTGAAACAAGCGCAAGCGGCGTATGACCTGGCTAAGGCCGACGGC
GACCGTTTCCAAGAGCTGTATGCAAGCGGTGTGGTTAGCAAGCAACGTCT
GGAGCAGGCGCAGACCAGCCGTGATCAGGCACAGGCCACGCTGAAGAGCG
CGCAGGATAAGCTGCGCCAATATCGTAGCGGCAATCGTGAACAAGACATT
GCACAGGCTAAGGCATCTCTGGAACAGGCCCAAGCTCAACTGGCCCAGGC
GGAACTGAACCTGCAGGACTCCACTCTGATCGCACCTTCTGACGGTACTT
TGCTGACGCGTGCGGTTGAACCGGGTACCGTGCTGAATGAGGGCGGTACG
GTTTTCACGGTCAGCCTGACGCGTCCGGTCTGGGTTCGTGCCTACGTCGA
TGAGCGTAACCTGGACCAGGCGCAACCAGGCCGTAAGGTTCTGCTGTATA
CCGACGGTCGCCCGGATAAACCTTACCACGGTCAAATTGGCTTTGTTTCC
CCGACGGCTGAGTTTACCCCGAAAACCGTCGAAACGCCGGACCTGCGTAC
CGACCTGGTCTACCGTCTGCGCATCGTCGTGACCGACGCGGATGACGCAT
TGCGTCAGGGCATGCCGGTGACCGTGCAGTTCGGCGACGAGGCTGGTCAT
GAGTAA

### SEQ ID NO:151

The protein sequence encoded by torA_ybhG_opt_hp1, integrated as part of the ybhGFSR operon at the ΔA0358-downstream locus in JCC2055, is:

MNNNDLFQASRRRFLAQLGGLTVAGMLGPSLLTPRRATAGGYWWYQSRQD
NGLTLYGNVDIRTVNLSFRVGGRVESLAVDEGDAIKAGQVLGELDHKPYE
IALMQAKAGVSVAQAQYDLMLAGYRNEEIAQAAAAVKQAQAAYDLAKADG
DRFQELYASGVVSKQRLEQAQTSRDQAQATLKSAQDKLRQYRSGNREQDI
AQAKASLEQAQAQLAQAELNLQDSTLTAPSDGTLLTRAVEPGTVLNEGGT
VFTVSLTRPVWVRAYVDERNLDQAQPGRKVLLYTDGRPDKPYHGQIGFVS
PTAEFTPKTVETPDLRTDLVYRLRIVVTDADDALRQGMPVTVQFGDEAGH
E

### SEQ ID NO:152

The DNA sequence encoding torA_ybhG_opt_hp2, integrated as part of the ybhGFSR operon at the ΔA0358-downstream locus in JCC2055, is:

ATGAACAACAACGATCTGTTTCAAGCAAGCCGCCGTCGCTTTCTGGCGCA
GCTGGGCGGCTTGACCGTCGCTGGCATGCTGGGTCCGAGCCTGCTGACGC
CACGCCGTGCAACCGCTGGTGGCTACTGGTGGTATCAAAGCCGCCAGGAT
AACGGTTTGACCCTGTATGGCAATGTTGATATTCGCACCGTCAACCTGTC
GTTCCGCGTGGGTGGCCGTGTGGAGAGCCTGGCCGTGGATGAAGGCGATG
CGATCAAAGCAGGTCAGGTCCTAGGTGAGCTGGATAGCGCCGAACTGCAG
GCATCCCTGGATGGTGCACAAGCCCGTATCAATGCGGCGCAGCAGCAGGT
TAATCAAGCACAGCTGCAAATCACCGTGATTGAAAACCAGATTACCGAGG
CACAGCTGACCCAACGCCAAGCACAGGATGACACTGCCGGTCGCGTTAAT
GCGGCACAAGCGAACGTGGCGGCAGCCAAGGCGCAACTGGCCCAGGCGCA
AGCGCAGGTCAAGCAGCTGGAAGCAGAGCTGGCCCTGGCGAAGGCAGACG
GTGACCGTTTCCAAGAACTGTACGCGAGCGGTGTGGTGAGCAAACAGCGT
CTGGAGCAAGCTCAAACCCAATATCTGAGCACGAAAGAGAATCTGGATGC
TCGTCGCGCGGTTGTTGCGGCAGCTGCGGAGCAAGTGAAAACCGCGGAGG
GTAACCTGACGCAAACTCAGGCGTCCCAGTTCAACCCAGACATTCAGTAC
CTGAGCACCAAAGAAAATCTGGACGCACGTCGTGCTGTCGTCGCTGCCGC
TGCAGAACAAGTTAAGACCGCCGAGGGTAACTTGACTCAGACCCAAGCGA
GCCAATTCAACCCGGACATTCGTGCAGTTCAAGTGCAGCGCCTGCAAACG
CAACTGGTCCAGGCGCAGGCCCAGCTGTCTGCGGCGCAAGCACAAGTTCA
GAATGCTCAGGCCAACTATAACGAGATCGCGGCGAACCTGCAGGACTCCA
CTCTGATCGCACCTTCTGACGGTACTTTGCTGACGCGTGCGGTTGAACCG
GGTACCGTGCTGAATGAGGGCGGTACGGTTTTCACGGTCAGCCTGACGCG
TCCGGTCTGGGTTCGTGCCTACGTCGATGAGCGTAACCTGGACCAGGCGC
AACCAGGCCGTAAGGTTCTGCTGTATACCGACGGTCGCCCGGATAAACCT
TACCACGGTCAAATTGGCTTTGTTTCCCCGACGGCTGAGTTTACCCCGAA
AACCGTCGAAACGCCGGACCTGCGTACCGACCTGGTCTACCGTCTGCGCA
TCGTCGTGACCGACGCGGATGACGCATTGCGTCAGGGCATGCCGGTGACC
GTGCAGTTCGGCGACGAGGCTGGTCATGAGTAA

### SEQ ID NO:153

The protein sequence encoded by torA_ybhG_opt_hp2, integrated as part of the ybhGFSR operon at the ΔA0358-downstream locus in JCC2055, is:

MNNNDLFQASRRRFLAQLGGLTVAGMLGPSLLTPRRATAGGYWWYQSRQD
NGLTLYGNVDIRTVNLSFRVGGRVESLAVDEGDAIKAGQVLGELDSAELQ
ASLDGAQARINAAQQQVNQAQLQITVIENQITEAQLTQRQAQDDTAGRVN
AAQANVAAAKAQLAQAQAQVKQLEAELALAKADGDRFQELYASGVVSKQR
LEQAQTQYLSTKENLDARRAVVAAAAEQVKTAEGNLTQTQASQFNPDIQY
LSTKENLDARRAVVAAAAEQVKTAEGNLTQTQASQFNPDIRAVQVQRLQT
QLVQAQAQLSAAQAQVQNAQANYNEIAANLQDSTLIAPSDGTLLTRAVEP
GTVLNEGGTVFTVSLTRPVWVRAYVDERNLDQAQPGRKVLLYTDGRPDKP
YHGQIGFVSPTAEFTPKTVETPDLRTDLVYRLRIVVTDADDALRQGMPVT
VQFGDEAGHE

### SEQ ID NO:154

The DNA sequence encoding torA_ybhG_opt_hp3, integrated as part of the ybhGFSR operon at the ΔA0358-downstream locus in JCC2055, is:

ATGAACAACAACGATCTGTTTCAAGCAAGCCGCCGTCGCTTTCTGGCGCA
GCTGGGCGGCTTGACCGTCGCTGGCATGCTGGGTCCGAGCCTGCTGACGC
CACGCCGTGCAACCGCTGGTGGCTACTGGTGGTATCAAAGCCGCCAGGAT
AACGGTTTGACCCTGTATGGCAATGTTGATATTCGCACCGTCAACCTGTC
GTTCCGCGTGGGTGGCCGTGTGGAGAGCCTGGCCGTGGATGAAGGCGATG
CGATCAAAGCAGGTCAGGTCCTAGGTGAGCTGGATAGCGCCGAACTGCAG
GCATCCCTGGATGGTGCACAAGCCCGTATCAATGCGGCGCAGCAGCAGGT
TAATCAAGCACAGCTGCAAATCACCGTGATTGAAAACCAGATTACCGAGG
CACAGCTGACCCAACGCCAAGCACAGGATGACACTGCCGGTCGCGTTAAT
GCGGCACAAGCGAACGTGGCGGCAGCCAAGGCGCAACTGGCCCAGGCGCA
AGCGCAGGTCAAGCAGCTGGAAGCAGAGCTGGCCTATGCGCAAAACTTTT
ACAATCGCCAGCAAGGTTTGTGGAAGAGCCGTACGATTAGCGCAAACGAT
CTGGAAAATGCGCGTTCTCAATATCTGAGCACGAAAGAGAATCTGGATGC
TCGTCGCGCGGTTGTTGCGGCAGCTGCGGAGCAAGTGAAAACCGCGGAGG
GTAACCTGACGCAAACTCAGGCGTCCCAGTTCAACCCAGACATTCAGTAC
CTGAGCACCAAAGAAAATCTGGACGCACGTCGTGCTGTCGTCGCTGCCGC
TGCAGAACAAGTTAAGACCGCCGAGGGTAACTTGACTCAGACCCAAGCGA
GCCAATTCAACCCGGACATTCGTGCAGTTCAAGTGCAGCGCCTGCAAACG
CAACTGGTCCAGGCGCAGGCCCAGCTGTCTGCGGCGCAAGCACAAGTTCA
GAATGCTCAGGCCAACTATAACGAGATCGCGGCGAACCTGCAGGACTCCA
CTCTGATCGCACCTTCTGACGGTACTTTGCTGACGCGTGCGGTTGAACCG
GGTACCGTGCTGAATGAGGGCGGTACGGTTTTCACGGTCAGCCTGACGCG
TCCGGTCTGGGTTCGTGCCTACGTCGATGAGCGTAACCTGGACCAGGCGC
AACCAGGCCGTAAGGTTCTGCTGTATACCGACGGTCGCCCGGATAAACCT
TACCACGGTCAAATTGGCTTTGTTTCCCCGACGGCTGAGTTTACCCCGAA
AACCGTCGAAACGCCGGACCTGCGTACCGACCTGGTCTACCGTCTGCGCA
TCGTCGTGACCGACGCGGATGACGCATTGCGTCAGGGCATGCCGGTGACC
GTGCAGTTCGGCGACGAGGCTGGTCATGAGTAA

### SEQ ID NO:155

The protein sequence encoded by torA_ybhG_opt_hp3, integrated as part of the ybhGFSR operon at the ΔA0358-downstream locus in JCC2055, is:

MNNNDLFQASRRRFLAQLGGLTVAGMLGPSLLTPRRATAGGYWWYQSRQD
NGLTLYGNVDIRTVNLSFRVGGRVESLAVDEGDAIKAGQVLGELDSAELQ
ASLDGAQARINAAQQQVNQAQLQITVIENQITEAQLTQRQAQDDTAGRVN
AAQANVAAAKAQLAQAQAQVKQLEAELAYAQNFYNRQQGLWKSRTISAND
LENARSQYLSTKENLDARRAVVAAAAEQVKTAEGNLTQTQASQFNPDIQY
LSTKENLDARRAVVAAAAEQVKTAEGNLTQTQASQFNPDIRAVQVQRLQT
QLVQAQAQLSAAQAQVQNAQANYNEIAANLQDSTLIAPSDGTLLTRAVEP
GTVLNEGGTVFTVSLTRPVWVRAYVDERNLDQAQPGRKVLLYTDGRPDKP
YHGQIGFVSPTAEFTPKTVETPDLRTDLVYRLRIVVTDADDALRQGMPVT
VQFGDEAGHE

### SEQ ID NO:156

The DNA sequence encoding torA_ybhG_opt_hp4, integrated as part of the ybhGFSR operon at the ΔA0358-downstream locus in JCC2055, is:

ATGAACAACAACGATCTGTTTCAAGCAAGCCGCCGTCGCTTTCTGGCGCA
GCTGGGCGGCTTGACCGTCGCTGGCATGCTGGGTCCGAGCCTGCTGACGC
CACGCCGTGCAACCGCTGGTGGCTACTGGTGGTATCAAAGCCGCCAGGAT
AACGGTTTGACCCTGTATGGCAATGTTGATATTCGCACCGTCAACCTGTC
GTTCCGCGTGGGTGGCCGTGTGGAGAGCCTGGCCGTGGATGAAGGCGATG
CGATCAAAGCAGGTCAGGTCCTAGGTGAGCTGGATCACAAACCATACGAA
ATCGCCCTGATGCAAGCCAAAGCGGGTGTTAGCGTGGCACAAGCGCAGTA
CGATCTGATGTTGGCGGGTTACCGCAATGAAGAGATTGCGCAGGCGGCAG
CGGCGGTGAAACAAGCGCAAGCGGCGTATGACTATGCGCAAAACTTTTAC
AATCGTTTCCAAGAGCTGTATGCAAGCGGTGTGGTTAGCAAGCAAGATCT
GGAAAATGCGCGTTCTAGCCGTGATCAGGCACAGGCCACGCTGAAGAGCG
CGCAGGATAAGCTGCGCCAATATCGTAGCGGCAATCGTGAACAAGACATT
GCACAGGCTAAGGCATCTCTGGAACAGGCCCAAGCTCAACTGGCCCAGGC
GGAACTGAACCTGCAGGACTCCACTCTGATCGCACCTTCTGACGGTACTT
TGCTGACGCGTGCGGTTGAACCGGGTACCGTGCTGAATGAGGGCGGTACG
GTTTTCACGGTCAGCCTGACGCGTCCGGTCTGGGTTCGTGCCTACGTCGA
TGAGCGTAACCTGGACCAGGCGCAACCAGGCCGTAAGGTTCTGCTGTATA
CCGACGGTCGCCCGGATAAACCTTACCACGGTCAAATTGGCTTTGTTTCC
CCGACGGCTGAGTTTACCCCGAAAACCGTCGAAACGCCGGACCTGCGTAC
CGACCTGGTCTACCGTCTGCGCATCGTCGTGACCGACGCGGATGACGCAT
TGCGTCAGGGCATGCCGGTGACCGTGCAGTTCGGCGACGAGGCTGGTCAT
GAGTAA

### SEQ ID NO:157

The protein sequence encoded by torA_ybhG_opt_hp4, integrated as part of the ybhGFSR operon at the ΔA0358-downstream locus in JCC2055, is:

MNNNDLFQASRRRFLAQLGGLTVAGMLGPSLLTPRRATAGGYWWYQSRQD
NGLTLYGNVDIRTVNLSFRVGGRVESLAVDEGDAIKAGQVLGELDHKPYE
IALMQAKAGVSVAQAQYDLMLAGYRNEEIAQAAAAVKQAQAAYDYAQNFY
NRFQELYASGVVSKQDLENARSSRDQAQATLKSAQDKLRQYRSGNREQDI
AQAKASLEQAQAQLAQAELNLQDSTLTAPSDGTLLTRAVEPGTVLNEGGT
VFTVSLTRPVWVRAYVDERNLDQAQPGRKVLLYTDGRPDKPYHGQIGFVS
PTAEFTPKTVETPDLRTDLVYRLRIVVTDADDALRQGMPVTVQFGDEAGH
E

### SEQ ID NO:158

The DNA sequence encoding A0318_ybhG_opt_hp1, integrated as part of the ybhGFSR operon at the ΔA0358-downstream locus in JCC2055, is:

ATGCAGAAGCAGCAGAACCTGGACTATTTCAGCCCGCAAGCGTTGGCGCT
GTGGGCAGCTATCGCCAGCCTGGGCGTTATGTCCCCAGCACACGCTGGTG
GCTACTGGTGGTATCAAAGCCGCCAGGATAACGGTTTGACCCTGTATGGC
AATGTTGATATTCGCACCGTCAACCTGTCGTTCCGCGTGGGTGGCCGTGT
GGAGAGCCTGGCCGTGGATGAAGGCGATGCGATCAAAGCAGGTCAGGTCC
TAGGTGAGCTGGATCACAAACCATACGAAATCGCCCTGATGCAAGCCAAA
GCGGGTGTTAGCGTGGCACAAGCGCAGTACGATCTGATGTTGGCGGGTTA
CCGCAATGAAGAGATTGCGCAGGCGGCAGCGGCGGTGAAACAAGCGCAAG
CGGCGTATGACCTGGCTAAGGCCGACGGCGACCGTTTCCAAGAGCTGTAT
GCAAGCGGTGTGGTTAGCAAGCAACGTCTGGAGCAGGCGCAGACCAGCCG
TGATCAGGCACAGGCCACGCTGAAGAGCGCGCAGGATAAGCTGCGCCAAT
ATCGTAGCGGCAATCGTGAACAAGACATTGCACAGGCTAAGGCATCTCTG
GAACAGGCCCAAGCTCAACTGGCCCAGGCGGAACTGAACCTGCAGGACTC
CACTCTGATCGCACCTTCTGACGGTACTTTGCTGACGCGTGCGGTTGAAC
CGGGTACCGTGCTGAATGAGGGCGGTACGGTTTTCACGGTCAGCCTGACG
CGTCCGGTCTGGGTTCGTGCCTACGTCGATGAGCGTAACCTGGACCAGGC
GCAACCAGGCCGTAAGGTTCTGCTGTATACCGACGGTCGCCCGGATAAAC
CTTACCACGGTCAAATTGGCTTTGTTTCCCCGACGGCTGAGTTTACCCCG
AAAACCGTCGAAACGCCGGACCTGCGTACCGACCTGGTCTACCGTCTGCG
CATCGTCGTGACCGACGCGGATGACGCATTGCGTCAGGGCATGCCGGTGA
CCGTGCAGTTCGGCGACGAGGCTGGTCATGAGTAA

### SEQ ID NO:159

The protein sequence encoded by A0318_ybhG_opt_hp1, integrated as part of the ybhGFSR operon at the ΔA0358-downstream locus in JCC2055, is:

MQKQQNLDYFSPQALALWAAIASLGVMSPAHAGGYWWYQSRQDNGLTLYG
NVDIRTVNLSFRVGGRVESLAVDEGDAIKAGQVLGELDHKPYEIALMQAK
AGVSVAQAQYDLMLAGYRNEEIAQAAAAVKQAQAAYDLAKADGDRFQELY
ASGVVSKQRLEQAQTSRDQAQATLKSAQDKLRQYRSGNREQDIAQAKASL
EQAQAQLAQAELNLQDSTLIAPSDGTLLTRAVEPGTVLNEGGTVFTVSLT
RPVWVRAYVDERNLDQAQPGRKVLLYTDGRPDKPYHGQIGFVSPTAEFTP
KTVETPDLRTDLVYRLRIVVTDADDALRQGMPVTVQFGDEAGHE

### SEQ ID NO:160

The DNA sequence encoding A0318_ybhG_opt_hp2, integrated as part of the ybhGFSR operon at the ΔA0358-downstream locus in JCC2055, is:

ATGCAGAAGCAGCAGAACCTGGACTATTTCAGCCCGCAAGCGTTGGCGCT
GTGGGCAGCTATCGCCAGCCTGGGCGTTATGTCCCCAGCACACGCTGGTG
GCTACTGGTGGTATCAAAGCCGCCAGGATAACGGTTTGACCCTGTATGGC
AATGTTGATATTCGCACCGTCAACCTGTCGTTCCGCGTGGGTGGCCGTGT
GGAGAGCCTGGCCGTGGATGAAGGCGATGCGATCAAAGCAGGTCAGGTCC
TAGGTGAGCTGGATAGCGCCGAACTGCAGGCATCCCTGGATGGTGCACAA
GCCCGTATCAATGCGGCGCAGCAGCAGGTTAATCAAGCACAGCTGCAAAT
CACCGTGATTGAAAACCAGATTACCGAGGCACAGCTGACCCAACGCCAAG
CACAGGATGACACTGCCGGTCGCGTTAATGCGGCACAAGCGAACGTGGCG
GCAGCCAAGGCGCAACTGGCCCAGGCGCAAGCGCAGGTCAAGCAGCTGGA
AGCAGAGCTGGCCCTGGCGAAGGCAGACGGTGACCGTTTCCAAGAACTGT
ACGCGAGCGGTGTGGTGAGCAAACAGCGTCTGGAGCAAGCTCAAACCCAA
TATCTGAGCACGAAAGAGAATCTGGATGCTCGTCGCGCGGTTGTTGCGGC
AGCTGCGGAGCAAGTGAAAACCGCGGAGGGTAACCTGACGCAAACTCAGG
CGTCCCAGTTCAACCCAGACATTCAGTACCTGAGCACCAAAGAAAATCTG
GACGCACGTCGTGCTGTCGTCGCTGCCGCTGCAGAACAAGTTAAGACCGC
CGAGGGTAACTTGACTCAGACCCAAGCGAGCCAATTCAACCCGGACATTC
GTGCAGTTCAAGTGCAGCGCCTGCAAACGCAACTGGTCCAGGCGCAGGCC
CAGCTGTCTGCGGCGCAAGCACAAGTTCAGAATGCTCAGGCCAACTATAA
CGAGATCGCGGCGAACCTGCAGGACTCCACTCTGATCGCACCTTCTGACG
GTACTTTGCTGACGCGTGCGGTTGAACCGGGTACCGTGCTGAATGAGGGC
GGTACGGTTTTCACGGTCAGCCTGACGCGTCCGGTCTGGGTTCGTGCCTA
CGTCGATGAGCGTAACCTGGACCAGGCGCAACCAGGCCGTAAGGTTCTGC
TGTATACCGACGGTCGCCCGGATAAACCTTACCACGGTCAAATTGGCTTT
GTTTCCCCGACGGCTGAGTTTACCCCGAAAACCGTCGAAACGCCGGACCT
GCGTACCGACCTGGTCTACCGTCTGCGCATCGTCGTGACCGACGCGGATG
ACGCATTGCGTCAGGGCATGCCGGTGACCGTGCAGTTCGGCGACGAGGCT
GGTCATGAGTAA

### SEQ ID NO:161

The protein sequence encoded by A0318_ybhG_opt_hp2, integrated as part of the ybhGFSR operon at the ΔA0358-downstream locus in JCC2055, is:

MQKQQNLDYFSPQALALWAAIASLGVMSPAHAGGYWWYQSRQDNGLTLYG
NVDIRTVNLSFRVGGRVESLAVDEGDAIKAGQVLGELDSAELQASLDGAQ
ARINAAQQQVNQAQLQITVIENQITEAQLTQRQAQDDTAGRVNAAQANVA
AAKAQLAQAQAQVKQLEAELALAKADGDRFQELYASGVVSKQRLEQAQTQ
YLSTKENLDARRAVVAAAAEQVKTAEGNLTQTQASQFNPDIQYLSTKENL
DARRAVVAAAAEQVKTAEGNLTQTQASQFNPDIRAVQVQRLQTQLVQAQA
QLSAAQAQVQNAQANYNEIAANLQDSTLIAPSDGTLLTRAVEPGTVLNEG
GTVFTVSLTRPVWVRAYVDERNLDQAQPGRKVLLYTDGRPDKPYHGQIGF
VSPTAEFTPKTVETPDLRTDLVYRLRIVVTDADDALRQGMPVTVQFGDEA
GHE

### SEQ ID NO:162

The DNA sequence encoding A0318_ybhG_opt_hp3, integrated as part of the ybhGFSR operon at the ΔA0358-downstream locus in JCC2055, is:

ATGCAGAAGCAGCAGAACCTGGACTATTTCAGCCCGCAAGCGTTGGCGCT
GTGGGCAGCTATCGCCAGCCTGGGCGTTATGTCCCCAGCACACGCTGGTG
GCTACTGGTGGTATCAAAGCCGCCAGGATAACGGTTTGACCCTGTATGGC
AATGTTGATATTCGCACCGTCAACCTGTCGTTCCGCGTGGGTGGCCGTGT
GGAGAGCCTGGCCGTGGATGAAGGCGATGCGATCAAAGCAGGTCAGGTCC
TAGGTGAGCTGGATAGCGCCGAACTGCAGGCATCCCTGGATGGTGCACAA
GCCCGTATCAATGCGGCGCAGCAGCAGGTTAATCAAGCACAGCTGCAAAT
CACCGTGATTGAAAACCAGATTACCGAGGCACAGCTGACCCAACGCCAAG
CACAGGATGACACTGCCGGTCGCGTTAATGCGGCACAAGCGAACGTGGCG
GCAGCCAAGGCGCAACTGGCCCAGGCGCAAGCGCAGGTCAAGCAGCTGGA
AGCAGAGCTGGCCTATGCGCAAAACTTTTACAATCGCCAGCAAGGTTTGT
GGAAGAGCCGTACGATTAGCGCAAACGATCTGGAAAATGCGCGTTCTCAA
TATCTGAGCACGAAAGAGAATCTGGATGCTCGTCGCGCGGTTGTTGCGGC
AGCTGCGGAGCAAGTGAAAACCGCGGAGGGTAACCTGACGCAAACTCAGG
CGTCCCAGTTCAACCCAGACATTCAGTACCTGAGCACCAAAGAAAATCTG
GACGCACGTCGTGCTGTCGTCGCTGCCGCTGCAGAACAAGTTAAGACCGC
CGAGGGTAACTTGACTCAGACCCAAGCGAGCCAATTCAACCCGGACATTC
GTGCAGTTCAAGTGCAGCGCCTGCAAACGCAACTGGTCCAGGCGCAGGCC
CAGCTGTCTGCGGCGCAAGCACAAGTTCAGAATGCTCAGGCCAACTATAA
CGAGATCGCGGCGAACCTGCAGGACTCCACTCTGATCGCACCTTCTGACG
GTACTTTGCTGACGCGTGCGGTTGAACCGGGTACCGTGCTGAATGAGGGC
GGTACGGTTTTCACGGTCAGCCTGACGCGTCCGGTCTGGGTTCGTGCCTA
CGTCGATGAGCGTAACCTGGACCAGGCGCAACCAGGCCGTAAGGTTCTGC
TGTATACCGACGGTCGCCCGGATAAACCTTACCACGGTCAAATTGGCTTT
GTTTCCCCGACGGCTGAGTTTACCCCGAAAACCGTCGAAACGCCGGACCT
GCGTACCGACCTGGTCTACCGTCTGCGCATCGTCGTGACCGACGCGGATG
ACGCATTGCGTCAGGGCATGCCGGTGACCGTGCAGTTCGGCGACGAGGCT
GGTCATGAGTAA

### SEQ ID NO:163

The protein sequence encoded by A0318_ybhG_opt_hp3, integrated as part of the ybhGFSR operon at the ΔA0358-downstream locus in JCC2055, is:

MQKQQNLDYFSPQALALWAAIASLGVMSPAHAGGYWWYQSRQDNGLTLYG
NVDIRTVNLSFRVGGRVESLAVDEGDAIKAGQVLGELDSAELQASLDGAQ
ARINAAQQQVNQAQLQITVIENQITEAQLTQRQAQDDTAGRVNAAQANVA
AAKAQLAQAQAQVKQLEAELAYAQNFYNRQQGLWKSRTISANDLENARSQ
YLSTKENLDARRAVVAAAAEQVKTAEGNLTQTQASQFNPDIQYLSTKENL
DARRAVVAAAAEQVKTAEGNLTQTQASQFNPDIRAVQVQRLQTQLVQAQA
QLSAAQAQVQNAQANYNEIAANLQDSTLIAPSDGTLLTRAVEPGTVLNEG
GTVFTVSLTRPVWVRAYVDERNLDQAQPGRKVLLYTDGRPDKPYHGQIGF
VSPTAEFTPKTVETPDLRTDLVYRLRIVVTDADDALRQGMPVTVQFGDEA
GHE

### SEQ ID NO:164

The DNA sequence encoding A0318_ybhG_opt_hp4, integrated as part of the ybhGFSR operon at the ΔA0358-downstream locus in JCC2055, is:

ATGCAGAAGCAGCAGAACCTGGACTATTTCAGCCCGCAAGCGTTGGCGCT
GTGGGCAGCTATCGCCAGCCTGGGCGTTATGTCCCCAGCACACGCTGGTG
GCTACTGGTGGTATCAAAGCCGCCAGGATAACGGTTTGACCCTGTATGGC
AATGTTGATATTCGCACCGTCAACCTGTCGTTCCGCGTGGGTGGCCGTGT
GGAGAGCCTGGCCGTGGATGAAGGCGATGCGATCAAAGCAGGTCAGGTCC
TAGGTGAGCTGGATCACAAACCATACGAAATCGCCCTGATGCAAGCCAAA
GCGGGTGTTAGCGTGGCACAAGCGCAGTACGATCTGATGTTGGCGGGTTA
CCGCAATGAAGAGATTGCGCAGGCGGCAGCGGCGGTGAAACAAGCGCAAG
CGGCGTATGACTATGCGCAAAACTTTTACAATCGTTTCCAAGAGCTGTAT
GCAAGCGGTGTGGTTAGCAAGCAAGATCTGGAAAATGCGCGTTCTAGCCG
TGATCAGGCACAGGCCACGCTGAAGAGCGCGCAGGATAAGCTGCGCCAAT
ATCGTAGCGGCAATCGTGAACAAGACATTGCACAGGCTAAGGCATCTCTG
GAACAGGCCCAAGCTCAACTGGCCCAGGCGGAACTGAACCTGCAGGACTC
CACTCTGATCGCACCTTCTGACGGTACTTTGCTGACGCGTGCGGTTGAAC
CGGGTACCGTGCTGAATGAGGGCGGTACGGTTTTCACGGTCAGCCTGACG
CGTCCGGTCTGGGTTCGTGCCTACGTCGATGAGCGTAACCTGGACCAGGC
GCAACCAGGCCGTAAGGTTCTGCTGTATACCGACGGTCGCCCGGATAAAC
CTTACCACGGTCAAATTGGCTTTGTTTCCCCGACGGCTGAGTTTACCCCG
AAAACCGTCGAAACGCCGGACCTGCGTACCGACCTGGTCTACCGTCTGCG
CATCGTCGTGACCGACGCGGATGACGCATTGCGTCAGGGCATGCCGGTGA
CCGTGCAGTTCGGCGACGAGGCTGGTCATGAGTAA

### SEQ ID NO:165

The protein sequence encoded by A0318_ybhG_opt_hp4, integrated as part of the ybhGFSR operon at the ΔA0358-downstream locus in JCC2055, is:

MQKQQNLDYFSPQALALWAAIASLGVMSPAHAGGYWWYQSRQDNGLTLYG
NVDIRTVNLSFRVGGRVESLAVDEGDAIKAGQVLGELDHKPYEIALMQAK
AGVSVAQAQYDLMLAGYRNEEIAQAAAAVKQAQAAYDYAQNFYNRFQELY
ASGVVSKQDLENARSSRDQAQATLKSAQDKLRQYRSGNREQDIAQAKASL
EQAQAQLAQAELNLQDSTLIAPSDGTLLTRAVEPGTVLNEGGTVFTVSLT
RPVWVRAYVDERNLDQAQPGRKVLLYTDGRPDKPYHGQIGFVSPTAEFTP
KTVETPDLRTDLVYRLRIVVTDADDALRQGMPVTVQFGDEAGHE

### SEQ ID NO:166

The DNA sequence encoding A0578_ybhG_opt_hp1, integrated as part of the ybhGFSR operon at the ΔA0358-downstream locus in JCC2055, is:

ATGCGTTTCTTTTGGTTTTTCCTGACGTTGCTGACCCTGAGCACCTGGCA
GCTGCCGGCGTGGGCGGGTGGCTACTGGTGGTATCAAAGCCGCCAGGATA
ACGGTTTGACCCTGTATGGCAATGTTGATATTCGCACCGTCAACCTGTCG
TTCCGCGTGGGTGGCCGTGTGGAGAGCCTGGCCGTGGATGAAGGCGATGC
GATCAAAGCAGGTCAGGTCCTAGGTGAGCTGGATCACAAACCATACGAAA
TCGCCCTGATGCAAGCCAAAGCGGGTGTTAGCGTGGCACAAGCGCAGTAC
GATCTGATGTTGGCGGGTTACCGCAATGAAGAGATTGCGCAGGCGGCAGC
GGCGGTGAAACAAGCGCAAGCGGCGTATGACCTGGCTAAGGCCGACGGCG
ACCGTTTCCAAGAGCTGTATGCAAGCGGTGTGGTTAGCAAGCAACGTCTG
GAGCAGGCGCAGACCAGCCGTGATCAGGCACAGGCCACGCTGAAGAGCGC
GCAGGATAAGCTGCGCCAATATCGTAGCGGCAATCGTGAACAAGACATTG
CACAGGCTAAGGCATCTCTGGAACAGGCCCAAGCTCAACTGGCCCAGGCG
GAACTGAACCTGCAGGACTCCACTCTGATCGCACCTTCTGACGGTACTTT
GCTGACGCGTGCGGTTGAACCGGGTACCGTGCTGAATGAGGGCGGTACGG
TTTTCACGGTCAGCCTGACGCGTCCGGTCTGGGTTCGTGCCTACGTCGAT
GAGCGTAACCTGGACCAGGCGCAACCAGGCCGTAAGGTTCTGCTGTATAC
CGACGGTCGCCCGGATAAACCTTACCACGGTCAAATTGGCTTTGTTTCCC
CGACGGCTGAGTTTACCCCGAAAACCGTCGAAACGCCGGACCTGCGTACC
GACCTGGTCTACCGTCTGCGCATCGTCGTGACCGACGCGGATGACGCATT
GCGTCAGGGCATGCCGGTGACCGTGCAGTTCGGCGACGAGGCTGGTCATG
AGTAA

### SEQ ID NO:167

The protein sequence encoded by A0578_ybhG_opt_hp1, integrated as part of the ybhGFSR operon at the ΔA0358-downstream locus in JCC2055, is:

MRFFWFFLTLLTLSTWQLPAWAGGYWWYQSRQDNGLTLYGNVDIRTVNLS
FRVGGRVESLAVDEGDAIKAGQVLGELDHKPYEIALMQAKAGVSVAQAQY
DLMLAGYRNEEIAQAAAAVKQAQAAYDLAKADGDRFQELYASGVVSKQRL
EQAQTSRDQAQATLKSAQDKLRQYRSGNREQDIAQAKASLEQAQAQLAQA
ELNLQDSTLIAPSDGTLLTRAVEPGTVLNEGGTVFTVSLTRPVWVRAYVD
ERNLDQAQPGRKVLLYTDGRPDKPYHGQIGFVSPTAEFTPKTVETPDLRT
DLVYRLRIVVTDADDALRQGMPVTVQFGDEAGHE

### SEQ ID NO:168

The DNA sequence encoding A0578_ybhG_opt_hp2, integrated as part of the ybhGFSR operon at the ΔA0358-downstream locus in JCC2055, is:

ATGATGAAAAAGCCGGTTGTTATCGGTTTGGCGGTGGTGGTTCTGGCAGC
AGTCGTTGCGGGTGGCTACTGGTGGTATCAAAGCCGCCAGGATAACGGTT
TGACCCTGTATGGCAATGTTGATATTCGCACCGTCAACCTGTCGTTCCGC
GTGGGTGGCCGTGTGGAGAGCCTGGCCGTGGATGAAGGCGATGCGATCAA
AGCAGGTCAGGTCCTAGGTGAGCTGGATAGCGCCGAACTGCAGGCATCCC
TGGATGGTGCACAAGCCCGTATCAATGCGGCGCAGCAGCAGGTTAATCAA
GCACAGCTGCAAATCACCGTGATTGAAAACCAGATTACCGAGGCACAGCT
GACCCAACGCCAAGCACAGGATGACACTGCCGGTCGCGTTAATGCGGCAC
AAGCGAACGTGGCGGCAGCCAAGGCGCAACTGGCCCAGGCGCAAGCGCAG
GTCAAGCAGCTGGAAGCAGAGCTGGCCCTGGCGAAGGCAGACGGTGACCG
TTTCCAAGAACTGTACGCGAGCGGTGTGGTGAGCAAACAGCGTCTGGAGC
AAGCTCAAACCCAATATCTGAGCACGAAAGAGAATCTGGATGCTCGTCGC
GCGGTTGTTGCGGCAGCTGCGGAGCAAGTGAAAACCGCGGAGGGTAACCT
GACGCAAACTCAGGCGTCCCAGTTCAACCCAGACATTCAGTACCTGAGCA
CCAAAGAAAATCTGGACGCACGTCGTGCTGTCGTCGCTGCCGCTGCAGAA
CAAGTTAAGACCGCCGAGGGTAACTTGACTCAGACCCAAGCGAGCCAATT
CAACCCGGACATTCGTGCAGTTCAAGTGCAGCGCCTGCAAACGCAACTGG
TCCAGGCGCAGGCCCAGCTGTCTGCGGCGCAAGCACAAGTTCAGAATGCT
CAGGCCAACTATAACGAGATCGCGGCGAACCTGCAGGACTCCACTCTGAT
CGCACCTTCTGACGGTACTTTGCTGACGCGTGCGGTTGAACCGGGTACCG
TGCTGAATGAGGGCGGTACGGTTTTCACGGTCAGCCTGACGCGTCCGGTC
TGGGTTCGTGCCTACGTCGATGAGCGTAACCTGGACCAGGCGCAACCAGG
CCGTAAGGTTCTGCTGTATACCGACGGTCGCCCGGATAAACCTTACCACG
GTCAAATTGGCTTTGTTTCCCCGACGGCTGAGTTTACCCCGAAAACCGTC
GAAACGCCGGACCTGCGTACCGACCTGGTCTACCGTCTGCGCATCGTCGT
GACCGACGCGGATGACGCATTGCGTCAGGGCATGCCGGTGACCGTGCAGT
TCGGCGACGAGGCTGGTCATGAGTAA

### SEQ ID NO:169

The protein sequence encoded by A0578_ybhG_opt_hp2, integrated as part of the ybhGFSR operon at the ΔA0358-downstream locus in JCC2055, is:

MMKKPVVIGLAVVVLAAVVAGGYWWYQSRQDNGLTLYGNVDIRTVNLSFR
VGGRVESLAVDEGDAIKAGQVLGELDSAELQASLDGAQARINAAQQQVNQ
AQLQITVIENQITEAQLTQRQAQDDTAGRVNAAQANVAAAKAQLAQAQAQ
VKQLEAELALAKADGDRFQELYASGVVSKQRLEQAQTQYLSTKENLDARR
AVVAAAAEQVKTAEGNLTQTQASQFNPDIQYLSTKENLDARRAVVAAAAE
QVKTAEGNLTQTQASQFNPDIRAVQVQRLQTQLVQAQAQLSAAQAQVQNA
QANYNEIAANLQDSTLIAPSDGTLLTRAVEPGTVLNEGGTVFTVSLTRPV
WVRAYVDERNLDQAQPGRKVLLYTDGRPDKPYHGQIGFVSPTAEFTPKTV
ETPDLRTDLVYRLRIVVTDADDALRQGMPVTVQFGDEAGHE

### SEQ ID NO:170

The DNA sequence encoding A0578_ybhG_opt_hp3, integrated as part of the ybhGFSR operon at the ΔA0358-downstream locus in JCC2055, is:

ATGCGTTTCTTTTGGTTTTTCCTGACGTTGCTGACCCTGAGCACCTGGCA
GCTGCCGGCGTGGGCGGGTGGCTACTGGTGGTATCAAAGCCGCCAGGATA
ACGGTTTGACCCTGTATGGCAATGTTGATATTCGCACCGTCAACCTGTCG
TTCCGCGTGGGTGGCCGTGTGGAGAGCCTGGCCGTGGATGAAGGCGATGC
GATCAAAGCAGGTCAGGTCCTAGGTGAGCTGGATAGCGCCGAACTGCAGG
CATCCCTGGATGGTGCACAAGCCCGTATCAATGCGGCGCAGCAGCAGGTT
AATCAAGCACAGCTGCAAATCACCGTGATTGAAAACCAGATTACCGAGGC
ACAGCTGACCCAACGCCAAGCACAGGATGACACTGCCGGTCGCGTTAATG
CGGCACAAGCGAACGTGGCGGCAGCCAAGGCGCAACTGGCCCAGGCGCAA
GCGCAGGTCAAGCAGCTGGAAGCAGAGCTGGCCTATGCGCAAAACTTTTA
CAATCGCCAGCAAGGTTTGTGGAAGAGCCGTACGATTAGCGCAAACGATC
TGGAAAATGCGCGTTCTCAATATCTGAGCACGAAAGAGAATCTGGATGCT
CGTCGCGCGGTTGTTGCGGCAGCTGCGGAGCAAGTGAAAACCGCGGAGGG
TAACCTGACGCAAACTCAGGCGTCCCAGTTCAACCCAGACATTCAGTACC
TGAGCACCAAAGAAAATCTGGACGCACGTCGTGCTGTCGTCGCTGCCGCT
GCAGAACAAGTTAAGACCGCCGAGGGTAACTTGACTCAGACCCAAGCGAG
CCAATTCAACCCGGACATTCGTGCAGTTCAAGTGCAGCGCCTGCAAACGC
AACTGGTCCAGGCGCAGGCCCAGCTGTCTGCGGCGCAAGCACAAGTTCAG
AATGCTCAGGCCAACTATAACGAGATCGCGGCGAACCTGCAGGACTCCAC
TCTGATCGCACCTTCTGACGGTACTTTGCTGACGCGTGCGGTTGAACCGG
GTACCGTGCTGAATGAGGGCGGTACGGTTTTCACGGTCAGCCTGACGCGT
CCGGTCTGGGTTCGTGCCTACGTCGATGAGCGTAACCTGGACCAGGCGCA
ACCAGGCCGTAAGGTTCTGCTGTATACCGACGGTCGCCCGGATAAACCTT
ACCACGGTCAAATTGGCTTTGTTTCCCCGACGGCTGAGTTTACCCCGAAA
ACCGTCGAAACGCCGGACCTGCGTACCGACCTGGTCTACCGTCTGCGCAT
CGTCGTGACCGACGCGGATGACGCATTGCGTCAGGGCATGCCGGTGACCG
TGCAGTTCGGCGACGAGGCTGGTCATGAGTAA

### SEQ ID NO:171

The protein sequence encoded by A0578_ybhG_opt_hp3, integrated as part of the ybhGFSR operon at the ΔA0358-downstream locus in JCC2055, is:

MRFFWFFLTLLTLSTWQLPAWAGGYWWYQSRQDNGLTLYGNVDIRTVNLS
FRVGGRVESLAVDEGDAIKAGQVLGELDSAELQASLDGAQARINAAQQQV
NQAQLQITVIENQITEAQLTQRQAQDDTAGRVNAAQANVAAAKAQLAQAQ
AQVKQLEAELAYAQNFYNRQQGLWKSRTISANDLENARSQYLSTKENLDA
RRAVVAAAAEQVKTAEGNLTQTQASQFNPDIQYLSTKENLDARRAVVAAA
AEQVKTAEGNLTQTQASQFNPDIRAVQVQRLQTQLVQAQAQLSAAQAQVQ
NAQANYNEIAANLQDSTLIAPSDGTLLTRAVEPGTVLNEGGTVFTVSLTR
PVWVRAYVDERNLDQAQPGRKVLLYTDGRPDKPYHGQIGFVSPTAEFTPK
TVETPDLRTDLVYRLRIVVTDADDALRQGMPVTVQFGDEAGHE

### SEQ ID NO:172

The DNA sequence encoding A0578_ybhG_opt_hp4, integrated as part of the ybhGFSR operon at the ΔA0358-downstream locus in JCC2055, is:

ATGCGTTTCTTTTGGTTTTTCCTGACGTTGCTGACCCTGAGCACCTGGCA
GCTGCCGGCGTGGGCGGGTGGCTACTGGTGGTATCAAAGCCGCCAGGATA
ACGGTTTGACCCTGTATGGCAATGTTGATATTCGCACCGTCAACCTGTCG
TTCCGCGTGGGTGGCCGTGTGGAGAGCCTGGCCGTGGATGAAGGCGATGC
GATCAAAGCAGGTCAGGTCCTAGGTGAGCTGGATCACAAACCATACGAAA
TCGCCCTGATGCAAGCCAAAGCGGGTGTTAGCGTGGCACAAGCGCAGTAC
GATCTGATGTTGGCGGGTTACCGCAATGAAGAGATTGCGCAGGCGGCAGC
GGCGGTGAAACAAGCGCAAGCGGCGTATGACTATGCGCAAAACTTTTACA
ATCGTTTCCAAGAGCTGTATGCAAGCGGTGTGGTTAGCAAGCAAGATCTG
GAAAATGCGCGTTCTAGCCGTGATCAGGCACAGGCCACGCTGAAGAGCGC
GCAGGATAAGCTGCGCCAATATCGTAGCGGCAATCGTGAACAAGACATTG
CACAGGCTAAGGCATCTCTGGAACAGGCCCAAGCTCAACTGGCCCAGGCG
GAACTGAACCTGCAGGACTCCACTCTGATCGCACCTTCTGACGGTACTTT
GCTGACGCGTGCGGTTGAACCGGGTACCGTGCTGAATGAGGGCGGTACGG
TTTTCACGGTCAGCCTGACGCGTCCGGTCTGGGTTCGTGCCTACGTCGAT
GAGCGTAACCTGGACCAGGCGCAACCAGGCCGTAAGGTTCTGCTGTATAC
CGACGGTCGCCCGGATAAACCTTACCACGGTCAAATTGGCTTTGTTTCCC
CGACGGCTGAGTTTACCCCGAAAACCGTCGAAACGCCGGACCTGCGTACC
GACCTGGTCTACCGTCTGCGCATCGTCGTGACCGACGCGGATGACGCATT
GCGTCAGGGCATGCCGGTGACCGTGCAGTTCGGCGACGAGGCTGGTCATG
AGTAA

### SEQ ID NO:173

The protein sequence encoded by A0578_ybhG_opt_hp4, integrated as part of the ybhGFSR operon at the ΔA0358-downstream locus in JCC2055, is:

MRFFWFFLTLLTLSTWQLPAWAGGYWWYQSRQDNGLTLYGNVDIRTVNLS
FRVGGRVESLAVDEGDAIKAGQVLGELDHKPYEIALMQAKAGVSVAQAQY
DLMLAGYRNEEIAQAAAAVKQAQAAYDYAQNFYNRFQELYASGVVSKQDL
ENARSSRDQAQATLKSAQDKLRQYRSGNREQDIAQAKASLEQAQAQLAQA
ELNLQDSTLIAPSDGTLLTRAVEPGTVLNEGGTVFTVSLTRPVWVRAYVD
ERNLDQAQPGRKVLLYTDGRPDKPYHGQIGFVSPTAEFTPKTVETPDLRT
DLVYRLRIVVTDADDALRQGMPVTVQFGDEAGHE

All ybhFSR variants, integrated at the ΔA0358-downstream locus in JCC2055 with the ybhG-hairpin panel, are indicated in Table 15 and Table 16.

### Example 9

### Set 1

OMP variant

### SEQ ID NO:174

>SYNPCC7002_A0585
MFAFRDFLTFSTGGLVVLSGGGVAIAQTTPPQIATPEPFIGQTPQA
PLPPLAAPSVESLDTAAFLPSLGGLSQPTTLAALPLPSPELNLSPT
AHLGTIQAPSPLLAQVDTTATPSPTTAIDVTLPTAETNQTIPLVQPLP
PDRVINEDLNQLLEPIDNPAVTVPQEATAVTTDNVVDLTLEETIRLAL
ERNETLQEARLNYDRSEELVREAIAAEYPNLSNQVDITRTDSANGEL
QARRLGGDNNATTAINGRLEVSYDIYTGGRRSAQIEAAQTQLQIAELD
IERLTEETRLAAAVNYYNLQSADAQVVIEQSSVFDATQSLRDATLLE
QAGLGTKFDVLRAEVELASAQQRLTRAEATQRTARRQLAQLLSLEP
TIDPRTADEINLAGRWEISLEETIVLALQNRQELRQQLLQREVDGYQE
RIALAAVRPLVSVFANYDVLEVFDDSLGPADGLTVGARMRWNFFDG
GAAAARANQEQVDQAIAENRFANQRNQIRLAVETAYYDFEASEQNIT
TAAAAVTLAEESLRLARLRFNAGVGTQTDVISAQTGLNTARGNYLQAV
TDYNRAFAQLKREVGLGDAVIAPAAP

YbhG variants

### SEQ ID NO:175

>YbhG_hp1
MMKKPVVIGLAVVVLAAVVAGGYWWYQSRQDNGLTLYGNVDIRTVNLSFR
VGGRVESLAVDEGDAIKAGQVLGELDHKPYEIALMQAKAGVSVAQAQYDL
MLAGYRNEEIAQAAAAVKQAQAAYDLAKADGDRFQELYASGVVSKQRLEQ
AQTSRDQAQATLKSAQDKLRQYRSGNREQDIAQAKASLEQAQAQLAQAEL
NLQDSTLIAPSDGTLLTRAVEPGTVLNEGGTVFTVSLTRPVWVRAYVDER
NLDQAQPGRKVLLYTDGRPDKPYHGQIGFVSPTAEFTPKTVETPDLRTDL
VYRLRIVVTDADDALRQGMPVTVQFGDEAGHE

### SEQ ID NO:176

>YbhG_hp2
MMKKPVVIGLAVVVLAAVVAGGYWWYQSRQDNGLTLYGNVDIRTVNLSFR
VGGRVESLAVDEGDAIKAGQVLGELDSAELQASLDGAQARINAAQQQVNQ
AQLQITVIENQITEAQLTQRQAQDDTAGRVNAAQANVAAAKAQLAQAQAQ
VKQLEAELALAKADGDRFQELYASGVVSKQRLEQAQTQYLSTKENLDARR
AVVAAAAEQVKTAEGNLTQTQASQFNPDIQYLSTKENLDARRAVVAAAAE
QVKTAEGNLTQTQASQFNPDIRAVQVQRLQTQLVQAQAQLSAAQAQVQNA
QANYNEIAANLQDSTLIAPSDGTLLTRAVEPGTVLNEGGTVFTVSLTRPV
WVRAYVDERNLDQAQPGRKVLLYTDGRPDKPYHGQIGFVSPTAEFTPKTV
ETPDLRTDLVYRLRIVVTDADDALRQGMPVTVQFGDEAGHE

### SEQ ID NO:177

>YbhG_hp4
MMKKPVVIGLAVVVLAAVVAGGYWWYQSRQDNGLTLYGNVDIRTVNLSFR
VGGRVESLAVDEGDAIKAGQVLGELDHKPYEIALMQAKAGVSVAQAQYDL
MLAGYRNEEIAQAAAAVKQAQAAYDYAQNFYNRFQELYASGVVSKQDLEN
ARSSRDQAQATLKSAQDKLRQYRSGNREQDIAQAKASLEQAQAQLAQAEL
NLQDSTLIAPSDGTLLTRAVEPGTVLNEGGTVFTVSLTRPVWVRAYVDER
NLDQAQPGRKVLLYTDGRPDKPYHGQIGFVSPTAEFTPKTVETPDLRTDL
VYRLRIVVTDADDALRQGMPVTVQFGDEAGHE

### SEQ ID NO:178

>torA_YbhG_hp1
MNNNDLFQASRRRFLAQLGGLTVAGMLGPSLLTPRRATAGGYWWYQSRQD
NGLTLYGNVDIRTVNLSFRVGGRVESLAVDEGDAIKAGQVLGELDHKPYE
IALMQAKAGVSVAQAQYDLMLAGYRNEEIAQAAAAVKQAQAAYDLAKADG
DRFQELYASGVVSKQRLEQAQTSRDQAQATLKSAQDKLRQYRSGNREQDI
AQAKASLEQAQAQLAQAELNLQDSTLIAPSDGTLLTRAVEPGTVLNEGGT
VFTVSLTRPVWVRAYVDERNLDQAQPGRKVLLYTDGRPDKPYHGQIGFVS
PTAEFTPKTVETPDLRTDLVYRLRIVVTDADDALRQGMPVTVQFGDEAG
HE

### SEQ ID NO:179

>torA_YbhG_hp2
MNNNDLFQASRRRFLAQLGGLTVAGMLGPSLLTPRRATAGGYWWYQSRQD
NGLTLYGNVDIRTVNLSFRVGGRVESLAVDEGDAIKAGQVLGELDSAELQ
ASLDGAQARINAAQQQVNQAQLQITVIENQITEAQLTQRQAQDDTAGRVN
AAQANVAAAKAQLAQAQAQVKQLEAELALAKADGDRFQELYASGVVSKQR
LEQAQTQYLSTKENLDARRAVVAAAAEQVKTAEGNLTQTQASQFNPDIQY
LSTKENLDARRAVVAAAAEQVKTAEGNLTQTQASQFNPDIRAVQVQRLQT
QLVQAQAQLSAAQAQVQNAQANYNEIAANLQDSTLIAPSDGTLLTRAVEP
GTVLNEGGTVFTVSLTRPVWVRAYVDERNLDQAQPGRKVLLYTDGRPDKP
YHGQIGFVSPTAEFTPKTVETPDLRTDLVYRLRIVVTDADDALRQGMPVT
VQFGDEAGHE

### SEQ ID NO:180

>torA_YbhG_hp4
MNNNDLFQASRRRFLAQLGGLTVAGMLGPSLLTPRRATAGGYWWYQSRQD
NGLTLYGNVDIRTVNLSFRVGGRVESLAVDEGDAIKAGQVLGELDHKPYE
IALMQAKAGVSVAQAQYDLMLAGYRNEEIAQAAAAVKQAQAAYDYAQNFY
NRFQELYASGVVSKQDLENARSSRDQAQATLKSAQDKLRQYRSGNREQDI
AQAKASLEQAQAQLAQAELNLQDSTLIAPSDGTLLTRAVEPGTVLNEGGT
VFTVSLTRPVWVRAYVDERNLDQAQPGRKVLLYTDGRPDKPYHGQIGFVS
PTAEFTPKTVETPDLRTDLVYRLRIVVTDADDALRQGMPVTVQFGDEAG
HE

### SEQ ID NO:181

>A0318_YbhG_hp1
MQKQQNLDYFSPQALALWAAIASLGVMSPAHAGGYWWYQSRQDNGLTLYG
NVDIRTVNLSFRVGGRVESLAVDEGDAIKAGQVLGELDHKPYEIALMQAK
AGVSVAQAQYDLMLAGYRNEEIAQAAAAVKQAQAAYDLAKADGDRFQELY
ASGVVSKQRLEQAQTSRDQAQATLKSAQDKLRQYRSGNREQDIAQAKASL
EQAQAQLAQAELNLQDSTLIAPSDGTLLTRAVEPGTVLNEGGTVFTVSLT
RPVWVRAYVDERNLDQAQPGRKVLLYTDGRPDKPYHGQIGFVSPTAEFTP
KTVETPDLRTDLVYRLRIVVTDADDALRQGMPVTVQFGDEAGHE

### SEQ ID NO:182

>A0318_YbhG_hp2
MQKQQNLDYFSPQALALWAAIASLGVMSPAHAGGYWWYQSRQDNGLTLYG
NVDIRTVNLSFRVGGRVESLAVDEGDAIKAGQVLGELDSAELQASLDGAQ
ARINAAQQQVNQAQLQITVIENQITEAQLTQRQAQDDTAGRVNAAQANVA
AAKAQLAQAQAQVKQLEAELALAKADGDRFQELYASGVVSKQRLEQAQTQ
YLSTKENLDARRAVVAAAAEQVKTAEGNLTQTQASQFNPDIQYLSTKENL
DARRAVVAAAAEQVKTAEGNLTQTQASQFNPDIRAVQVQRLQTQLVQAQA
QLSAAQAQVQNAQANYNEIAANLQDSTLIAPSDGTLLTRAVEPGTVLNEG
GTVFTVSLTRPVWVRAYVDERNLDQAQPGRKVLLYTDGRPDKPYHGQIGF
VSPTAEFTPKTVETPDLRTDLVYRLRIVVTDADDALRQGMPVTVQFGDEA
GHE

### SEQ ID NO:183

>A0318_YbhG_hp4
MQKQQNLDYFSPQALALWAAIASLGVMSPAHAGGYWWYQSRQDNGLTLYG
NVDIRTVNLSFRVGGRVESLAVDEGDAIKAGQVLGELDHKPYEIALMQAK
AGVSVAQAQYDLMLAGYRNEEIAQAAAAVKQAQAAYDYAQNFYNRFQELY
ASGVVSKQDLENARSSRDQAQATLKSAQDKLRQYRSGNREQDIAQAKASL
EQAQAQLAQAELNLQDSTLIAPSDGTLLTRAVEPGTVLNEGGTVFTVSLT
RPVWVRAYVDERNLDQAQPGRKVLLYTDGRPDKPYHGQIGFVSPTAEFTP
KTVETPDLRTDLVYRLRIVVTDADDALRQGMPVTVQFGDEAGHE

### SEQ ID NO:184

>A0578_YbhG_hp1
MRFFWFFLTLLTLSTWQLPAWAGGYWWYQSRQDNGLTLYGNVDIRTVNLS
FRVGGRVESLAVDEGDAIKAGQVLGELDHKPYEIALMQAKAGVSVAQAQY
DLMLAGYRNEEIAQAAAAVKQAQAAYDLAKADGDRFQELYASGVVSKQRL
EQAQTSRDQAQATLKSAQDKLRQYRSGNREQDIAQAKASLEQAQAQLAQA
ELNLQDSTLIAPSDGTLLTRAVEPGTVLNEGGTVFTVSLTRPVWVRAYVD
ERNLDQAQPGRKVLLYTDGRPDKPYHGQIGFVSPTAEFTPKTVETPDLRT
DLVYRLRIVVTDADDALRQGMPVTVQFGDEAGHE

### SEQ ID NO:185

>A0578_YbhG_hp2
MMKKPVVIGLAVVVLAAVVAGGYWWYQSRQDNGLTLYGNVDIRTVNLSFR
VGGRVESLAVDEGDAIKAGQVLGELDSAELQASLDGAQARINAAQQQVNQ
AQLQITVIENQITEAQLTQRQAQDDTAGRVNAAQANVAAAKAQLAQAQAQ
VKQLEAELALAKADGDRFQELYASGVVSKQRLEQAQTQYLSTKENLDARR
AVVAAAAEQVKTAEGNLTQTQASQFNPDIQYLSTKENLDARRAVVAAAAE
QVKTAEGNLTQTQASQFNPDIRAVQVQRLQTQLVQAQAQLSAAQAQVQNA
QANYNEIAANLQDSTLIAPSDGTLLTRAVEPGTVLNEGGTVFTVSLTRPV
WVRAYVDERNLDQAQPGRKVLLYTDGRPDKPYHGQIGFVSPTAEFTPKTV
ETPDLRTDLVYRLRIVVTDADDALRQGMPVTVQFGDEAGHE

### SEQ ID NO:186

>A0578_YbhG_hp4
MRFFWFFLTLLTLSTWQLPAWAGGYWWYQSRQDNGLTLYGNVDIRTVNLS
FRVGGRVESLAVDEGDAIKAGQVLGELDHKPYEIALMQAKAGVSVAQAQY
DLMLAGYRNEEIAQAAAAVKQAQAAYDYAQNFYNRFQELYASGVVSKQDL
ENARSSRDQAQATLKSAQDKLRQYRSGNREQDIAQAKASLEQAQAQLAQA
ELNLQDSTLIAPSDGTLLTRAVEPGTVLNEGGTVFTVSLTRPVWVRAYVD
ERNLDQAQPGRKVLLYTDGRPDKPYHGQIGFVSPTAEFTPKTVETPDLRT
DLVYRLRIVVTDADDALRQGMPVTVQFGDEAGHE

### Set 2

OMP variants

### SEQ ID NO:187

>Hybrid_A0585
MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDLTLEETIRLALERNETLQEARLN
YDRSEELVREAIAAEYPNLSNQVDITRTDSANGELQARRLGGDNNATTAI
NGRLEVSYDIYTGGRRSAQIEAAQTQLQIAELDIERLTEETRLAAAVNYY
NLQSADAQVVIEQSSVFDATQQLDQTTQRFNVGLVAITDVQNARAELASA
QQRLTRAEATQRTARRQLAQLLSLEPTIDPRTADEINLAGRWEISLEETI
VLALQNRQELRQQLLQREVDGYQERIALAAVRPLVSVFANYDVLEVFDDS
LGPADGLTVGARMRWNFFDGGAAAARANQEQVDQAIAENRFANQRNQIRL
AVETAYYDFEASEQNITTAAAAVTLAEESLDAMEAGYSVGTRTIVDVLDA
TTGLNTARGNYLQAVTDYNRAFAQLKREVGLGDAVIAPAAP

### SEQ ID NO:188

>Hybrid_1761
MAAFLYRLSFLSALAIAAHGVTPPTAIAELAEATTAEPTPTVAQATTPPA
TTPTTTPAPGPVKEVVPDANLLKELQANPNPFQLPNQPNQVKTEALQPLT
LEQALNLARLNNPQIQVRQLQVQQRQAALRGTEAALYPTLGLQGTAGYQQ
NGTRLNVTEGTPTQPTGSSLFTTLGESSIGATLNLNYTIFDFVRGAQLAA
SRDQVTQAELDLEAALEDLQLTVSEAYYRLQNADQLVRIARESVVASERQ
LDQTTQRFNVGLVAITDVQNARAQLAQDQQNLVDSIGNQDKARRALVQAL
NLPQNVNVLTADPVELAAPWNLSLDESIVLAFQNRPELEREVLQRNISYN
QAQAARGQVLPQLGLQASYGVNGAINSNLRSGSQALTFPSPTLTNTSSYN
YSIGLVLNVPLFDGGLANANAQQQELNGQIAEQNFVLTRNQIRTDVETAF
YDLQTNLANIGTTRKAVEQARESLDAMEAGYSVGTRTIVDVLDATTDLTR
AEANALNAITAYNLALARIKRAVSNVNNLARAGG

### SEQ ID NO:189

>TolC
MKKLLPILIGLSLSGFSSLSQAENLMQVYQQARLSNPELRKSAADRDAAF
EKINEARSPLLPQLGLGADYTYSNGYRDANGINSNATSASLQLTQSIFDM
SKWRALTLQEKAAGIQDVTYQTDQQTLILNTATAYFNVLNAIDVLSYTQA
QKEAIYRQLDQTTQRFNVGLVAITDVQNARAQYDTVLANEVTARNNLDNA
VEQLRQITGNYYPELAALNVENFKTDKPQPVNALLKEAEKRNLSLLQARL
SQDLAREQIRQAQDGHLPTLDLTASTGISDTSYSGSKTRGAAGTQYDDSN
MGQNKVGLSFSLPIYQGGMVNSQVKQAQYNFVGASEQLESAHRSVVQTVR
SSFNNINASISSINAYKQAVVSAQSSLDAMEAGYSVGTRTIVDVLDATTT
LYNAKQELANARYNYLINQLNIKSALGTLNEQDLLALNNALSKPVSTNPE
NVAPQTPEQNAIADGYAPDSPAPVVQQTSARTTTSNGHNPFRN

### SEQ ID NO:190

>A0585_TolC
MFAFRDFLTFSTGGLVVLSGGGVAIAENLMQVYQQARLSNPELRKSAADR
DAAFEKINEARSPLLPQLGLGADYTYSNGYRDANGINSNATSASLQLTQS
IFDMSKWRALTLQEKAAGIQDVTYQTDQQTLILNTATAYFNVLNAIDVLS
YTQAQKEAIYRQLDQTTQRFNVGLVAITDVQNARAQYDTVLANEVTARNN
LDNAVEQLRQITGNYYPELAALNVENFKTDKPQPVNALLKEAEKRNLSLL
QARLSQDLAREQIRQAQDGHLPTLDLTASTGISDTSYSGSKTRGAAGTQY
DDSNMGQNKVGLSFSLPIYQGGMVNSQVKQAQYNFVGASEQLESAHRSVV
QTVRSSFNNINASISSINAYKQAVVSAQSSLDAMEAGYSVGTRTIVDVLD
ATTTLYNAKQELANARYNYLINQLNIKSALGTLNEQDLLALNNALSKPVS
TNPENVAPQTPEQNAIADGYAPDSPAPVVQQTSARTTTSNGHNPFRN

### SEQ ID NO:191

>A0585_TolC_A0318C
MFAFRDFLTFSTGGLVVLSGGGVAIAENLMQVYQQARLSNPELRKSAADR
DAAFEKINEARSPLLPQLGLGADYTYSNGYRDANGINSNATSASLQLTQS
IFDMSKWRALTLQEKAAGIQDVTYQTDQQTLILNTATAYFNVLNAIDVLS
YTQAQKEAIYRQLDQTTQRFNVGLVAITDVQNARAQYDTVLANEVTARNN
LDNAVEQLRQITGNYYPELAALNVENFKTDKPQPVNALLKEAEKRNLSLL
QARLSQDLAREQIRQAQDGHLPTLDLTASTGISDTSYSGSKTRGAAGTQY
DDSNMGQNKVGLSFSLPIYQGGMVNSQVKQAQYNFVGASEQLESAHRSVV
QTVRSSFNNINASISSINAYKQAVVSAQSSLDAMEAGYSVGTRTIVDVLD
ATTTLYNAKQELANARYNYLINQLNIKSALGTLNEQDLLALNNALSKPVS
TNPENVAPQTPEQNAIADGYAPDSPAPVVQQTSARTTTSNGHNPFRNRIH
FGIGERF

### SEQ ID NO:192

>A0585_TolC_A0585C
MFAFRDFLTFSTGGLVVLSGGGVAIAENLMQVYQQARLSNPELRKSAADR
DAAFEKINEARSPLLPQLGLGADYTYSNGYRDANGINSNATSASLQLTQS
IFDMSKWRALTLQEKAAGIQDVTYQTDQQTLILNTATAYFNVLNAIDVLS
YTQAQKEAIYRQLDQTTQRFNVGLVAITDVQNARAQYDTVLANEVTARNN
LDNAVEQLRQITGNYYPELAALNVENFKTDKPQPVNALLKEAEKRNLSLL
QARLSQDLAREQIRQAQDGHLPTLDLTASTGISDTSYSGSKTRGAAGTQY
DDSNMGQNKVGLSFSLPIYQGGMVNSQVKQAQYNFVGASEQLESAHRSVV
QTVRSSFNNINASISSINAYKQAVVSAQSSLDAMEAGYSVGTRTIVDVLD
ATTTLYNAKQELANARYNYLINQLNIKSALGTLNEQDLLALNNALSKPVS
TNPENVAPQTPEQNAIADGYAPDSPAPVVQQTSARTTTSNGHNPFRNGDA
VIAPAAP

### SEQ ID NO:193

>A0585_ProNterm_TolC
MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDENLMQVYQQARLSNPELRKSAAD
RDAAFEKINEARSPLLPQLGLGADYTYSNGYRDANGINSNATSASLQLTQ
SIFDMSKWRALTLQEKAAGIQDVTYQTDQQTLILNTATAYFNVLNAIDVL
SYTQAQKEAIYRQLDQTTQRFNVGLVAITDVQNARAQYDTVLANEVTARN
NLDNAVEQLRQITGNYYPELAALNVENFKTDKPQPVNALLKEAEKRNLSL
LQARLSQDLAREQIRQAQDGHLPTLDLTASTGISDTSYSGSKTRGAAGTQ
YDDSNMGQNKVGLSFSLPIYQGGMVNSQVKQAQYNFVGASEQLESAHRSV
VQTVRSSFNNINASISSINAYKQAVVSAQSSLDAMEAGYSVGTRTIVDVL
DATTTLYNAKQELANARYNYLINQLNIKSALGTLNEQDLLALNNALSKPV
STNPENVAPQTPEQNAIADGYAPDSPAPVVQQTSARTTTSNGHNPFRN

### SEQ ID NO:194

>A0585_ProNTerm_TolC_A0318C
MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDENLMQVYQQARLSNPELRKSAAD
RDAAFEKINEARSPLLPQLGLGADYTYSNGYRDANGINSNATSASLQLTQ
SIFDMSKWRALTLQEKAAGIQDVTYQTDQQTLILNTATAYFNVLNAIDVL
SYTQAQKEAIYRQLDQTTQRFNVGLVAITDVQNARAQYDTVLANEVTARN
NLDNAVEQLRQITGNYYPELAALNVENFKTDKPQPVNALLKEAEKRNLSL
LQARLSQDLAREQIRQAQDGHLPTLDLTASTGISDTSYSGSKTRGAAGTQ
YDDSNMGQNKVGLSFSLPIYQGGMVNSQVKQAQYNFVGASEQLESAHRSV
VQTVRSSFNNINASISSINAYKQAVVSAQSSLDAMEAGYSVGTRTIVDVL
DATTTLYNAKQELANARYNYLINQLNIKSALGTLNEQDLLALNNALSKPV
STNPENVAPQTPEQNAIADGYAPDSPAPVVQQTSARTTTSNGHNPFRNRI
HFGIGERF

### SEQ ID NO:195

>A0585_ProNTerm_TolC_A0585C
MFAFRDFLTFSTGGLVVLSGGGVAIAQTTPPQIATPEPFIGQTPQAPLPP
LAAPSVESLDTAAFLPSLGGLSQPTTLAALPLPSPELNLSPTAHLGTIQA
PSPLLAQVDTTATPSPTTAIDVTLPTAETNQTIPLVQPLPPDRVINEDLN
QLLEPIDNPAVTVPQEATAVTTDNVVDENLMQVYQQARLSNPELRKSAAD
RDAAFEKINEARSPLLPQLGLGADYTYSNGYRDANGINSNATSASLQLTQ
SIFDMSKWRALTLQEKAAGIQDVTYQTDQQTLILNTATAYFNVLNAIDVL
SYTQAQKEAIYRQLDQTTQRFNVGLVAITDVQNARAQYDTVLANEVTARN
NLDNAVEQLRQITGNYYPELAALNVENFKTDKPQPVNALLKEAEKRNLSL
LQARLSQDLAREQIRQAQDGHLPTLDLTASTGISDTSYSGSKTRGAAGTQ
YDDSNMGQNKVGLSFSLPIYQGGMVNSQVKQAQYNFVGASEQLESAHRSV
VQTVRSSFNNINASISSINAYKQAVVSAQSSLDAMEAGYSVGTRTIVDVL
DATTTLYNAKQELANARYNYLINQLNIKSALGTLNEQDLLALNNALSKPV
STNPENVAPQTPEQNAIADGYAPDSPAPVVQQTSARTTTSNGHNPFRNGD
AVIAPAAP

### SEQ ID NO:196

>A0318_TolC
MQKQQNLDYFSPQALALWAAIASLGVMSPAHAENLMQVYQQARLSNPELR
KSAADRDAAFEKINEARSPLLPQLGLGADYTYSNGYRDANGINSNATSAS
LQLTQSIFDMSKWRALTLQEKAAGIQDVTYQTDQQTLILNTATAYFNVLN
AIDVLSYTQAQKEAIYRQLDQTTQRFNVGLVAITDVQNARAQYDTVLANE
VTARNNLDNAVEQLRQITGNYYPELAALNVENFKTDKPQPVNALLKEAEK
RNLSLLQARLSQDLAREQIRQAQDGHLPTLDLTASTGISDTSYSGSKTRG
AAGTQYDDSNMGQNKVGLSFSLPIYQGGMVNSQVKQAQYNFVGASEQLES
AHRSVVQTVRSSFNNINASISSINAYKQAVVSAQSSLDAMEAGYSVGTRT
IVDVLDATTTLYNAKQELANARYNYLINQLNIKSALGTLNEQDLLALNNA
LSKPVSTNPENVAPQTPEQNAIADGYAPDSPAPVVQQTSARTTTSNGHNP
FRN

### SEQ ID NO:197

>A0318_ProNTerm_TolC
MQKQQNLDYFSPQALALWAAIASLGVMSPAHAEPRSEGSHSDPLVPTATQ
VVVPALPVEDVAPTAAPASQTPAPQSENLAQSSTQAVTSPVAQAQEAPQD
SNLPQLYAQQQGNPNAQQANPENLMQVYQQARLSNPELRKSAADRDAAFE
KINEARSPLLPQLGLGADYTYSNGYRDANGINSNATSASLQLTQSIFDMS
KWRALTLQEKAAGIQDVTYQTDQQTLILNTATAYFNVLNAIDVLSYTQAQ
KEAIYRQLDQTTQRFNVGLVAITDVQNARAQYDTVLANEVTARNNLDNAV
EQLRQITGNYYPELAALNVENFKTDKPQPVNALLKEAEKRNLSLLQARLS
QDLAREQIRQAQDGHLPTLDLTASTGISDTSYSGSKTRGAAGTQYDDSNM
GQNKVGLSFSLPIYQGGMVNSQVKQAQYNFVGASEQLESAHRSVVQTVRS
SFNNINASISSINAYKQAVVSAQSSLDAMEAGYSVGTRTIVDVLDATTTL
YNAKQELANARYNYLINQLNIKSALGTLNEQDLLALNNALSKPVSTNPEN
VAPQTPEQNAIADGYAPDSPAPVVQQTSARTTTSNGHNPFRN

### SEQ ID NO:198

>A0318_ProNTerm_TolC_A0318C
MQKQQNLDYFSPQALALWAAIASLGVMSPAHAEPRSEGSHSDPLVPTATQ
VVVPALPVEDVAPTAAPASQTPAPQSENLAQSSTQAVTSPVAQAQEAPQD
SNLPQLYAQQQGNPNAQQANPENLMQVYQQARLSNPELRKSAADRDAAFE
KINEARSPLLPQLGLGADYTYSNGYRDANGINSNATSASLQLTQSIFDMS
KWRALTLQEKAAGIQDVTYQTDQQTLILNTATAYFNVLNAIDVLSYTQAQ
KEAIYRQLDQTTQRFNVGLVAITDVQNARAQYDTVLANEVTARNNLDNAV
EQLRQITGNYYPELAALNVENFKTDKPQPVNALLKEAEKRNLSLLQARLS
QDLAREQIRQAQDGHLPTLDLTASTGISDTSYSGSKTRGAAGTQYDDSNM
GQNKVGLSFSLPIYQGGMVNSQVKQAQYNFVGASEQLESAHRSVVQTVRS
SFNNINASISSINAYKQAVVSAQSSLDAMEAGYSVGTRTIVDVLDATTTL
YNAKQELANARYNYLINQLNIKSALGTLNEQDLLALNNALSKPVSTNPEN
VAPQTPEQNAIADGYAPDSPAPVVQQTSARTTTSNGHNPFRNRIHFGIGE
RF

### SEQ ID NO:199

>A0318_ProNTerm_TolC_A0585C
MQKQQNLDYFSPQALALWAAIASLGVMSPAHAEPRSEGSHSDPLVPT
ATQVVVPALPVEDVAPTAAPASQTPAPQSENLAQSSTQAVTSPVAQA
QEAPQDSNLPQLYAQQQGNPNAQQANPENLMQVYQQARLSNPELRK
SAADRDAAFEKINEARSPLLPQLGLGADYTYSNGYRDANGINSNATSA
SLQLTQSIFDMSKWRALTLQEKAAGIQDVTYQTDQQTLILNTATAYFNVL
NAIDVLSYTQAQKEAIYRQLDQTTQRFNVGLVAITDVQNARAQYDTVLAN
EVTARNNLDNAVEQLRQITGNYYPELAALNVENFKTDKPQPVNALLKEA
EKRNLSLLQARLSQDLAREQIRQAQDGHLPTLDLTASTGISDTSYSGSK
TRGAAGTQYDDSNMGQNKVGLSFSLPIYQGGMVNSQVKQAQYNFVGA
SEQLESAHRSVVQTVRSSFNNINASISSINAYKQAVVSAQSSLDAMEAG
YSVGTRTIVDVLDATTTLYNAKQELANARYNYLINQLNIKSALGTLNEQD
LLALNNALSKPVSTNPENVAPQTPEQNAIADGYAPDSPAPVVQQTSARTT
TSNGHNPFRNGDAVIAPAAP

YbhG variants

### SEQ ID NO:200

>YbhG
MMKKPVVIGLAVVVLAAVVAGGYWWYQSRQDNGLTLYGNVDIRTVNLS
FRVGGRVESLAVDEGDAIKAGQVLGELDHKPYEIALMQAKAGVSVAQA
QYDLMLAGYRNEEIAQAAAAVKQAQAAYDYAQNFYNRQQGLWKSRTIS
ANDLENARSSRDQAQATLKSAQDKLRQYRSGNREQDIAQAKASLEQA
QAQLAQAELNLQDSTLIAPSDGTLLTRAVEPGTVLNEGGTVFTVSLTRP
VWVRAYVDERNLDQAQPGRKVLLYTDGRPDKPYHGQIGFVSPTAEFT
PKTVETPDLRTDLVYRLRIVVTDADDALRQGMPVTVQFGDEAGHE

### SEQ ID NO:201

>TorA_YbhG
MNNNDLFQASRRRFLAQLGGLTVAGMLGPSLLTPRRATAGGYWWYQ
SRQDNGLTLYGNVDIRTVNLSFRVGGRVESLAVDEGDAIKAGQVLGE
LDHKPYEIALMQAKAGVSVAQAQYDLMLAGYRNEEIAQAAAAVKQAQ
AAYDYAQNFYNRQQGLWKSRTISANDLENARSSRDQAQATLKSAQDK
LRQYRSGNREQDIAQAKASLEQAQAQLAQAELNLQDSTLIAPSDGTLL
TRAVEPGTVLNEGGTVFTVSLTRPVWVRAYVDERNLDQAQPGRKVLL
YTDGRPDKPYHGQIGFVSPTAEFTPKTVETPDLRTDLVYRLRIVVTDADD
ALRQGMPVTVQFGDEAGHE

### SEQ ID NO:202

>A0578_YbhG
MRFFWFFLTLLTLSTWQLPAWAGGYWWYQSRQDNGLTLYGNVDIRTVN
LSFRVGGRVESLAVDEGDAIKAGQVLGELDHKPYEIALMQAKAGVSVA
QAQYDLMLAGYRNEEIAQAAAAVKQAQAAYDYAQNFYNRQQGLWKSRT
ISANDLENARSSRDQAQATLKSAQDKLRQYRSGNREQDIAQAKASLEQ
AQAQLAQAELNLQDSTLIAPSDGTLLTRAVEPGTVLNEGGTVFTVSLTRP
VWVRAYVDERNLDQAQPGRKVLLYTDGRPDKPYHGQIGFVSPTAEFTP
KTVETPDLRTDLVYRLRIVVTDADDALRQGMPVTVQFGDEAGHE

### SEQ ID NO:203

>A0318_YbhG
MQKQQNLDYFSPQALALWAAIASLGVMSPAHAGGYWWYQSRQDNGLT
LYGNVDIRTVNLSFRVGGRVESLAVDEGDAIKAGQVLGELDHKPYEIALM
QAKAGVSVAQAQYDLMLAGYRNEEIAQAAAAVKQAQAAYDYAQNFYNRQ
QGLWKSRTISANDLENARSSRDQAQATLKSAQDKLRQYRSGNREQDIAQ
AKASLEQAQAQLAQAELNLQDSTLIAPSDGTLLTRAVEPGTVLNEGGTVF
TVSLTRPVWVRAYVDERNLDQAQPGRKVLLYTDGRPDKPYHGQIGFVSP
TAEFTPKTVETPDLRTDLVYRLRIVVTDADDALRQGMPVTVQFGDEAGHE

### SEQ ID NO:204

>YbhG_hp3
MMKKPVVIGLAVVVLAAVVAGGYWWYQSRQDNGLTLYGNVDIRTVNLS
FRVGGRVESLAVDEGDAIKAGQVLGELDSAELQASLDGAQARINAAQQ
QVNQAQLQITVIENQITEAQLTQRQAQDDTAGRVNAAQANVAAAKAQLA
QAQAQVKQLEAELAYAQNFYNRQQGLWKSRTISANDLENARSQYLSTK
ENLDARRAVVAAAAEQVKTAEGNLTQTQASQFNPDIQYLSTKENLDARR
AVVAAAAEQVKTAEGNLTQTQASQFNPDIRAVQVQRLQTQLVQAQAQLSA
AQAQVQNAQANYNEIAANLQDSTLIAPSDGTLLTRAVEPGTVLNEGGTVF
TVSLTRPVWVRAYVDERNLDQAQPGRKVLLYTDGRPDKPYHGQIGFVSPT
AEFTPKTVETPDLRTDLVYRLRIVVTDADDALRQGMPVTVQFGDEAGHE

### SEQ ID NO:205

>TorA_YbhG_hp3
MNNNDLFQASRRRFLAQLGGLTVAGMLGPSLLTPRRATAGGYWWYQSR
QDNGLTLYGNVDIRTVNLSFRVGGRVESLAVDEGDAIKAGQVLGELDSAE
LQASLDGAQARINAAQQQVNQAQLQITVIENQITEAQLTQRQAQDDTAGR
VNAAQANVAAAKAQLAQAQAQVKQLEAELAYAQNFYNRQQGLWKSRTISA
NDLENARSQYLSTKENLDARRAVVAAAAEQVKTAEGNLTQTQASQFNPDI
QYLSTKENLDARRAVVAAAAEQVKTAEGNLTQTQASQFNPDIRAVQVQRL
QTQLVQAQAQLSAAQAQVQNAQANYNEIAANLQDSTLIAPSDGTLLTRAV
EPGTVLNEGGTVFTVSLTRPVWVRAYVDERNLDQAQPGRKVLLYTDGRP
DKPYHGQIGFVSPTAEFTPKTVETPDLRTDLVYRLRIVVTDADDALRQGM
PVTVQFGDEAGHE

### SEQ ID NO:206

>A0318_YbhG_hp3
MQKQQNLDYFSPQALALWAAIASLGVMSPAHAGGYWWYQSRQDNGLT
LYGNVDIRTVNLSFRVGGRVESLAVDEGDAIKAGQVLGELDSAELQASL
DGAQARINAAQQQVNQAQLQITVIENQITEAQLTQRQAQDDTAGRVNAAQ
ANVAAAKAQLAQAQAQVKQLEAELAYAQNFYNRQQGLWKSRTISANDLE
NARSQYLSTKENLDARRAVVAAAAEQVKTAEGNLTQTQASQFNPDIQYLS
TKENLDARRAVVAAAAEQVKTAEGNLTQTQASQFNPDIRAVQVQRLQTQL
VQAQAQLSAAQAQVQNAQANYNEIAANLQDSTLIAPSDGTLLTRAVEPGT
VLNEGGTVFTVSLTRPVWVRAYVDERNLDQAQPGRKVLLYTDGRPDKPY
HGQIGFVSPTAEFTPKTVETPDLRTDLVYRLRIVVTDADDALRQGMPVTV
QFGDEAGHE

### SEQ ID NO:207

>A0578_YbhG_hp3
MRFFWFFLTLLTLSTWQLPAWAGGYWWYQSRQDNGLTLYGNVDIRTVN
LSFRVGGRVESLAVDEGDAIKAGQVLGELDSAELQASLDGAQARINAAQ
QQVNQAQLQITVIENQITEAQLTQRQAQDDTAGRVNAAQANVAAAKAQLA
QAQAQVKQLEAELAYAQNFYNRQQGLWKSRTISANDLENARSQYLSTKE
NLDARRAVVAAAAEQVKTAEGNLTQTQASQFNPDIQYLSTKENLDARRAV
VAAAAEQVKTAEGNLTQTQASQFNPDIRAVQVQRLQTQLVQAQAQLSAAQ
AQVQNAQANYNEIAANLQDSTLIAPSDGTLLTRAVEPGTVLNEGGTVFTV
SLTRPVWVRAYVDERNLDQAQPGRKVLLYTDGRPDKPYHGQIGFVSPTAE
FTPKTVETPDLRTDLVYRLRIVVTDADDALRQGMPVTVQFGDEAGHE

### Sets 1 and 2

YbhF variant

### SEQ ID NO:208

>YbhF
MNDAVITLNGLEKRFPGMDKPAVAPLDCTIHAGYVTGLVGPDGAGK
TTLMRMLAGLLKPDSGSATVIGFDPIKNDGALHAVLGYMPQKFGLYED
LTVMENLNLYADLRSVTGEARKQTFARLLEFTSLGPFTGRLAGKLSGG
MKQKLGLACTLVGEPKVLLLDEPGVGVDPISRRELWQMVHELAGEGM
LILWSTSYLDEAEQCRDVLLMNEGELLYQGEPKALTQTMAGRSFLMTS
PHEGNRKLLQRALKLPQVSDGMIQGKSVRLILKKEATPDDIRHADGMPE
ININETTPRFEDAFIDLLGGAGTSESPLGAILHTVEGTPGETVIEAKELT
KKFGDFAATDHVNFAVKRGEIFGLLGPNGAGKSTTFKMMCGLLVPTS
GQALVLGMDLKESSGKARQHLGYMAQKFSLYGNLTVEQNLRFFSGV
YGLRGRAQNEKISRMSEAFGLKSIASHATDELPLGFKQRLALACSLMH
EPDILFLDEPTSGVDPLTRREFWLHINSMVEKGVTVMVTTHFMDEAEY
CDRIGLVYRGKLIASGTPDDLKAQSANDEQPDPTMEQAFIQLIHDWDK
EHSNE

YbhS, YbhR variants

### SEQ ID NO:209

>YbhS
MSNPILSWRRVRALCVKETRQIVRDPSSWLIAVVIPLLLLFIFGYGINLD
SSKLRVGILLEQRSEAALDFTHTMTGSPYIDATISDNRQELIAKMQAGK
IRGLVVIPVDFAEQMERANATAPIQVITDGSEPNTANFVQGYVEGIWQ
IWQMQRAEDNGQTFEPLIDVQTRYWFNPAAISQHFIIPGAVTIIMTVIGA
ILTSLVVAREWERGTMEALLSTEITRTELLLCKLIPYYFLGMLAMLLCM
LVSVFILGVPYRGSLLILFFISSLFLLSTLGMGLLISTITRNQFNAAQVA
LNAAFLPSIMLSGFIFQIDSMPAVIRAVTYIIPARYFVSTLQSLFLAGNI
PVVLVVNVLFLIASAVMFIGLTWLKTKRRLD

### SEQ ID NO:210

>YbhR
MFHRLWTLIRKELQSLLREPQTRAILILPVLIQVILFPFAATLEVTNATI
AIYDEDNGEHSVELTQRFARASAFTHVLLLKSPQEIRPTIDTQKALLLV
RFPADFSRKLDTFQTAPLQLILDGRNSNSAQIAANYLQQIVKNYQQEL
LEGKPKPNNSELVVRNWYNPNLDYKWFVVPSLIAMITTIGVMIVTSLS
VAREREQGTLDQLLVSPLTTWQIFIGKAVPALIVATFQATIVLAIGIWAY
QIPFAGSLALFYFTMVIYGLSLVGFGLLISSLCSTQQQAFIGVFVFMMP
AILLSGYVSPVENMPVWLQNLTWINPIRHFTDITKQIYLKDASLDIVWNS
LWPLLVITATTGSAAYAMFRRKVM

### SEQ ID NO:211

>sll0041_Nin_PLS_YbhS
MQAPTQSGGLSLRNKAVLIALLIGLIPAGVIGGLNLSSVDRLPVPQTEQQ
VKDSTTKQIRDQILIGLLVTAVGAAFVAYWMVGENTKAQTALALKAKSNP
ILSWRRVRALCVKETRQIVRDPSSWLIAVVIPLLLLFIFGYGINLDSSKL
RVGILLEQRSEAALDFTHTMTGSPYIDATISDNRQELIAKMQAGKIRGLV
VIPVDFAEQMERANATAPIQVITDGSEPNTANFVQGYVEGIWQIWQMQRA
EDNGQTFEPLIDVQTRYWFNPAAISQHFIIPGAVTIIMTVIGAILTSLVV
AREWERGTMEALLSTEITRTELLLCKLIPYYFLGMLAMLLCMLVSVFILG
VPYRGSLLILFFISSLFLLSTLGMGLLISTITRNQFNAAQVALNAAFLPS
IMLSGFIFQIDSMPAVIRAVTYIIPARYFVSTLQSLFLAGNIPVVLVVNV
LFLIASAVMFIGLTWLKTKRRLD

### SEQ ID NO:212

>sll0041_Nin_PLS_YbhR
MQAPTQSGGLSLRNKAVLIALLIGLIPAGVIGGLNLSSVDRLPVPQTEQQ
VKDSTTKQIRDQILIGLLVTAVGAAFVAYWMVGENTKAQTALALKAKFHR
LWTLIRKELQSLLREPQTRAILILPVLIQVILFPFAATLEVTNATIAIYD
EDNGEHSVELTQRFARASAFTHVLLLKSPQEIRPTIDTQKALLLVRFPA
DFSRKLDTFQTAPLQLILDGRNSNSAQIAANYLQQIVKNYQQELLEGKP
KPNNSELVVRNWYNPNLDYKWFVVPSLIAMITTIGVMIVTSLSVARERE
QGTLDQLLVSPLTTWQIFIGKAVPALIVATFQATIVLAIGIWAYQIPFAG
SLALFYFTMVIYGLSLVGFGLLISSLCSTQQQAFIGVFVFMMPAILLSGY
VSPVENMPVWLQNLTWINPIRHFTDITKQIYLKDASLDIVWNSLWPLL
VITATTGSAAYAMFRRKVM

### SEQ ID NO:213

>slr1044_Nin_PLS_YbhS
MFLGWFTNASLFRKQIYMAIASGVFSGFAVLVLGSIVGLGGTPKDVPAP
SGETTTEAPAEGAPAEGQAPSQTPEEEPGKPSLLNLAFLTAIATAIGV
FLINRLLMQQIKSIIDDLQSNPILSWRRVRALCVKETRQIVRDPSSWLIA
VVIPLLLLFIFGYGINLDSSKLRVGILLEQRSEAALDFTHTMTGSPYIDA
TISDNRQELIAKMQAGKIRGLVVIPVDFAEQMERANATAPIQVITDGSEP
NTANFVQGYVEGIWQIWQMQRAEDNGQTFEPLIDVQTRYWFNPAAISQH
FIIPGAVTIIMTVIGAILTSLVVAREWERGTMEALLSTEITRTELLLCKL
IPYYFLGMLAMLLCMLVSVFILGVPYRGSLLILFFISSLFLLSTLGMGL
LISTITRNQFNAAQVALNAAFLPSIMLSGFIFQIDSMPAVIRAVTYIIPA
RYFVSTLQSLFLAGNIPVVLVVNVLFLIASAVMFIGLTWLKTKRRLD

### SEQ ID NO:214

>slr1044_Nin_PLS_YbhR
MFLGWFTNASLFRKQIYMAIASGVFSGFAVLVLGSIVGLGGTPKDVPA
PSGETTTEAPAEGAPAEGQAPSQTPEEEPGKPSLLNLAFLTAIATAI
GVFLINRLLMQQIKSIIDDLQFHRLWTLIRKELQSLLREPQTRAILILPV
LIQVILFPFAATLEVTNATIAIYDEDNGEHSVELTQRFARASAFTHVLLL
KSPQEIRPTIDTQKALLLVRFPADFSRKLDTFQTAPLQLILDGRNSNSA
QIAANYLQQIVKNYQQELLEGKPKPNNSELVVRNWYNPNLDYKWFVV
PSLIAMITTIGVMIVTSLSVAREREQGTLDQLLVSPLTTWQIFIGKAVPA
LIVATFQATIVLAIGIWAYQIPFAGSLALFYFTMVIYGLSLVGFGLLISS
LCSTQQQAFIGVFVFMMPAILLSGYVSPVENMPVWLQNLTWINPIRHF
TDITKQIYLKDASLDIVWNSLWPLLVITATTGSAAYAMFRRKVM

Additional embodiments are described in the claims.

What is claimed is:

1.-81. (canceled)

82. A method for producing hydrocarbons, comprising:
(i) culturing an engineered photosynthetic microorganism in a culture medium, wherein said engineered photosynthetic microorganism comprises (i) genes encoding a recombinant acyl-ACP reductase enzyme (AAR) and a recombinant alkanal deformylative monooxygenase (ADM) enzyme, and (ii) one or more recombinant genes encoding one or more protein components of a recombinant hydrocarbon ATP-binding cassette (ABC) efflux pump system; and
(ii) exposing said engineered photosynthetic microorganism to light and an inorganic carbon source, wherein said exposure results in the conversion of said inorganic carbon source by said engineered photosynthetic microorganism into n-alkanes, wherein said n-alkanes are secreted into said culture medium in an amount greater than that secreted by an otherwise identical photosynthetic microorganism, cultured under identical conditions, but lacking said recombinant genes.

83. The engineered photosynthetic microorganism of claim 82, wherein said one or more protein components are selected from the group consisting of YbhG, YbhF, YbhS, YbhR, YhiI, RbbA, YhhJ, TolC and TolC homolog protein components.

84. The method of claim 82, wherein said n-alkanes comprise predominantly n-heptadecane, n-pentadecane or a combination thereof.

85. The method of claim 82, further comprising isolating at least one of said n-alkanes, an n-alkene or an n-alkanol from said culture medium.

86. The method of claim 82, wherein at least one of said recombinant genes is encoded on a plasmid.

87. The method of claim 82, wherein at least one of said recombinant genes is incorporated into the genome of said engineered photosynthetic microorganism.

88. The method of claim 82, wherein at least one of said recombinant genes is present in multiple copies in said engineered photosynthetic microorganism.

89. The method of claim 82 wherein at least two of said recombinant genes are part of an operon, and wherein the expression of said genes is controlled by a single promoter.

90. The method of claim 82, wherein at least 95% of said n-alkanes are n-pentadecane and n-heptadecane.

91. The method of claim 82, wherein the expression of at least one of said recombinant genes is controlled by one or more inducible promoters.

92. The method of claim 91, wherein at least one promoter is a urea-repressible, nitrate-inducible promoter.

93. The method of claim 91, wherein said promoter is a nirA-type promoter.

94. The method of claim 93, wherein said nirA-type promoter is P(nir07) or P(nir09).

95. The engineered photosynthetic microorganism of claim 82, wherein said one or more protein components are selected from the group consisting of YbhG, YbhF, YbhS, YbhR, YhiI, RbbA, YhhJ and TolC protein components, wherein the leader sequences of said YbhG, YbhS, YbhR, YhiI, TolC and TolC homolog protein components are non-native leader sequences.

96. The engineered photosynthetic microorganism of claim 82, wherein said one or more protein components are selected from the group consisting of YbhG, YbhF, YbhS, YbhR, YhiI, RbbA, YhhJ, TolC and TolC homolog protein components, wherein the leader sequences of said YbhG, YhiI, TolC and TolC homolog protein components are leader sequences native to said photosynthetic microorganism.

97. The engineered photosynthetic microorganism of any of claims 83, 95 or 96, wherein said TolC homolog protein is selected from the group consisting of SYNPCC7002_A0585 or Synpcc7942_{—}1761.
